(19) United States
(12) Patent Application Publication
(10) Pub. No.: US 2020/0017881 A1
(43) Pub. Date: Jan. 16, 2020

(54) Programmable Oncolytic Virus Vaccine System and Method

(71) Applicants: Beijing Syngentech Co., Ltd., Beijing (CN); Tsinghua University, Beijing (CN)

(72) Inventors: Zhen Xie, Beijiing (CN); Huiya Huang, Beijing (CN); Yiqi Liu, Beijing (CN); Weixi Liao, Beijing (CN)

(73) Assignees: Beijing Syngentech Co., Ltd., Beijing (CN); Tsinghua University, Beijing (CN)

(21) Appl. No.: 16/470,520

(22) PCT Filed: Aug. 4, 2017
(86) PCT No.: PCT/CN2017/096043

§ 371 Date: Sep. 21, 2019

(30) Foreign Application Priority Data
Mar. 24, 2017 (CN) 201710184736.2

(51) Int. Cl. C12N 15/86; A61K 35/761; A61K 39/00

(52) U.S. Cl. CPC C12N 15/86; A61K 35/761; A61K 2039/5256; C12N 2710/10043; C12N 2710/10034; A61K 39/0011

(57) ABSTRACT

An expression system, comprising: a first nucleic acid molecule having a cell-specific promoter; a second nucleic acid molecule which encodes a transcriptional activator; a third nucleic acid molecule having a first recognition sequence of the transcriptional activator; a fourth nucleic acid molecule having a first promoter and a first regulatory element; a fifth nucleic acid molecule which encodes a first regulatory protein; a sixth nucleic acid molecule having a second recognition sequence of the transcriptional activator; a seventh nucleic acid molecule having a second promoter and a second regulatory element; an eighth nucleic acid molecule which encodes a second regulatory protein; as well as a ninth nucleic acid molecule which is configured to conditionally inhibit the expression of the first regulatory protein, and a tenth nucleic acid molecule which is configured to conditionally inhibit the expression of the second regulatory protein; the first regulatory element is adapted to inhibit the function of the first promoter by means of binding to the second regulatory protein, and the second regulatory element is adapted to inhibit the function of the second promoter by means of binding to the first regulatory protein.

### PRIORITY INFORMATION

This application claims priority to and the benefit of the patent application Serial No. 201710184736.2 filed with the National Intellectual Property Administration of PRC on Mar. 24, 2017, the entire content of which is incorporated herein by reference.

### FIELD

The present disclosure relates to the field of biomedicine. In particular, the present disclosure relates to an expression system and use thereof, and more particularly, to an expression system, a recombinant virus, a recombinant cell, and use of the gene expression system, the recombinant virus and the recombinant cell in the preparation of a medicament. Further, the present disclosure also relates to a method of expressing a protein of interest by using the expression system.

### BACKGROUND

Oncolytic viruses refer to a type of viruses that have the ability of replication and packaging so as to achieve tumor killing. At present, most studies have showed that some naturally occurring attenuated viruses after engineering modification can be specifically expressed and packaged in tumor cells, thus realizing oncolytic effect. There are two main principles for recognizing tumor cells via oncolytic viruses. Firstly, the oncolytic viruses selectively infect tumor cells due to inactivation or defect of tumor suppressor genes in target cells. Secondly, tumor-specific promoters are selected to regulate the expression of key genes of oncolytic viruses such that the oncolytic viruses replicate in large amounts and express toxic proteins in tumor cells to destroy the tumor cells, and/or simultaneously secrete cytokines to stimulate the autoimmune system, thus attacking tumor cells. Corresponding oncolytic virus cannot replicate in normal cells of an organism, thereby having no killing ability, thus the oncolytic virus has a higher anti-tumor effect and a lower side effect. In recent decades, oncolytic viral treatment has gained extensive attentions, and relevant researches have made great progress. Currently, adenovirus, Herpes Simplex Virus-1 (HSV-1), Newcastle disease virus and the like have been engineered to oncolytic viruses. In 2006, oncolytic adenovirus products (Gendicine and Oncorine) have been used in clinical treatment in China, mainly for the treatment of head and neck cancer, sinus cancer and the like. Gendicine and Oncorine have a similar mechanism, that is, deleting the E1B-55 kD region of human type-5 adenovirus, such that the human type-5 adenovirus can reproduce in p53 gene-mutated cancer cells and kill tumor cells, thereby generating therapeutic oncolytic effects. However, clinical data shows that the therapeutic effect of these two oncolytic adenoviruses based on the single p53 gene mutation are not ideal. Further, JX-594 of the US biotherapeutic company Jennerex is an engineered vaccinia virus. It is shown that primary liver cancer patients have an extended median life-span reaching 14.1 months after high-dose injection of the engineered vaccinia virus, compared to 6.7 months of life-span after low-dose injection in the phase II clinical trial completed in 2013. Furthermore, the genetically engineered herpes simplex virus OncoVEX GM-CSF developed by biotechnology company BioVex, which is the first oncolytic virus product on market in the US and Europe approved by the FDA in October 2015, can selectively kill tumor cells while expressing and secreting GM-CSF to initiate the generation of systematic immune response in body, thus killing residual tumor cells and their metastatic sites. The clinical results of metastatic melanoma in phase II trial published by BioVex in 2009 show that 26% of 50 patients exhibit improvement after treatment and 8 patients are totally recovered. Amgen, acquiring the company BioVex at $1 billion in 2011 for promoting the research of phase III clinical trial, published the efficacy data of OncoVEX in March 2013, which demonstrates the drug OncoVEX is capable of decreasing the tumor size of advanced patients successfully, and exhibits stronger efficacy than other similar drugs in the phase III clinical trial involving more than 400 patients.

The oncolytic virus indeed exhibits efficacy on treatment of tumors based on the above research results, however, some deficiencies still exist.

Currently, there are 52 known human adenoviruses, named as Adl˜Ad52, where Ad2 is the most common. The transcriptional pattern of adenovirus has a very distinct feature, where adenoviral genome has at least five known transcription units, including the E1 region involved in cell transformation and located in the left side of the adenoviral genome, which can be subdivided into E1A and E1B; the E2 region, encoding DNA binding protein and involved in viral replication; the E3 region, encoding a glycoprotein that appears on the surface of host cells; the E4 region, located at the right end of the Ad2 genome and regulated by the DNA binding protein encoded by the E2 region; and the fifth transcription unit, synthesizing the Ad2 protein IV in the metaphase of viral infection. After the adenoviral genome enters cell nucleus, cellular transcription factor firstly binds to the enhancer upstream of the E1A region, expressing the E1A protein, which regulates cellular metabolism and allows viral DNA to be replicated in the cell easily, as well as activates promoters of other early genes (E1B, E2A, E2B, E3 and E4), where E2B drives the expression of three additional transcriptional units of early genes involving in viral replication (i.e. precursor terminal protein (pTP), single-stranded DNA binding protein (ssDBP) and DNA polymerase (DNA pol)), these three gene expression products tightly binding to a complex, interacting with at least three cellular proteins to initiate replication of the viral genome. The following engineering strategies can be made according to the analysis of adenoviral genome and transcriptional pattern: regulating E1A expression to regulate the replication and packaging of adenovirus in target cells; removing some non-essential genes relating to viral packaging to reduce toxicity of virus on non-target cells while increasing the packaging capacity of virus (such as, E3, E4 and the like); replacing the coat protein of adenovirus, thus changing the targeting of adenovirus to specific cells and tissues.

However, the existing research results of oncolytic adenovirus show the presence of deficiencies of the current regulation system such as monotony, poor specificity, low safety and the like as well as the cumbersome method for engineering adenovirus. Therefore, how to design a more rigorous and safer regulation system and how to realize the assembly of oncolytic adenovirus in a quick and effective way is needed to be solved urgently in the aspect of design and construction of oncolytic adenovirus.

### SUMMARY

The present disclosure aims at solving several of the aforementioned technical problems to at least some extent.

Based on the research achievements in the inventors' laboratory, the present inventors have designed and constructed a gene circuit that is suitable for adenovirus regulation and responsive to different microenvironments. In this gene circuit, the present inventors utilize multiple levels of biomarkers. Specifically, first, a specific promoter is used as a master switch for regulating the gene circuit, that is, employing the specific promoter to regulate the expression of a master activator, thus further regulating the expression of the adenovirus E1A gene; second, microRNA target sequence is used to respond to the expression characteristics of microRNA under different microenvironments, which acts as a secondary switch for regulating the gene circuit; third, a close-loop gene circuit is employed to construct a mutually repressive switch, such a mutually repressive close-loop switch can repression respond to changes of microenvironment more efficiently while effectively avoiding leakage; fourth, the E1B gene is removed, thus the engineered adenovirus has an improved ability of distinguishing P53 deficient tumor cells from normal cells due to the absence of E1B gene; fifth, the E3 gene is removed, thus reducing the toxicity of oncolytic adenovirus on normal cells while increasing the packaging capacity of adenoviral vectors.

In general, type-2 and type-5 human adenoviruses have a genome length of 36 K, thus it will be very difficult and time consuming to genetically modify the adenovirus by using traditional plasmid construction methods like cleavage, ligation and the like. In order to solve this problem, the present inventors designed a method for rapidly constructing the adenovirus constructed by the present gene circuit, comprising the following three steps. In the first step, a primary element library is constructed, where the required components are constructed into corresponding plasmids. Such a primary element library comprises three gene parts, including a repression element library A, mainly expressing the repression components in one side of the gene circuit, the E1A gene of adenovirus and effector genes co-expressed with the adenovirus E1A gene (such as an immune factor gene or a killer gene); a repression element library B, mainly expressing the repression gene in the other side of the gene circuit, configured to regulate the effective reversal of the gene circuit, thus enhancing the safety of the switch; and a specific promoter library, being the master switch which regulates the downstream gene of the gene circuit via tissue or tumor-specific promoters. In the second step, after determination of biomarkers and effectors to be expressed, corresponding plasmids selected from the primary element library are assembled into a gene circuit rapidly through the Golden Gate method. In the third step, the gene circuit is loaded into the engineered adenoviral vector (removal of the E1 gene to facilitate the artificial regulation of adenovirus; and removal of part E3 sequence to enlarge the packaging capacity).

Carrying and simultaneously expressing multiple genes is another remarkable feature of oncolytic adenovirus. In the present disclosure, one or more cellular immune-related genes like IL-2, GM-CSF, anti-PD-1 scFv, anti-PD-L1 scFv, and fusion proteins between these genes and the like are simultaneously expressed by oncolytic adenovirus through the disclosure by the present inventors. The oncolytic adenovirus will trigger systematic immune responses when attacking tumor cells, while having a risk of causing an immune overreaction due to the presence of immune-related genes. Thus, the therapeutic effect of the oncolytic viruses will be strongly influenced by the immune-related genes carried, the corresponding administration dose and the administration routes. In the present disclosure, the inventors have attempted to model the infecting process of adenovirus against target cells by means of bioinformatics method, and studied the properties of oncolytic adenovirus at killing tumor cells, so as to improve the efficacy of oncolytic adenovirus as much as possible.

In a first aspect, the present disclosure in embodiments provides an expression system. According to an embodiment of the present disclosure, the expression system comprises:

a first nucleic acid molecule, incorporating a cell-specific promoter;

a second nucleic acid molecule, operably linked to the first nucleic acid molecule and encoding a transcriptional activator;

a third nucleic acid molecule, incorporating a first recognition sequence of the transcriptional activator;

a fourth nucleic acid molecule, operably linked to the third nucleic acid molecule and incorporating a first promoter and a first regulatory element;

a fifth nucleic acid molecule, operably linked to the fourth nucleic acid molecule and encoding a first regulatory protein;

a sixth nucleic acid molecule, incorporating a second recognition sequence of the transcriptional activator;

a seventh nucleic acid molecule, operably linked to the sixth nucleic acid molecule and incorporating a second promoter and a second regulatory element;

an eighth nucleic acid molecule, operably linked to the seventh nucleic acid and encoding a second regulatory protein; and

at least one selected from the group consisting of:

a ninth nucleic acid molecule, operably linked to the fifth nucleic acid molecule and configured to conditionally inhibit expression of the first regulatory protein;

a tenth nucleic acid molecule, operably linked to the eighth nucleic acid molecule and configured to conditionally inhibit expression of the second regulatory protein,

wherein

the first regulatory element is adapted to inhibit the function of the first promoter by binding to the second regulatory protein, and

the second regulatory element is adapted to inhibit the function of the second promoter by binding to the first regulatory protein.

With the expression system according to the embodiment of the present disclosure, the gene of interest can be specifically expressed under a specific cellular environment while being regulated in a mutual repression way, with strong control, high efficiency and specificity.

In embodiments of the present disclosure, the expression system may further comprise at least one of additional technical features as follows.

In embodiments of the present disclosure, the cell-specific promoter is a tumor cell-specific promoter. The tumor cell-specific promoter is at least one selected from the group consisting of an alpha-fetoprotein-specific promoter, a Survivin promoter, a human telomerase reverse transcriptase gene promoter, a cholecystokinin A receptor gene promoter, a carcinoembryonic antigen promoter, a proto-oncogene human epidermal growth factor receptor 2 promoter, a prostaglandin endoxygenase reductase 2 promoter, a chemokine receptor-4, an E2F-1 gene promoter, a mucin promoter, a prostate specific antigen, a human tyrosinase-related protein 1, and a tyrosinase promoter. The expression system in the embodiments can be initiated in the microenvironment of specific tumor cells under the control of the tumor cell-specific promoters described as above, thereby further enhancing the specificity of gene expression and regulation.

In embodiments of the present disclosure, the transcriptional activator is at least one selected from the group consisting of Gal4VP16, Gal4VP64, Gal4esn, dCas9-VP16, dCas9-VP64, dCas9-VPR, dCas9-VTR and rtTA.

In embodiments of the present disclosure, the first recognition sequence and the second recognition sequence are each independently selected from at least one of 5×UAS, 7×tetO and a target sequence of dCas9.

In embodiments of the present disclosure, the first promoter and the second promoter are each independently selected from a miniCMV and a TATA box.

In embodiments of the present disclosure, the first regulatory protein and the second regulatory protein are each independently selected from at least one of LacI, tetR, zinc finger, KRAB, tetR-KRAB and dCas9-KRAB.

In embodiments of the present disclosure, the first regulatory element and the second regulatory element are each independently selected from at least one of tetO, LacO, zinc finger target site and a target sequence of dCas9.

In an embodiment of the present disclosure, the first regulatory protein is Lad, and the second regulatory element comprises a plurality of repeated LacO sequences, wherein at least one of the pluralities of repeated LacO sequences is set downstream of the second promoter. The Lad after expressed can specifically binds to the LacO sequence, thereby inhibiting the function of the second promoter. The LacI/LacO repression system according to the embodiment is capable of effectively inhibiting the expression of downstream genes of the second promoter as demonstrated by experiments.

In an embodiment of the present disclosure, the second regulatory protein is tetR-KRAB, and the first regulatory element comprises a plurality of repeated tetO sequences, wherein at least one of the pluralities of repeated tetO sequences is set downstream of the first promoter. The tetR-KRAB/tetO repression system according to the embodiment is capable of effectively inhibiting the expression of downstream genes of the first promoter.

In an embodiment of the present disclosure, at least one of the fifth nucleic acid molecule and the ninth nucleic acid molecule further comprises a sequence encoding a protein of interest. With the expression system according to the embodiment of the present disclosure, the protein of interest can be specifically expressed in a specific cellular microenvironment, while being regulated in a mutual repression way. According to a specific embodiment of the present disclosure, the expression system exhibits a significant increased specificity and regulation on the expression of the protein of interest.

In an embodiment of the present disclosure, the fifth nucleic acid molecule comprises a sequence encoding the protein of interest, and the protein of interest comprises at least one selected from a viral replication and packaging protein and an immune effector. Optionally, the viral replication and packaging protein and the immune effector may be present in the form of a fusion protein. The viral replication and packaging protein can effectively ensure the survival and replication of the expression system vector in a host. The expression of the immune effector can effectively activate the immune system in body, thereby promoting the immune killing to specific cells such as tumor cells.

In an embodiment of the present disclosure, the virus replication and packaging protein comprises at least one selected from the group consisting of an adenovirus E1 gene, an adenovirus E1A gene, an adenovirus E1B gene, an adenovirus E2 gene, and an adenovirus E4 gene.

In an embodiment of the present disclosure, the immune effector comprises at least one sequence selected from the group consisting of an inhibitory sequence that antagonizes PD-1 gene, an inhibitory sequence that antagonizes PD-L1 gene, an inhibitory sequence that antagonizes CTLA4 gene, an inhibitory sequence that antagonizes Tim-3 gene, GM-CSF, IL-2, IL-12 and IL-15. Optionally, the immune effector as described above may be present in the form of a fusion protein.

In an embodiment of the present disclosure, the protein of interest and the first regulatory protein are expressed in the form of a fusion protein, and the protein of interest and the first regulatory protein are linked by a cleavable linker peptide. The protein of interest and the first regulatory protein are regulated and expressed by a same promoter, and are cleaved at the linker peptide after expressed, thus the protein of interest and the first regulatory protein are separated, and function independently of each other.

In an embodiment of the present disclosure, the ninth nucleic acid molecule and the tenth nucleic acid molecule independently inhibit expression of the first regulatory protein or the second regulatory protein via RNA interference, wherein microRNA is a specific microRNA expressed in different cellular microenvironments, and the ninth or tenth nucleic acid molecule is a specific target sequence of the microRNA. The microRNA expressed in a specific microenvironment can specifically act on the target sequence via RNA interference, thus the expression of the first regulatory protein or the second regulatory protein can be specifically regulated. In the embodiment of the present disclosure, the ninth nucleic acid molecule comprises a nucleic acid sequence specifically recognized by a first microRNA, the first microRNA being a normal cell-specific microRNA; and the tenth nucleic acid molecule comprises a nucleic acid sequence specifically recognized by a second microRNA, the second microRNA being an abnormal cell-specific microRNA. Further, the first regulatory protein is expressed in abnormal cells and is not expressed or low-expressed in normal cells, while the second regulatory protein is expressed in normal cells and is not expressed or low-expressed in abnormal cells.

In a specific embodiment of the present disclosure, the first microRNA comprises at least one selected from the group consisting of miR199a, miR95, miR125, miR25b, Let-7, miR143, miR145 and miR200C. The microRNA as described above is expressed in normal liver cells.

In a specific embodiment of the present disclosure, the second microRNA comprises at least one selected from the group consisting of miR21, miR223, miR224, miR221, miR18, miR214, miR146a and miR1792. The second microRNA as described above is a microRNA specifically expressed in hepatoma cells (such as HepG2, Huh7, and PLC). Furthermore, the first regulatory protein is expressed in hepatoma cells and is not expressed or low-expressed in normal cells, while the second regulatory protein is expressed in normal cells and is not expressed or low-expressed in hepatoma cells.

In an embodiment of the present disclosure, the first nucleic acid molecule and the second nucleic acid molecule are loaded in a first expression vector; the third nucleic acid molecule, the fourth nucleic acid molecule, the fifth nucleic acid molecule and optionally the ninth nucleic acid molecule are loaded in a second expression vector; and the sixth nucleic acid molecule, the seventh nucleic acid molecule, the eighth nucleic acid molecule and optionally the tenth nucleic acid molecules are loaded in a third expression vector. The first, second and third expression vectors serve as a loading vector for the expression system so as to regulate the specific expression of the gene of interest in a suitable microenvironment, such as a cell.

The selection of the expression vector is not particularly limiting as long as the expression system can exhibit its function in a suitable microenvironment. According to a specific embodiment of the present disclosure, the first expression vector, the second expression vector and the third expression vector are each independently selected from at least one of

plasmids, viruses, stable cell lines, and other carriers such as nanomaterials, liposomes, molecularly coupled vectors, naked DNAs, chromosomal vectors and polymers.

In an embodiment of the present disclosure, the virus comprises at least one selected from the group consisting of an adenovirus, a vaccinia virus, a herpes virus and a retrovirus.

In an embodiment of the present disclosure, the first expression vector, the second expression vector and the third expression vector are loaded in one same vector. It should be noted that the connection order of the first expression vector, the second expression vector, and the third expression vector is not particularly limited as long as it does not affect the realization of the biological function of the expression system. According to a specific embodiment of the present disclosure, the problem of extremely low co-transfection efficiency of multiple large fragment vectors can be effectively solved by loading on one same expression vector.

In an embodiment of the present disclosure, the one same vector is an adenoviral vector. As a gene therapy vector, adenovirus has advantages of a wide range of hosts, low pathogenicity to humans, infection and gene expression in proliferating and non-proliferating cells, high titer, homology with human genes, no mutagenicity after insertion, as well as amplifying in a suspension and simultaneously expressing multiple genes.

In a specific embodiment of the present disclosure, the first expression vector comprises: BsaI, AFP III, Gal4VP16 and BsaI from the 5′ end to the 3′ end (BsaI-AFP III-Gal4VP16-BsaI).

In an embodiment of the present disclosure, the first expression vector carries a nucleic acid having the nucleotide sequence shown in SEQ ID NO: 1.

(SEQ ID NO: 1)
GGTCTCTGCTCCAGATTGAATTATTTGCCTGTCATACAGCTAATAATTGACCATAAGACA
ATTAGATTTAAATTAGTTTTGAATCTTTCTAATACCAAAGTTCAGTTTACTGTTCCATGTT
GCTTCTGAGTGGCTTCACAGACTTATGAAAAAGTAAACGGAATCAGAATTACATCAAT
GCAAAAGCATTGCTGTGAACTCTGTACTTAGGACTAAACTTTGAGCAATAACACATATA
GATTGAGGATTGTTTGCTGTTAGTATACAAACTCTGGTTCAAAGCTCC
TCTTTATTGCTTGTCTTGGAAAATTTGCTGTTCTTCATGGTTTCTCTTTTCACTGCTATCT
ATTTTTCTCAACCACTCACATGGCTACAAAAGCTTCCTGATTAATAATTACACTAAGTCA
ATAGGCATAGAGCCAGGACTGTTTGGGTAAACTGGTCACTTTATCTTAAACTAAATATAT
CCAAAACTGAACATGTACTTAGTTACTAAGTCTTTGACTTTATCTCATTCATACCACTCA
GCTTTATCCAGGCCACTTATTTGACAGTATTATTGCGAAAACTTCCTATCTAGAAGTTTG
AGGAGAATATTTGTTATATTTGCAAAATAAAATAAGTTTGCAAGTTTTTTTTTTCTGCCC
CAAAGAGCTCTGTGTCCTTGAACATAAAATACAAATAACCGCTATGCTGTTAATTATTG
ACAAATGTCCCATTTTCAACCTAAGGAAATACCATAAAGTAACAGATATACCAACAAAA
GGTTACTAGTTAACAGGCATTGCCTGAAAAGAGTATAAAAGAATTTCAGCATGATTTTC
CATATTGTGCTTCCACCACTGCCAATAACAAAATAACTAGCAAGGATCCGGCCACCATG
GCCCCCCCGACCGATGTCAGCCTGGGGGACGAGCTCCACTTAGACGGCGAGGACGTG
GCGATGGCGCATGCCGACGCGCTAGACGATTTCGATCTGGACATGTTGGGGGACGGGG
ATTCCCCGGGTCCGGGATTTACCCCCCACGACTCCGCCCCCTACGGCGCTCTGGATATG
GCCGACTTCGAGTTTGAGCAGATGTTTACCGATGCCCTTGGAATTGACGAGTACGGTG
GGACGCGTATGAAGCTACTGTCTTCTATCGAACAAGCATGCGATATTTGCCGACTTAAA
AAGCTCAAGTGCTCCAAAGAAAAACCGAAGTGCGCCAAGTGTCTGAAGAACAACTG
GGAGTGTCGCTACTCTCCCAAAACCAAAAGGTCTCCGCTGACTAGGGCACATCTGACA
GAAGTGGAATCAAGGCTAGAAAGACTGGAACAGCTATTTCTACTGATTTTTCCTCGAG
AAGACCTTGACATGATTTTGAAAATGGATTCTTTACAGGATATAAAAGCATTGTTAACA
GGATTATTTGTACAAGATAATGTGAATAAAGATGCCGTCACAGATAGATTGGCTTCAGT
GGAGACTGATATGCCTCTAACATTGAGACAGCATAGAATAAGTGCGACATCATCATCGG
AAGAGAGTAGTAACAAAGGTCAAAGACAGTTGACTGTATAATTCACTCCTCAGGTGCA
GGCTGCCTATCAGAAGGTGGTGGCTGGTGTGGCCAATGCCCTGGCTCACAAATACCAC
TGAGATCTTTTTCCCTCTGCCAAAAATTATGGGGACATCATGAAGCCCCTTGAGCATCT
GACTTCTGGCTAATAAAGGAAATTTATTTTCATTGCAATAGTGTGTTGGAATTTTTTGTG
TCTCTCACTCGGAAGGACATATGGGAGGGCAAATCATTTAAAACATCAGAATGAGTATT
TGGTTTAGAGTTTGGCAACATATGCCCATATGCTGGCTGCCATGAACAAAGGTTGGCTA
TAAAGAGGTCATCAGTATATGAAACAGCCCCCTGCTGTCCATTCCTTATTCCATAGAAA
AGCCTTGACTTGAGGTTAGATTTTTTTTATATTTTGTTTTGTGTTATTTTTTTCTTTAACAT
CCCTAAAATTTTCCTTACATGTTTTACTAGCCAGATTTTTCCTCCTCTCCTGACTACTCC
CAGTCATAGCTGTCCCTCTT
CTCTTATGGAGATCGGAGAAAGAGGTAATTTAATTAAGTCGATGAGACC

In a specific embodiment of the present disclosure, the second expression vector comprises: BsaI, 5×UAS, tetO, miniCMV, tetO, E1A, 2A, an immune effect factor (Effector), Lad, microRNA199a specific recognition sequence (target site) and BsaI from the 5′ end to the 3′ end (BsaI-5×UAS-tetO-miniCMV-tetO-E1A-2A-Effector-LacI-miR199a target site-BsaI). In a specific embodiment of the present disclosure, the effector specifically comprises IL-2, hGM-CSF, mGM-CSF, anti-PD-1 scFv, anti-PD-L1 scFv, IL-2-anti-PD-1 scfv fusion protein, hGM-CSF-anti-PD-lscfv fusion protein, mGM-CSF-anti-PD-lscfv fusion protein, IL-2-anti-PD-L1scfv fusion protein, hGM-CSF—anti-PD-L1scfv fusion protein, mGM-CSF-anti-PD-L1scfv fusion protein.

In an embodiment of the present disclosure, the second expression vector carries a nucleic acid having the nucleotide sequence selected from the group consisting of SEQ ID NOS: 2-7.

(SEQ ID NO: 2)
GGTCTCTCTATTTAATTAAGTAACTATAACGGTCGCTCCGAATTTCTCGAGTTAATTAAG
ATTACGCCAAGCTACGGGCGGAGTACTGTCCTCCGAGCGGAGTACTGTCCTCCGAGCG
GAGTACTGTCCTCCGAGCGGAGTACTGTCCTCCGAGCGGAGTTCTGTCCTCCGAGCGG
AGACTCTAGACTCCCTATCAGTGATAGAGATAGGCGTGTACGGTGGGAGGCCTATATAA
GCAGAGCTCGTTTAGTGAACCGTCAGATCGCTCCCTATCAGTGATAGAGAGAATTCGA
CCGCCACCATGTCGGAATTCATGAGACATATTATCTGCCACGGAGGTGTTATTACCGAA
GAAATGGCCGCCAGTCTTTTGGACCAGCTGATCGAAGAGGTACTGGCTGATAATCTTC
CACCTCCTAGCCATTTTGAACCACCTACCCTTCACGAACTGTATGATTTAGACGTGACG
GCCCCCGAAGATCCCAACGAGGAGGCGGTTTCGCAGATTTTTCCCGACTCTGTAATGT
TGGCGGTGCAGGAAGGGATTGACTTACTCACTTTTCCGCCGGCGCCCGGTTCTCCGGA
GCCGCCTCACCTTTCCCGGCAGCCCGAGCAGCCGGAGCAGAGAGCCTTGGGTCCGGT
TTCTATGCCAAACCTTGTACCGGAGGTGATCGATCTTACCTGCCACGAGGCTGGCTTTC
CACCCAGTGACGACGAGGATGAAGAGGGTGAGGAGTTTGTGTTAGATTATGTGGAGC
ACCCCGGGCACGGTTGCAGGTCTTGTCATTATCACCGGAGGAATACGGGGGACCCAGA
TATTATGTGTTCGCTTTGCTATATGAGGACCTGTGGCATGTTTGTCTACAGTAAGTGAAA
ATTATGGGCAGTGGGTGATAGAGTGGTGGGTTTGGTGTGGTAATTTTTTTTTTAATTTTT
ACAGTTTTGTGGTTTAAAGAATTTTGTATTGTGATTTTTTTAAAAGGTCCTGTGTCTGAA
CCTGAGCCTGAGCCCGAGCCAGAACCGGAGCCTGCAAGACCTACCCGCCGTCCTAAA
ATGGCGCCTGCTATCCTGAGACGCCCGACATCACCTGTGTCTAGAGAATGCAATAGTAG
TACGGATAGCTGTGACTCCGGTCCTTCTAACACACCTCCTGAGATACACCCGGTGGTCC
CGCTGTGCCCCATTAAACCAGTTGCCGTGAGAG
TTGGTGGGCGTCGCCAGGCTGTGGAATGTATCGAGGACTTGCTTAACGAGCCTGGGCA
ACCTTTGGACTTGAGCTGTAAACGCCCCAGGCCACTCGAGGGTACCGGGTCCGGAGCT
ACAAATTTTTCCCTCCTCAAACAGGCTGGAGATGTCGAAGAAAATCCTGGGCCTACCG
GTCCCATGGTGAGCAAGGGCGAGGAGCTGTTCACCGGGGTGGTGCCCATCCTGGTCG
AGCTGGACGGCGACGTAAACGGCCACAAGTTCAGCGTGAGGGGCGAGGGCGAGGGC
GATGCCACCAACGGCAAGCTGACCCTGAAGTTCATCTGCACCACCGGCAAGCTGCCC
GTGCCCTGGCCCACCCTCGTGACCACCCTGAGCCACGGCGTGCAGTGCTTCGCCCGCT
ACCCCGACCACATGAAGCAGCACGACTTCTTCAAGTCCGCCATGCCCGAAGGCTACGT
CCAGGAGCGCACCATCTTCTTCAAGGACGACGGCACCTACAAGACCCGCGCCGAGGT
GAAGTTCGAGGGCGACACCCTAGTGAACCGCATCGAGCTGAAGGGCGTCGACTTCAA
GGAGGACGGCAACATCCTGGGGCACAAGCTGGAGTACAACTTCAACAGCCACAACAT
CTATATCATGGCCGTCAAGCAGAAGAACGGCATCAAGGTGAACTTCAAGATCCGCCAC
AACGTGGAGGACGGCAGCGTGCAGCTCGCCGACCACTACCAGCAGAACACCCCCATC
GGCGACGGCCCCGTGCTGCTGCCCGACAGCCACTACCTGAGCACCCAGTCCGTGCTG
AGCAAAGACCCCAACGAGAAGCGCGATCACATGGTCCTGCTGGAGTTCCGCACCGCC
GCCGGGATCACTCTCGGCATGGACGAGCTGTACAAGAGATCTGAGGGCCGCGGCAGC
CTGCTGACCTGCGGCGACGTGGAGGAAAACCCCGGCCCCGGATCCATGAAACCAGTA
ACGTTATACGATGTCGCAGAGTATGCCGGTGTCTCTTATCAGACCGTTTCCCGCGTGGT
GAACCAGGCCAGCCACGTTTCTGCGAAAACGCGGGAAAAAGTGGAAGCGGCGATGG
CGGAGCTGAATTACATTCCCAACCGCGTGGCACAACAACTGGCGGGCAAACAGTCGT
TGCTGATTGGCGTTGCCACCTCCAGTCTGGCCCTGCACGCGCCGTCGCAAATTGTCGC
GGCGATTAAATCTCGCGCCGATCAACTGGGTGCCAGCGTGGTGGTGTCGATGGTAGAA
CGAAGCGGCGTCGAAGCCTGTAAAGCGGCGGTGCACAATCTTCTCGCGCAACGCGTC
AGTGGGCTGATCATTAACTATCCGCTGGATGACCAGGATGCCATTGCTGTGGAAGCTGC
CTGCACTAATGTTCCGGCGTTATTTCTTGATGTCTCTGACCAGACACCCATCAACAGTA
TTATTTTCTCCCATGAAGACGGTACGCGACTGGGCGTGGAGCATCTGGTCGCATTGGGT
CACCAGCAAATCGCGCTGTTAGCGGGCCCATTAAGTTCTGTCTCGGCGCGTCTGCGTC
TGGCTGGCTGGCATAAATATCTCACTCGCAATCAAATTCAGCCGATAGCGGAACGGGA
AGGCGACTGGAGTGCCATGTCCGGTTTTCAACAAACCATGCAAATGCTGAATGAGGGC
ATCGTTCCCACTGCGATGCTGGTTGCCAACGATCAGATGGCGCTGGGCGCAATGCGCG
CCATTACCGAGTCCGGGCTGCGCGTTGGTGCGGATATCTCGGTAGTGGGATACGACGAT
ACCGAAGACAGCTCATGTTATATCCCGCCGTTAACCACCATCA
AACAGGATTTTCGCCTGCTGGGGCAAACCAGCGTGGACCGCTTGCTGCAACTCTCTCA
GGGCCAGGCGGTGAAGGGCAATCAGCTGTTGCCCGTCTCACTGGTGAAAAGAAAAAC
CACCCTGGCGCCCAATACGCAAACCGCCTCTCCCCGCGCGTTGGCCGATTCATTAATGC
AGCTGGCACGACAGGTTTCCCGACTGGAAAGCGGGCAGAGCAGCCTGAGGCCTCCTA
AGAAGAAGAGGAAGGTTGCGGCCGCTAACCAATGTGCAGACTACTGTTAACCAATGT
GCAGACTACTGTGAATTCTAACCAATGTGCAGACTACTGTTAACCAATGTGCAGACTAC
TGTAAGCTTCGAATTGAGTAGTGCTTTCTACTTTATGAGTAGTGCTTTCTACTTTATGAG
TAGTGCTTTCTACTTTATGAGTAGTGCTTTCTACTTTATGGTCGACAATACTAGTTAAGA
AATGAGACC.
(SEQ ID NO: 3)
GGTCTCTCTATTTAATTAAGTAACTATAACGGTCGCTCCGAATTTCTCGAGTTAATTA
AGATTACGCCAAGCTACGGGCGGAGTACTGTCCTCCGAGCGGAGTACTGTCCTCCGAG
CGGAGTACTGTCCTCCGAGCGGAGTACTGTCCTCCGAGCGGAGTTCTGTCCTCCGAGC
GGAGACTCTAGACTCCCTATCAGTGATAGAGATAGGCGTGTACGGTGGGAGGCCTATAT
AAGCAGAGCTCGTTTAGTGAACCGTCAGATCGCTCCCTATCAGTGATAGAGAGAATTC
GACCGCCACCATGTCGGAATTCATGAGACATATTATCTGCCACGGAGGTGTTATTACCG
AAGAAATGGCCGCCAGTCTTTTGGACCAGCTGATCGAAGAGGTACTGGCTGATAATCT
TCCACCTCCTAGCCATTTTGAACCACCTACCCTTCACGAACTGTATGATTTAGACGTGA
CGGCCCCCGAAGATCCCAACGAGGAGGCGGTTTCGCAGATTTTTCCCGACTCTGTAAT
GTTGGCGGTGCAGGAAGGGATTGACTTACTCACTTTTCCGCCGGCGCCCGGTTCTCCG
GAGCCGCCTCACCTTTCCCGGCAGCCCGAGCAGCCGGAGCAGAGAGCCTTGGGTCCG
GTTTCTATGCCAAACCTTGTACCGGAGGTGATCGATCTTACCTGCCACGAGGCTGGCTT
TCCACCCAGTGACGACGAGGATGAAGAGGGTGAGGAGTTTGTGTTAGATTATGTGGAG
CACCCCGGGCACGGTTGCAGGTCTTGTCATTATCACCGGAGGAATACGGGGGACCCAG
ATATTATGTGTTCGCTTTGCTATATGAGGACCTGTGGCATGTTTGTCTACAGTAAGTGAA
AATTATGGGCAGTGGGTGATAGAGTGGTGGGTTTGGTGTGGTAATTTTTTTTTTAATTTT
TACAGTTTTGTGGTTTAAAGAATTTTGTATTGTGATTTTTTTAAAAGGTCCTGTGTCTGA
ACCTGAGCCTGAGCCCGAGCCAGAACCGGAGCCTGCAAGACCTACCCGCCGTCCTAA
AATGGCGCCTGCTATCCTGAGACGCCCGACATCACCTGTGTCTAGAGAATGCAATAGTA
GTACGGATAGCTGTGACTCCGGTCCTTCTAACACACCTCCTGAGATACACCCGGTGGT
CCCGCTGTGCCCCATTAAACCAGTTGCCGTGAGAGTTGGTGGGCGTCGCCAGGCTGTG
GAATGTATCGAGGACTTGCTTAACGAGCCTGGGCAACCTTTGGACTTGAGCTGTAAAC
GCCCCAGGCCACTCGAGGAGGGCCGCGGCAGCC
TGCTGACCTGCGGCGACGTGGAGGAAAACCCCGGCCCCACCGGTCACCATGGATGTA
CAGGATGCAACTCCTGTCTTGCATTGCACTAAGTCTTGCACTTGTCACAAACAGTGCA
CCTACTTCAAGTTCTACAAAGAAAACACAGCTACAACTGGAGCATTTACTGCTGGATTT
ACAGATGATTTTGAATGGAATTAATAATTACAAGAATCCCAAACTCACCGCGATGCTCA
CAGCTAAGTTTGCCATGCCCAAGAAGGCCACAGAACTGAAACATCTTCAGTGTCTAGA
AGAAGCACTCAAACCTCTGGAGGAAGTGCTAAATTTAGCTCAAAGCAAAAACTTTCA
CTTAAGACCCAGGGACTTAATCAGCAATATCAACGTAATAGTTCTGGAACTAAAGGGAT
CTGAAACAACATTCATGTGTGAATATGCTGATGAGACAGCAACCATTGTAGAATTTCTG
AACAGATGGATTACCTTTTGTCAAAGCATCATCTCAACACTGACTTGAAGATCTGAGG
GCCGCGGCAGCCTGCTGACCTGCGGCGACGTGGAGGAAAACCCCGGCCCCGGATCCA
TGAAACCAGTAACGTTATACGATGTCGCAGAGTATGCCGGTGTCTCTTATCAGACCGTT
TCCCGCGTGGTGAACCAGGCCAGCCACGTTTCTGCGAAAACGCGGGAAAAAGTGGAA
GCGGCGATGGCGGAGCTGAATTACATTCCCAACCGCGTGGCACAACAACTGGCGGGC
AAACAGTCGTTGCTGATTGGCGTTGCCACCTCCAGTCTGGCCCTGCACGCGCCGTCGC
AAATTGTCGCGGCGATTAAATCTCGCGCCGATCAACTGGGTGCCAGCGTGGTGGTGTC
GATGGTAGAACGAAGCGGCGTCGAAGCCTGTAAAGCGGCGGTGCACAATCTTCTCGC
GCAACGCGTCAGTGGGCTGATCATTAACTATCCGCTGGATGACCAGGATGCCATTGCTG
TGGAAGCTGCCTGCACTAATGTTCCGGCGTTATTTCTTGATGTCTCTGACCAGACACCC
ATCAACAGTATTATTTTCTCCCATGAAGACGGTACGCGACTGGGCGTGGAGCATCTGGT
CGCATTGGGTCACCAGCAAATCGCGCTGTTAGCGGGCCCATTAAGTTCTGTCTCGGCG
CGTCTGCGTCTGGCTGGCTGGCATAAATATCTCACTCGCAATCAAATTCAGCCGATAGC
GGAACGGGAAGGCGACTGGAGTGCCATGTCCGGTTTTCAACAAACCATGCAAATGCT
GAATGAGGGCATCGTTCCCACTGCGATGCTGGTTGCCAACGATCAGATGGCGCTGGGC
GCAATGCGCGCCATTACCGAGTCCGGGCTGCGCGTTGGTGCGGATATCTCGGTAGTGG
GATACGACGATACCGAAGACAGCTCATGTTATATCCCGCCGTTAACCACCATCAAACAG
GATTTTCGCCTGCTGGGGCAAACCAGCGTGGACCGCTTGCTGCAACTCTCTCAGGGCC
AGGCGGTGAAGGGCAATCAGCTGTTGCCCGTCTCACTGGTGAAAAGAAAAACCACCC
TGGCGCCCAATACGCAAACCGCCTCTCCCCGCGCGTTGGCCGATTCATTAATGCAGCT
GGCACGACAGGTTTCCCGACTGGAAAGCGGGCAGAGCAGCCTGAGGCCTCCTAAGAA
GAAGAGGAAGGTTGCGGCCGCTAACCAATGTGCAGACTACTGTTAACCAATGTGCAG
ACTACTGTGAATTCTAACCAATGTGCAGACTACTGTTAACCAATGTGCAGACTACTGTA
AGCTTCGAATTGAGTAGTGCTTTCTACTTTATGAGTAGTGCTTTCTACTTTATGAG
TAGTGCTTTCTACTTTATGAGTAGTGCTTTCTACTTTATGGTCGACAATACTAGTTAAGA
AATGAGACC.
(SEQ ID NO: 4)
GGTCTCTCTATTTAATTAAGTAACTATAACGGTCGCTCCGAATTTCTCGAGTTAATTA
AGATTACGCCAAGCTACGGGCGGAGTACTGTCCTCCGAGCGGAGTACTGTCCTCCGAG
CGGAGTACTGTCCTCCGAGCGGAGTACTGTCCTCCGAGCGGAGTTCTGTCCTCCGAGC
GGAGACTCTAGACTCCCTATCAGTGATAGAGATAGGCGTGTACGGTGGGAGGCCTATAT
AAGCAGAGCTCGTTTAGTGAACCGTCAGATCGCTCCCTATCAGTGATAGAGAGAATTC
GACCGCCACCATGTCGGAATTCATGAGACATATTATCTGCCACGGAGGTGTTATTACCG
AAGAAATGGCCGCCAGTCTTTTGGACCAGCTGATCGAAGAGGTACTGGCTGATAATCT
TCCACCTCCTAGCCATTTTGAACCACCTACCCTTCACGAACTGTATGATTTAGACGTGA
CGGCCCCCGAAGATCCCAACGAGGAGGCGGTTTCGCAGATTTTTCCCGACTCTGTAAT
GTTGGCGGTGCAGGAAGGGATTGACTTACTCACTTTTCCGCCGGCGCCCGGTTCTCCG
GAGCCGCCTCACCTTTCCCGGCAGCCCGAGCAGCCGGAGCAGAGAGCCTTGGGTCCG
GTTTCTATGCCAAACCTTGTACCGGAGGTGATCGATCTTACCTGCCACGAGGCTGGCTT
TCCACCCAGTGACGACGAGGATGAAGAGGGTGAGGAGTTTGTGTTAGATTATGTGGAG
CACCCCGGGCACGGTTGCAGGTCTTGTCATTATCACCGGAGGAATACGGGGGACCCAG
ATATTATGTGTTCGCTTTGCTATATGAGGACCTGTGGCATGTTTGTCTACAGTAAGTGAA
AATTATGGGCAGTGGGTGATAGAGTGGTGGGTTTGGTGTGGTAATTTTTTTTTTAATTTT
TACAGTTTTGTGGTTTAAAGAATTTTGTATTGTGATTTTTTTAAAAGGTCCTGTGTCTGA
ACCTGAGCCTGAGCCCGAGCCAGAACCGGAGCCTGCAAGACCTACCCGCCGTCCTAA
AATGGCGCCTGCTATCCTGAGACGCCCGACATCACCTGTGTCTAGAGAATGCAATAGTA
GTACGGATAGCTGTGACTCCGGTCCTTCTAACACACCTCCTGAGATACACCCGGTGGT
CCCGCTGTGCCCCATTAAACCAGTTGCCGTGAGAGTTGGTGGGCGTCGCCAGGCTGTG
GAATGTATCGAGGACTTGCTTAACGAGCCTGGGCAACCTTTGGACTTGAGCTGTAAAC
GCCCCAGGCCACTCGAGGGTACCGGGTCCGGAGCTACAAATTTTTCCCTCCTCAAACA
GGCTGGAGATGTCGAAGAAAATCCTGGGCCTACCGGTCACCATGGATGTGGCTGCAGA
GCCTGCTGCTCTTGGGCACTGTGGCCTGCAGCATCTCTGCACCCGCCCGCTCGCCCAG
CCCCAGCACGCAGCCCTGGGAGCATGTGAATGCCATCCAGGAGGCCCGGCGTCTCCTG
AACCTGAGTAGAGACACTGCTGCTGAGATGAATGAAACAGTAGAAGTCATCTCAGAA
ATGTTTGACCTCCAGGAGCCGACCTGCCTACAGACCCGCCTGGAGCTGTACAAGCAG
GGCCTGCGGGGCAGCCTCACCAAGCTCAAGGGCCCCTTGACCATGATGGCCAGCCAC
TACAAGCAGCACTGCCCTCCAACCCCGGAA
ACTTCCTGTGCAACCCAGATTATCACCTTTGAAAGTTTCAAAGAGAACCTGAAGGACT
TTCTGCTTGTCATCCCCTTTGACTGCTGGGAGCCAGTCCAGGAGTGAAGATCTGAGGG
CCGCGGCAGCCTGCTGACCTGCGGCGACGTGGAGGAAAACCCCGGCCCCGGATCCAT
GAAACCAGTAACGTTATACGATGTCGCAGAGTATGCCGGTGTCTCTTATCAGACCGTTT
CCCGCGTGGTGAACCAGGCCAGCCACGTTTCTGCGAAAACGCGGGAAAAAGTGGAA
GCGGCGATGGCGGAGCTGAATTACATTCCCAACCGCGTGGCACAACAACTGGCGGGC
AAACAGTCGTTGCTGATTGGCGTTGCCACCTCCAGTCTGGCCCTGCACGCGCCGTCGC
AAATTGTCGCGGCGATTAAATCTCGCGCCGATCAACTGGGTGCCAGCGTGGTGGTGTC
GATGGTAGAACGAAGCGGCGTCGAAGCCTGTAAAGCGGCGGTGCACAATCTTCTCGC
GCAACGCGTCAGTGGGCTGATCATTAACTATCCGCTGGATGACCAGGATGCCATTGCTG
TGGAAGCTGCCTGCACTAATGTTCCGGCGTTATTTCTTGATGTCTCTGACCAGACACCC
ATCAACAGTATTATTTTCTCCCATGAAGACGGTACGCGACTGGGCGTGGAGCATCTGGT
CGCATTGGGTCACCAGCAAATCGCGCTGTTAGCGGGCCCATTAAGTTCTGTCTCGGCG
CGTCTGCGTCTGGCTGGCTGGCATAAATATCTCACTCGCAATCAAATTCAGCCGATAGC
GGAACGGGAAGGCGACTGGAGTGCCATGTCCGGTTTTCAACAAACCATGCAAATGCT
GAATGAGGGCATCGTTCCCACTGCGATGCTGGTTGCCAACGATCAGATGGCGCTGGGC
GCAATGCGCGCCATTACCGAGTCCGGGCTGCGCGTTGGTGCGGATATCTCGGTAGTGG
GATACGACGATACCGAAGACAGCTCATGTTATATCCCGCCGTTAACCACCATCAAACAG
GATTTTCGCCTGCTGGGGCAAACCAGCGTGGACCGCTTGCTGCAACTCTCTCAGGGCC
AGGCGGTGAAGGGCAATCAGCTGTTGCCCGTCTCACTGGTGAAAAGAAAAACCACCC
TGGCGCCCAATACGCAAACCGCCTCTCCCCGCGCGTTGGCCGATTCATTAATGCAGCT
GGCACGACAGGTTTCCCGACTGGAAAGCGGGCAGAGCAGCCTGAGGCCTCCTAAGAA
GAAGAGGAAGGTTGCGGCCGCTAACCAATGTGCAGACTACTGTTAACCAATGTGCAG
ACTACTGTGAATTCTAACCAATGTGCAGACTACTGTTAACCAATGTGCAGACTACTGTA
AGCTTCGAATTGAGTAGTGCTTTCTACTTTATGAGTAGTGCTTTCTACTTTATGAGTAGT
GCTTTCTACTTTATGAGTAGTGCTTTCTACTTTATGGTCGACAATACTAGTTAAGAAATG
AGACC.
(SEQ ID NO: 5)
GGTCTCTCTATTTAATTAAGTAACTATAACGGTCGCTCCGAATTTCTCGAGTTAATTA
AGATTACGCCAAGCTACGGGCGGAGTACTGTCCTCCGAGCGGAGTACTGTCCTCCGAG
CGGAGTACTGTCCTCCGAGCGGAGTACTGTCCTCCGAGCGGAGTTCTGTCCTCCGAGC
GGAGACTCTAGACTCCCTATCAGTGATAGAGATAGGCGTGTACGGTGGGAGGCCTATAT
AAGCAGAGCTCGTTTAGTGAACCGTCAGATCGCTCCCTATCAGTGATAGAG
AGAATTCGACCGCCACCATGTCGGAATTCATGAGACATATTATCTGCCACGGAGGTGTT
ATTACCGAAGAAATGGCCGCCAGTCTTTTGGACCAGCTGATCGAAGAGGTACTGGCTG
ATAATCTTCCACCTCCTAGCCATTTTGAACCACCTACCCTTCACGAACTGTATGATTTAG
ACGTGACGGCCCCCGAAGATCCCAACGAGGAGGCGGTTTCGCAGATTTTTCCCGACTC
TGTAATGTTGGCGGTGCAGGAAGGGATTGACTTACTCACTTTTCCGCCGGCGCCCGGT
TCTCCGGAGCCGCCTCACCTTTCCCGGCAGCCCGAGCAGCCGGAGCAGAGAGCCTTG
GGTCCGGTTTCTATGCCAAACCTTGTACCGGAGGTGATCGATCTTACCTGCCACGAGGC
TGGCTTTCCACCCAGTGACGACGAGGATGAAGAGGGTGAGGAGTTTGTGTTAGATTAT
GTGGAGCACCCCGGGCACGGTTGCAGGTCTTGTCATTATCACCGGAGGAATACGGGGG
ACCCAGATATTATGTGTTCGCTTTGCTATATGAGGACCTGTGGCATGTTTGTCTACAGTA
AGTGAAAATTATGGGCAGTGGGTGATAGAGTGGTGGGTTTGGTGTGGTAATTTTTTTTT
TAATTTTTACAGTTTTGTGGTTTAAAGAATTTTGTATTGTGATTTTTTTAAAAGGTCCTG
TGTCTGAACCTGAGCCTGAGCCCGAGCCAGAACCGGAGCCTGCAAGACCTACCCGCC
GTCCTAAAATGGCGCCTGCTATCCTGAGACGCCCGACATCACCTGTGTCTAGAGAATG
CAATAGTAGTACGGATAGCTGTGACTCCGGTCCTTCTAACACACCTCCTGAGATACACC
CGGTGGTCCCGCTGTGCCCCATTAAACCAGTTGCCGTGAGAGTTGGTGGGCGTCGCCA
GGCTGTGGAATGTATCGAGGACTTGCTTAACGAGCCTGGGCAACCTTTGGACTTGAGC
TGTAAACGCCCCAGGCCACTCGAGGGTACCGGGTCCGGAGCTACAAATTTTTCCCTCC
TCAAACAGGCTGGAGATGTCGAAGAAAATCCTGGGCCTACCGGTCACCATGGATGTGG
CTGCAGAATTTACTTTTCCTGGGCATTGTGGTCTACAGCCTCTCAGCACCCACCCGCTC
ACCCATCACTGTCACCCGGCCTTGGAAGCATGTAGAGGCCATCAAAGAAGCCCTGAAC
CTCCTGGATGACATGCCTGTCACGTTGAATGAAGAGGTAGAAGTCGTCTCTAACGAGT
TCTCCTTCAAGAAGCTAACATGTGTGCAGACCCGCCTGAAGATATTCGAGCAGGGTCT
ACGGGGCAATTTCACCAAACTCAAGGGCGCCTTGAACATGACAGCCAGCTACTACCA
GACATACTGCCCCCCAACTCCGGAAACGGACTGTGAAACACAAGTTACCACCTATGCG
GATTTCATAGACAGCCTTAAAACCTTTCTGACTGATATCCCCTTTGAATGCAAAAAACC
AGGCCAAAAATGAAGATCTGAGGGCCGCGGCAGCCTGCTGACCTGCGGCGACGTGGA
GGAAAACCCCGGCCCCGGATCCATGAAACCAGTAACGTTATACGATGTCGCAGAGTAT
GCCGGTGTCTCTTATCAGACCGTTTCCCGCGTGGTGAACCAGGCCAGCCACGTTTCTG
CGAAAACGCGGGAAAAAGTGGAAGCGGCGATGGCGGAGCTGAATTACATTCCCAACC
GCGTGGCACAACAACTGGCGGGCAAACAGTCGTTGCTGATTGGCGTTGCCACCTCCA
GTCTGGCCCTGCACGCGCCGTCGCAAATTGTCGC
GGCGATTAAATCTCGCGCCGATCAACTGGGTGCCAGCGTGGTGGTGTCGATGGTAGAA
CGAAGCGGCGTCGAAGCCTGTAAAGCGGCGGTGCACAATCTTCTCGCGCAACGCGTC
AGTGGGCTGATCATTAACTATCCGCTGGATGACCAGGATGCCATTGCTGTGGAAGCTGC
CTGCACTAATGTTCCGGCGTTATTTCTTGATGTCTCTGACCAGACACCCATCAACAGTA
TTATTTTCTCCCATGAAGACGGTACGCGACTGGGCGTGGAGCATCTGGTCGCATTGGGT
CACCAGCAAATCGCGCTGTTAGCGGGCCCATTAAGTTCTGTCTCGGCGCGTCTGCGTC
TGGCTGGCTGGCATAAATATCTCACTCGCAATCAAATTCAGCCGATAGCGGAACGGGA
AGGCGACTGGAGTGCCATGTCCGGTTTTCAACAAACCATGCAAATGCTGAATGAGGGC
ATCGTTCCCACTGCGATGCTGGTTGCCAACGATCAGATGGCGCTGGGCGCAATGCGCG
CCATTACCGAGTCCGGGCTGCGCGTTGGTGCGGATATCTCGGTAGTGGGATACGACGAT
ACCGAAGACAGCTCATGTTATATCCCGCCGTTAACCACCATCAAACAGGATTTTCGCCT
GCTGGGGCAAACCAGCGTGGACCGCTTGCTGCAACTCTCTCAGGGCCAGGCGGTGAA
GGGCAATCAGCTGTTGCCCGTCTCACTGGTGAAAAGAAAAACCACCCTGGCGCCCAA
TACGCAAACCGCCTCTCCCCGCGCGTTGGCCGATTCATTAATGCAGCTGGCACGACAG
GTTTCCCGACTGGAAAGCGGGCAGAGCAGCCTGAGGCCTCCTAAGAAGAAGAGGAA
GGTTGCGGCCGCTAACCAATGTGCAGACTACTGTTAACCAATGTGCAGACTACTGTGA
ATTCTAACCAATGTGCAGACTACTGTTAACCAATGTGCAGACTACTGTAAGCTTCGAAT
TGAGTAGTGCTTTCTACTTTATGAGTAGTGCTTTCTACTTTATGAGTAGTGCTTTCTACT
TTATGAGTAGTGCTTTCTACTTTATGGTCGACAATACTAGTTAAGAAATGAGACC.
(SEQ ID NO: 6)
GGTCTCTCTATTTAATTAAGTAACTATAACGGTCGCTCCGAATTTCTCGAGTTAATTA
AGATTACGCCAAGCTACGGGCGGAGTACTGTCCTCCGAGCGGAGTACTGTCCTCCGAG
CGGAGTACTGTCCTCCGAGCGGAGTACTGTCCTCCGAGCGGAGTTCTGTCCTCCGAGC
GGAGACTCTAGACTCCCTATCAGTGATAGAGATAGGCGTGTACGGTGGGAGGCCTATAT
AAGCAGAGCTCGTTTAGTGAACCGTCAGATCGCTCCCTATCAGTGATAGAGAGAATTC
GACCGCCACCATGTCGGAATTCATGAGACATATTATCTGCCACGGAGGTGTTATTACCG
AAGAAATGGCCGCCAGTCTTTTGGACCAGCTGATCGAAGAGGTACTGGCTGATAATCT
TCCACCTCCTAGCCATTTTGAACCACCTACCCTTCACGAACTGTATGATTTAGACGTGA
CGGCCCCCGAAGATCCCAACGAGGAGGCGGTTTCGCAGATTTTTCCCGACTCTGTAAT
GTTGGCGGTGCAGGAAGGGATTGACTTACTCACTTTTCCGCCGGCGCCCGGTTCTCCG
GAGCCGCCTCACCTTTCCCGGCAGCCCGAGCAGCCGGAGCAGAGAGCCTTGGGTCCG
GTTTCTATGCCAAACCTTGTACCGGAGGTGATCGATCTTACCTGCC
ACGAGGCTGGCTTTCCACCCAGTGACGACGAGGATGAAGAGGGTGAGGAGTTTGTGT
TAGATTATGTGGAGCACCCCGGGCACGGTTGCAGGTCTTGTCATTATCACCGGAGGAAT
ACGGGGGACCCAGATATTATGTGTTCGCTTTGCTATATGAGGACCTGTGGCATGTTTGT
CTACAGTAAGTGAAAATTATGGGCAGTGGGTGATAGAGTGGTGGGTTTGGTGTGGTAA
TTTTTTTTTTAATTTTTACAGTTTTGTGGTTTAAAGAATTTTGTATTGTGATTTTTTTAAA
AGGTCCTGTGTCTGAACCTGAGCCTGAGCCCGAGCCAGAACCGGAGCCTGCAAGACC
TACCCGCCGTCCTAAAATGGCGCCTGCTATCCTGAGACGCCCGACATCACCTGTGTCTA
GAGAATGCAATAGTAGTACGGATAGCTGTGACTCCGGTCCTTCTAACACACCTCCTGA
GATACACCCGGTGGTCCCGCTGTGCCCCATTAAACCAGTTGCCGTGAGAGTTGGTGGG
CGTCGCCAGGCTGTGGAATGTATCGAGGACTTGCTTAACGAGCCTGGGCAACCTTTGG
ACTTGAGCTGTAAACGCCCCAGGCCACTCGAGGGTACCGGGTCCGGAGCTACAAATTT
TTCCCTCCTCAAACAGGCTGGAGATGTCGAAGAAAATCCTGGGCCTACCGGTCCCATG
AGTGTGCCCACTCAGGTCCTGGGGTTGCTGCTGCTGTGGCTTACAGACGCTCGCTGCC
AGGTGCAGCTGGTGGAGAGCGGCGGCGGCGTGGTGCAGCCCGGCAGGAGCCTGAGG
CTGGACTGCAAGGCCAGCGGCATCACCTTCAGCAACAGCGGCATGCACTGGGTGAGG
CAGGCCCCCGGCAAGGGCCTGGAGTGGGTGGCCGTGATCTGGTACGACGGCAGCAAG
AGGTACTACGCCGACAGCGTGAAGGGCAGGTTCACCATCAGCAGGGACAACAGCAAG
AACACCCTGTTCCTGCAGATGAACAGCCTGAGGGCCGAGGACACCGCCGTGTACTAC
TGCGCCACCAACGACGACTACTGGGGCCAGGGCACCCTGGTGACCGTGAGCAGCGGT
GGAGGCGGTTCAGGCGGAGGTGGCTCTGGCGGTGGCGGATCGGAGATCGTGCTGACC
CAGAGCCCCGCCACCCTGAGCCTGAGCCCCGGCGAGAGGGCCACCCTGAGCTGCAGG
GCCAGCCAGAGCGTGAGCAGCTACCTGGCCTGGTACCAGCAGAAGCCCGGCCAGGCC
CCCAGGCTGCTGATCTACGACGCCAGCAACAGGGCCACCGGCATCCCCGCCAGGTTC
AGCGGCAGCGGCAGCGGCACCGACTTCACCCTGACCATCAGCAGCCTGGAGCCCGAG
GACTTCGCCGTGTACTACTGCCAGCAGAGCAGCAACTGGCCCAGGACCTTCGGCCAG
GGCACCAAGGTGGAGATCAAGAGATCTGAGGGCCGCGGCAGCCTGCTGACCTGCGGC
GACGTGGAGGAAAACCCCGGCCCCGGATCCATGAAACCAGTAACGTTATACGATGTCG
CAGAGTATGCCGGTGTCTCTTATCAGACCGTTTCCCGCGTGGTGAACCAGGCCAGCCA
CGTTTCTGCGAAAACGCGGGAAAAAGTGGAAGCGGCGATGGCGGAGCTGAATTACAT
TCCCAACCGCGTGGCACAACAACTGGCGGGCAAACAGTCGTTGCTGATTGGCGTTGC
CACCTCCAGTCTGGCCCTGCACGCGCCGTCGCAAATTGTCGCGGCGATTAAATCTCGC
GCCGATCAACTGGGTGCCAGCGTGGTGGTGTCGATGGTAGA
ACGAAGCGGCGTCGAAGCCTGTAAAGCGGCGGTGCACAATCTTCTCGCGCAACGCGT
CAGTGGGCTGATCATTAACTATCCGCTGGATGACCAGGATGCCATTGCTGTGGAAGCTG
CCTGCACTAATGTTCCGGCGTTATTTCTTGATGTCTCTGACCAGACACCCATCAACAGT
ATTATTTTCTCCCATGAAGACGGTACGCGACTGGGCGTGGAGCATCTGGTCGCATTGGG
TCACCAGCAAATCGCGCTGTTAGCGGGCCCATTAAGTTCTGTCTCGGCGCGTCTGCGT
CTGGCTGGCTGGCATAAATATCTCACTCGCAATCAAATTCAGCCGATAGCGGAACGGG
AAGGCGACTGGAGTGCCATGTCCGGTTTTCAACAAACCATGCAAATGCTGAATGAGGG
CATCGTTCCCACTGCGATGCTGGTTGCCAACGATCAGATGGCGCTGGGCGCAATGCGC
GCCATTACCGAGTCCGGGCTGCGCGTTGGTGCGGATATCTCGGTAGTGGGATACGACG
ATACCGAAGACAGCTCATGTTATATCCCGCCGTTAACCACCATCAAACAGGATTTTCGC
CTGCTGGGGCAAACCAGCGTGGACCGCTTGCTGCAACTCTCTCAGGGCCAGGCGGTG
AAGGGCAATCAGCTGTTGCCCGTCTCACTGGTGAAAAGAAAAACCACCCTGGCGCCC
AATACGCAAACCGCCTCTCCCCGCGCGTTGGCCGATTCATTAATGCAGCTGGCACGAC
AGGTTTCCCGACTGGAAAGCGGGCAGAGCAGCCTGAGGCCTCCTAAGAAGAAGAGG
AAGGTTGCGGCCGCTAACCAATGTGCAGACTACTGTTAACCAATGTGCAGACTACTGT
GAATTCTAACCAATGTGCAGACTACTGTTAACCAATGTGCAGACTACTGTAAGCTTCGA
ATTGAGTAGTGCTTTCTACTTTATGAGTAGTGCTTTCTACTTTATGAGTAGTGCTTTCTA
CTTTATGAGTAGTGCTTTCTACTTTATGGTCGACAATACTAGTTAAGAAATGAGACC.
(SEQ ID NO: 7)
GGTCTCTCTATTTAATTAAGTAACTATAACGGTCGCTCCGAATTTCTCGAGTTAATTA
AGATTACGCCAAGCTACGGGCGGAGTACTGTCCTCCGAGCGGAGTACTGTCCTCCGAG
CGGAGTACTGTCCTCCGAGCGGAGTACTGTCCTCCGAGCGGAGTTCTGTCCTCCGAGC
GGAGACTCTAGACTCCCTATCAGTGATAGAGATAGGCGTGTACGGTGGGAGGCCTATAT
AAGCAGAGCTCGTTTAGTGAACCGTCAGATCGCTCCCTATCAGTGATAGAGAGAATTC
GACCGCCACCATGTCGGAATTCATGAGACATATTATCTGCCACGGAGGTGTTATTACCG
AAGAAATGGCCGCCAGTCTTTTGGACCAGCTGATCGAAGAGGTACTGGCTGATAATCT
TCCACCTCCTAGCCATTTTGAACCACCTACCCTTCACGAACTGTATGATTTAGACGTGA
CGGCCCCCGAAGATCCCAACGAGGAGGCGGTTTCGCAGATTTTTCCCGACTCTGTAAT
GTTGGCGGTGCAGGAAGGGATTGACTTACTCACTTTTCCGCCGGCGCCCGGTTCTCCG
GAGCCGCCTCACCTTTCCCGGCAGCCCGAGCAGCCGGAGCAGAGAGCCTTGGGTCCG
GTTTCTATGCCAAACCTTGTACCGGAGGTGATCGATCTTACCTGCCACGAGGCTGGCTT
TCCACCCAGTGACGACGAGGATGAAGAGGGTGAGGAGTTTGTGT
TAGATTATGTGGAGCACCCCGGGCACGGTTGCAGGTCTTGTCATTATCACCGGAGGAAT
ACGGGGGACCCAGATATTATGTGTTCGCTTTGCTATATGAGGACCTGTGGCATGTTTGT
CTACAGTAAGTGAAAATTATGGGCAGTGGGTGATAGAGTGGTGGGTTTGGTGTGGTAA
TTTTTTTTTTAATTTTTACAGTTTTGTGGTTTAAAGAATTTTGTATTGTGATTTTTTTAAA
AGGTCCTGTGTCTGAACCTGAGCCTGAGCCCGAGCCAGAACCGGAGCCTGCAAGACC
TACCCGCCGTCCTAAAATGGCGCCTGCTATCCTGAGACGCCCGACATCACCTGTGTCTA
GAGAATGCAATAGTAGTACGGATAGCTGTGACTCCGGTCCTTCTAACACACCTCCTGA
GATACACCCGGTGGTCCCGCTGTGCCCCATTAAACCAGTTGCCGTGAGAGTTGGTGGG
CGTCGCCAGGCTGTGGAATGTATCGAGGACTTGCTTAACGAGCCTGGGCAACCTTTGG
ACTTGAGCTGTAAACGCCCCAGGCCACTCGAGGGTACCGGGTCCGGAGCTACAAATTT
TTCCCTCCTCAAACAGGCTGGAGATGTCGAAGAAAATCCTGGGCCTACCGGTCCCATG
AGTGTGCCCACTCAGGTCCTGGGGTTGCTGCTGCTGTGGCTTACAGACGCTCGCTGCG
AGGTGCAGCTGGTGGAGAGCGGCGGCGGCCTGGTGCAGCCCGGCGGCAGCCTGAGG
CTGAGCTGCGCCGCCAGCGGCTTCACCTTCAGCGACAGCTGGATCCACTGGGTGAGG
CAGGCCCCCGGCAAGGGCCTGGAGTGGGTGGCCTGGATCAGCCCCTACGGCGGCAGC
ACCTACTACGCCGACAGCGTGAAGGGCAGGTTCACCATCAGCGCCGACACCAGCAAG
AACACCGCCTACCTGCAGATGAACAGCCTGAGGGCCGAGGACACCGCCGTGTACTAC
TGCGCCAGGAGGCACTGGCCCGGCGGCTTCGACTACTGGGGCCAGGGCACCCTGGTG
ACCGTGAGCAGCGGTGGAGGCGGTTCAGGCGGAGGTGGCTCTGGCGGTGGCGGATCG
GACATCCAGATGACCCAGAGCCCCAGCAGCCTGAGCGCCAGCGTGGGCGACAGGGTG
ACCATCACCTGCAGGGCCAGCCAGGACGTGAGCACCGCCGTGGCCTGGTACCAGCAG
AAGCCCGGCAAGGCCCCCAAGCTGCTGATCTACAGCGCCAGCTTCCTGTACAGCGGC
GTGCCCAGCAGGTTCAGCGGCAGCGGCAGCGGCACCGACTTCACCCTGACCATCAGC
AGCCTGCAGCCCGAGGACTTCGCCACCTACTACTGCCAGCAGTACCTGTACCACCCCG
CCACCTTCGGCCAGGGCACCAAGGTGGAGATCAAGAGATCTGAGGGCCGCGGCAGCC
TGCTGACCTGCGGCGACGTGGAGGAAAACCCCGGCCCCGGATCCATGAAACCAGTAA
CGTTATACGATGTCGCAGAGTATGCCGGTGTCTCTTATCAGACCGTTTCCCGCGTGGTG
AACCAGGCCAGCCACGTTTCTGCGAAAACGCGGGAAAAAGTGGAAGCGGCGATGGC
GGAGCTGAATTACATTCCCAACCGCGTGGCACAACAACTGGCGGGCAAACAGTCGTT
GCTGATTGGCGTTGCCACCTCCAGTCTGGCCCTGCACGCGCCGTCGCAAATTGTCGCG
GCGATTAAATCTCGCGCCGATCAACTGGGTGCCAGCGTGGTGGTGTCGATGGTAGAAC
GAAGCGGCGTCGAAGCCTGTAAAGCGGCGGTGCACAATCTTCTCG
CGCAACGCGTCAGTGGGCTGATCATTAACTATCCGCTGGATGACCAGGATGCCATTGCT
GTGGAAGCTGCCTGCACTAATGTTCCGGCGTTATTTCTTGATGTCTCTGACCAGACACC
CATCAACAGTATTATTTTCTCCCATGAAGACGGTACGCGACTGGGCGTGGAGCATCTGG
TCGCATTGGGTCACCAGCAAATCGCGCTGTTAGCGGGCCCATTAAGTTCTGTCTCGGC
GCGTCTGCGTCTGGCTGGCTGGCATAAATATCTCACTCGCAATCAAATTCAGCCGATAG
CGGAACGGGAAGGCGACTGGAGTGCCATGTCCGGTTTTCAACAAACCATGCAAATGC
TGAATGAGGGCATCGTTCCCACTGCGATGCTGGTTGCCAACGATCAGATGGCGCTGGG
CGCAATGCGCGCCATTACCGAGTCCGGGCTGCGCGTTGGTGCGGATATCTCGGTAGTG
GGATACGACGATACCGAAGACAGCTCATGTTATATCCCGCCGTTAACCACCATCAAACA
GGATTTTCGCCTGCTGGGGCAAACCAGCGTGGACCGCTTGCTGCAACTCTCTCAGGGC
CAGGCGGTGAAGGGCAATCAGCTGTTGCCCGTCTCACTGGTGAAAAGAAAAACCACC
CTGGCGCCCAATACGCAAACCGCCTCTCCCCGCGCGTTGGCCGATTCATTAATGCAGCT
GGCACGACAGGTTTCCCGACTGGAAAGCGGGCAGAGCAGCCTGAGGCCTCCTAAGAA
GAAGAGGAAGGTTGCGGCCGCTAACCAATGTGCAGACTACTGTTAACCAATGTGCAG
ACTACTGTGAATTCTAACCAATGTGCAGACTACTGTTAACCAATGTGCAGACTACTGTA
AGCTTCGAATTGAGTAGTGCTTTCTACTTTATGAGTAGTGCTTTCTACTTTATGAGTAGT
GCTTTCTACTTTATGAGTAGTGCTTTCTACTTTATGGTCGACAATACTAGTTAAGAAATG
AGACC.

wherein SEQ ID NO: 2 is the sequence of BsaI-5×UAS-tetO-miniCMV-tetO-E1A-2A-EBFP-2A-LacI-miR199a target site-BsaI;

SEQ ID NO: 3 is the sequence of BsaI-5×UAS-tetO-miniCMV-tetO-E1A-2A-hIL-2-2A-LacI-miR199a target site-BsaI;

SEQ ID NO: 4 is the sequence of BsaI-5×UAS-tetO-miniCMV-tetO-E1A-2A-hGM-CSF-2A-LacI-miR199a target site-BsaI;

SEQ ID NO: 5 is the sequence of BsaI-5×UAS-tetO-miniCMV-tetO-E1A-2A-mGM-CSF-2A-LacI-miR199a target site-BsaI;

SEQ ID NO: 6 is the sequence of BsaI-5×UAS-tetO-miniCMV-tetO-E1A-2A-anti-PD-lscFv-2A-LacI-miR199a target site-BsaI; and

SEQ ID NO: 7 is the sequence of BsaI-5×UAS-tetO-miniCMV-tetO-E1A-2A-anti-PD-L1scFv-2A-LacI-miR199a target site-BsaI.

In a specific embodiment of the present disclosure, the third expression vector comprises: BsaI, 5×UAS, LacO, miniCMV, LacO, tetR-KRAB, microRNA21 specific recognition sequence (target site) and BsaI from the 5′ end to the 3′ end

(BsaI-5×UAS-LacO-miniCMV-LacO-tetR-KRAB-microRNA21 target site-BsaI).

In an embodiment of the present disclosure, the third expression vector carries a nucleic acid having the nucleotide sequence set forth in SEQ ID NO: 8.

(SEQ ID NO: 8)
GGTCTCCGAAATCGCCAAGCTACGGGCGGAGTACTGTCCTCCGAGCGGAGTACTG
TCCTCCGAGCGGAGTACTGTCCTCCGAGCGGAGTACTGTCCTCCGAGCGGAGTACTGT
CCTCCGAGCGGAGACTCTAGAAATTGTGAGCGGATAACAATTTAGGCGTGTACGGTGG
GAGGCCTATATAAGCAGAGCTCGTTTAGTGAACCGTCAGATCGCCAGCTGACAAAATT
GTGAGCGCTCACAATTACTAGAAGATCTTGAATTCGCCACCATGGCTAGATTAGATAAA
AGTAAAGTGATTAACAGCGCATTAGAGCTGCTTAATGAGGTCGGAATCGAAGGTTTAA
CAACCCGTAAACTCGCCCAGAAGCTAGGTGTAGAGCAGCCTACATTGTATTGGCATGT
AAAAAATAAGCGGGCTTTGCTCGACGCCTTAGCCATTGAGATGTTAGATAGGCACCATA
CTCACTTTTGCCCTTTAGAAGGGGAAAGCTGGCAAGATTTTTTACGTAATAACGCTAAA
AGTTTTAGATGTGCTTTACTAAGTCATCGCGATGGAGCAAAAGTACATTTAGGTACACG
GCCTACAGAAAAACAGTATGAAACTCTCGAAAATCAATTAGCCTTTTTATGCCAACAA
GGTTTTTCACTAGAGAATGCATTATATGCACTCAGCGCTGTGGGGCATTTTACTTTAGGT
TGCGTATTGGAAGATCAAGAGCATCAAGTCGCTAAAGAAGAAAGGGAAACACCTACT
ACTGATAGTATGCCGCCATTATTACGACAAGCTATCGAATTATTTGATCACCAAGGTGCA
GAGCCAGCCTTCTTATTCGGCCTTGAATTGATCATATGCGGATTAGAAAAACAACTTAA
ATGTGAAAGTGGGTCGCCAAAAAAGAAGAGAAAGGTCGACGGCGGTGGTGCTTTGTC
TCCTCAGCACTCTGCTGTCACTCAAGGAAGTATCATCAAGAACAAGGAGGGCATGGAT
GCTAAGTCACTAACTGCCTGGTCCCGGACACTGGTGACCTTCAAGGATGTATTTGTGG
ACTTCACCAGGGAGGAGTGGAAGCTGCTGGACACTGCTCAGCAGATCGTGTACAGAA
ATGTGATGCTGGAGAACTATAAGAACCTGGTTTCCTTGGGTTATCAGCTTACTAAGCCA
GATGTGATCCTCCGGTTGGAGAAGGGAGAAGAGCCCTGGCTGGTGGAGAGAGAAATT
CACCAAGAGACCCATCCTGATTCAGAGACTGCATTTGAAATCAAATCATCAGTTTAATA
CAAGGCGGCCGCAAATCAACATCAGTCTGATAAGCTATCAACATCAGTCTGATAAGCTA
TCAACATCAGTCTGATAAGCTATCAACATCAGTCTGATAAGCTAAAGCTTCGAATTCTG
ATAATCAGCCATACCACATTTGTAGAGGTTTTACTTGCTTTAAAAAACCTCCCACACCT
CCCCCTGAACCTGAAACATAAAATGAATGCAATTGTTGTTGTTAACTTGTTTATTGCAG
CTTATAATGGTTACAAATAAAGCAATAGCATCACAAATTTCACAAATAAAGCATTTTTTT
CACTGCAGCTCTGAGACC.

In a specific embodiment of the present disclosure, the adenovirus comprises: a first inverted terminal repeat sequence (ITR), a packaging signal, AFPIII, Gal4VP16, 5×UAS, tetO, miniCMV, tetO, E1A, 2A, Effector, 2A, Lad, miR 199a target site, 5×UAS, LacO, miniCMV, LacO, tetR-KRAB, miR21 target site, an adenovirus E2 gene region, an adenovirus E3 gene region (removal of region 28922-30801), an adenovirus E4 gene region, and a second inverted terminal repeat sequence (ITR) from the 5′ end to the 3′ end. The above adenovirus when injected into a tumor mouse model by the present inventors can significantly inhibit the growth of tumor in mouse. The above adenovirus can be used as a safe and effective oncolytic virus vaccine to specifically kill related tumors in a safe and effective way.

In an embodiment of the present disclosure, the adenoviral vector carries a nucleic acid having the nucleotide sequences selected from the group consisting of SEQ ID NOS: 9-14.

(SEQ ID NO: 9)
GTAACTATAACGGTCGCTCCGAATTTCTCGAGTTAATTAAGATTACGCCAAGCTACG
GGCGGAGTACTGTCCTCCGAGCGGAGTACTGTCCTCCGAGCGGAGTACTGTCCTCCGA
GCGGAGTACTGTCCTCCGAGCGGAGTTCTGTCCTCCGAGCGGAGACTCTAGACTCCCT
ATCAGTGATAGAGATAGGCGTGTACGGTGGGAGGCCTATATAAGCAGAGCTCGTTTAGT
GAACCGTCAGATCGCTCCCTATCAGTGATAGAGAGAATTCGACCGCCACCATGTCGGA
ATTCATGAGACATATTATCTGCCACGGAGGTGTTATTACCGAAGAAATGGCCGCCAGTC
TTTTGGACCAGCTGATCGAAGAGGTACTGGCTGATAATCTTCCACCTCCTAGCCATTTT
GAACCACCTACCCTTCACGAACTGTATGATTTAGACGTGACGGCCCCCGAAGATCCCA
ACGAGGAGGCGGTTTCGCAGATTTTTCCCGACTCTGTAATGTTGGCGGTGCAGGAAGG
GATTGACTTACTCACTTTTCCGCCGGCGCCCGGTTCTCCGGAGCCGCCTCACCTTTCCC
GGCAGCCCGAGCAGCCGGAGCAGAGAGCCTTGGGTCCGGTTTCTATGCCAAACCTTG
TACCGGAGGTGATCGATCTTACCTGCCACGAGGCTGGCTTTCCACCCAGTGACGACGA
GGATGAAGAGGGTGAGGAGTTTGTGTTAGATTATGTGGAGCACCCCGGGCACGGTTGC
AGGTCTTGTCATTATCACCGGAGGAATACGGGGGACCCAGATATTATGTGTTCGCTTTG
CTATATGAGGACCTGTGGCATGTTTGTCTACAGTAAGTGAAAATTATGGGCAGTGGGTG
ATAGAGTGGTGGGTTTGGTGTGGTAATTTTTTTTTTAATTTTTACAGTTTTGTGGTTTAA
AGAATTTTGTATTGTGATTTTTTTAAAAGGTCCTGTGTCTGAACCTGAGCCTGAGCCCG
AGCCAGAACCGGAGCCTGCAAGACCTACCCGCCGTCCTAAAATGGCGCCTGCTATCCT
GAGACGCCCGACATCACCTGTGTCTAGAGAATGCAATAGTAGTACGGATAGCTGTGAC
TCCGGTCCTTCTAACACACCTCCTGAGATACACCCGGTGGTCCCGCTGTGCCCCATTAA
ACCAGTTGCCGTGAGAGTTGGTGGGCGTCGCCAGGCTGTGGAATGTATCGAGGACTTG
CTTAACGAGCCTGGGCAACCTTTGGACTTGAGCTGTAAACGCCCCAGGCCACTCGAG
GGTACCGGGTCCGGAGCTACAAATTTTTCCCTCCTCAAACAGGCTGGAGATGTCGAA
GAAAATCCTGGGCCTACCGGTCCCATGGTGAGCAAGGGCGAGGAGCTGTTCACCGGG
GTGGTGCCCATCCTGGTCGAGCTGGACGGCGACGTAAACGGCCACAAGTTCAGCGTG
AGGGGCGAGGGCGAGGGCGATGCCACCAACGGCAAGCTGACCCTGAAGTTCATCTGC
ACCACCGGCAAGCTGCCCGTGCCCTGGCCCACCCTCGTGACCACCCTGAGCCACGGC
GTGCAGTGCTTCGCCCGCTACCCCGACCACATGAAGCAGCACGACTTCTTCAAGTCCG
CCATGCCCGAAGGCTACGTCCAGGAGCGCACCATCTTCTTCAAGGACGACGGCACCTA
CAAGACCCGCGCCGAGGTGAAGTTCGAGGGCGACACCCTAGTGAACCGCATCGAGCT
GAAGGGCGTCGACTTCAAGGAGGACGGCAACATCCTGGGGCACAAGCTGGAGTACA
ACTTCAACAGCCACAACATCTATATCATGGCCGTCAAGCAGAAGAACGGCATCAAGGT
GAACTTCAAGATCCGCCACAACGTGGAGGACGGCAGCGTGCAGCTCGCCGACCACTA
CCAGCAGAACACCCCCATCGGCGACGGCCCCGTGCTGCTGCCCGACAGCCACTACCT
GAGCACCCAGTCCGTGCTGAGCAAAGACCCCAACGAGAAGCGCGATCACATGGTCCT
GCTGGAGTTCCGCACCGCCGCCGGGATCACTCTCGGCATGGACGAGCTGTACAAGAG
ATCTGAGGGCCGCGGCAGCCTGCTGACCTGCGGCGACGTGGAGGAAAACCCCGGCCC
CGGATCCATGAAACCAGTAACGTTATACGATGTCGCAGAGTATGCCGGTGTCTCTTATC
AGACCGTTTCCCGCGTGGTGAACCAGGCCAGCCACGTTTCTGCGAAAACGCGGGAAA
AAGTGGAAGCGGCGATGGCGGAGCTGAATTACATTCCCAACCGCGTGGCACAACAAC
TGGCGGGCAAACAGTCGTTGCTGATTGGCGTTGCCACCTCCAGTCTGGCCCTGCACGC
GCCGTCGCAAATTGTCGCGGCGATTAAATCTCGCGCCGATCAACTGGGTGCCAGCGTG
GTGGTGTCGATGGTAGAACGAAGCGGCGTCGAAGCCTGTAAAGCGGCGGTGCACAAT
CTTCTCGCGCAACGCGTCAGTGGGCTGATCATTAACTATCCGCTGGATGACCAGGATGC
CATTGCTGTGGAAGCTGCCTGCACTAATGTTCCGGCGTTATTTCTTGATGTCTCTGACC
AGACACCCATCAACAGTATTATTTTCTCCCATGAAGACGGTACGCGACTGGGCGTGGA
GCATCTGGTCGCATTGGGTCACCAGCAAATCGCGCTGTTAGCGGGCCCATTAAGTTCT
GTCTCGGCGCGTCTGCGTCTGGCTGGCTGGCATAAATATCTCACTCGCAATCAAATTCA
GCCGATAGCGGAACGGGAAGGCGACTGGAGTGCCATGTCCGGTTTTCAACAAACCAT
GCAAATGCTGAATGAGGGCATCGTTCCCACTGCGATGCTGGTTGCCAACGATCAGATG
GCGCTGGGCGCAATGCGCGCCATTACCGAGTCCGGGCTGCGCGTTGGTGCGGATATCT
CGGTAGTGGGATACGACGATACCGAAGACAGCTCATGTTATATCCCGCCGTTAACCACC
ATCAAACAGGATTTTCGCCTGCTGGGGCAAACCAGCGTGGACCGCTTGCTGCAACTCT
CTCAGGGCCAGGCGGTGAAGGGCAATCAGCTGTTGCCCGTCTCACTGGTGAAAAGAA
AAACCACCCTGGCGCCCAATACGCAAACCGCCTCTCCCCGCGCGTTGGCCGATTCATT
AATGCAGCTGGCACGACAGGTTTCCCGACTGGAAAGCGGGCAGAGCAGCCTGAGGCC
TCCTAAGAAGAAGAGGAAGGTTGCGGCCGCTAACCAATGTGCAGACTACTGTTAACC
AATGTGCAGACTACTGTGAATTCTAACCAATGTGCAGACTACTGTTAACCAATGTGCAG
ACTACTGTAAGCTTCGAATTGAGTAGTGCTTTCTACTTTATGAGTAGTGCTTTCTACTTT
ATGAGTAGTGCTTTCTACTTTATGAGTAGTGCTTTCTACTTTATGGTCGACAATACTAGT
TAAGAAATCGCCAAGCTACGGGCGGAGTACTGTCCTCCGAGCGGAGTACTGTCCTCCG
AGCGGAGTACTGTCCTCCGAGCGGAGTACTGTCCTCCGAGCGGAGTACTGTCCTCCGA
GCGGAGACTCTAGAAATTGTGAGCGGATAACAATTTAGGCGTGTACGGTGGGAGGCCT
ATATAAGCAGAGCTCGTTTAGTGAACCGTCAGATCGCCAGCTGACAAAATTGTGAGCG
CTCACAATTACTAGAAGATCTTGAATTCGCCACCATGGCTAGATTAGATAAAAGTAAAG
TGATTAACAGCGCATTAGAGCTGCTTAATGAGGTCGGAATCGAAGGTTTAACAACCCG
TAAACTCGCCCAGAAGCTAGGTGTAGAGCAGCCTACATTGTATTGGCATGTAAAAAATA
AGCGGGCTTTGCTCGACGCCTTAGCCATTGAGATGTTAGATAGGCACCATACTCACTTT
TGCCCTTTAGAAGGGGAAAGCTGGCAAGATTTTTTACGTAATAACGCTAAAAGTTTTA
GATGTGCTTTACTAAGTCATCGCGATGGAGCAAAAGTACATTTAGGTACACGGCCTACA
GAAAAACAGTATGAAACTCTCGAAAATCAATTAGCCTTTTTATGCCAACAAGGTTTTTC
ACTAGAGAATGCATTATATGCACTCAGCGCTGTGGGGCATTTTACTTTAGGTTGCGTATT
GGAAGATCAAGAGCATCAAGTCGCTAAAGAAGAAAGGGAAACACCTACTACTGATAG
TATGCCGCCATTATTACGACAAGCTATCGAATTATTTGATCACCAAGGTGCAGAGCCAG
CCTTCTTATTCGGCCTTGAATTGATCATATGCGGATTAGAAAAACAACTTAAATGTGAA
AGTGGGTCGCCAAAAAAGAAGAGAAAGGTCGACGGCGGTGGTGCTTTGTCTCCTCAG
CACTCTGCTGTCACTCAAGGAAGTATCATCAAGAACAAGGAGGGCATGGATGCTAAGT
CACTAACTGCCTGGTCCCGGACACTGGTGACCTTCAAGGATGTATTTGTGGACTTCAC
CAGGGAGGAGTGGAAGCTGCTGGACACTGCTCAGCAGATCGTGTACAGAAATGTGAT
GCTGGAGAACTATAAGAACCTGGTTTCCTTGGGTTATCAGCTTACTAAGCCAGATGTGA
TCCTCCGGTTGGAGAAGGGAGAAGAGCCCTGGCTGGTGGAGAGAGAAATTCACCAAG
AGACCCATCCTGATTCAGAGACTGCATTTGAAATCAAATCATCAGTTTAATACAAGGCG
GCCGCAAATCAACATCAGTCTGATAAGCTATCAACATCAGTCTGATAAGCTATCAACAT
CAGTCTGATAAGCTATCAACATCAGTCTGATAAGCTAAAGCTTCGAATTCTGATAATCA
GCCATACCACATTTGTAGAGGTTTTACTTGCTTTAAAAAACCTCCCACACCTCCCCCTG
AACCTGAAACATAAAATGAATGCAATTGTTGTTGTTAACTTGTTTATTGCAGCTTATAAT
GGTTACAAATAAAGCAATAGCATCACAAATTTCACAAATAAAGCATTTTTTTCACTGCA
GCTCCAGATTGAATTATTTGCCTGTCATACAGCTAATAATTGACCATAAGACAATTAGAT
TTAAATTAGTTTTGAATCTTTCTAATACCAAAGTTCAGTTTACTGTTCCATGTTGCTTCT
GAGTGGCTTCACAGACTTATGAAAAAGTAAACGGAATCAGAATTACATCAATGCAAAA
GCATTGCTGTGAACTCTGTACTTAGGACTAAACTTTGAGCAATAACACATATAGATTGA
GGATTGTTTGCTGTTAGTATACAAACTCTGGTTCAAAGCTCCTCTTTATTGCTTGTCTTG
GAAAATTTGCTGTTCTTCATGGTTTCTCTTTTCACTGCTATCTATTTTTCTCAACCACTC
ACATGGCTACAAAAGCTTCCTGATTAATAATTACACTAAGTCAATAGGCATAGAGCCAG
GACTGTTTGGGTAAACTGGTCACTTTATCTTAAACTAAATATATCCAAAACTGAACATGT
ACTTAGTTACTAAGTCTTTGACTTTATCTCATTCATACCACTCAGCTTTATCCAGGCCAC
TTATTTGACAGTATTATTGCGAAAACTTCCTATCTAGAAGTTTGAGGAGAATATTTGTTA
TATTTGCAAAATAAAATAAGTTTGCAAGTTTTTTTTTTCTGCCCCAAAGAGCTCTGTGT
CCTTGAACATAAAATACAAATAACCGCTATGCTGTTAATTATTGACAAATGTCCCATTTT
CAACCTAAGGAAATACCATAAAGTAACAGATATACCAACAAAAGGTTACTAGTTAACA
GGCATTGCCTGAAAAGAGTATAAAAGAATTTCAGCATGATTTTCCATATTGTGCTTCCA
CCACTGCCAATAACAAAATAACTAGCAAGGATCCGGCCACCATGGCCCCCCCGACCGA
TGTCAGCCTGGGGGACGAGCTCCACTTAGACGGCGAGGACGTGGCGATGGCGCATGC
CGACGCGCTAGACGATTTCGATCTGGACATGTTGGGGGACGGGGATTCCCCGGGTCCG
GGATTTACCCCCCACGACTCCGCCCCCTACGGCGCTCTGGATATGGCCGACTTCGAGTT
TGAGCAGATGTTTACCGATGCCCTTGGAATTGACGAGTACGGTGGGACGCGTATGAAG
CTACTGTCTTCTATCGAACAAGCATGCGATATTTGCCGACTTAAAAAGCTCAAGTGCTC
CAAAGAAAAACCGAAGTGCGCCAAGTGTCTGAAGAACAACTGGGAGTGTCGCTACTC
TCCCAAAACCAAAAGGTCTCCGCTGACTAGGGCACATCTGACAGAAGTGGAATCAAG
GCTAGAAAGACTGGAACAGCTATTTCTACTGATTTTTCCTCGAGAAGACCTTGACATGA
TTTTGAAAATGGATTCTTTACAGGATATAAAAGCATTGTTAACAGGATTATTTGTACAAG
ATAATGTGAATAAAGATGCCGTCACAGATAGATTGGCTTCAGTGGAGACTGATATGCCT
CTAACATTGAGACAGCATAGAATAAGTGCGACATCATCATCGGAAGAGAGTAGTAACA
AAGGTCAAAGACAGTTGACTGTATAATTCACTCCTCAGGTGCAGGCTGCCTATCAGAA
GGTGGTGGCTGGTGTGGCCAATGCCCTGGCTCACAAATACCACTGAGATCTTTTTCCCT
CTGCCAAAAATTATGGGGACATCATGAAGCCCCTTGAGCATCTGACTTCTGGCTAATAA
AGGAAATTTATTTTCATTGCAATAGTGTGTTGGAATTTTTTGTGTCTCTCACTCGGAAGG
ACATATGGGAGGGCAAATCATTTAAAACATCAGAATGAGTATTTGGTTTAGAGTTTGGC
AACATATGCCCATATGCTGGCTGCCATGAACAAAGGTTGGCTATAAAGAGGTCATCAGT
ATATGAAACAGCCCCCTGCTGTCCATTCCTTATTCCATAGAAAAGCCTTGACTTGAGGT
TAGATTTTTTTTATATTTTGTTTTGTGTTATTTTTTTCTTTAACATCCCTAAAATTTTCCTT
ACATGTTTTACTAGCCAGATTTTTCCTCCTCTCCTGACTACTCCCAGTCATAGCTGTCCC
TCTTCTCTTATGGAGATCGGAGAAAGAGGTAAT.
(SEQ ID NO: 10)
GTAACTATAACGGTCGCTCCGAATTTCTCGAGTTAATTAAGATTACGCCAAGCTACG
GGCGGAGTACTGTCCTCCGAGCGGAGTACTGTCCTCCGAGCGGAGTACTGTCCTCCGA
GCGGAGTACTGTCCTCCGAGCGGAGTTCTGTCCTCCGAGCGGAGACTCTAGACTCCCT
ATCAGTGATAGAGATAGGCGTGTACGGTGGGAGGCCTATATAAGCAGAGCTCGTTTAGT
GAACCGTCAGATCGCTCCCTATCAGTGATAGAGAGAATTCGACCGCCACCATGTCGGA
ATTCATGAGACATATTATCTGCCACGGAGGTGTTATTACCGAAGAAATGGCCGCCAGTC
TTTTGGACCAGCTGATCGAAGAGGTACTGGCTGATAATCTTCCACCTCCTAGCCATTTT
GAACCACCTACCCTTCACGAACTGTATGATTTAGACGTGACGGCCCCCGAAGATCCCA
ACGAGGAGGCGGTTTCGCAGATTTTTCCCGACTCTGTAATGTTGGCGGTGCAGGAAGG
GATTGACTTACTCACTTTTCCGCCGGCGCCCGGTTCTCCGGAGCCGCCTCACCTTTCCC
GGCAGCCCGAGCAGCCGGAGCAGAGAGCCTTGGGTCCGGTTTCTATGCCAAACCTTG
TACCGGAGGTGATCGATCTTACCTGCCACGAGGCTGGCTTTCCACCCAGTGACGACGA
GGATGAAGAGGGTGAGGAGTTTGTGTTAGATTATGTGGAGCACCCCGGGCACGGTTGC
AGGTCTTGTCATTATCACCGGAGGAATACGGGGGACCCAGATATTATGTGTTCGCTTTG
CTATATGAGGACCTGTGGCATGTTTGTCTACAGTAAGTGAAAATTATGGGCAGTGGGTG
ATAGAGTGGTGGGTTTGGTGTGGTAATTTTTTTTTTAATTTTTACAGTTTTGTGGTTTAA
AGAATTTTGTATTGTGATTTTTTTAAAAGGTCCTGTGTCTGAACCTGAGCCTGAGCCCG
AGCCAGAACCGGAGCCTGCAAGACCTACCCGCCGTCCTAAAATGGCGCCTGCTATCCT
GAGACGCCCGACATCACCTGTGTCTAGAGAATGCAATAGTAGTACGGATAGCTGTGAC
TCCGGTCCTTCTAACACACCTCCTGAGATACACCCGGTGGTCCCGCTGTGCCCCATTAA
ACCAGTTGCCGTGAGAGTTGGTGGGCGTCGCCAGGCTGTGGAATGTATCGAGGACTTG
CTTAACGAGCCTGGGCAACCTTTGGACTTGAGCTGTAAACGCCCCAGGCCACTCGAG
GGTACCGGGTCCGGAGCTACAAATTTTTCCCTCCTCAAACAGGCTGGAGATGTCGAAG
AAAATCCTGGGCCTACCGGTCCCATGTACAGGATGCAACTCCTGTCTTGCATTGCACTA
AGTCTTGCACTTGTCACAAACAGTGCACCTACTTCAAGTTCTACAAAGAAAACACAGC
TACAACTGGAGCATTTACTGCTGGATTTACAGATGATTTTGAATGGAATTAATAATTACA
AGAATCCCAAACTCACCGCGATGCTCACAGCTAAGTTTGCCATGCCCAAGAAGGCCAC
AGAACTGAAACATCTTCAGTGTCTAGAAGAAGCACTCAAACCTCTGGAGGAAGTG
CTAAATTTAGCTCAAAGCAAAAACTTTCACTTAAGACCCAGGGACTTAATCAGCAATAT
CAACGTAATAGTTCTGGAACTAAAGGGATCTGAAACAACATTCATGTGTGAATATGCTG
ATGAGACAGCAACCATTGTAGAATTTCTGAACAGATGGATTACCTTTTGTCAAAGCATC
ATCTCAACACTGACTTGAAGATCTGAGGGCCGCGGCAGCCTGCTGACCTGCGGCGAC
GTGGAGGAAAACCCCGGCCCCGGATCCATGAAACCAGTAACGTTATACGATGTCGCAG
AGTATGCCGGTGTCTCTTATCAGACCGTTTCCCGCGTGGTGAACCAGGCCAGCCACGT
TTCTGCGAAAACGCGGGAAAAAGTGGAAGCGGCGATGGCGGAGCTGAATTACATTCC
CAACCGCGTGGCACAACAACTGGCGGGCAAACAGTCGTTGCTGATTGGCGTTGCCAC
CTCCAGTCTGGCCCTGCACGCGCCGTCGCAAATTGTCGCGGCGATTAAATCTCGCGCC
GATCAACTGGGTGCCAGCGTGGTGGTGTCGATGGTAGAACGAAGCGGCGTCGAAGCC
TGTAAAGCGGCGGTGCACAATCTTCTCGCGCAACGCGTCAGTGGGCTGATCATTAACT
ATCCGCTGGATGACCAGGATGCCATTGCTGTGGAAGCTGCCTGCACTAATGTTCCGGC
GTTATTTCTTGATGTCTCTGACCAGACACCCATCAACAGTATTATTTTCTCCCATGAAGA
CGGTACGCGACTGGGCGTGGAGCATCTGGTCGCATTGGGTCACCAGCAAATCGCGCTG
TTAGCGGGCCCATTAAGTTCTGTCTCGGCGCGTCTGCGTCTGGCTGGCTGGCATAAATA
TCTCACTCGCAATCAAATTCAGCCGATAGCGGAACGGGAAGGCGACTGGAGTGCCATG
TCCGGTTTTCAACAAACCATGCAAATGCTGAATGAGGGCATCGTTCCCACTGCGATGC
TGGTTGCCAACGATCAGATGGCGCTGGGCGCAATGCGCGCCATTACCGAGTCCGGGCT
GCGCGTTGGTGCGGATATCTCGGTAGTGGGATACGACGATACCGAAGACAGCTCATGTT
ATATCCCGCCGTTAACCACCATCAAACAGGATTTTCGCCTGCTGGGGCAAACCAGCGT
GGACCGCTTGCTGCAACTCTCTCAGGGCCAGGCGGTGAAGGGCAATCAGCTGTTGCC
CGTCTCACTGGTGAAAAGAAAAACCACCCTGGCGCCCAATACGCAAACCGCCTCTCC
CCGCGCGTTGGCCGATTCATTAATGCAGCTGGCACGACAGGTTTCCCGACTGGAAAGC
GGGCAGAGCAGCCTGAGGCCTCCTAAGAAGAAGAGGAAGGTTGCGGCCGCTAACCA
ATGTGCAGACTACTGTTAACCAATGTGCAGACTACTGTGAATTCTAACCAATGTGCAGA
CTACTGTTAACCAATGTGCAGACTACTGTAAGCTTCGAATTGAGTAGTGCTTTCTACTTT
ATGAGTAGTGCTTTCTACTTTATGAGTAGTGCTTTCTACTTTATGAGTAGTGCTTTCTAC
TTTATGGTCGACAATACTAGTTAAGAAATCGCCAAGCTACGGGCGGAGTACTGTCCTCC
GAGCGGAGTACTGTCCTCCGAGCGGAGTACTGTCCTCCGAGCGGAGTACTGTCCTCCG
AGCGGAGTACTGTCCTCCGAGCGGAGACTCTAGAAATTGTGAGCGGATAACAATTTAG
GCGTGTACGGTGGGAGGCCTATATAAGCAGAGCTCGTTTAGTGAACCGTCAGATCGCC
AGCTGACAAAATTGTGAGCGCTCACAATTACTAGAAGATCTTGAATTCGCCACCATGG
CTAGATTAGATAAAAGTAAAGTGATTAACAGCGCATTAGAGCTGCTTAATGAGGTCGGA
ATCGAAGGTTTAACAACCCGTAAACTCGCCCAGAAGCTAGGTGTAGAGCAGCCTACAT
TGTATTGGCATGTAAAAAATAAGCGGGCTTTGCTCGACGCCTTAGCCATTGAGATGTTA
GATAGGCACCATACTCACTTTTGCCCTTTAGAAGGGGAAAGCTGGCAAGATTTTTTACG
TAATAACGCTAAAAGTTTTAGATGTGCTTTACTAAGTCATCGCGATGGAGCAAAAGTAC
ATTTAGGTACACGGCCTACAGAAAAACAGTATGAAACTCTCGAAAATCAATTAGCCTTT
TTATGCCAACAAGGTTTTTCACTAGAGAATGCATTATATGCACTCAGCGCTGTGGGGCA
TTTTACTTTAGGTTGCGTATTGGAAGATCAAGAGCATCAAGTCGCTAAAGAAGAAAGG
GAAACACCTACTACTGATAGTATGCCGCCATTATTACGACAAGCTATCGAATTATTTGAT
CACCAAGGTGCAGAGCCAGCCTTCTTATTCGGCCTTGAATTGATCATATGCGGATTAGA
AAAACAACTTAAATGTGAAAGTGGGTCGCCAAAAAAGAAGAGAAAGGTCGACGGCG
GTGGTGCTTTGTCTCCTCAGCACTCTGCTGTCACTCAAGGAAGTATCATCAAGAACAA
GGAGGGCATGGATGCTAAGTCACTAACTGCCTGGTCCCGGACACTGGTGACCTTCAAG
GATGTATTTGTGGACTTCACCAGGGAGGAGTGGAAGCTGCTGGACACTGCTCAGCAGA
TCGTGTACAGAAATGTGATGCTGGAGAACTATAAGAACCTGGTTTCCTTGGGTTATCAG
CTTACTAAGCCAGATGTGATCCTCCGGTTGGAGAAGGGAGAAGAGCCCTGGCTGGTG
GAGAGAGAAATTCACCAAGAGACCCATCCTGATTCAGAGACTGCATTTGAAATCAAAT
CATCAGTTTAATACAAGGCGGCCGCAAATCAACATCAGTCTGATAAGCTATCAACATCA
GTCTGATAAGCTATCAACATCAGTCTGATAAGCTATCAACATCAGTCTGATAAGCTAAA
GCTTCGAATTCTGATAATCAGCCATACCACATTTGTAGAGGTTTTACTTGCTTTAAAAAA
CCTCCCACACCTCCCCCTGAACCTGAAACATAAAATGAATGCAATTGTTGTTGTTAACT
TGTTTATTGCAGCTTATAATGGTTACAAATAAAGCAATAGCATCACAAATTTCACAAATA
AAGCATTTTTTTCACTGCAGCTCCAGATTGAATTATTTGCCTGTCATACAGCTAATAATT
GACCATAAGACAATTAGATTTAAATTAGTTTTGAATCTTTCTAATACCAAAGTTCAGTTT
ACTGTTCCATGTTGCTTCTGAGTGGCTTCACAGACTTATGAAAAAGTAAACGGAATCA
GAATTACATCAATGCAAAAGCATTGCTGTGAACTCTGTACTTAGGACTAAACTTTGAGC
AATAACACATATAGATTGAGGATTGTTTGCTGTTAGTATACAAACTCTGGTTCAAAGCT
CCTCTTTATTGCTTGTCTTGGAAAATTTGCTGTTCTTCATGGTTTCTCTTTTCACTGCTAT
CTATTTTTCTCAACCACTCACATGGCTACAAAAGCTTCCTGATTAATAATTACACTAAGT
CAATAGGCATAGAGCCAGGACTGTTTGGGTAAACTGGTCACTTTATCTTAAACTAAATA
TATCCAAAACTGAACATGTACTTAGTTACTAAGTCTTTGACTTTATCTCATTCATACCAC
TCAGCTTTATCCAGGCCACTTATTTGACAGTATTATTGCGAAAACTTCCTATCTAGAAGT
TTGAGGAGAATATTTGTTATATTTGCAAAATAAAATAAGTTTGCAAGTTTTTTTTTTCTG
CCCCAAAGAGCTCTGTGTCCTTGAACATAAAATACAAATAACCGCTATGCTGTTAATTA
TTGACAAATGTCCCATTTTCAACCTAAGGAAATACCATAAAGTAACAGATATACCAACA
AAAGGTTACTAGTTAACAGGCATTGCCTGAAAAGAGTATAAAAGAATTTCAGCATGAT
TTTCCATATTGTGCTTCCACCACTGCCAATAACAAAATAACTAGCAAGGATCCGGCCAC
CATGGCCCCCCCGACCGATGTCAGCCTGGGGGACGAGCTCCACTTAGACGGCGAGGA
CGTGGCGATGGCGCATGCCGACGCGCTAGACGATTTCGATCTGGACATGTTGGGGGAC
GGGGATTCCCCGGGTCCGGGATTTACCCCCCACGACTCCGCCCCCTACGGCGCTCTGG
ATATGGCCGACTTCGAGTTTGAGCAGATGTTTACCGATGCCCTTGGAATTGACGAGTAC
GGTGGGACGCGTATGAAGCTACTGTCTTCTATCGAACAAGCATGCGATATTTGCCGACT
TAAAAAGCTCAAGTGCTCCAAAGAAAAACCGAAGTGCGCCAAGTGTCTGAAGAACA
ACTGGGAGTGTCGCTACTCTCCCAAAACCAAAAGGTCTCCGCTGACTAGGGCACATCT
GACAGAAGTGGAATCAAGGCTAGAAAGACTGGAACAGCTATTTCTACTGATTTTTCCT
CGAGAAGACCTTGACATGATTTTGAAAATGGATTCTTTACAGGATATAAAAGCATTGTT
AACAGGATTATTTGTACAAGATAATGTGAATAAAGATGCCGTCACAGATAGATTGGCTT
CAGTGGAGACTGATATGCCTCTAACATTGAGACAGCATAGAATAAGTGCGACATCATCA
TCGGAAGAGAGTAGTAACAAAGGTCAAAGACAGTTGACTGTATAATTCACTCCTCAGG
TGCAGGCTGCCTATCAGAAGGTGGTGGCTGGTGTGGCCAATGCCCTGGCTCACAAATA
CCACTGAGATCTTTTTCCCTCTGCCAAAAATTATGGGGACATCATGAAGCCCCTTGAGC
ATCTGACTTCTGGCTAATAAAGGAAATTTATTTTCATTGCAATAGTGTGTTGGAATTTTT
TGTGTCTCTCACTCGGAAGGACATATGGGAGGGCAAATCATTTAAAACATCAGAATGA
GTATTTGGTTTAGAGTTTGGCAACATATGCCCATATGCTGGCTGCCATGAACAAAGGTT
GGCTATAAAGAGGTCATCAGTATATGAAACAGCCCCCTGCTGTCCATTCCTTATTCCATA
GAAAAGCCTTGACTTGAGGTTAGATTTTTTTTATATTTTGTTTTGTGTTATTTTTTTCTTT
AACATCCCTAAAATTTTCCTTACATGTTTTACTAGCCAGATTTTTCCTCCTCTCCTGACT
ACTCCCAGTCATAGCTGTCCCTCTTCTCTTATGGAGATCGGAGAAAGAGGTAAT.
(SEQ ID NO: 11)
GTAACTATAACGGTCGCTCCGAATTTCTCGAGTTAATTAAGATTACGCCAAGCTACG
GGCGGAGTACTGTCCTCCGAGCGGAGTACTGTCCTCCGAGCGGAGTACTGTCCTCCGA
GCGGAGTACTGTCCTCCGAGCGGAGTTCTGTCCTCCGAGCGGAGACTCTAGACTCCCT
ATCAGTGATAGAGATAGGCGTGTACGGTGGGAGGCCTATATAAGCAGAGCTCGTTTAGT
GAACCGTCAGATCGCTCCCTATCAGTGATAGAGAGAATTCGACCGCCACCATGTCGG
AATTCATGAGACATATTATCTGCCACGGAGGTGTTATTACCGAAGAAATGGCCGCCAGT
CTTTTGGACCAGCTGATCGAAGAGGTACTGGCTGATAATCTTCCACCTCCTAGCCATTT
TGAACCACCTACCCTTCACGAACTGTATGATTTAGACGTGACGGCCCCCGAAGATCCC
AACGAGGAGGCGGTTTCGCAGATTTTTCCCGACTCTGTAATGTTGGCGGTGCAGGAAG
GGATTGACTTACTCACTTTTCCGCCGGCGCCCGGTTCTCCGGAGCCGCCTCACCTTTCC
CGGCAGCCCGAGCAGCCGGAGCAGAGAGCCTTGGGTCCGGTTTCTATGCCAAACCTT
GTACCGGAGGTGATCGATCTTACCTGCCACGAGGCTGGCTTTCCACCCAGTGACGACG
AGGATGAAGAGGGTGAGGAGTTTGTGTTAGATTATGTGGAGCACCCCGGGCACGGTTG
CAGGTCTTGTCATTATCACCGGAGGAATACGGGGGACCCAGATATTATGTGTTCGCTTT
GCTATATGAGGACCTGTGGCATGTTTGTCTACAGTAAGTGAAAATTATGGGCAGTGGGT
GATAGAGTGGTGGGTTTGGTGTGGTAATTTTTTTTTTAATTTTTACAGTTTTGTGGTTTA
AAGAATTTTGTATTGTGATTTTTTTAAAAGGTCCTGTGTCTGAACCTGAGCCTGAGCCC
GAGCCAGAACCGGAGCCTGCAAGACCTACCCGCCGTCCTAAAATGGCGCCTGCTATCC
TGAGACGCCCGACATCACCTGTGTCTAGAGAATGCAATAGTAGTACGGATAGCTGTGA
CTCCGGTCCTTCTAACACACCTCCTGAGATACACCCGGTGGTCCCGCTGTGCCCCATTA
AACCAGTTGCCGTGAGAGTTGGTGGGCGTCGCCAGGCTGTGGAATGTATCGAGGACTT
GCTTAACGAGCCTGGGCAACCTTTGGACTTGAGCTGTAAACGCCCCAGGCCACTCGA
GGGTACCGGGTCCGGAGCTACAAATTTTTCCCTCCTCAAACAGGCTGGAGATGTCGAA
GAAAATCCTGGGCCTACCGGTCCCATGTGGCTGCAGAGCCTGCTGCTCTTGGGCACTG
TGGCCTGCAGCATCTCTGCACCCGCCCGCTCGCCCAGCCCCAGCACGCAGCCCTGGG
AGCATGTGAATGCCATCCAGGAGGCCCGGCGTCTCCTGAACCTGAGTAGAGACACTGC
TGCTGAGATGAATGAAACAGTAGAAGTCATCTCAGAAATGTTTGACCTCCAGGAGCCG
ACCTGCCTACAGACCCGCCTGGAGCTGTACAAGCAGGGCCTGCGGGGCAGCCTCACC
AAGCTCAAGGGCCCCTTGACCATGATGGCCAGCCACTACAAGCAGCACTGCCCTCCA
ACCCCGGAAACTTCCTGTGCAACCCAGATTATCACCTTTGAAAGTTTCAAAGAGAACC
TGAAGGACTTTCTGCTTGTCATCCCCTTTGACTGCTGGGAGCCAGTCCAGGAGTGAAG
ATCTGAGGGCCGCGGCAGCCTGCTGACCTGCGGCGACGTGGAGGAAAACCCCGGCCC
CGGATCCATGAAACCAGTAACGTTATACGATGTCGCAGAGTATGCCGGTGTCTCTTATC
AGACCGTTTCCCGCGTGGTGAACCAGGCCAGCCACGTTTCTGCGAAAACGCGGGAAA
AAGTGGAAGCGGCGATGGCGGAGCTGAATTACATTCCCAACCGCGTGGCACAACAAC
TGGCGGGCAAACAGTCGTTGCTGATTGGCGTTGCCACCTCCAGTCTGGCCCTGCACGC
GCCGTCGCAAATTGTCGCGGCGATTAAATCTCGCGCCGATCAACTGGGTGCCAGCGTG
GTGGTGTCGATGGTAGAACGAAGCGGCGTCGAAGCCTGTAAAGCGGCGGTGCACAAT
CTTCTCGCGCAACGCGTCAGTGGGCTGATCATTAACTATCCGCTGGATGACCAGGATGC
CATTGCTGTGGAAGCTGCCTGCACTAATGTTCCGGCGTTATTTCTTGATGTCTCTGACC
AGACACCCATCAACAGTATTATTTTCTCCCATGAAGACGGTACGCGACTGGGCGTGGA
GCATCTGGTCGCATTGGGTCACCAGCAAATCGCGCTGTTAGCGGGCCCATTAAGTTCT
GTCTCGGCGCGTCTGCGTCTGGCTGGCTGGCATAAATATCTCACTCGCAATCAAATTCA
GCCGATAGCGGAACGGGAAGGCGACTGGAGTGCCATGTCCGGTTTTCAACAAACCAT
GCAAATGCTGAATGAGGGCATCGTTCCCACTGCGATGCTGGTTGCCAACGATCAGATG
GCGCTGGGCGCAATGCGCGCCATTACCGAGTCCGGGCTGCGCGTTGGTGCGGATATCT
CGGTAGTGGGATACGACGATACCGAAGACAGCTCATGTTATATCCCGCCGTTAACCACC
ATCAAACAGGATTTTCGCCTGCTGGGGCAAACCAGCGTGGACCGCTTGCTGCAACTCT
CTCAGGGCCAGGCGGTGAAGGGCAATCAGCTGTTGCCCGTCTCACTGGTGAAAAGAA
AAACCACCCTGGCGCCCAATACGCAAACCGCCTCTCCCCGCGCGTTGGCCGATTCATT
AATGCAGCTGGCACGACAGGTTTCCCGACTGGAAAGCGGGCAGAGCAGCCTGAGGCC
TCCTAAGAAGAAGAGGAAGGTTGCGGCCGCTAACCAATGTGCAGACTACTGTTAACC
AATGTGCAGACTACTGTGAATTCTAACCAATGTGCAGACTACTGTTAACCAATGTGCAG
ACTACTGTAAGCTTCGAATTGAGTAGTGCTTTCTACTTTATGAGTAGTGCTTTCTACTTT
ATGAGTAGTGCTTTCTACTTTATGAGTAGTGCTTTCTACTTTATGGTCGACAATACTAGT
TAAGAAATCGCCAAGCTACGGGCGGAGTACTGTCCTCCGAGCGGAGTACTGTCCTCCG
AGCGGAGTACTGTCCTCCGAGCGGAGTACTGTCCTCCGAGCGGAGTACTGTCCTCCGA
GCGGAGACTCTAGAAATTGTGAGCGGATAACAATTTAGGCGTGTACGGTGGGAGGCCT
ATATAAGCAGAGCTCGTTTAGTGAACCGTCAGATCGCCAGCTGACAAAATTGTGAGCG
CTCACAATTACTAGAAGATCTTGAATTCGCCACCATGGCTAGATTAGATAAAAGTAAAG
TGATTAACAGCGCATTAGAGCTGCTTAATGAGGTCGGAATCGAAGGTTTAACAACCCG
TAAACTCGCCCAGAAGCTAGGTGTAGAGCAGCCTACATTGTATTGGCATGTAAAAAATA
AGCGGGCTTTGCTCGACGCCTTAGCCATTGAGATGTTAGATAGGCACCATACTCACTTT
TGCCCTTTAGAAGGGGAAAGCTGGCAAGATTTTTTACGTAATAACGCTAAAAGTTTTA
GATGTGCTTTACTAAGTCATCGCGATGGAGCAAAAGTACATTTAGGTACACGGCCTACA
GAAAAACAGTATGAAACTCTCGAAAATCAATTAGCCTTTTTATGCCAACAAGGTTTTTC
ACTAGAGAATGCATTATATGCACTCAGCGCTGTGGGGCATTTTACTTTAGGTTGCGTATT
GGAAGATCAAGAGCATCAAGTCGCTAAAGAAGAAAGGGAAACACCTACTACTGATAG
TATGCCGCCATTATTACGACAAGCTATCGAATTATTTGATCACCAAGGTGCAGAGCCAG
CCTTCTTATTCGGCCTTGAATTGATCATATGCGGATTAGAAAAACAACTTAAATGTGAA
AGTGGGTCGCCAAAAAAGAAGAGAAAGGTCGACGGCGGTGGTGCTTTGTCTCCTCAG
CACTCTGCTGTCACTCAAGGAAGTATCATCAAGAACAAGGAGGGCATGGATGCTAAGT
CACTAACTGCCTGGTCCCGGACACTGGTGACCTTCAAGGATGTATTTGTGGACTTCAC
CAGGGAGGAGTGGAAGCTGCTGGACACTGCTCAGCAGATCGTGTACAGAAATGTGAT
GCTGGAGAACTATAAGAACCTGGTTTCCTTGGGTTATCAGCTTACTAAGCCAGATGTGA
TCCTCCGGTTGGAGAAGGGAGAAGAGCCCTGGCTGGTGGAGAGAGAAATTCACCAAG
AGACCCATCCTGATTCAGAGACTGCATTTGAAATCAAATCATCAGTTTAATACAAGGCG
GCCGCAAATCAACATCAGTCTGATAAGCTATCAACATCAGTCTGATAAGCTATCAACAT
CAGTCTGATAAGCTATCAACATCAGTCTGATAAGCTAAAGCTTCGAATTCTGATAATCA
GCCATACCACATTTGTAGAGGTTTTACTTGCTTTAAAAAACCTCCCACACCTCCCCCTG
AACCTGAAACATAAAATGAATGCAATTGTTGTTGTTAACTTGTTTATTGCAGCTTATAAT
GGTTACAAATAAAGCAATAGCATCACAAATTTCACAAATAAAGCATTTTTTTCACTGCA
GCTCCAGATTGAATTATTTGCCTGTCATACAGCTAATAATTGACCATAAGACAATTAGAT
TTAAATTAGTTTTGAATCTTTCTAATACCAAAGTTCAGTTTACTGTTCCATGTTGCTTCT
GAGTGGCTTCACAGACTTATGAAAAAGTAAACGGAATCAGAATTACATCAATGCAAAA
GCATTGCTGTGAACTCTGTACTTAGGACTAAACTTTGAGCAATAACACATATAGATTGA
GGATTGTTTGCTGTTAGTATACAAACTCTGGTTCAAAGCTCCTCTTTATTGCTTGTCTTG
GAAAATTTGCTGTTCTTCATGGTTTCTCTTTTCACTGCTATCTATTTTTCTCAACCACTC
ACATGGCTACAAAAGCTTCCTGATTAATAATTACACTAAGTCAATAGGCATAGAGCCAG
GACTGTTTGGGTAAACTGGTCACTTTATCTTAAACTAAATATATCCAAAACTGAACATGT
ACTTAGTTACTAAGTCTTTGACTTTATCTCATTCATACCACTCAGCTTTATCCAGGCCAC
TTATTTGACAGTATTATTGCGAAAACTTCCTATCTAGAAGTTTGAGGAGAATATTTGTTA
TATTTGCAAAATAAAATAAGTTTGCAAGTTTTTTTTTTCTGCCCCAAAGAGCTCTGTGT
CCTTGAACATAAAATACAAATAACCGCTATGCTGTTAATTATTGACAAATGTCCCATTTT
CAACCTAAGGAAATACCATAAAGTAACAGATATACCAACAAAAGGTTACTAGTTAACA
GGCATTGCCTGAAAAGAGTATAAAAGAATTTCAGCATGATTTTCCATATTGTGCTTCCA
CCACTGCCAATAACAAAATAACTAGCAAGGATCCGGCCACCATGGCCCCCCCGACCGA
TGTCAGCCTGGGGGACGAGCTCCACTTAGACGGCGAGGACGTGGCGATGGCGCATGC
CGACGCGCTAGACGATTTCGATCTGGACATGTTGGGGGACGGGGATTCCCCGGGTCCG
GGATTTACCCCCCACGACTCCGCCCCCTACGGCGCTCTGGATATGGCCGACTTCGAGTT
TGAGCAGATGTTTACCGATGCCCTTGGAATTGACGAGTACGGTGGGACGCGTATGAAG
CTACTGTCTTCTATCGAACAAGCATGCGATATTTGCCGACTTAAAAAGCTCAAGTGCTC
CAAAGAAAAACCGAAGTGCGCCAAGTGTCTGAAGAACAACTGGGAGTGTCGCTACTC
TCCCAAAACCAAAAGGTCTCCGCTGACTAGGGCACATCTGACAGAAGTGGAATCAAG
GCTAGAAAGACTGGAACAGCTATTTCTACTGATTTTTCCTCGAGAAGACCTTGACATGA
TTTTGAAAATGGATTCTTTACAGGATATAAAAGCATTGTTAACAGGATTATTTGTACAAG
ATAATGTGAATAAAGATGCCGTCACAGATAGATTGGCTTCAGTGGAGACTGATATGCCT
CTAACATTGAGACAGCATAGAATAAGTGCGACATCATCATCGGAAGAGAGTAGTAACA
AAGGTCAAAGACAGTTGACTGTATAATTCACTCCTCAGGTGCAGGCTGCCTATCAGAA
GGTGGTGGCTGGTGTGGCCAATGCCCTGGCTCACAAATACCACTGAGATCTTTTTCCCT
CTGCCAAAAATTATGGGGACATCATGAAGCCCCTTGAGCATCTGACTTCTGGCTAATAA
AGGAAATTTATTTTCATTGCAATAGTGTGTTGGAATTTTTTGTGTCTCTCACTCGGAAGG
ACATATGGGAGGGCAAATCATTTAAAACATCAGAATGAGTATTTGGTTTAGAGTTTGGC
AACATATGCCCATATGCTGGCTGCCATGAACAAAGGTTGGCTATAAAGAGGTCATCAGT
ATATGAAACAGCCCCCTGCTGTCCATTCCTTATTCCATAGAAAAGCCTTGACTTGAGGT
TAGATTTTTTTTATATTTTGTTTTGTGTTATTTTTTTCTTTAACATCCCTAAAATTTTCCTT
ACATGTTTTACTAGCCAGATTTTTCCTCCTCTCCTGACTACTCCCAGTCATAGCTGTCCC
TCTTCTCTTATGGAGATCGGAGAAAGAGGTAAT.
(SEQ ID NO: 12)
GTAACTATAACGGTCGCTCCGAATTTCTCGAGTTAATTAAGATTACGCCAAGCTACG
GGCGGAGTACTGTCCTCCGAGCGGAGTACTGTCCTCCGAGCGGAGTACTGTCCTCCGA
GCGGAGTACTGTCCTCCGAGCGGAGTTCTGTCCTCCGAGCGGAGACTCTAGACTCCCT
ATCAGTGATAGAGATAGGCGTGTACGGTGGGAGGCCTATATAAGCAGAGCTCGTTTAGT
GAACCGTCAGATCGCTCCCTATCAGTGATAGAGAGAATTCGACCGCCACCATGTCGGA
ATTCATGAGACATATTATCTGCCACGGAGGTGTTATTACCGAAGAAATGGCCGCCAGTC
TTTTGGACCAGCTGATCGAAGAGGTACTGGCTGATAATCTTCCACCTCCTAGCCATTTT
GAACCACCTACCCTTCACGAACTGTATGATTTAGACGTGACGGCCCCCGAAGATCCCA
ACGAGGAGGCGGTTTCGCAGATTTTTCCCGACTCTGTAATGTTGGCGGTGCAGGAAGG
GATTGACTTACTCACTTTTCCGCCGGCGCCCGGTTCTCCGGAGCCGCCTCACCTTTCCC
GGCAGCCCGAGCAGCCGGAGCAGAGAGCCTTGGGTCCGGTTTCTATGCCAAACCTTG
TACCGGAGGTGATCGATCTTACCTGCCACGAGGCTGGCTTTCCACCCAGTGACGACGA
GGATGAAGAGGGTGAGGAGTTTGTGTTAGATTATGTGGAGCACCCCGGGCACGGTTGC
AGGTCTTGTCATTATCACCGGAGGAATACGGGGGACCCAGATATTATGTGTTCGCTTTG
CTATATGAGGACCTGTGGCATGTTTGTCTACAGTAAGTGAAAATTATGGGCAGTGGGT
GATAGAGTGGTGGGTTTGGTGTGGTAATTTTTTTTTTAATTTTTACAGTTTTGTGGTTTA
AAGAATTTTGTATTGTGATTTTTTTAAAAGGTCCTGTGTCTGAACCTGAGCCTGAGCCC
GAGCCAGAACCGGAGCCTGCAAGACCTACCCGCCGTCCTAAAATGGCGCCTGCTATCC
TGAGACGCCCGACATCACCTGTGTCTAGAGAATGCAATAGTAGTACGGATAGCTGTGA
CTCCGGTCCTTCTAACACACCTCCTGAGATACACCCGGTGGTCCCGCTGTGCCCCATTA
AACCAGTTGCCGTGAGAGTTGGTGGGCGTCGCCAGGCTGTGGAATGTATCGAGGACTT
GCTTAACGAGCCTGGGCAACCTTTGGACTTGAGCTGTAAACGCCCCAGGCCACTCGA
GGGTACCGGGTCCGGAGCTACAAATTTTTCCCTCCTCAAACAGGCTGGAGATGTCGAA
GAAAATCCTGGGCCTACCGGTCCCATGTGGCTGCAGAATTTACTTTTCCTGGGCATTGT
GGTCTACAGCCTCTCAGCACCCACCCGCTCACCCATCACTGTCACCCGGCCTTGGAAG
CATGTAGAGGCCATCAAAGAAGCCCTGAACCTCCTGGATGACATGCCTGTCACGTTGA
ATGAAGAGGTAGAAGTCGTCTCTAACGAGTTCTCCTTCAAGAAGCTAACATGTGTGCA
GACCCGCCTGAAGATATTCGAGCAGGGTCTACGGGGCAATTTCACCAAACTCAAGGGC
GCCTTGAACATGACAGCCAGCTACTACCAGACATACTGCCCCCCAACTCCGGAAACGG
ACTGTGAAACACAAGTTACCACCTATGCGGATTTCATAGACAGCCTTAAAACCTTTCTG
ACTGATATCCCCTTTGAATGCAAAAAACCAGGCCAAAAATGAAGATCTGAGGGCCGCG
GCAGCCTGCTGACCTGCGGCGACGTGGAGGAAAACCCCGGCCCCGGATCCATGAAAC
CAGTAACGTTATACGATGTCGCAGAGTATGCCGGTGTCTCTTATCAGACCGTTTCCCGC
GTGGTGAACCAGGCCAGCCACGTTTCTGCGAAAACGCGGGAAAAAGTGGAAGCGGC
GATGGCGGAGCTGAATTACATTCCCAACCGCGTGGCACAACAACTGGCGGGCAAACA
GTCGTTGCTGATTGGCGTTGCCACCTCCAGTCTGGCCCTGCACGCGCCGTCGCAAATT
GTCGCGGCGATTAAATCTCGCGCCGATCAACTGGGTGCCAGCGTGGTGGTGTCGATGG
TAGAACGAAGCGGCGTCGAAGCCTGTAAAGCGGCGGTGCACAATCTTCTCGCGCAAC
GCGTCAGTGGGCTGATCATTAACTATCCGCTGGATGACCAGGATGCCATTGCTGTGGAA
GCTGCCTGCACTAATGTTCCGGCGTTATTTCTTGATGTCTCTGACCAGACACCCATCAA
CAGTATTATTTTCTCCCATGAAGACGGTACGCGACTGGGCGTGGAGCATCTGGTCGCAT
TGGGTCACCAGCAAATCGCGCTGTTAGCGGGCCCATTAAGTTCTGTCTCGGCGCGTCT
GCGTCTGGCTGGCTGGCATAAATATCTCACTCGCAATCAAATTCAGCCGATAGCGGAAC
GGGAAGGCGACTGGAGTGCCATGTCCGGTTTTCAACAAACCATGCAAATGCTGAATGA
GGGCATCGTTCCCACTGCGATGCTGGTTGCCAACGATCAGATGGCGCTGGGCGCAATG
CGCGCCATTACCGAGTCCGGGCTGCGCGTTGGTGCGGATATCTCGGTAGTGGGATACG
ACGATACCGAAGACAGCTCATGTTATATCCCGCCGTTAACCACCATCAAACAGGATTTT
CGCCTGCTGGGGCAAACCAGCGTGGACCGCTTGCTGCAACTCTCTCAGGGCCAGGCG
GTGAAGGGCAATCAGCTGTTGCCCGTCTCACTGGTGAAAAGAAAAACCACCCTGGCG
CCCAATACGCAAACCGCCTCTCCCCGCGCGTTGGCCGATTCATTAATGCAGCTGGCAC
GACAGGTTTCCCGACTGGAAAGCGGGCAGAGCAGCCTGAGGCCTCCTAAGAAGAAG
AGGAAGGTTGCGGCCGCTAACCAATGTGCAGACTACTGTTAACCAATGTGCAGACTAC
TGTGAATTCTAACCAATGTGCAGACTACTGTTAACCAATGTGCAGACTACTGTAAGCTT
CGAATTGAGTAGTGCTTTCTACTTTATGAGTAGTGCTTTCTACTTTATGAGTAGTGCTTT
CTACTTTATGAGTAGTGCTTTCTACTTTATGGTCGACAATACTAGTTAAGAAATCGCCAA
GCTACGGGCGGAGTACTGTCCTCCGAGCGGAGTACTGTCCTCCGAGCGGAGTACTGTC
CTCCGAGCGGAGTACTGTCCTCCGAGCGGAGTACTGTCCTCCGAGCGGAGACTCTAGA
AATTGTGAGCGGATAACAATTTAGGCGTGTACGGTGGGAGGCCTATATAAGCAGAGCT
CGTTTAGTGAACCGTCAGATCGCCAGCTGACAAAATTGTGAGCGCTCACAATTACTAG
AAGATCTTGAATTCGCCACCATGGCTAGATTAGATAAAAGTAAAGTGATTAACAGCGCA
TTAGAGCTGCTTAATGAGGTCGGAATCGAAGGTTTAACAACCCGTAAACTCGCCCAGA
AGCTAGGTGTAGAGCAGCCTACATTGTATTGGCATGTAAAAAATAAGCGGGCTTTGCTC
GACGCCTTAGCCATTGAGATGTTAGATAGGCACCATACTCACTTTTGCCCTTTAGAAGG
GGAAAGCTGGCAAGATTTTTTACGTAATAACGCTAAAAGTTTTAGATGTGCTTTACTAA
GTCATCGCGATGGAGCAAAAGTACATTTAGGTACACGGCCTACAGAAAAACAGTATGA
AACTCTCGAAAATCAATTAGCCTTTTTATGCCAACAAGGTTTTTCACTAGAGAATGCAT
TATATGCACTCAGCGCTGTGGGGCATTTTACTTTAGGTTGCGTATTGGAAGATCAAGAG
CATCAAGTCGCTAAAGAAGAAAGGGAAACACCTACTACTGATAGTATGCCGCCATTATT
ACGACAAGCTATCGAATTATTTGATCACCAAGGTGCAGAGCCAGCCTTCTTATTCGGCC
TTGAATTGATCATATGCGGATTAGAAAAACAACTTAAATGTGAAAGTGGGTCGCCAAA
AAAGAAGAGAAAGGTCGACGGCGGTGGTGCTTTGTCTCCTCAGCACTCTGCTGTCAC
TCAAGGAAGTATCATCAAGAACAAGGAGGGCATGGATGCTAAGTCACTAACTGCCTGG
TCCCGGACACTGGTGACCTTCAAGGATGTATTTGTGGACTTCACCAGGGAGGAGTGGA
AGCTGCTGGACACTGCTCAGCAGATCGTGTACAGAAATGTGATGCTGGAGAACTATAA
GAACCTGGTTTCCTTGGGTTATCAGCTTACTAAGCCAGATGTGATCCTCCGGTTGGAGA
AGGGAGAAGAGCCCTGGCTGGTGGAGAGAGAAATTCACCAAGAGACCCATCCTGATT
CAGAGACTGCATTTGAAATCAAATCATCAGTTTAATACAAGGCGGCCGCAAATCAACA
TCAGTCTGATAAGCTATCAACATCAGTCTGATAAGCTATCAACATCAGTCTGATAAGCTA
TCAACATCAGTCTGATAAGCTAAAGCTTCGAATTCTGATAATCAGCCATACCACATTTG
TAGAGGTTTTACTTGCTTTAAAAAACCTCCCACACCTCCCCCTGAACCTGAAACATAAA
ATGAATGCAATTGTTGTTGTTAACTTGTTTATTGCAGCTTATAATGGTTACAAATAAAGC
AATAGCATCACAAATTTCACAAATAAAGCATTTTTTTCACTGCAGCTCCAGATTGAATTA
TTTGCCTGTCATACAGCTAATAATTGACCATAAGACAATTAGATTTAAATTAGTTTTGAA
TCTTTCTAATACCAAAGTTCAGTTTACTGTTCCATGTTGCTTCTGAGTGGCTTCACAGA
CTTATGAAAAAGTAAACGGAATCAGAATTACATCAATGCAAAAGCATTGCTGTGAACT
CTGTACTTAGGACTAAACTTTGAGCAATAACACATATAGATTGAGGATTGTTTGCTGTTA
GTATACAAACTCTGGTTCAAAGCTCCTCTTTATTGCTTGTCTTGGAAAATTTGCTGTTCT
TCATGGTTTCTCTTTTCACTGCTATCTATTTTTCTCAACCACTCACATGGCTACAAAAGC
TTCCTGATTAATAATTACACTAAGTCAATAGGCATAGAGCCAGGACTGTTTGGGTAAAC
TGGTCACTTTATCTTAAACTAAATATATCCAAAACTGAACATGTACTTAGTTACTAAGTC
TTTGACTTTATCTCATTCATACCACTCAGCTTTATCCAGGCCACTTATTTGACAGTATTAT
TGCGAAAACTTCCTATCTAGAAGTTTGAGGAGAATATTTGTTATATTTGCAAAATAAAAT
AAGTTTGCAAGTTTTTTTTTTCTGCCCCAAAGAGCTCTGTGTCCTTGAACATAAAATAC
AAATAACCGCTATGCTGTTAATTATTGACAAATGTCCCATTTTCAACCTAAGGAAATACC
ATAAAGTAACAGATATACCAACAAAAGGTTACTAGTTAACAGGCATTGCCTGAAAAGA
GTATAAAAGAATTTCAGCATGATTTTCCATATTGTGCTTCCACCACTGCCAATAACAAAA
TAACTAGCAAGGATCCGGCCACCATGGCCCCCCCGACCGATGTCAGCCTGGGGGACG
AGCTCCACTTAGACGGCGAGGACGTGGCGATGGCGCATGCCGACGCGCTAGACGATTT
CGATCTGGACATGTTGGGGGACGGGGATTCCCCGGGTCCGGGATTTACCCCCCACGAC
TCCGCCCCCTACGGCGCTCTGGATATGGCCGACTTCGAGTTTGAGCAGATGTTTACCGA
TGCCCTTGGAATTGACGAGTACGGTGGGACGCGTATGAAGCTACTGTCTTCTATCGAA
CAAGCATGCGATATTTGCCGACTTAAAAAGCTCAAGTGCTCCAAAGAAAAACCGAAG
TGCGCCAAGTGTCTGAAGAACAACTGGGAGTGTCGCTACTCTCCCAAAACCAAAAGG
TCTCCGCTGACTAGGGCACATCTGACAGAAGTGGAATCAAGGCTAGAAAGACTGGAA
CAGCTATTTCTACTGATTTTTCCTCGAGAAGACCTTGACATGATTTTGAAAATGGATTCT
TTACAGGATATAAAAGCATTGTTAACAGGATTATTTGTACAAGATAATGTGAATAAAGAT
GCCGTCACAGATAGATTGGCTTCAGTGGAGACTGATATGCCTCTAACATTGAGACAGCA
TAGAATAAGTGCGACATCATCATCGGAAGAGAGTAGTAACAAAGGTCAAAGACAGTT
GACTGTATAATTCACTCCTCAGGTGCAGGCTGCCTATCAGAAGGTGGTGGCTGGTGTG
GCCAATGCCCTGGCTCACAAATACCACTGAGATCTTTTTCCCTCTGCCAAAAATTATGG
GGACATCATGAAGCCCCTTGAGCATCTGACTTCTGGCTAATAAAGGAAATTTATTTTCA
TTGCAATAGTGTGTTGGAATTTTTTGTGTCTCTCACTCGGAAGGACATATGGGAGGGCA
AATCATTTAAAACATCAGAATGAGTATTTGGTTTAGAGTTTGGCAACATATGCCCATATG
CTGGCTGCCATGAACAAAGGTTGGCTATAAAGAGGTCATCAGTATATGAAACAGCCCC
CTGCTGTCCATTCCTTATTCCATAGAAAAGCCTTGACTTGAGGTTAGATTTTTTTTATATT
TTGTTTTGTGTTATTTTTTTCTTTAACATCCCTAAAATTTTCCTTACATGTTTTACTAGCC
AGATTTTTCCTCCTCTCCTGACTACTCCCAGTCATAGCTGTCCCTCTTCTCTTATGGAGA
TCGGAGAAAGAGGTAAT.
(SEQ ID NO: 13)
GTAACTATAACGGTCGCTCCGAATTTCTCGAGTTAATTAAGATTACGCCAAGCTACG
GGCGGAGTACTGTCCTCCGAGCGGAGTACTGTCCTCCGAGCGGAGTACTGTCCTCCGA
GCGGAGTACTGTCCTCCGAGCGGAGTTCTGTCCTCCGAGCGGAGACTCTAGACTCCCT
ATCAGTGATAGAGATAGGCGTGTACGGTGGGAGGCCTATATAAGCAGAGCTCGTTTAGT
GAACCGTCAGATCGCTCCCTATCAGTGATAGAGAGAATTCGACCGCCACCATGTCGGA
ATTCATGAGACATATTATCTGCCACGGAGGTGTTATTACCGAAGAAATGGCCGCCAGTC
TTTTGGACCAGCTGATCGAAGAGGTACTGGCTGATAATCTTCCACCTCCTAGCCATTTT
GAACCACCTACCCTTCACGAACTGTATGATTTAGACGTGACGGCCCCCGAAGATCCCA
ACGAGGAGGCGGTTTCGCAGATTTTTCCCGACTCTGTAATGTTGGCGGTGCAGGAAGG
GATTGACTTACTCACTTTTCCGCCGGCGCCCGGTTCTCCGGAGCCGCCTCACCTTTCCC
GGCAGCCCGAGCAGCCGGAGCAGAGAGCCTTGGGTCCGGTTTCTATGCCAAACCTTG
TACCGGAGGTGATCGATCTTACCTGCCACGAGGCTGGCTTTCCACCCAGTGACGACGA
GGATGAAGAGGGTGAGGAGTTTGTGTTAGATTATGTGGAGCACCCCGGGCACGGTTGC
AGGTCTTGTCATTATCACCGGAGGAATACGGGGGACCCAGATATTATGTGTTCGCTTTG
CTATATGAGGACCTGTGGCATGTTTGTCTACAGTAAGTGAAAATTATGGGCAGTGGGTG
ATAGAGTGGTGGGTTTGGTGTGGTAATTTTTTTTTTAATTTTTACAGTTTTGTGGTTTAA
AGAATTTTGTATTGTGATTTTTTTAAAAGGTCCTGTGTCTGAACCTGAGCCTGAGCCCG
AGCCAGAACCGGAGCCTGCAAGACCTACCCGCCGTCCTAAAATGGCGCCTGCTATCCT
GAGACGCCCGACATCACCTGTGTCTAGAGAATGCAATAGTAGTACGGATAGCTGTGAC
TCCGGTCCTTCTAACACACCTCCTGAGATACACCCGGTGGTCCCGCTGTGCCCCATTAA
ACCAGTTGCCGTGAGAGTTGGTGGGCGTCGCCAGGCTGTGGAATGTATCGAGGACTTG
CTTAACGAGCCTGGGCAACCTTTGGACTTGAGCTGTAAACGCCCCAGGCCACTCGAG
GGTACCGGGTCCGGAGCTACAAATTTTTCCCTCCTCAAACAGGCTGGAGATGTCGAAG
AAAATCCTGGGCCTACCGGTCCCATGAGTGTGCCCACTCAGGTCCTGGGGTTGCTGCT
GCTGTGGCTTACAGACGCTCGCTGCCAGGTGCAGCTGGTGGAGAGCGGCGGCGGCG
TGGTGCAGCCCGGCAGGAGCCTGAGGCTGGACTGCAAGGCCAGCGGCATCACCTTCA
GCAACAGCGGCATGCACTGGGTGAGGCAGGCCCCCGGCAAGGGCCTGGAGTGGGTG
GCCGTGATCTGGTACGACGGCAGCAAGAGGTACTACGCCGACAGCGTGAAGGGCAGG
TTCACCATCAGCAGGGACAACAGCAAGAACACCCTGTTCCTGCAGATGAACAGCCTG
AGGGCCGAGGACACCGCCGTGTACTACTGCGCCACCAACGACGACTACTGGGGCCAG
GGCACCCTGGTGACCGTGAGCAGCGGTGGAGGCGGTTCAGGCGGAGGTGGCTCTGGC
GGTGGCGGATCGGAGATCGTGCTGACCCAGAGCCCCGCCACCCTGAGCCTGAGCCCC
GGCGAGAGGGCCACCCTGAGCTGCAGGGCCAGCCAGAGCGTGAGCAGCTACCTGGC
CTGGTACCAGCAGAAGCCCGGCCAGGCCCCCAGGCTGCTGATCTACGACGCCAGCAA
CAGGGCCACCGGCATCCCCGCCAGGTTCAGCGGCAGCGGCAGCGGCACCGACTTCAC
CCTGACCATCAGCAGCCTGGAGCCCGAGGACTTCGCCGTGTACTACTGCCAGCAGAG
CAGCAACTGGCCCAGGACCTTCGGCCAGGGCACCAAGGTGGAGATCAAGAGATCTGA
GGGCCGCGGCAGCCTGCTGACCTGCGGCGACGTGGAGGAAAACCCCGGCCCCGGATC
CATGAAACCAGTAACGTTATACGATGTCGCAGAGTATGCCGGTGTCTCTTATCAGACCG
TTTCCCGCGTGGTGAACCAGGCCAGCCACGTTTCTGCGAAAACGCGGGAAAAAGTGG
AAGCGGCGATGGCGGAGCTGAATTACATTCCCAACCGCGTGGCACAACAACTGGCGG
GCAAACAGTCGTTGCTGATTGGCGTTGCCACCTCCAGTCTGGCCCTGCACGCGCCGTC
GCAAATTGTCGCGGCGATTAAATCTCGCGCCGATCAACTGGGTGCCAGCGTGGTGGTG
TCGATGGTAGAACGAAGCGGCGTCGAAGCCTGTAAAGCGGCGGTGCACAATCTTCTC
GCGCAACGCGTCAGTGGGCTGATCATTAACTATCCGCTGGATGACCAGGATGCCATTG
CTGTGGAAGCTGCCTGCACTAATGTTCCGGCGTTATTTCTTGATGTCTCTGACCAGACA
CCCATCAACAGTATTATTTTCTCCCATGAAGACGGTACGCGACTGGGCGTGGAGCATCT
GGTCGCATTGGGTCACCAGCAAATCGCGCTGTTAGCGGGCCCATTAAGTTCTGTCTCG
GCGCGTCTGCGTCTGGCTGGCTGGCATAAATATCTCACTCGCAATCAAATTCAGCCGAT
AGCGGAACGGGAAGGCGACTGGAGTGCCATGTCCGGTTTTCAACAAACCATGCAAAT
GCTGAATGAGGGCATCGTTCCCACTGCGATGCTGGTTGCCAACGATCAGATGGCGCTG
GGCGCAATGCGCGCCATTACCGAGTCCGGGCTGCGCGTTGGTGCGGATATCTCGGTAG
TGGGATACGACGATACCGAAGACAGCTCATGTTATATCCCGCCGTTAACCACCATCAAA
CAGGATTTTCGCCTGCTGGGGCAAACCAGCGTGGACCGCTTGCTGCAACTCTCTCAGG
GCCAGGCGGTGAAGGGCAATCAGCTGTTGCCCGTCTCACTGGTGAAAAGAAAAACCA
CCCTGGCGCCCAATACGCAAACCGCCTCTCCCCGCGCGTTGGCCGATTCATTAATGCA
GCTGGCACGACAGGTTTCCCGACTGGAAAGCGGGCAGAGCAGCCTGAGGCCTCCTAA
GAAGAAGAGGAAGGTTGCGGCCGCTAACCAATGTGCAGACTACTGTTAACCAATGTG
CAGACTACTGTGAATTCTAACCAATGTGCAGACTACTGTTAACCAATGTGCAGACTACT
GTAAGCTTCGAATTGAGTAGTGCTTTCTACTTTATGAGTAGTGCTTTCTACTTTATGAGT
AGTGCTTTCTACTTTATGAGTAGTGCTTTCTACTTTATGGTCGACAATACTAGTTAAGAA
ATCGCCAAGCTACGGGCGGAGTACTGTCCTCCGAGCGGAGTACTGTCCTCCGAGCGGA
GTACTGTCCTCCGAGCGGAGTACTGTCCTCCGAGCGGAGTACTGTCCTCCGAGCGGAG
ACTCTAGAAATTGTGAGCGGATAACAATTTAGGCGTGTACGGTGGGAGGCCTATATAAG
CAGAGCTCGTTTAGTGAACCGTCAGATCGCCAGCTGACAAAATTGTGAGCGCTCACAA
TTACTAGAAGATCTTGAATTCGCCACCATGGCTAGATTAGATAAAAGTAAAGTGATTAA
CAGCGCATTAGAGCTGCTTAATGAGGTCGGAATCGAAGGTTTAACAACCCGTAAACTC
GCCCAGAAGCTAGGTGTAGAGCAGCCTACATTGTATTGGCATGTAAAAAATAAGCGGG
CTTTGCTCGACGCCTTAGCCATTGAGATGTTAGATAGGCACCATACTCACTTTTGCCCTT
TAGAAGGGGAAAGCTGGCAAGATTTTTTACGTAATAACGCTAAAAGTTTTAGATGTGC
TTTACTAAGTCATCGCGATGGAGCAAAAGTACATTTAGGTACACGGCCTACAGAAAAA
CAGTATGAAACTCTCGAAAATCAATTAGCCTTTTTATGCCAACAAGGTTTTTCACTAGA
GAATGCATTATATGCACTCAGCGCTGTGGGGCATTTTACTTTAGGTTGCGTATTGGAAGA
TCAAGAGCATCAAGTCGCTAAAGAAGAAAGGGAAACACCTACTACTGATAGTATGCCG
CCATTATTACGACAAGCTATCGAATTATTTGATCACCAAGGTGCAGAGCCAGCCTTCTTA
TTCGGCCTTGAATTGATCATATGCGGATTAGAAAAACAACTTAAATGTGAAAGTGGGTC
GCCAAAAAAGAAGAGAAAGGTCGACGGCGGTGGTGCTTTGTCTCCTCAGCACTCTGC
TGTCACTCAAGGAAGTATCATCAAGAACAAGGAGGGCATGGATGCTAAGTCACTAACT
GCCTGGTCCCGGACACTGGTGACCTTCAAGGATGTATTTGTGGACTTCACCAGGGAGG
AGTGGAAGCTGCTGGACACTGCTCAGCAGATCGTGTACAGAAATGTGATGCTGGAGA
ACTATAAGAACCTGGTTTCCTTGGGTTATCAGCTTACTAAGCCAGATGTGATCCTCCGG
TTGGAGAAGGGAGAAGAGCCCTGGCTGGTGGAGAGAGAAATTCACCAAGAGACCCA
TCCTGATTCAGAGACTGCATTTGAAATCAAATCATCAGTTTAATACAAGGCGGCCGCAA
ATCAACATCAGTCTGATAAGCTATCAACATCAGTCTGATAAGCTATCAACATCAGTCTG
ATAAGCTATCAACATCAGTCTGATAAGCTAAAGCTTCGAATTCTGATAATCAGCCATACC
ACATTTGTAGAGGTTTTACTTGCTTTAAAAAACCTCCCACACCTCCCCCTGAACCTGAA
ACATAAAATGAATGCAATTGTTGTTGTTAACTTGTTTATTGCAGCTTATAATGGTTACAA
ATAAAGCAATAGCATCACAAATTTCACAAATAAAGCATTTTTTTCACTGCAGCTCCAGA
TTGAATTATTTGCCTGTCATACAGCTAATAATTGACCATAAGACAATTAGATTTAAATTA
GTTTTGAATCTTTCTAATACCAAAGTTCAGTTTACTGTTCCATGTTGCTTCTGAGTGGCT
TCACAGACTTATGAAAAAGTAAACGGAATCAGAATTACATCAATGCAAAAGCATTGCT
GTGAACTCTGTACTTAGGACTAAACTTTGAGCAATAACACATATAGATTGAGGATTGTT
TGCTGTTAGTATACAAACTCTGGTTCAAAGCTCCTCTTTATTGCTTGTCTTGGAAAATTT
GCTGTTCTTCATGGTTTCTCTTTTCACTGCTATCTATTTTTCTCAACCACTCACATGGCTA
CAAAAGCTTCCTGATTAATAATTACACTAAGTCAATAGGCATAGAGCCAGGACTGTTTG
GGTAAACTGGTCACTTTATCTTAAACTAAATATATCCAAAACTGAACATGTACTTAGTTA
CTAAGTCTTTGACTTTATCTCATTCATACCACTCAGCTTTATCCAGGCCACTTATTTGAC
AGTATTATTGCGAAAACTTCCTATCTAGAAGTTTGAGGAGAATATTTGTTATATTTGCAA
AATAAAATAAGTTTGCAAGTTTTTTTTTTCTGCCCCAAAGAGCTCTGTGTCCTTGAACA
TAAAATACAAATAACCGCTATGCTGTTAATTATTGACAAATGTCCCATTTTCAACCTAAG
GAAATACCATAAAGTAACAGATATACCAACAAAAGGTTACTAGTTAACAGGCATTGCCT
GAAAAGAGTATAAAAGAATTTCAGCATGATTTTCCATATTGTGCTTCCACCACTGCCAA
TAACAAAATAACTAGCAAGGATCCGGCCACCATGGCCCCCCCGACCGATGTCAGCCTG
GGGGACGAGCTCCACTTAGACGGCGAGGACGTGGCGATGGCGCATGCCGACGCGCTA
GACGATTTCGATCTGGACATGTTGGGGGACGGGGATTCCCCGGGTCCGGGATTTACCC
CCCACGACTCCGCCCCCTACGGCGCTCTGGATATGGCCGACTTCGAGTTTGAGCAGAT
GTTTACCGATGCCCTTGGAATTGACGAGTACGGTGGGACGCGTATGAAGCTACTGTCTT
CTATCGAACAAGCATGCGATATTTGCCGACTTAAAAAGCTCAAGTGCTCCAAAGAAAA
ACCGAAGTGCGCCAAGTGTCTGAAGAACAACTGGGAGTGTCGCTACTCTCCCAAAAC
CAAAAGGTCTCCGCTGACTAGGGCACATCTGACAGAAGTGGAATCAAGGCTAGAAAG
ACTGGAACAGCTATTTCTACTGATTTTTCCTCGAGAAGACCTTGACATGATTTTGAAAA
TGGATTCTTTACAGGATATAAAAGCATTGTTAACAGGATTATTTGTACAAGATAATGTGA
ATAAAGATGCCGTCACAGATAGATTGGCTTCAGTGGAGACTGATATGCCTCTAACATTG
AGACAGCATAGAATAAGTGCGACATCATCATCGGAAGAGAGTAGTAACAAAGGTCAA
AGACAGTTGACTGTATAATTCACTCCTCAGGTGCAGGCTGCCTATCAGAAGGTGGTGG
CTGGTGTGGCCAATGCCCTGGCTCACAAATACCACTGAGATCTTTTTCCCTCTGCCAAA
AATTATGGGGACATCATGAAGCCCCTTGAGCATCTGACTTCTGGCTAATAAAGGAAATT
TATTTTCATTGCAATAGTGTGTTGGAATTTTTTGTGTCTCTCACTCGGAAGGACATATGG
GAGGGCAAATCATTTAAAACATCAGAATGAGTATTTGGTTTAGAGTTTGGCAACATATG
CCCATATGCTGGCTGCCATGAACAAAGGTTGGCTATAAAGAGGTCATCAGTATATGAAA
CAGCCCCCTGCTGTCCATTCCTTATTCCATAGAAAAGCCTTGACTTGAGGTTAGATTTTT
TTTATATTTTGTTTTGTGTTATTTTTTTCTTTAACATCCCTAAAATTTTCCTTACATGTTTT
ACTAGCCAGATTTTTCCTCCTCTCCTGACTACTCCCAGTCATAGCTGTCCCTCTTCTCTT
ATGGAGATCGGAGAAAGAGGTAAT.
(SEQ ID NO: 14)
GTAACTATAACGGTCGCTCCGAATTTCTCGAGTTAATTAAGATTACGCCAAGCTACG
GGCGGAGTACTGTCCTCCGAGCGGAGTACTGTCCTCCGAGCGGAGTACTGTCCTCCGA
GCGGAGTACTGTCCTCCGAGCGGAGTTCTGTCCTCCGAGCGGAGACTCTAGACTCCCT
ATCAGTGATAGAGATAGGCGTGTACGGTGGGAGGCCTATATAAGCAGAGCTCGTTTAGT
GAACCGTCAGATCGCTCCCTATCAGTGATAGAGAGAATTCGACCGCCACCATGTCGGA
ATTCATGAGACATATTATCTGCCACGGAGGTGTTATTACCGAAGAAATGGCCGCCAGTC
TTTTGGACCAGCTGATCGAAGAGGTACTGGCTGATAATCTTCCACCTCCTAGCCATTTT
GAACCACCTACCCTTCACGAACTGTATGATTTAGACGTGACGGCCCCCGAAGATCCCA
ACGAGGAGGCGGTTTCGCAGATTTTTCCCGACTCTGTAATGTTGGCGGTGCAGGAAGG
GATTGACTTACTCACTTTTCCGCCGGCGCCCGGTTCTCCGGAGCCGCCTCACCTTTCCC
GGCAGCCCGAGCAGCCGGAGCAGAGAGCCTTGGGTCCGGTTTCTATGCCAAACCTTG
TACCGGAGGTGATCGATCTTACCTGCCACGAGGCTGGCTTTCCACCCAGTGACGACGA
GGATGAAGAGGGTGAGGAGTTTGTGTTAGATTATGTGGAGCACCCCGGGCACGGTTGC
AGGTCTTGTCATTATCACCGGAGGAATACGGGGGACCCAGATATTATGTGTTCGCTTTG
CTATATGAGGACCTGTGGCATGTTTGTCTACAGTAAGTGAAAATTATGGGCAGTGGGTG
ATAGAGTGGTGGGTTTGGTGTGGTAATTTTTTTTTTAATTTTTACAGTTTTGTGGTTTAA
AGAATTTTGTATTGTGATTTTTTTAAAAGGTCCTGTGTCTGAACCTGAGCCTGAGCCCG
AGCCAGAACCGGAGCCTGCAAGACCTACCCGCCGTCCTAAAATGGCGCCTGCTATCCT
GAGACGCCCGACATCACCTGTGTCTAGAGAATGCAATAGTAGTACGGATAGCTGTGAC
TCCGGTCCTTCTAACACACCTCCTGAGATACACCCGGTGGTCCCGCTGTGCCCCATTAA
ACCAGTTGCCGTGAGAGTTGGTGGGCGTCGCCAGGCTGTGGAATGTATCGAGGACTTG
CTTAACGAGCCTGGGCAACCTTTGGACTTGAGCTGTAAACGCCCCAGGCCACTCGAG
GGTACCGGGTCCGGAGCTACAAATTTTTCCCTCCTCAAACAGGCTGGAGATGTCGAAG
AAAATCCTGGGCCTACCGGTCCCATGAGTGTGCCCACTCAGGTCCTGGGGTTGCTGCT
GCTGTGGCTTACAGACGCTCGCTGCGAGGTGCAGCTGGTGGAGAGCGGCGGCGGCCT
GGTGCAGCCCGGCGGCAGCCTGAGGCTGAGCTGCGCCGCCAGCGGCTTCACCTTCAG
CGACAGCTGGATCCACTGGGTGAGGCAGGCCCCCGGCAAGGGCCTGGAGTGGGTGGC
CTGGATCAGCCCCTACGGCGGCAGCACCTACTACGCCGACAGCGTGAAGGGCAGGTT
CACCATCAGCGCCGACACCAGCAAGAACACCGCCTACCTGCAGATGAACAGCCTGA
GGGCCGAGGACACCGCCGTGTACTACTGCGCCAGGAGGCACTGGCCCGGCGGCTTCG
ACTACTGGGGCCAGGGCACCCTGGTGACCGTGAGCAGCGGTGGAGGCGGTTCAGGCG
GAGGTGGCTCTGGCGGTGGCGGATCGGACATCCAGATGACCCAGAGCCCCAGCAGCC
TGAGCGCCAGCGTGGGCGACAGGGTGACCATCACCTGCAGGGCCAGCCAGGACGTGA
GCACCGCCGTGGCCTGGTACCAGCAGAAGCCCGGCAAGGCCCCCAAGCTGCTGATCT
ACAGCGCCAGCTTCCTGTACAGCGGCGTGCCCAGCAGGTTCAGCGGCAGCGGCAGCG
GCACCGACTTCACCCTGACCATCAGCAGCCTGCAGCCCGAGGACTTCGCCACCTACTA
CTGCCAGCAGTACCTGTACCACCCCGCCACCTTCGGCCAGGGCACCAAGGTGGAGAT
CAAGAGATCTGAGGGCCGCGGCAGCCTGCTGACCTGCGGCGACGTGGAGGAAAACCC
CGGCCCCGGATCCATGAAACCAGTAACGTTATACGATGTCGCAGAGTATGCCGGTGTCT
CTTATCAGACCGTTTCCCGCGTGGTGAACCAGGCCAGCCACGTTTCTGCGAAAACGCG
GGAAAAAGTGGAAGCGGCGATGGCGGAGCTGAATTACATTCCCAACCGCGTGGCACA
ACAACTGGCGGGCAAACAGTCGTTGCTGATTGGCGTTGCCACCTCCAGTCTGGCCCTG
CACGCGCCGTCGCAAATTGTCGCGGCGATTAAATCTCGCGCCGATCAACTGGGTGCCA
GCGTGGTGGTGTCGATGGTAGAACGAAGCGGCGTCGAAGCCTGTAAAGCGGCGGTGC
ACAATCTTCTCGCGCAACGCGTCAGTGGGCTGATCATTAACTATCCGCTGGATGACCAG
GATGCCATTGCTGTGGAAGCTGCCTGCACTAATGTTCCGGCGTTATTTCTTGATGTCTCT
GACCAGACACCCATCAACAGTATTATTTTCTCCCATGAAGACGGTACGCGACTGGGCG
TGGAGCATCTGGTCGCATTGGGTCACCAGCAAATCGCGCTGTTAGCGGGCCCATTAAG
TTCTGTCTCGGCGCGTCTGCGTCTGGCTGGCTGGCATAAATATCTCACTCGCAATCAAA
TTCAGCCGATAGCGGAACGGGAAGGCGACTGGAGTGCCATGTCCGGTTTTCAACAAA
CCATGCAAATGCTGAATGAGGGCATCGTTCCCACTGCGATGCTGGTTGCCAACGATCA
GATGGCGCTGGGCGCAATGCGCGCCATTACCGAGTCCGGGCTGCGCGTTGGTGCGGAT
ATCTCGGTAGTGGGATACGACGATACCGAAGACAGCTCATGTTATATCCCGCCGTTAAC
CACCATCAAACAGGATTTTCGCCTGCTGGGGCAAACCAGCGTGGACCGCTTGCTGCAA
CTCTCTCAGGGCCAGGCGGTGAAGGGCAATCAGCTGTTGCCCGTCTCACTGGTGAAA
AGAAAAACCACCCTGGCGCCCAATACGCAAACCGCCTCTCCCCGCGCGTTGGCCGATT
CATTAATGCAGCTGGCACGACAGGTTTCCCGACTGGAAAGCGGGCAGAGCAGCCTGA
GGCCTCCTAAGAAGAAGAGGAAGGTTGCGGCCGCTAACCAATGTGCAGACTACTGTT
AACCAATGTGCAGACTACTGTGAATTCTAACCAATGTGCAGACTACTGTTAACCAATGT
GCAGACTACTGTAAGCTTCGAATTGAGTAGTGCTTTCTACTTTATGAGTAGTGCTTTCTA
CTTTATGAGTAGTGCTTTCTACTTTATGAGTAGTGCTTTCTACTTTATGGTCGACAATACT
AGTTAAGAAATCGCCAAGCTACGGGCGGAGTACTGTCCTCCGAGCGGAGTACTGTCCT
CCGAGCGGAGTACTGTCCTCCGAGCGGAGTACTGTCCTCCGAGCGGAGTACTGTCCTC
CGAGCGGAGACTCTAGAAATTGTGAGCGGATAACAATTTAGGCGTGTACGGTGGGAG
GCCTATATAAGCAGAGCTCGTTTAGTGAACCGTCAGATCGCCAGCTGACAAAATTGTG
AGCGCTCACAATTACTAGAAGATCTTGAATTCGCCACCATGGCTAGATTAGATAAAAGT
AAAGTGATTAACAGCGCATTAGAGCTGCTTAATGAGGTCGGAATCGAAGGTTTAACAA
CCCGTAAACTCGCCCAGAAGCTAGGTGTAGAGCAGCCTACATTGTATTGGCATGTAAA
AAATAAGCGGGCTTTGCTCGACGCCTTAGCCATTGAGATGTTAGATAGGCACCATACTC
ACTTTTGCCCTTTAGAAGGGGAAAGCTGGCAAGATTTTTTACGTAATAACGCTAAAAG
TTTTAGATGTGCTTTACTAAGTCATCGCGATGGAGCAAAAGTACATTTAGGTACACGGC
CTACAGAAAAACAGTATGAAACTCTCGAAAATCAATTAGCCTTTTTATGCCAACAAGG
TTTTTCACTAGAGAATGCATTATATGCACTCAGCGCTGTGGGGCATTTTACTTTAGGTTG
CGTATTGGAAGATCAAGAGCATCAAGTCGCTAAAGAAGAAAGGGAAACACCTACTAC
TGATAGTATGCCGCCATTATTACGACAAGCTATCGAATTATTTGATCACCAAGGTGCAGA
GCCAGCCTTCTTATTCGGCCTTGAATTGATCATATGCGGATTAGAAAAACAACTTAAAT
GTGAAAGTGGGTCGCCAAAAAAGAAGAGAAAGGTCGACGGCGGTGGTGCTTTGTCTC
CTCAGCACTCTGCTGTCACTCAAGGAAGTATCATCAAGAACAAGGAGGGCATGGATGC
TAAGTCACTAACTGCCTGGTCCCGGACACTGGTGACCTTCAAGGATGTATTTGTGGAC
TTCACCAGGGAGGAGTGGAAGCTGCTGGACACTGCTCAGCAGATCGTGTACAGAAAT
GTGATGCTGGAGAACTATAAGAACCTGGTTTCCTTGGGTTATCAGCTTACTAAGCCAGA
TGTGATCCTCCGGTTGGAGAAGGGAGAAGAGCCCTGGCTGGTGGAGAGAGAAATTCA
CCAAGAGACCCATCCTGATTCAGAGACTGCATTTGAAATCAAATCATCAGTTTAATACA
AGGCGGCCGCAAATCAACATCAGTCTGATAAGCTATCAACATCAGTCTGATAAGCTATC
AACATCAGTCTGATAAGCTATCAACATCAGTCTGATAAGCTAAAGCTTCGAATTCTGAT
AATCAGCCATACCACATTTGTAGAGGTTTTACTTGCTTTAAAAAACCTCCCACACCTCC
CCCTGAACCTGAAACATAAAATGAATGCAATTGTTGTTGTTAACTTGTTTATTGCAGCT
TATAATGGTTACAAATAAAGCAATAGCATCACAAATTTCACAAATAAAGCATTTTTTTCA
CTGCAGCTCCAGATTGAATTATTTGCCTGTCATACAGCTAATAATTGACCATAAGACAAT
TAGATTTAAATTAGTTTTGAATCTTTCTAATACCAAAGTTCAGTTTACTGTTCCATGTTG
CTTCTGAGTGGCTTCACAGACTTATGAAAAAGTAAACGGAATCAGAATTACATCAATG
CAAAAGCATTGCTGTGAACTCTGTACTTAGGACTAAACTTTGAGCAATAACACATATAG
ATTGAGGATTGTTTGCTGTTAGTATACAAACTCTGGTTCAAAGCTCCTCTTTATTGCTTG
TCTTGGAAAATTTGCTGTTCTTCATGGTTTCTCTTTTCACTGCTATCTATTTTTCTCAACC
ACTCACATGGCTACAAAAGCTTCCTGATTAATAATTACACTAAGTCAATAGGCATAGAG
CCAGGACTGTTTGGGTAAACTGGTCACTTTATCTTAAACTAAATATATCCAAAACTGAA
CATGTACTTAGTTACTAAGTCTTTGACTTTATCTCATTCATACCACTCAGCTTTATCCAGG
CCACTTATTTGACAGTATTATTGCGAAAACTTCCTATCTAGAAGTTTGAGGAGAATATTT
GTTATATTTGCAAAATAAAATAAGTTTGCAAGTTTTTTTTTTCTGCCCCAAAGAGCTCTG
TGTCCTTGAACATAAAATACAAATAACCGCTATGCTGTTAATTATTGACAAATGTCCCAT
TTTCAACCTAAGGAAATACCATAAAGTAACAGATATACCAACAAAAGGTTACTAGTTAA
CAGGCATTGCCTGAAAAGAGTATAAAAGAATTTCAGCATGATTTTCCATATTGTGCTTC
CACCACTGCCAATAACAAAATAACTAGCAAGGATCCGGCCACCATGGCCCCCCCGACC
GATGTCAGCCTGGGGGACGAGCTCCACTTAGACGGCGAGGACGTGGCGATGGCGCAT
GCCGACGCGCTAGACGATTTCGATCTGGACATGTTGGGGGACGGGGATTCCCCGGGTC
CGGGATTTACCCCCCACGACTCCGCCCCCTACGGCGCTCTGGATATGGCCGACTTCGA
GTTTGAGCAGATGTTTACCGATGCCCTTGGAATTGACGAGTACGGTGGGACGCGTATG
AAGCTACTGTCTTCTATCGAACAAGCATGCGATATTTGCCGACTTAAAAAGCTCAAGTG
CTCCAAAGAAAAACCGAAGTGCGCCAAGTGTCTGAAGAACAACTGGGAGTGTCGCTA
CTCTCCCAAAACCAAAAGGTCTCCGCTGACTAGGGCACATCTGACAGAAGTGGAATC
AAGGCTAGAAAGACTGGAACAGCTATTTCTACTGATTTTTCCTCGAGAAGACCTTGAC
ATGATTTTGAAAATGGATTCTTTACAGGATATAAAAGCATTGTTAACAGGATTATTTGTA
CAAGATAATGTGAATAAAGATGCCGTCACAGATAGATTGGCTTCAGTGGAGACTGATAT
GCCTCTAACATTGAGACAGCATAGAATAAGTGCGACATCATCATCGGAAGAGAGTAGT
AACAAAGGTCAAAGACAGTTGACTGTATAATTCACTCCTCAGGTGCAGGCTGCCTATC
AGAAGGTGGTGGCTGGTGTGGCCAATGCCCTGGCTCACAAATACCACTGAGATCTTTT
TCCCTCTGCCAAAAATTATGGGGACATCATGAAGCCCCTTGAGCATCTGACTTCTGGCT
AATAAAGGAAATTTATTTTCATTGCAATAGTGTGTTGGAATTTTTTGTGTCTCTCACTCG
GAAGGACATATGGGAGGGCAAATCATTTAAAACATCAGAATGAGTATTTGGTTTAGAG
TTTGGCAACATATGCCCATATGCTGGCTGCCATGAACAAAGGTTGGCTATAAAGAGGTC
ATCAGTATATGAAACAGCCCCCTGCTGTCCATTCCTTATTCCATAGAAAAGCCTTGACTT
GAGGTTAGATTTTTTTTATATTTTGTTTTGTGTTATTTTTTTCTTTAACATCCCTAAAATTT
TCCTTACATGTTTTACTAGCCAGATTTTTCCTCCTCTCCTGACTACTCCCAGTCATAGCT
GTCCCTCTTCTCTTATGGAGATCGGAGAAAGAGGTAAT.

wherein SEQ ID NO: 9 is the sequence of an EBFP-bearing nucleic acid carried by an adenoviral vector;

SEQ ID NO: 10 is the sequence of an hIL-2-bearing nucleic acid carried by an adenoviral vector;

SEQ ID NO: 11 is the sequence of an hGMCSF-bearing nucleic acid carried by an adenoviral vector;

SEQ ID NO: 12 is the sequence of an mGMCSF-bearing nucleic acid carried by an adenoviral vector;

SEQ ID NO: 13 is the sequence of an anti-PD-1 scFv-bearing nucleic acid carried by an adenoviral vector; and

SEQ ID NO: 14 is the sequence of an anti-PD-L1 scFv-bearing nucleic acid carried by an adenoviral vector.

In an embodiment of the present disclosure, the adenovirus is obtained by the following steps: removing the adenoviral E1 gene and a part of the adenoviral E3 gene associated with adenoviral replication and packaging from an Adenoviral vector; inserting the adenoviral E1A gene into a gene circuit by a stepwise Golden Gate method; and incorporating the gene circuit into the adenoviral vector by the Gateway or Gibson method. The method of obtaining adenovirus as described above enables rapidly engineering of a complex and large-fragment oncolytic adenoviral vector.

In a second aspect, the present disclosure in embodiments provides a recombinant virus. In an embodiment of the present disclosure, the recombinant virus comprises:

a first nucleic acid molecule, comprising a tumor cell-specific promoter, the tumor cell-specific promoter being an alpha-fetoprotein-specific promoter;

a second nucleic acid molecule, operably linked to the first nucleic acid molecule and encoding a transcriptional activator, the transcriptional activator being Gal4VP16;

a third nucleic acid molecule, comprising a first recognition sequence of the transcriptional activator, the first recognition sequence being 5×UAS;

a fourth nucleic acid molecule, operably linked to the third nucleic acid molecule and comprising a first promoter and a first regulatory element, wherein the first promoter is a miniCMV, and the first regulatory element comprises a plurality of repeated tetO sequences, and at least one of the pluralities of repeated tetO sequences is set downstream of the first promoter;

a fifth nucleic acid molecule, operably linked to the fourth nucleic acid molecule and encoding a first regulatory protein, the first regulatory protein being Lad,

wherein the fifth nucleic acid molecule further comprises a sequence encoding a protein of interest, the protein of interest comprises a viral replication protein and an immune effector,

wherein the immune effector is expressed in the form of an individual protein or a fusion protein, the viral replication protein and the immune effector are linked by a cleavable linker peptide, the protein of interest and the first regulatory protein are expressed in the form of a fusion protein, and the protein of interest and the first regulatory protein are linked by a cleavable linker peptide;

a sixth nucleic acid molecule, comprising a second recognition sequence of the transcriptional activator, the second recognition sequence being 5×UAS;

a seventh nucleic acid molecule, operably linked to the sixth nucleic acid molecule and comprising a second promoter and a second regulatory element, wherein the second promoter is a miniCMV, the second regulatory element comprises a plurality of repeated LacO sequences, at least one of the pluralities of repeated LacO sequences is inserted downstream of the second promoter;

an eighth nucleic acid molecule, operably linked to the seventh nucleic acid molecule and encoding a second regulatory protein, the second regulatory protein being tetR-KRAB;

a ninth nucleic acid molecule, operably linked to the fifth nucleic acid molecule and configured to conditionally inhibit expression of the first regulatory protein, wherein the ninth nucleic acid molecule comprises a nucleic acid sequence specifically recognized by a first microRNA, the first microRNA being a normal cell-specific microRNA; and

a tenth nucleic acid molecule, operably linked to the eighth nucleic acid molecule and configured to conditionally inhibit expression of the second regulatory protein, wherein the tenth nucleic acid molecule comprises a nucleic acid sequence specifically recognized by a second microRNA, the second microRNA being a tumor cell-specific microRNA,

wherein the first regulatory element is adapted to inhibit the function of the first promoter by binding to the second regulatory protein, and the second regulatory element is adapted to inhibit the function of the second promoter by binding to the first regulatory protein. With the recombinant virus according to the embodiment of the present disclosure, the first regulatory protein Lad and the protein of interest are specifically expressed in tumor cells and the second regulatory protein tetR-KRAB is not expressed or low-expressed specifically in tumor cells under the coregulation of the alpha-fetoprotein-specific promoter, the ninth nucleic acid molecule and the tenth nucleic acid molecule, thus the tetR-KRAB-mediated inhibition mechanism on the first promoter miniCMV is released, therefore the first regulatory protein Lad and the protein of interest are effectively expressed under the control of the first promoter miniCMV, and the function of the second promoter miniCMV is effectively inhibited via the LacI-mediated inhibition mechanism, and the expression of tetR-KRAB is further inhibited. Furthermore, proteins like the target protein Lad are specifically expressed in tumor cells and the proteins like tetR-KRAB are not specifically expressed in tumor cells by using the recombinant virus according to the embodiment of the present disclosure, with high efficiency and specificity.

In embodiments of the present disclosure, the recombinant virus may further comprise at least one of additional technical features as follows.

In an embodiment of the present disclosure, the recombinant virus comprises at least one selected from the group consisting of a retrovirus, an adenovirus, a herpes virus and a vaccinia virus.

In an embodiment of the present disclosure, the recombinant virus is an adenovirus. As mentioned above, adenovirus as a gene therapy vector has advantages of a wide range of hosts, low pathogenicity to humans, infection and gene expression in proliferating and non-proliferating cells, high titer, homology to human genes, and no mutagenicity after insertion, as well as amplifying in a suspension and simultaneously expressing multiple genes.

In an embodiment of the present disclosure, the immune effector comprises at least one sequence selected from the group consisting of an inhibitory sequence that antagonizes PD-1 gene, an inhibitory sequence that antagonizes PD-L1 gene, an inhibitory sequence that antagonizes CTLA4 gene, an inhibitory sequence that antagonizes Tim-3 gene, IL-2, IL-12, IL-15 and GM-CSF. Optionally, the immune effector may be present in the form of a fusion protein.

In a third aspect, the present disclosure in embodiments provides a recombinant cell. In an embodiment of the present disclosure, the recombinant cell comprises the expression system as described above. The recombinant cell according to the embodiment of the present disclosure can effectively activate the systemic immune response in human body, and attack xenogeneic cells such as tumor cells, with high safety and specificity.

In embodiments of the present disclosure, the recombinant cell may further comprise at least one of additional technical features as follows.

In an embodiment of the present disclosure, at least a portion of the expression system is integrated into the genome of the recombinant cell. The expression system replicates as the recombinant cell genome replicates, where the expression system can still effectively regulate the expression of the protein of interest.

In a fourth aspect, the present disclosure in embodiments provides use of the aforementioned expression system, the aforementioned recombinant virus, and the aforementioned recombinant cells in the preparation of a medicament for the treatment of a cancer. The expression system described in the present disclosure allows the protein of interest to be specifically expressed in tumor cells, and the medicament in the embodiment exhibits stronger efficacy, specificity and safety on the treatment of cancer.

In embodiments of the present disclosure, the use as described above can further comprise at least one of additional technical features as follows.

In an embodiment of the present disclosure, the cancer comprises liver cancer, lung cancer, colorectal cancer, melanoma, breast cancer or prostate cancer. The inventors have found that the medicament in the embodiment has a more significant efficacy on the treatment of liver cancer, lung cancer, colorectal cancer, melanoma, breast cancer and prostate cancer.

In a fifth aspect, the present disclosure in embodiments provides a method of expressing a protein of interest by use of an expression system, wherein the expression system is the expression system as described above. In an embodiment of the present disclosure, the method comprises: (1) providing a fifth nucleic acid molecule which comprises a nucleic acid sequence encoding the protein of interest; and (2) inhibiting expression of a second regulatory protein by a tenth nucleic acid molecule so as to express the protein of interest.

With the method of expressing a protein of interest as described above in the embodiment, the second regulatory protein-mediated inhibition mechanism on the first promoter is released, thus the protein of interest is effectively expressed in specific cells under the coaction of the cell-specific promoter and the second promoter.

In embodiments of the present disclosure, the above method can further comprise at least one of additional technical features as follows.

In an embodiment of the present disclosure, the expression is carried out in a cell. The cells can provide a microenvironment for the expression of the protein of interest, and the expression of the protein of interest in the cell is more efficient.

In an embodiment of the present disclosure, the tenth nucleic acid molecule comprises a nucleic acid sequence specifically recognized by a second microRNA, and the step (2) further comprises contacting the second microRNA with the tenth nucleic acid molecule.

In a sixth aspect, the present disclosure in embodiments provides a method of expressing a protein of interest by use of an expression system, wherein the expression system is the expression system as described above. In an embodiment of the present disclosure, the method comprises: (1) providing an eighth nucleic acid molecule which comprises a nucleic acid sequence encoding a first protein of interest, and providing a fifth nucleic acid molecule which comprises a nucleic acid sequence encoding a second protein of interest; and (2) expressing the first protein of interest or the second protein of interest by inhibiting expression of the first regulatory protein by the ninth nucleic acid molecule so as to express the first protein of interest; or inhibiting expression of the second regulatory protein by the tenth nucleic acid molecule so as to express the second protein of interest. With the method of expressing a protein of interest as described above in the embodiment, the second regulatory protein-mediated inhibition mechanism on the first promoter is released, thus the second protein of interest is effectively expressed in specific cells under the coaction of the cell-specific promoter and the first promoter; or the first regulatory protein-mediated inhibition mechanism on the second promoter is released, thus the first protein of interest is effectively expressed in specific cells under the coaction of the cell-specific promoter and the second promoter.

It should be noted that the fusion protein described herein refers to proteins that are co-transcribed under the control of a same promoter, including a fusion protein that is not linked between proteins, or a fusion protein that is linked by other linker peptides (such as GGGS or 2A sequences).

### BRIEF DESCRIPTION OF THE DRAWINGS

FIG. 1 is a schematic diagram showing an initial structure of an adenovirus engineered by the Gateway system.

FIG. 2 is a schematic diagram showing an initial structure of an adenovirus engineered by the Golden Gate system.

FIG. 3 is a schematic diagram showing a mutually repressive switch according to an embodiment of the present disclosure.

FIG. 4 is a graph showing the expression level of alpha-fetoprotein-specific promoter (hAFP) in cell lines Chang, HepG2, Huh7, Hep3B, PLC and Hepa1-6 by the fluorescence quantitative PCR method according to an embodiment of the present disclosure.

FIG. 5 is a schematic diagram showing the modification of human AFP promoter according to an embodiment of the present disclosure.

FIG. 6 is a graph showing the results of transient transfection of a transmission system of the modified AFP promoter to Chang and HepG2 according to an embodiment of the present disclosure.

FIG. 7 is a schematic diagram showing microRNA detection systems according to an embodiment of the present disclosure.

FIG. 8 is a graph showing the significantly different expression of microRNA markers in different cell lines according to an embodiment of the present disclosure.

FIG. 9 is a schematic diagram of a mutually repressive switch based on Lad and tetR-KRAB genes according to an embodiment of the present disclosure.

FIG. 10 is a graph showing the effective reversal of the mutually repressive switch in response to artificially synthesized shRNA signals according to an embodiment of the present disclosure.

FIG. 11 (a)-(d) are schematic diagrams showing the mutually repressive switch in response to different activators according to some embodiments of the present disclosure.

FIG. 12 is a schematic diagram showing the first step of the CGGA (Cascade Golden-Gate and Gateway/Gibson Assemble method) according to an embodiment of the present disclosure.

FIG. 13 is a schematic diagram showing the second step of the CGGA (Cascade Golden-Gate and Gateway/Gibson Assemble method) according to an embodiment of the present disclosure.

FIG. 14 is a schematic diagram showing the third step of the CGGA (Cascade Golden-Gate and Gateway/Gibson Assemble method) according to an embodiment of the present disclosure.

FIG. 15 (a)-(c) are graphs showing the detection of adenovirus at a cellular level according to some embodiments of the present disclosure.

FIG. 16 is a graph showing activities of cytokines (expressed by a plasmid vector) detected in vitro according to some embodiments of the present disclosure.

FIG. 17 (a)-(c) are graphs showing activities of cytokines (expressed by a viral vector) detected in vitro according to some embodiments of the present disclosure.

FIG. 18 (a)-(d) are schematic diagrams showing the treatment of nude mouse model bearing HepG2, Huh7 and Hepa1-6 respectively by using an oncolytic adenovirus according to some embodiments of the present disclosure.

FIG. 19 (a)-(i) are schematic diagrams showing the treatment of C57 mouse model bearing Hepa1-6 by using an oncolytic adenovirus according to an embodiment of the present disclosure.

FIG. 20 (a)&(b) are graphs showing improved expression of IFN-γ and Ki67 markers among the tumor filtrating T cells after the treatment with oncolytic adenovirus according to an embodiment of the present disclosure.

FIG. 21 is a graph showing the re-challenge of mouse hepatoma Hepa1-6 cells on mouse after treatment according to an embodiment of the present disclosure.

FIG. 22 shows a model of tumor-virus-effector system according to an embodiment of the present disclosure.

FIG. 23 is a graph showing the minimal tumor size from an initial state to a final state over time under different initial tumor size, viral titer and viral replication rate during the simulation of the tumor-virus system according to an embodiment of the present disclosure.

FIG. 24 is a graph showing the tumor size in the final state under the different inhibitory effects of immune cells on tumors and viruses when simulated by the tumor-virus-effector system according to an embodiment of the present disclosure.

### DETAILED DESCRIPTION

Embodiments of the present disclosure are described in detail below. The embodiments described below are illustrative only and are not to be construed as limiting the present disclosure.

Expression System

In a first aspect of the present disclosure, provided in embodiments is an expression system. According to an embodiment of the present disclosure, the expression system comprises:

a first nucleic acid molecule, incorporating a cell-specific promoter;

a second nucleic acid molecule, operably linked to the first nucleic acid molecule and encoding a transcriptional activator;

a third nucleic acid molecule, incorporating a first recognition sequence of the transcriptional activator;

a fourth nucleic acid molecule, operably linked to the third nucleic acid molecule and incorporating a first promoter and a first regulatory element;

a fifth nucleic acid molecule, operably linked to the fourth nucleic acid molecule and encoding a first regulatory protein;

a sixth nucleic acid molecule, incorporating a second recognition sequence of the transcriptional activator;

a seventh nucleic acid molecule, operably linked to the sixth nucleic acid molecule and incorporating a second promoter and a second regulatory element;

an eighth nucleic acid molecule, operably linked to the seventh nucleic acid and encoding a second regulatory protein; and

at least one selected from the group consisting of:

a ninth nucleic acid molecule, operably linked to the fifth nucleic acid molecule and configured to conditionally inhibit expression of the first regulatory protein;

a tenth nucleic acid molecule, operably linked to the eighth nucleic acid molecule and configured to conditionally inhibit expression of the second regulatory protein,

wherein

the first regulatory element is adapted to inhibit the function of the first promoter by binding to the second regulatory protein, and

the second regulatory element is adapted to inhibit the function of the second promoter by binding to the first regulatory protein.

With the expression system according to the embodiment of the present disclosure, the gene of interest can be specifically expressed under a specific cellular environment while being regulated in a mutual repression way, with strong control, high efficiency and specificity.

According to an embodiment of the present disclosure, the cell-specific promoter is a tumor cell-specific promoter. The tumor cell-specific promoter is at least one selected from the group consisting of an alpha-fetoprotein-specific promoter (AFP), a Survivin gene promoter (SUR), a human telomerase reverse transcriptase (hTERT) gene promoter, a cholecystokinin A receptor gene promoter (CCKAR promoter), a carcinoembryonic antigen promoter (CEA promoter), a proto-oncogene human epidermal growth factor receptor 2 promoter (Human epidermal growth factor receptor-2, HER2), a prostaglandin oxidase reductase 2 promoter (Cyclooxygenase 2, COX2), a chemokine receptor-4 (CXCR4), an E2F-1 gene promoter (E2F-1 promoter), a mucin promoter (Mucin1MUC1), a prostate specific antigen (PSA), a human tyrosine related protein 1 (TRP1) and a tyrosinase (Tyr) promoter. The expression system in the embodiment can be initiated in the microenvironment of specific tumor cells under the control of the tumor cell-specific promoters described as above, thereby further enhancing the specificity of gene expression and regulation.

According to an embodiment of the present disclosure, the transcriptional activator is at least one selected from the group consisting of Gal4VP16, Gal4esn, Gal4VP64, dCas9-VP16, dCas9-VP64, dCas9-VPR, dCas9-VTR and rtTA. The yeast Ga14-UAS gene expression system is one of the most well understood transcriptional regulatory systems of eukaryotic cells, and the Gal4 gene encodes a transcriptional activator protein in yeast (Saccharomyces cerevisiae). Gal4 can recognize a 17 bp of sequence in the upstream activation sequence (UAS) of gene promoter, that is, 5′-CGGRNNRCYNYNYNCNCCG-3′ (R represents purine, Y represents pyrimidine, and N represents any deoxynucleotide). Gal4 interacts with Ga180 which binds to galactose metabolites. Gal4 comprises two domains (a DNA binding domain (N-terminal domain) and an activation domain (C-terminal domain)) and further comprises a zinc cluster domain (specifically a Zn(2)-Cys(6) dual core cluster domain), which usually functions in a form of a homodimer. The core sequence in the UAS sequence that binds to the GAL4 protein is a four-copy tandem repeat, each copy containing 17 base pairs (i.e. the sequence 5′-CGGAGTACTGTCGTGGG-3′). Ga14, present in mammalian cells, activates target genes only when UAS is recognized, thus initiating the transcription of downstream genes, with good specificity and inducibility. The activation domain of Gal4 protein can be replaced with the activation domain of herpes simplex virus VP16 protein or 4 copies of VP16 (VP64). The formed fusion transcription factor increases the transcriptional activation activity of Gal4 protein while maintaining the binding specificity of Gal4 protein. The tetracycline-inducing system is a bacterial-derived transcriptional activation system for eukaryotic cells, including tetracycline response elements and tetracycline-inducing proteins. The Tet response element (TRE) contains 7 repeats of 19-nucleotide tetracycline operon sequence (i.e. 5′-TCCCTATCAGTGATAGAGA-3′), which can be recognized by tetracycline-inducing proteins. The tetracycline-inducing system includes a tetracycline-inducing repression system and a tetracycline-inducing activation system. The tetracycline-inducing repression system comprises a tetracycline response element and a tetracycline repressor tetR. When tetracycline is absent, the expression of downstream genes is activated by the binding of the repressor to the response element; and when tetracycline is present, the repressor binds to tetracycline, thus inhibiting the expression of downstream genes. The tetracycline-inducing activation system comprises a tetracycline response element and a reverse tetracycline-controlled transactivator rtTA. When tetracycline is absent, rtTA cannot bind to the TRE element, thus cannot activate the expression of downstream genes; while when tetracycline is present, rtTA binds to TRE under the action of tetracycline, thus activating the expression of downstream genes.

Nuclease deactivated Cas9 (dCas9) is a mutant form of Cas9, which can recognize a target sequence via gRNA mediation, only with specific recognition and binding on DNA and without cleavage activity on target sequence. When dCas9 is fusion-expressed along with activation elements, the expression of target genes can be effectively activated.

According to an embodiment of the present disclosure, the first recognition sequence and the second recognition sequence are each independently selected from at least one of 5×UAS, 7×tetO and a target sequence of dCas9. 5×UAS is 5 copies of response elements of Gal4VP16 regulatory system. The core sequence in the UAS sequence that binds to the Gal4 protein is a four-copy tandem repeat, each copy containing 17 base pairs (sequence 5′-CGGAGTACTGTCGTGGG-3′). 7×tetO is 7 copies of TREs which are a response element of tetracycline regulatory system. The Tet response element (TRE) contains 7 repeats of 19-nucleotide tetracycline operon sequence (5′-TCCCTATCAGTGATAGAGA-3′), which can be recognized by tetracycline-inducing proteins.

According to an embodiment of the present disclosure, the first promoter and the second promoter are miniCMV. 5×UAS-miniCMV is a synthetic inducible promoter. 5×UAS is a recognition sequence of Gal4 protein, and miniCMV is a part of promoter sequence of Cytomegalovirus. TRE is also an inducible promoter. For 7×tetO-miniCMV, tetO is a recognition sequence of the response element of tetracycline system.

According to an embodiment of the present disclosure, the first regulatory protein and the second regulatory protein are each independently selected from at least one of LacI, tetR, zinc finger, KRAB and dCas9-KRAB. Lactose repressor (LacI) binds to the major groove of lactose operon (LacO) through recognizing specific sequences via the helix-turn-helix element of its own DNA-binding domain, as well as tightly binding to base sequence on the minor groove of LacO via symmetry-related α-helix residues. Such a tight binding causes high affinity binding of RNA polymerase to promoter region and prevents DNA extension, thereby inhibiting mRNA transcription and expression of downstream genes. The tetracycline repressor (TetR) contains a conserved helix-turn-helix DNA binding region (forming a homodimer) which binds to the tetracycline operon (tetO), thus the transcriptional expression of downstream genes is inhibited. Zinc finger protein consists of a ring containing about 30 amino acids and a Zn²⁺ coordinated to 4 Cys proteins (or 2 Cys) and 2 His proteins on the ring, forming a structure in a shape of a finger. Zinc finger proteins are a class of proteins that play an important role in gene regulation. Zinc finger proteins can be mainly classified into C2H2 type, C4 type and C6 type depending to their conserved domains. Zinc finger can regulate gene expression, cell differentiation and embryonic development at transcriptional and translational levels by specifically binding to target sequences of DNA, RNA, DNA-RNA molecules, or to its own or other zinc finger proteins. The Krüppel associated box (KRAB) domain is a class of transcriptional repression regions which is present in nearly 400 zinc finger proteins in humans. KRAB generally consists of 75 amino acid residues and its smallest effective inhibition region consists of 45 amino acids, which plays its role through amphipathic helical connection between proteins. KRAB can be divided into two regions (A box and B box) which are encoded by different exons, where A box plays a central role in transcriptional repression, and B box enhances the transcriptional repression of A box.

Nuclease deactivated Cas9 (dCas9) is a mutant form of Cas9, which can recognize a target sequence via gRNA mediation, only with specific recognition and binding activities on DNA sequences and without cleavage activity on target sequences. When dCas9 is fusion-expressed along with repression regions, the expression of target genes can be effectively inhibited.

According to an embodiment of the present disclosure, the first regulatory element and the second regulatory element are each independently selected from at least one of a tetO, a LacO, a zinc finger target site, and a target sequence of dCas9. The tetracycline operon (tetO) can be recognized by tetracycline repressors to inhibit the expression of downstream genes. The lactose operon (LacO) can be recognized by lactose repressors (Lad) to inhibit the expression of downstream genes. The zinc finger target site can be recognized by zinc finger proteins to regulate the expression of downstream genes. The dCas9 target sequence can be recognized by dCas9-KRAB with the aid of a complementary gRNA to inhibit the expression of downstream genes.

According to an embodiment of the present disclosure, the first regulatory protein is LacI, and the second regulatory element comprises a plurality of repeated LacO sequences, at least one of the pluralities of repeated LacO sequences being set downstream of the second promoter. The expressed LacI can specifically bind to LacO sequence, thereby inhibiting the function of the second promoter. According to the LacI/LacO repression system of the embodiment of the present disclosure, Lactose repressor (Lad) binds to the major groove of lactose operon (LacO) through recognizing specific sequences via the helix-turn-helix element of its own DNA-binding domain, as well as tightly binds to base sequence on the minor groove of LacO via symmetry-related α-helix residues. Such a tight binding causes high affinity binding of RNA polymerase to promoter regions and prevents DNA extension, thereby inhibiting mRNA transcription and expression of downstream genes. According to an embodiment of the present disclosure, two sides of miniCMV sequence in 5×UAS-miniCMV promoter are each inserted with LacO by the inventors, which can effectively inhibit the expression of downstream genes of the promoter as shown by experiments. However, the inventors do not exclude other repression systems which can be used at those locations.

According to an embodiment of the present disclosure, the second regulatory protein is tetR-KRAB, and the first regulatory element comprises a plurality of repeated tetO sequences, at least one of the pluralities of repeated tetO sequences being set downstream of the first promoter. According to the tetR-KRAB/tetO repression system in the embodiment of the present disclosure, the tetracycline repressor (TetR) contains a conserved helix-turn-helix DNA binding region (forming a homodimer), which binds to tetracycline operon (tetO) to inhibit the transcriptional expression of downstream genes. In this embodiment, two sides of miniCMV sequence in 5×UAS-miniCMV promoter are each inserted with tetO by inventors, which can effectively inhibit the expression of downstream genes of the promoter as shown by experiments. The switch system comprising the first regulatory protein and the second regulatory protein can effectively respond to corresponding input signals, thus effectively distinguishing different cell lines. However, the inventors do not exclude other repression systems which can be used at those locations.

According to an embodiment of the present disclosure, at least one of the fifth nucleic acid molecule and the ninth nucleic acid molecule further comprises a sequence encoding a protein of interest. With the expression system according to the embodiment of the present disclosure, the protein of interest can be specifically expressed in a specific cellular microenvironment, while being regulated in a mutual repression way. According to a specific embodiment of the present disclosure, the expression system exhibits a significant increased specificity and regulation on the expression of the protein of interest.

According to an embodiment of the present disclosure, the fifth nucleic acid molecule comprises a sequence encoding the protein of interest, and the protein of interest comprises at least one selected from the group consisting of a viral replication and packaging protein, and an immune effector. The viral replication and packaging protein can effectively ensure the survival and replication of the expression system vector in a host. The expression of the immune effector can effectively activate the immune system in body, thereby promoting the immune killing to specific cells, such as tumor cells.

According to an embodiment of the present disclosure, the virus replication and packaging protein comprises at least one selected from the group consisting of an adenovirus E1 gene, an adenovirus E1A gene, an adenovirus E1B gene, an adenovirus E2 gene and an adenovirus E4 gene. According to the timing of transcription, adenoviral genes are mainly divided into early expression genes (E1˜4) and late expression genes (L1˜5). After the adenoviral genome enters the nucleus, the cellular transcription factor firstly binds to the enhancer located upstream of the E1A region, expressing E1A protein which regulates cellular metabolism and makes the viral DNA replicated in cells more easily. The E1A protein also can activate promoters of other early genes (E1B, E2A, E2B, E3 and E4), where E2B drives the expression of three additional transcriptional units of early genes involving in viral replication (i.e. a precursor terminal protein (pTP), a single-stranded DNA binding protein (ssDBP) and a DNA polymerase (DNA pol)). Expression products of these three genes are tightly bound to be a complex, which interacts with at least three cellular proteins to initiate viral genome replication. The expression products of E1 region gene are divided into E1A and E1B. E1A mainly consists of two components, that is 289R (or 13S) and 243R (or 12S). The main function of the E1A protein is in regulating cellular metabolism, thus making cells more susceptible to viral replication. E1B19K is homologous to the expression product of Bcl-2 gene, and can prevent cells from apoptosis or necrosis by inactivating and clearing Bax family members. The E1B55K gene product can down-regulate the transcription level of p53 gene, with other adenoviral genes (such as E4 or f6) involved. The E1B55K gene product is also involved in viral replication, transcription of viral late mRNAs, and transportation of viral RNAs. The expression products of E2 region gene are divided into E2A and E2B, where E2A is a DNA binding protein (DBP), and E2B mainly has two products (i.e. a precursor terminal protein (pTP) and a viral DNA polymerase (pol) respectively). Such three proteins at least interact with three intracellular factors to initiate adenoviral DNA replication as well as transcription and translation of viral late genes. The gene products of E4 region gene are usually referred as orf 1 to 6/7, mainly involving in metabolism of viral messenger RNA, as well as promoting viral DNA replication and shutting down protein synthesis in a host. Studies have found that some E4 products can bind to DNA-activated protein kinases to prevent viral DNA from concatenating. Since this kinase can activate p53 gene, it is believed that some products of E4 region gene can inhibit cell apoptosis. Many products of E1B and E4 genes are involved in antagonizing the E1A protein. For example, E4 inhibits the activation of E2F promoter via E1A.

According to an embodiment of the present disclosure, the immune effector comprises at least one sequence selected from the group consisting of an inhibitory sequence that antagonizes the PD-1 gene, an inhibitory sequence that antagonizes the PD-L1 gene, an inhibitory sequence that antagonizes the CTLA4 gene, an inhibitory sequence that antagonizes the Tim-3 gene, GM-CSF, IL-2, IL-12, IL-15 or a fusion expression form of these factors. Programmed death 1 (programmed death receptor 1, PD-1) is an important immunosuppressive molecule belonging to a member of the CD28 superfamily, which is originally cloned from apoptotic mouse T cell hybridoma 2B4.11. PD-1 is an important inhibitory molecule on the surface of T cells, and its intracellular domain comprises an immunoreceptor tyrosine inhibitory motif (ITIM) and an immunoreceptor tyrosine transfer motif (ITSM). ITSM mediates the recruitment of protein tyrosine phosphatase family phosphatase and the inhibition of T cell activation signals. Ligands of ITSM are PD-L1 and PD-L2, which mainly play a major role in inhibiting activation of T cells in the tumor microenvironment during immune responses of immune system. Programmed cell death 1 ligand (PD-L1), also known as a cluster of differentiation 274 (CD274) or a B7 homolog (B7 homolog 1, B7-H1), is a human protein in body that is encoded by CD274 gene. PD-L1 is expressed inductively in a variety of tumor cells and hematopoietic cells in tumor cell microenvironments, and its expression level is positively correlated with the malignancy of tumors. PD-1 antibody and PD-L1 antibody can block the binding of PD-1 to PD-L1. However, tumor cells in tissues can escape from immune system through the PD-1/PD-L1 pathway. Cytotoxic T lymphocyte associate protein-4, short for CTLA-4, is a type of co-stimulatory molecule expressed on the surface of T cells. CTLA-4 is capable of specifically binding to CD80/CD86 on the surface of APC to activate downstream signals during activation of T cells, similar to the function of CD28. Studies have found that cells that predominantly express CTLA-4 among T cells are regulatory T cells (Treg), which is a class of T cells that negatively regulates cellular immunity. In the absence of CTLA-4 receptor, mice exhibit T cell overexpression, thus accompanied with severe autoimmune diseases. The above results show that Treg needs CTLA-4 to exercise its function. In addition, CTLA-4 is also expressed in conT cells, which plays a role in inhibiting the signaling of T cell activation. T cell immunoglobulin and mucin-domain containing molecule (TIM) gene family is a novel gene family discovered by Mclntire in 2001 in search of mouse asthma susceptibility genes, which is further identified by genomic analysis and positional cloning. The TIM family gene contains immunoglobulin V region and mucin region. Tim-3 is an important member of the TIM family and expresses a negative regulatory molecule on the surface of activated Th1 cells. Currently, it is found that TIM3 is expressed in CD8⁺ T cells, Th17 cells, Treg, NK cells and other lymphocyte subsets. Granulocyte-macrophage Colony Stimulating Factor (GM-CSF) is a monomeric glycoprotein cytokine secreted by macrophages, T cells, NK cells and the like, which stimulates stem cells to produce granules cells (neutrophils, eosinophils, basophils and monocytes). GM-CSF also affects mature cells in the immune system, such as inhibiting the transfer of neutrophils and altering expression of receptor on the cell surface. This factor also can inhibit fungal infections by activating macrophages. Interleukin 2 (IL-2) can mediate cytokines between white blood cells and white blood cells as well as between white blood cells and other cells. IL-2 is mainly produced by activated T cells, and acts on local target cells in a manner of autocrine and paracrine, which is a major cytokine involving in immune response, thus exhibiting a significant immune effect. IL-2 can also promote T cell proliferation and produce cytokines, promote B cell proliferation and secrete Ig, activate macrophages and enhance activation and proliferation of NK cells. In addition, IL-2 also has a negative regulation effect, which can induce apoptosis of T cells which activate Ag, limit the intensity of immune response, and avoid obvious immune damage. Interleukin-12 (IL-12) is a kind of interleukin produced by dendritic cells, macrophages, neutrophils and human B lymphoblasts (nc-37) in response to antigen stimulation. IL-12, called T cell stimulating factor, participates in differentiation of naive cells into Th1 cells, and stimulates the production of interferon gamma (IFN-γ) and tumor necrosis factor alpha (TNF-α). IL-12 plays an important role in regulating the activities of natural killer cells and T lymphocytes. IL-12 mediates enhanced cytotoxic activity of NK cells and CD8+ cytotoxic T lymphocytes. IL-12 also has anti-angiogenic activity, indicating it can block the formation of new blood vessels. IL-12 increases the production of gamma-interferon (INF-γ) thus increasing the induction of protein 10 (IP-10 or CXCL10), where IP-10 mediates anti-angiogenesis. IL-12 has been tested as a potential anti-cancer drug because of its ability of inducing immune response and anti-angiogenesis. IL-15 is a factor recently discovered which can be produced by a variety of cells, such as activated monocytes-macrophages, epidermal cells, fibroblasts and the like. The molecular structure of IL-15 is much similar to that of IL-2, thus IL-15 is capable of exerting a biological activity similar to IL-2 based on the binding of β-chain and γ-chain of IL-2 receptor to target cells. IL-15 can induce proliferation and differentiation of B cells, which is the only cytokine that can partially substitute IL-2 to induce initial antibody production. IL-15 can stimulate proliferation of T cells and NK cells, induce LAK cell activity, and can also interact with IL-12 to synergistically stimulate NK cells so as to produce IFN-γ.

According to an embodiment of the present disclosure, the protein of interest and the first regulatory protein are co-expressed under the control of one same promoter, and the protein of interest and the first regulatory protein are linked by a cleavable linker peptide. The protein of interest and the first regulatory protein are regulated and expressed under the one same promoter, and are further cleaved at the linker peptide after expression. The protein of interest is separated from the first regulatory protein, and the protein of interest and the first regulatory protein function independently of each other.

According to an embodiment of the present disclosure, the ninth nucleic acid molecule and the tenth nucleic acid molecule independently inhibit expression of the first regulatory protein or the second regulatory protein, respectively, via RNA interference. MicroRNA is a specific microRNA expressed in different cellular microenvironments, and the ninth or tenth nucleic acid molecule is a specific target sequence of microRNA. The specific effect between the microRNA expressed in a specific microenvironment and its target sequence can be realized via RNA interference, thereby specifically regulating the expression of the first regulatory protein or the second regulatory protein. MicroRNAs (miRNAs) are a class of endogenous small RNAs having about 20-24 nucleotides, and several miRNAs can regulate one same gene. The expression of a gene can be finely regulated by a combination of several miRNAs. MicroRNA has many forms, including a pri-miRNA in an original form, which is of a length of about 300-1000 bases; a pre-miRNA, in a form of microRNA precursor, which is formed by processing of the pri-miRNA and is of a length of about 70-90 bases; and a mature miRNA of about 20 to 24 nt, which is generated by digestion of the pre-miRNA with Dicer. The RNA-induced silencing complex (RISC) inhibits the expression of target genes. The effect of microRNA-RISC on mRNAs of target genes has always been largely dependent on the complementation degree between the microRNA-RISC and the target gene transcript sequence, which is performed in three ways. In a first way, the mRNA molecules of target gene are cleaved, that is the miRNAs which are completely complementary to a target gene (having closely similar mechanism and function to those of siRNAs) are cleaved. In plants, most of miRNAs of target genes are cleaved by the method described above, and the molecules without poly(A) are added with Uracils (Us) at the 3′ end and are rapidly degraded while the molecules with poly(A) can be stably present for a time period (such as Arabidopsis miR-171). Currently, one miRNA is found to be completely complementary to three potential target genes in plants, even although it is unclear whether these genes are targets of the miRNA. However, it is found for the first time that miRNA can be completely complementary to its potential targets, suggesting miRNA may have a mechanism similar as siRNA. In a second way, the translation of target gene is inhibited. miRNAs are not completely complement to target genes, thus inhibiting the translation of target genes, without affecting the stability of mRNAs. Such a miRNA is the most widely discovered miRNA, like nematode lin-4, however few miRNAs in plants inhibit target genes in this way. The third way refers to binding inhibition, that is incorporating the two mechanisms in the above two ways. When miRNAs are completely complementary to target genes, they are cleaved directly. When miRNAs are not completely complementary to target genes, they participate in regulation of gene expression.

According to an embodiment of the present disclosure, the ninth nucleic acid molecule comprises a nucleic acid sequence specifically recognized by a first microRNA, and the tenth nucleic acid molecule comprises a nucleic acid sequence that specifically recognized by a second microRNA, where the first microRNA is a normal cell-specific microRNA, and the second microRNA is an abnormal cell-specific microRNA. Further, the first regulatory protein is expressed in abnormal cells and is not expressed or low-expressed in normal cells, while the second regulatory protein is expressed in normal cells and is not expressed or low-expressed in abnormal cells.

According to still another specific embodiment of the present disclosure, the first microRNA comprises at least one selected from the group consisting of miR199a, miR95, miR125, miRamir25b, Let-7, miR143, miR145 and miR200C. The microRNA as described above is expressed in normal liver cells.

According to still another specific embodiment of the present disclosure, the second microRNA comprises at least one selected from the group consisting of miR21, miR223, miR224, miR221, miR18, miR214, miR146a and miR1792 expressed in hepatoma cells (HepG2, Huh7 and PLC). The second microRNA described above is a microRNA specifically expressed in a hepatoma cell. Further, the first regulatory protein is expressed in liver cancer cells and is not expressed or low-expressed in normal cells, while the second regulatory protein is expressed in normal cells and is not expressed or low-expressed in liver cancer cells.

According to an embodiment of the present disclosure, the first nucleic acid molecule and the second nucleic acid molecule are loaded in a first expression vector; the third nucleic acid molecule, the fourth nucleic acid molecule, the fifth nucleic acid molecule, and optionally the ninth nucleic acid molecule are loaded in a second expression vector; and the sixth nucleic acid molecule, the seventh nucleic acid molecule, the eighth nucleic acid molecule, and optionally the tenth nucleic acid molecule are loaded in a third expression vector. The first, second and third expression vectors serve as a loading vector for the expression system to regulate the specific expression of the gene of interest in a suitable microenvironment, such as a cell.

The selection of the expression vector is not particularly limited as long as the function of the expression system in a suitable microenvironment can be achieved. According to a particular embodiment of the present disclosure, the first expression vector, the second expression vector and the third expression vector are each independently selected from at least one of the following:

plasmids, viruses, stable cell lines, and other material carriers such as nano-materials, liposomes, molecular-conjugated vectors, naked DNAs, chromosomal vectors and polymers. The liposome carrier is a liposome-DNA complex formed by packaging DNA with an artificial bilayer phospholipid. The liposome carrier is non-toxic and non-antigenic, has a large capacity, and can prevent target gene from nuclease degradation due to the encapsulation of liposome carrier. Such a liposome carrier can be used alone or in combination with other carriers, and target gene can be introduced into a specific site by intravenously injecting the liposome carrier encapsulating the target gene, with disadvantages of short duration of expression and unable to pass through cell membrane barrier. The molecular-conjugated vector consists of three parts: DNA, DNA binding factor and ligand.

According to an embodiment of the present disclosure, the virus comprises at least one selected from the group consisting of an adenovirus, a vaccinia virus and a retrovirus.

According to an embodiment of the present disclosure, the first expression vector, the second expression vector and the third expression vector are one same vector. According to a specific embodiment of the present disclosure, the components of the expression system in the present disclosure are loaded into the one same expression vector by the present inventors through the Cascade Golden-Gate Gibson/Gateway Assemble Method, thus effectively solving the problem of very low transfection efficiency when a plurality of large fragments are transfected.

According to an embodiment of the present disclosure, the one same vector is an adenovirus. Adenovirus as a gene therapy vector has the following advantages. 1) Adenovirus can be used on a wide range of hosts and has low pathogenicity to humans, thus adenoviral vector systems are widely used for the expression of human and non-human proteins. Further, adenoviruses can infect a range of mammalian cells and therefore can be useful in expression of recombinant proteins in most mammalian cells and tissues. It is particularly noted that adenoviruses are epitheliophilic, and most of human tumors are derived from epithelial cells. In addition, the replication genes and pathogenic genes of adenovirus are clear enough and the antibody against adenovirus has been prevalent in human populations (70-80% of adults having neutralizing antibodies against adenovirus). Humans produce slight self-limiting symptoms only after infected with wild-type adenovirus, and the treatment with ribavirin is effective. 2) Adenovirus can infect and express gene in proliferating and non-proliferating cells. Retrovirus can only infect proliferating cells, and DNA transfection cannot be performed in non-proliferating cells, thus cells are necessary to be kept in incubation. Adenovirus can infect almost all cell types, except for some lymphoma cells against adenovirus. Adenovirus is the best system for studying gene expression in non-proliferating primary cells, which allows direct comparison between the results obtained from transformed cells and primary cells. 3) Adenovirus can effectively proliferate and has a high titer. The adenovirus system can produce a titer from 10¹⁰ to 10¹¹ VP/mlL and can be up to 10¹³VP/mL after concentration, thus this feature makes it very suitable for gene therapy. 4) Adenovirus is homologous to human genes. Human viruses are generally used as vectors and human cells are used as hosts for adenoviral vector systems, which provide an ideal environment for accurate post-translational processing and proper folding of human proteins. Most of human proteins can achieve high expression levels and are fully functioning. 5) Adenovirus does not integrate into chromosomes and does not cause mutagenicity after insertion. Retrovirus will be randomly integrated into the host's chromosome(s), causing gene inactivation or oncogene activation. However, adenovirus does not integrate into the chromosome in almost all known cells except for egg cells, and thus does not interfere with other genes in hosts. The egg cell integrated with a single copy of adenovirus is a good system for producing transgenic animals with specific characteristics. 6) Adenovirus can be amplified in suspension culture medium. 293 cells can be adjusted for suspension culture, thus allowing large amplification of adenovirus. A large number of facts have shown that suspension of 293 cells can express recombinant proteins in a 1-20 L bioreactor. 7) Adenovirus has the ability of expressing multiple genes simultaneously. This is the first expression system which is designed to express multiple genes in a same cell line or tissue. The simplest method for construction of the expression system comprises inserting a double-expression cassette containing two genes into an adenoviral transfer vector, or co-transfecting a cell strain of interest with different recombinant viruses to express proteins respectively. Relative co-expression of individual recombinant proteins can be correctly estimated by determining the MOI ratio of different recombinant viruses.

According to a specific embodiment of the present disclosure, the adenovirus as described above was injected into a tumor mouse model by the present inventors, with significant inhibition to the growth of mouse tumor. The adenovirus as described above can be used as a safe and effective oncolytic virus vaccine so as to specifically kill related tumors in a safe and effective way.

According to an embodiment of the present disclosure, the adenovirus is obtained by steps: removing the adenoviral E1 gene and a part of adenoviral E3 gene associated with adenoviral replication and packaging from an adenoviral vector; inserting the adenoviral E1A gene into a gene circuit by a stepwise Golden Gate method; and incorporating the gene circuit into the adenoviral vector by the Gateway or Gibson method. Specifically, the gene circuit recognizing cancer cells is inserted into an adenoviral vector by the present inventors through three steps. First, marker elements associated with distinguishing of cancer cells from normal cells (such as a tumor-specific promoter, a gene circuit-related repression element, and a cancer cell-distinguishing miRNA recognition sequence) are constructed into a primary vector plasmid, thereby forming a primary element library, where two ends of the primary elements are each inserted with the recognition site of Esp3I so as to make multiple primary elements constructed into one secondary expression system by the Golden Gate method. Second, an expression system library is constructed, where multiple primary elements selected from the primary element library are assembled into three expression systems by the Golden Gate method, such three expression systems including a tumor-specific promoter system, a first regulatory element-mediated repression system and a second regulatory element-mediated repression system, with two ends of each expression system inserted with the recognition site of BsaI endonuclease. Third, a complete gene circuit is formed by randomly assembling the three expression systems through the Golden Gate method. Two ends of the gene circuit are each inserted with Gibson homologous sequence or Gateway recognition site for further experiments. Finally, the gene circuit is constructed into an adenoviral vector (with E1 gene and part of E3 gene removed) by the Gibson or Gateway method. The method of obtaining an adenovirus as described above ensures rapid modification of a complex oncolytic adenoviral vector with large fragments.

Recombinant Virus

In a second aspect of the present disclosure, provided in embodiments is a recombinant virus. According to an embodiment of the present disclosure, the recombinant virus comprises:

a first nucleic acid molecule, comprising a tumor cell-specific promoter, the tumor cell-specific promoter being an alpha-fetoprotein-specific promoter;

a second nucleic acid molecule, operably linked to the first nucleic acid molecule and encoding a transcriptional activator, the transcriptional activator being Gal4VP16;

a third nucleic acid molecule, comprising a first recognition sequence of the transcriptional activator, the first recognition sequence being 5×UAS;

a fourth nucleic acid molecule, operably linked to the third nucleic acid molecule and comprising a first promoter and a first regulatory element, wherein the first promoter is a miniCMV, and the first regulatory element comprises a plurality of repeated tetO sequences, and at least one of the pluralities of repeated tetO sequences is set downstream of the first promoter;

a fifth nucleic acid molecule, operably linked to the fourth nucleic acid molecule and encoding a first regulatory protein, the first regulatory protein being LacI,

wherein the fifth nucleic acid molecule further comprises a sequence encoding a protein of interest, the protein of interest comprises a viral replication protein and an immune effector,

wherein the immune effector and the viral replication protein are co-expressed, the immune effectors are linked by a cleavable linker peptide, the protein of interest and the first regulatory protein are regulated and co-expressed by one same promoter, and the protein of interest and the first regulatory protein are linked by a cleavable linker peptide;

a sixth nucleic acid molecule, comprising a second recognition sequence of the transcriptional activator, the second recognition sequence being 5×UAS;

a seventh nucleic acid molecule, operably linked to the sixth nucleic acid molecule and comprising a second promoter and a second regulatory element, wherein the second promoter is a miniCMV, the second regulatory element comprises a plurality of repeated LacO sequences, at least one of the pluralities of repeated LacO sequences is inserted downstream of the second promoter;

an eighth nucleic acid molecule, operably linked to the seventh nucleic acid molecule and encoding a second regulatory protein, the second regulatory protein being tetR-KRAB;

a ninth nucleic acid molecule, operably linked to the fifth nucleic acid molecule and configured to conditionally inhibit expression of the first regulatory protein, wherein the ninth nucleic acid molecule comprises a nucleic acid sequence specifically recognized by a first microRNA, the first microRNA being a normal cell-specific microRNA; and

a tenth nucleic acid molecule, operably linked to the eighth nucleic acid molecule and configured to conditionally inhibit expression of the second regulatory protein, wherein the tenth nucleic acid molecule comprises a nucleic acid sequence specifically recognized by a second microRNA, the second microRNA being a tumor cell-specific microRNA,

wherein the first regulatory element is adapted to inhibit the function of the first promoter by binding to the second regulatory protein, and the second regulatory element is adapted to inhibit the function of the second promoter by binding to the first regulatory protein.

With the recombinant virus according to the embodiment of the present disclosure, the first regulatory protein Lad and the protein of interest are specifically expressed in tumor cells and the second regulatory protein tetR-KRAB is not expressed or low-expressed specifically in tumor cells under the co-regulation of the alpha-fetoprotein-specific promoter, the ninth nucleic acid molecule and the tenth nucleic acid molecule, thus the tetR-KRAB-mediated inhibition mechanism on the first promoter miniCMV is released, therefore the first regulatory protein Lad and the protein of interest are effectively expressed under the control of the first promoter miniCMV, and the function of the second promoter miniCMV is effectively inhibited via the LacI-mediated inhibition mechanism, and the expression of tetR-KRAB is further inhibited. Further, proteins (like the protein of interest E1A and the first regulatory protein Lad) are more specifically expressed in tumor cells and the proteins like the second regulatory protein tetR-KRABs are not specifically expressed in tumor cells by using the recombinant virus according to the embodiment of the present disclosure, with high efficiency and specificity.

According to an embodiment of the present disclosure, the recombinant virus is at least one selected from the group consisting of a retrovirus, an adenovirus, a herpes virus and a vaccinia virus. (1) Retroviral vector has characteristics such as a similar structure and infection process to those of common retrovirus, stable expression of gene of interest in transformed target cells, no spread after infection of target cells, high efficiency of pseudovirus in infecting target cells, and no infection of non-proliferating cells. (2) Adenoviral vector has characteristics of a wide range of hosts, no dependence on host proliferation at expression of adenoviral protein, high virus titer, stable recombinant, no induction to tumor, high safety, no envelope protein, not easily inactivated by complement, direct application in body and no integration into chromosome. (3) Herpes simplex virus vector has characteristics of high titer, large capacity, infection into both proliferating and non-proliferating cells, non-integration, and long-term existence and stable expression.

According to an embodiment of the present disclosure, the recombinant virus is an adenovirus. As mentioned above, adenovirus as a gene therapy vector has many advantages.

1. Adenovirus is useful for a wide range of hosts and has low pathogenicity to humans, thus adenoviral vector systems are widely used for the expression of human and non-human proteins. Further, adenovirus can infect a range of mammalian cells and therefore can be useful in expression of recombinant proteins in most mammalian cells and tissues. It is particularly noted that adenoviruses are epitheliophilic, and most of human tumors are derived from epithelial cells. In addition, the replication genes and pathogenic genes of adenovirus are clear enough and the antibody against adenovirus has been prevalent in human population (70-80% of adults having neutralizing antibodies against adenovirus). Human produces slight self-limiting symptoms only after infected with wild-type adenovirus, and the treatment with ribavirin is effective.

2. Adenovirus can infect and express gene in proliferating and non-proliferating cells. Retrovirus can only infect proliferating cells, and DNA transfection cannot be performed in non-proliferating cells, thus cells are necessary to be kept in incubation. Adenovirus can infect almost all cell types, except for some lymphoma cells against adenovirus. Adenovirus is the best system for studying gene expression in non-proliferating primary cells, which allows direct comparison between the results obtained from transformed cells and primary cells.

3. Adenovirus can effectively proliferate and has a high titer. The adenovirus system can produce a titer from 10¹⁰ to 10¹¹ VP/mlL and can be up to 10¹³VP/mL after concentration, thus this feature makes it very suitable for gene therapy.

4. Adenovirus is homologous to human genes. Human viruses are generally used as vectors and human cells are used as hosts for adenoviral vector systems, which provide an ideal environment for accurate post-translational processing and proper folding of human proteins. Most of human proteins can achieve high expression level and are fully functioned.

5. Adenovirus does not integrate into chromosomes and does not cause mutagenicity after insertion. Retrovirus will be randomly integrated into the host's chromosome(s), causing gene inactivation or oncogene activation. However, adenovirus does not integrate into the chromosome in almost all known cells except for egg cells, and thus does not interfere with other genes in hosts. The egg cell integrated with a single copy of adenovirus is a good system for producing transgenic animals with specific characteristics.

6. Adenovirus can be amplified in suspension culture medium. 293 cells can be adjusted for suspension culture, thus allowing large amplification of adenovirus. A large number of facts have shown that suspension of 293 cells can express recombinant proteins in a 1-20 L bioreactor.

7. Adenovirus has the ability of expressing multiple genes simultaneously. This is the first expression system which is designed to express multiple genes in a same cell line or tissue. The simplest method for construction of the expression system comprises inserting a double-expression cassette containing two genes into an adenoviral transfer vector, or co-transfecting a cell strain of interest with different recombinant viruses to express proteins respectively. Relative co-expression of individual recombinant proteins can be correctly estimated by determining the MOI ratio of different recombinant viruses.

According to an embodiment of the present disclosure, the immune effector comprises at least one sequence selected from the group consisting of an inhibitory sequence that antagonizes PD-1 gene, an inhibitory sequence that antagonizes PD-L1 gene, an inhibitory sequence that antagonizes CTLA4 gene, an inhibitory sequence that antagonizes Tim-3 gene, IL-2, IL-12, IL-15 and GM-CSF, or a fusion expression form of these factors. Programmed death 1 (programmed death receptor 1, PD-1) is an important immunosuppressive molecule belonging to a member of CD28 superfamily, which is originally cloned from apoptotic mouse T cell hybridoma 2B4.11. PD-1 is an important inhibitory molecule on the surface of T cells, and its intracellular domain comprises an immunoreceptor tyrosine inhibitory motif (ITIM) and an immunoreceptor tyrosine transfer motif (ITSM). ITSM mediates the recruitment of protein tyrosine phosphatase family phosphatase and the inhibition of T cell activation signals. Ligands of ITSM are PD-L1 and PD-L2, which mainly play a major role in suppressing activation of T cells in the tumor microenvironment during immune response of immune system. Programmed cell death 1 ligand (PD-L1), also known as cluster of differentiation 274 (CD274) or B7 homolog (B7 homolog 1, B7-H1), is a human protein in body that is encoded by CD274 gene. PD-L1 is expressed inductively in a variety of tumor cells and hematopoietic cells in tumor cell microenvironments, and its expression level is positively correlated with the malignancy of some tumors. PD-1 antibody and PD-L1 antibody can block the binding of PD-1 to PD-L1. However, tumor cells in tissues can escape from immune system through the PD-1/PD-L1 pathway. Cytotoxic T lymphocyte associate protein-4, short for CTLA-4, is a type of co-stimulatory molecule expressed on the surface of T cells. CTLA-4 is capable of specifically binding to CD80/CD86 on the surface of APC to activate downstream signals during activation of T cells, similar to the function of CD28. Studies have found that cells that predominantly express CTLA-4 among T cells are regulatory T cells (Treg), which is a class of T cells that negatively regulate cellular immunity. In the absence of CTLA-4 receptor, mice exhibit T cell overexpression, thus accompanied with severe autoimmune diseases. The above results show that Treg needs CTLA-4 to exercise its function. In addition, CTLA-4 is also expressed in conT cells, which plays a role in inhibiting the signaling of T cell activation. T cell immunoglobulin and mucin-domain containing molecule (TIM) gene family is a novel gene family discovered by Mclntire in 2001 in search of mouse asthma susceptibility genes, which is further identified by genomic analysis and positional cloning. The TIM family gene contains immunoglobulin V region and mucin region. TIM-3 is an important member of the TIM family and expresses a negative regulatory molecule on the surface of activated Th1 cells. Currently, it is found that TIM3 is expressed in CD8⁺ T cells, Th17 cells, Treg, NK cells and other lymphocyte subsets. Granulocyte-macrophage Colony Stimulating Factor (GM-CSF) is a monomeric glycoprotein cytokine secreted by macrophages, T cells, NK cells and the like, which stimulates stem cells to produce granules cells (neutrophils, eosinophils, basophils and monocytes). GM-CSF also affects mature cells in the immune system, such as inhibiting the transfer of neutrophils and altering expression of receptor on the cell surface. This factor also can inhibit fungal infections by activating macrophages. Interleukin 2 (IL-2) can mediate cytokines between white blood cells and white blood cells as well as between white blood cells and other cells. IL-2 is mainly produced by activated T cells, and acts on local target cells in a manner of autocrine and paracrine, which is a major cytokine involving in immune response, thus exhibiting a significant immune effect. IL-2 can also promote T cell proliferation and produce cytokines, promote B cell proliferation and secrete Ig, activate macrophages and enhance activation and proliferation of NK cells. In addition, IL-2 also has a negative regulation effect, which can induce apoptosis of T cells which activate Ag, limit the intensity of immune response, and avoid obvious immune damage. Interleukin-12 (IL-12) is a kind of interleukin produced by dendritic cells, macrophages, neutrophils and human B lymphoblasts (nc-37) in response to antigen stimulation. IL-12, called T cell stimulating factor, participates in the differentiation of naive cells into Th1 cells, and stimulates the production of interferon gamma (IFN-γ) and tumor necrosis factor alpha (TNF-α). IL-12 plays an important role in regulating the activities of natural killer cells and T lymphocytes. IL-12 mediates enhanced cytotoxic activity of NK cells and CD8+ cytotoxic T lymphocytes. IL-12 also has anti-angiogenic activity, indicating it can block the formation of new blood vessels. IL-12 increases the production of gamma-interferon (INF-γ) thus increasing the induction of protein 10 (IP-10 or CXCL10), where IP-10 mediates anti-angiogenesis. IL-12 has been tested as a potential anti-cancer drug because of its ability of inducing immune response and anti-angiogenesis. IL-15 is a factor recently discovered which can be produced by a variety of cells, such as activated monocytes-macrophages, epidermal cells, fibroblasts and the like. The molecular structure of IL-15 is much similar to that of IL-2, thus IL-15 is capable of exerting a biological activity similar to IL-2 based on the binding of β-chain and γ-chain of IL-2 receptor to target cells. IL-15 can induce proliferation and differentiation of B cells, which is the only cytokine that can partially replace IL-2 to induce initial antibody production. IL-15 can stimulate proliferation of T cells and NK cells, induce LAK cell activity, and can also interact with IL-12 to synergistically stimulate NK cells so as to produce IFN-γ.

Recombinant Cell

In a third aspect of the present disclosure, provided in embodiments is a recombinant cell. According to an embodiment of the present disclosure, the recombinant cell comprises an expression system as described above. The recombinant cell according to the embodiment of the present disclosure can effectively activate the systemic immune response in the human body, and attacks xenogeneic cells such as tumor cells, with high safety and specificity.

According to an embodiment of the present disclosure, at least a portion of the expression system is integrated into the genome of the recombinant cell. The expression system replicates as the recombinant cell genome replicates, and the expression system can regulate the expression of proteins of interest constantly and effectively.

Use in the Preparation of a Medicament

In a fourth aspect of the present disclosure, provided in embodiments is use of the aforementioned expression system, the aforementioned recombinant virus, and the aforementioned recombinant cell in the preparation of a medicament for treating a cancer. The expression system described in the present disclosure can specifically express a protein of interest in tumor cells. The drug according to the embodiment of the present disclosure can treat cancers in a more effective, specific and safer way. In this embodiment, the present inventors have designed a gene circuit in response to multiple targets to regulate the expression of the key gene E1A associated with adenoviral transcription according to the concept of synthetic biology. At the same time, such a co-expression can activate cytokine or antibody gene which is related to a systemic immune response. First, the gene circuit designed by the present inventors can regulate the packaging of adenoviruses at multiple levels, unlike traditional oncolytic adenoviruses. In the first step, a tumor-specific promoter is used by the present inventors to regulate the expression of the master switch Gal4VP16 in the gene circuit. In the second step, the target sequence of microRNA in the gene circuit can distinguish tumor cells from non-tumor cells in response to microRNA expression in different cell lines. In the third step, a stable mutually repressive switch is used in the designed gene circuit by the present inventors, which can efficiently respond to external input signals such as cell-specific promoters and microRNA signals, and the gene circuit can further enlarge the difference of input signals and be more efficient in distinguishing tumor cells from non-tumor cells. In the fourth step, the expression system in the adenoviral vector designed by the present inventors does not comprise the adenoviral E1B gene, where the E1B gene can interact with the P53 gene in normal cells to ensure the successful adenovirus proliferation in normal cells. The adenovirus without the E1B gene could not proliferate in normal cells expressing P53, while it can still efficiently proliferate in tumor cells lacking the P53 gene. Therefore, the adenoviral vector in the embodiment is more specific and safer than the traditional oncolytic adenovirus through multi-level regulation. In addition, this engineered adenovirus is used as a vector to carry genes expressing a variety of cytokines and antibodies in the embodiment. Thus, the adenovirus not only exhibits the oncolytic effect, but also can activate the systemic immune response by using the co-expressed factors, with improved efficacy of oncolytic adenovirus.

Specifically, the present application constructs a gene circuit to regulate the specific expression and packaging of oncolytic adenovirus in hepatocellular carcinoma cells by synthetic biology means. Except for having oncolytic efficacy, the adenovirus constructed by the present inventors is also loaded with genes expressing cytokines which can activate immune response in the body. Such a cytokine once expressed can stimulate immune response inhibiting tumor growth to treat tumors. The expression system designed in the present application has great innovation and technical advantages as follows.

Efficient and safe: The gene circuit designed in this application can respond to biomarkers distinguishing normal cells from cancer cells at multi-levels and comprises a mutually repressive close-loop gene circuit to regulate the replication of adenovirus in different cells, thus is capable of responding to microenvironments in different cell lines more sensitively while reducing the effects of gene expression noise.

Efficient: The gene circuit in this application is loaded with genes expressing a variety of cytokines and/or antibodies, thus an effective immune response can be induced in the cells of interest.

Modularization of gene circuit: Biomarkers involved in the gene circuit are modularized to modules, which are each modelled into different module libraries containing biomarkers against different diseases, thus a disease-specific gene circuit can be constructed by assembling relative modules rapidly.

Accuracy: It is possible for inventors to replace different tumor markers based on rapid assembly technology, thus gene circuits directing different patients can be designed for precise treatment.

According to an embodiment of the present disclosure, the cancer comprises liver cancer, lung cancer, colorectal cancer, melanoma, breast cancer or prostate cancer. The inventors have found that the drug in the embodiment of the present disclosure has a more remarkable therapeutic effect on liver cancer, lung cancer, colorectal cancer, melanoma, breast cancer and prostate cancer.

According to a specific embodiment of the present disclosure, the inventors have found that the expression of microRNAs in different tumor cell lines is shown in Table 1. The upward arrow indicates microRNAs that are highly expressed in cancer cells compared to normal cells, while the downward arrow indicates microRNAs that are low-expressed in cancer cells compared to normal cells.

TABLE 1

| | |
|---|---|
| Hepatocellular carcinoma | miR-21↑, 18↑, 224↑, 199↓, 195↓, 200↓, 125↓ |
| Lung cancer | Let-7↓, miR-17-92↑ |
| Breast cancer | miR125b↓, 145↓, 21↓, 155↓ |
| Brain tumor | miR-21↑, 221↑, 181↓, |
| Colorectal cancer | miR143↓, 145↓ |
| Lymphoma | miR155↑, miR-17-92↑ |
| Melanoma | miR21↑, 221↑, 214↑146a↑137↓200c↓let-7↓ |

Method of Expressing a Protein of Interest by Using an Expression System

In a fifth aspect of the present disclosure, provided in embodiments is a method of expressing a protein of interest by using an expression system. The expression system is the expression system as described above. According to an embodiment of the present disclosure, the method comprises: (1) providing a fifth nucleic acid molecule which comprises a nucleic acid sequence encoding the protein of interest; and (2) inhibiting expression of a second regulatory protein by a tenth nucleic acid molecule so as to express the protein of interest. With the method of expressing a protein of interest by using an expression system in the embodiment, the second regulatory protein-mediated inhibition mechanism on the first promoter is released, thus the protein of interest is effectively expressed in specific cells under the co-action of the cell-specific promoter and the first promoter.

According to an embodiment of the present disclosure, the expression is carried out in cells. The cells can provide a microenvironment for the expression of proteins of interest, thus the proteins of interest can be expressed in cells more efficiently.

According to an embodiment of the present disclosure, the tenth nucleic acid molecule comprises a nucleic acid sequence specifically recognized by the second microRNA, and the method further comprises contacting the second microRNA with the tenth nucleic acid molecule in the step (2). MicroRNAs (miRNAs) are a class of endogenous small RNAs having about 20-24 nucleotides, and several miRNAs can regulate one same gene. The expression of a gene can be finely regulated by a combination of several miRNAs. MicroRNA has many forms, including a pri-miRNA in an original form, which is of a length of about 300-1000 bases; a pre-miRNA, in a form of microRNA precursor, which is formed by processing of the pri-miRNA and is of a length of about 70-90 bases; and a mature miRNA of about 20 to 24 nt, which is generated by digestion of the pre-miRNA with Dicer. The RNA-induced silencing complex (RISC) inhibits the expression of target genes. The effect of microRNA-RISC on target gene mRNA has always been largely dependent on the complementation degree between the microRNA-RISC and the target gene transcript sequence, which is performed in three ways. In a first way, the mRNA molecules of target gene are cleaved, that is the miRNAs which are completely complementary to target gene (having closely similar mechanism and function to those of siRNAs) are cleaved. In plants, most of miRNAs of target genes are cleaved by the method described above, and the molecules without poly(A) are added with Uracils (Us) at the 3′ end and are rapidly degraded while the molecules with poly(A) can be stably present for a time period (such as Arabidopsis miR-171). Currently, one miRNA is found to be completely complementary to three potential target genes in plants, even although it is unclear whether these genes are targets of the miRNA. However, it is found for the first time that miRNA can be completely complementary to its potential targets, suggesting miRNA may have a mechanism similar as siRNA. In a second way, the translation of target gene is inhibited. MiRNAs are not completely complement to target genes, thus inhibiting the translation of target genes, without affecting the stability of mRNAs. Such a miRNA is the most widely discovered miRNA, like nematode lin-4, however few miRNAs in plants inhibit target genes in this way. The third way refers to binding inhibition, that is incorporating the two mechanisms in the above two ways. When miRNAs are completely complementary to target genes, they are cleaved directly. When miRNAs are not completely complementary to target genes, they participate in regulation of gene expression.

In a sixth aspect of the present disclosure, provided in embodiments is a method of expressing a protein of interest by using an expression system. The expression system is the expression system as described above. According to an embodiment of the present disclosure, the method comprises: (1) providing an eighth nucleic acid molecule which comprises a nucleic acid sequence encoding a first protein of interest, and providing a fifth nucleic acid molecule which comprises a nucleic acid sequence encoding a second protein of interest; and (2) inhibiting expression of the first regulatory protein by the ninth nucleic acid molecule so as to express the first protein of interest; or inhibiting expression of the second regulatory protein by the tenth nucleic acid molecule so as to express the second protein of interest. With the method of expressing a protein of interest by using an expression system in the embodiment, the second regulatory protein-mediated inhibition mechanism on the first promoter is released, thus the second protein of interest is effectively expressed in specific cells under the coaction of the cell-specific promoter and the first promoter, or the first regulatory protein-mediated inhibition mechanism on the second promoter is released, thus the first protein of interest is effectively expressed in specific cells under the coaction of the cell-specific promoter and the second promoter.

Experimental embodiments of the present disclosure are described in detail below. The embodiments described below are illustrative only and are not to be construed as limiting the present disclosure. Where specific techniques or conditions are not indicated in the examples, they are carried out according to the techniques or conditions described in the literature in the art or in accordance with the product specifications. The used reagents or instruments not indicated by the manufacturer are all conventional products that can be obtained commercially.

Experimental Materials and Methods

1. Cell Culture and Transfection

HEK293 (293-H) cell line was purchased from Invitrogen Co., Ltd. Human hepatoma cell line HepG2, normal liver cells Chang and Hepa1-6 were purchased from ATCC. Human hepatoma cell line Huh7 was purchased from BeNa Co., Ltd. The cells were cultured in high-glucose Dulbecco's Modified Eagle's Medium containing 4.5 g/L glucose, 0.045 unit/mL penicillin, 0.045 g/mL streptomycin and 10% FBS (purchased from the Invitrogen Co., Ltd.) (i.e. DMEM complete medium) at 37° C., 100% humidity and 5% CO₂ concentration.

Cell transfection was conducted as follows. About 7.5×10⁴ HEK293 cells in 0.5 mL of the high-glucose DMEM complete medium were seeded into individual wells of 24-well plates (Falcon) and incubated for 24 hours, followed by replacing the medium with fresh DMEM complete medium and addition of Attractene transfection reagent (Qiagen) or Lipofectamine LTX (Life Technologies) for transfection. Specifically, 1.5 μL of Attractene was added into an amount of mixed DNA plasmids, stood at room temperature for 15 minutes, and then added into corresponding wells if Attractene was used. However, if Lipofectamine LTX was used, an appropriate amount of Plus reagent was firstly added into mixed DNA plasmids and stood at room temperature for 5 minutes, then 1.25 μL of Lipofectamine LTX was added and stood at room temperature for another 25 minutes prior to transferred into the 24-well plates. pDT7004 (pUBI-linker-NOS), which contains a maize ubiquitin promoter (UBI) followed by a NOS terminator with no protein-coding sequences between UBI and NOS, was used to ensure that the amount of plasmid DNA in different experiment groups was equal. Cells after transfection were cultured for 48 hours before flow cytometry analysis or some experiments related to cytokine detection.

2. Adenoviral Vector

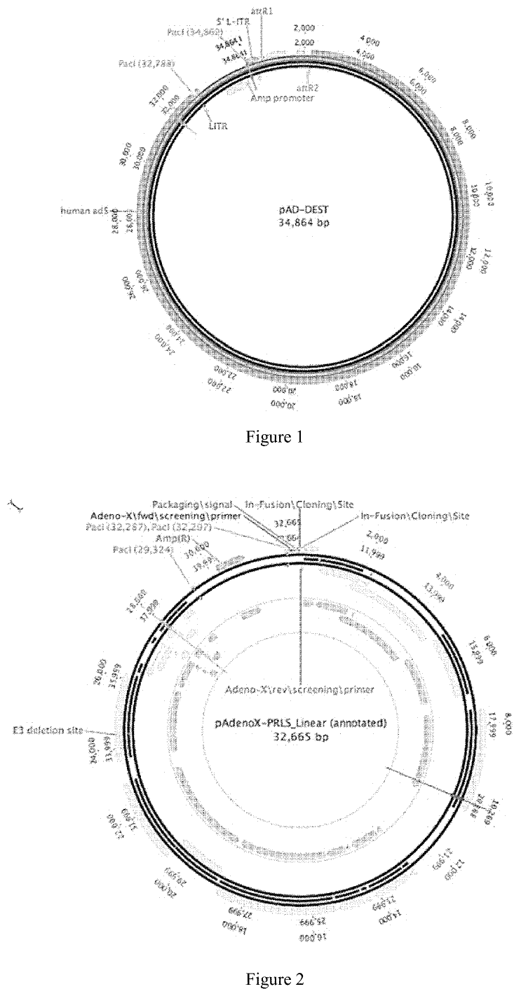

Two initial adenoviral vector sequences are used in this example, that is ViraPower™ Adenoviral Gateway™ Expression Kit (K49300 Invitrogen) and Adeno-X-Adenoviral System 3 (632266 Clontech). The structures of the two adenoviral vectors are shown in FIG. 1 and FIG. 2 respectively.

3. Plasmid Construction

The plasmids related to truncated AFP promoter derivatives were constructed. AFP enhancer and AFP promoter sequences were directly obtained from the genome of hepatoma cell line under specifically designed PCR primers via PCR amplification. Point mutations in the AFP promoter sequence were obtained by using the overlap extension PCR method. The AFP enhancer and AFP promoter sequences were constructed into an entry clone, with a specifically designed recognition sequence of LR Clonase at both ends of the entry clone. The specific promoter and the activator gene Gal4VP16 were constructed into one same vector by the LR reaction. The target sequence of microRNA was inserted into the CMV-EYFP vector (XbaI/SalI) through primer annealing, enzyme digestion and ligation. The primary element library of the expression system was constructed through the Golden Gate method. The type IIs restriction enzyme (such as BsaI, BbsI, BsmBI, SapI and the like) is a specific class of restriction enzyme where its recognition site is a non-palindromic and symmetrical sequence and its cleavage site is located outside the recognition site, which is different from the type II restriction enzyme most commonly used in molecular biology where its recognition site is a palindromic and symmetrical sequence and its cleavage site overlaps with the recognition sequence. Thus, when two fragments digested by the type IIs restriction enzyme are ligated in subsequent digestion and ligation reactions, they will not be cleaved again due to the absence of previous recognition sites, thus achieving the effect of welding. For the Golden Gate cloning method, different cleavage site sequences are artificially designed outside the recognition sequences because of such a characteristic of the type IIs restriction enzyme, by which one same type IIs restriction enzyme is capable of generating different sticky ends, thereby ensuring of assembling multiple fragments at one time, and thus overcoming the deficiency of traditional multi-fragment assembly such as use of multiple different restriction enzymes. The biggest feature of this Golden Gate cloning method is in that no additional “scar” is introduced and thus achieving the seamless ligation of DNA fragments, compared with the traditional digestion-ligation method. The type IIs restriction enzyme used in the primary element library in this example is Esp3I. A second logic circuit library of the expression system was also constructed by the Golden Gate method, with the type IIs restriction enzyme BsaI used. The second logic circuit was then inserted into the adenoviral vector sequence of the ViraPower™ Adenoviral Gateway™ Expression Kit by the Gateway method, or inserted into the adenoviral vector sequence of Adeno-X Adenoviral System 3 by Gibson cloning. The Gateway technique is based on the well-studied phage λ site-specific recombination system (attB×attP→attL×attR), which consists of BP and LR reactions. The BP reaction is a recombination reaction between an attB DNA fragment or an expression clone and an attP donor vector, thus creating an entry clone. The LR reaction is a recombination reaction between an attL entry clone and an attR destination vector, which is used to insert a sequence of interest into one or more destination vectors in parallel reactions. The present inventors employed the LR reaction in this example. Gibson assembly technique, also known as “Gibson thermostatic one-step assembly method”, is a DNA assembly method created by Gibson et al. at the J. CraigVenter Institute in the United States of America, where a plurality of DNA fragments having overlapping end sequences are assembled in vitro under the synergistic effect of T5 exonuclease, DNA polymerase and ligase. Among them, T5 exonuclease has 5→3′ exonuclease activity, and can cleave the DNA fragment having overlapping end sequences from the 5′ end of one strand of the DNA fragment, thus generating an overhanging end having the overlapping sequence at the 3′ end of the complementary strand which is specifically annealed at 50° C. (T5 exonuclease is gradually inactivated at this temperature), and finally a complete DNA molecule is formed in the presence of DNA polymerase and Taq ligase, achieving seamless assembly.

4. Cellular RNA Extraction, Reverse Transcription and Quantitative PCR Reaction

Cellular RNA was extracted by the Trizol method. 1 mL of Trizol was added into 5×10⁶ cells pelleted by centrifugation, followed by repeatedly pipetting with a pipette or vigorous shaking to lyse cells. After standing at room temperature of 15-30° C. for 5 minutes, 0.2 mL of chloroform was added, and the mixture was shaken vigorously for 15 seconds, kept at room temperature for another 2 to 3 minutes, and centrifuged at 12,000×g and 4° C. for 15 minutes. 0.5 mL of isopropanol was added into the separated upper aqueous layer, left at room temperature for 10 minutes, and centrifuged at 12,000×g and 4° C. for 10 minutes, followed by adding 1 mL of 75% ethanol, vortexed and centrifugation at 7500×g and 4° C. for 5 minutes. The supernatant was discarded, and the precipitated RNA was naturally dried at room temperature. The dried RNA was dissolved in RNase-free water. The reverse transcription experiment was performed by using the QuantiTect Rev. Transcription Kit (Cat No./ID: 205310 QIAGEN). The quantitative PCR primers are qAFP for: CAAAGCTGAAAATGCAGTTGAATG (SEQ ID NO: 15), qAFP rev: TTCCCCATCCTGCAGACAATCC (SEQ ID NO: 16), GAPDH for: AGAAGGCTGGGGCTCATTTG (SEQ ID NO: 17), and GAPDH rev: AGGGGCCATCCACAGTCTTC (SEQ ID NO: 18). Fluorescent dye is PowerUp™ SYBR® Green Master Mix (A25741Thermo Fisher).

5. Packaging, Purification and Titration of Adenovirus

The endotoxin-free recombinant adenoviral plasmid with high purity was extracted. HEK293 cells were transfected with the linearized adenovirus DNA which was digested by PacI overnight, followed by replacing the culture medium with the complete medium. Cells along with 1 mL of culture medium were collected into a 15 mL centrifuge tube after one week of culture (fresh medium added depending on the cell growth state during the culture), which were then lysed with three consecutive freeze-thaw cycles at 37° C. via liquid nitrogen, and centrifuged at 2000 rpm for 5 minutes. The supernatant was obtained as the primary virus solution. After three consecutive generations of transfection, the obtained adenoviruses were seeded into 10 of 15 cm plates for amplification, and further purified by the combination of CsCl density gradient centrifugation and dialysis. The CsCl density gradient was prepared by adding 2 mL of a CsCl solution in a density of 1.4 g/mL, further adding 3 mL of a CsCl solution in a density of 1.3 g/mL slowly, and then adding 5 mL of the virus suspension. After centrifugation at 20000 rpm and 4° C. for 10 hours, the adenoviruses concentrated in the CsCl solution in a density between 1.3 g/mL and 1.4 g/mL were collected into dialysis bags which have been boiled with 10 mM EDTA-Na₂ for 10 minutes before use. The dialysis buffer is prepared by using 50 g sucrose, 10 mL 1 M Tris-HCl and 2 mL 1 M MgCl₂, made up to 1 L with water and adjusted to pH 8.0. After stirring at 4° C. overnight and changing the dialysis buffer twice, the adenovirus was collected for determination of virus titer. The viral titer was detected by quantifying the viral DNA extracted from the infected cells. Specifically, about 2×10⁶ HeLa cells/well were seeded into 6-well tissue culture plates (with the cell coverage rate reaching up 90-100% after one day of incubation) and infected with 5 μL of the virus stock diluted with 500 μL of serum-free medium in duplicate, followed by incubation at 37° C. for 3 to 6 hours, removal of culture medium, and washed twice with 1 mL PBS. The cells were lysed by adding 500 μL of fresh NP-40 lysis buffer (including 0.65% NP-40 (Calbiochem, Billerica, Mass., USA), 150 mM NaCl, 10 mM Tris, pH 8.0), incubating at room temperature for 5-10 minutes with pipetting up and down 10-15 times for fully lysis, and then centrifuging at 2000×g for 3 minutes for virus precipitation. After the supernatant was discarded, 1 mL of NP-40 lysis buffer was added followed by vortexed briefly and centrifugation at 2000×g for 3 minutes. The supernatant was discarded again and the precipitate was suspended in 200 μL of PBS. During the DNA isolation and purification, DNAs were extracted by using the DNeasy Blood & Tissue Kit (QIAgen, Valencia, Calif., USA) for subsequent quantitative reactions. The quantitative primers are L2 Forward Primer: TTGTGGTTCTTGCAGATATGGC (SEQ ID NO: 19), and L2 Reverse Primer: TCGGAATCCCGGCACC (SEQ ID NO: 20).

A standard curve was prepared. The PCR L2 target sequence was constructed into T vectors, which were diluted to 1×10⁹ to 1×10² copy/μL as a standard sequence. The dilution formula is shown as: [Plasmid concentration (μg/μL)×Avogadro's number×10-6]/[number of nucleotides×lbp molecular weight]=particle copies/μL

6. Cell Experiment

Adenovirus was detected for its recognition and killing abilities to different cells. Chang cells (1×10⁴ cells/well), and HepG2 and Huh7 cells (5×10⁴ cells/well respectively) were seeded into 96-well plates and infected with adenoviral vectors at seven Multiplicity of Infection (MOI) gradients in ten-fold dilutions from 10² to 10⁻⁴, with cell viability measured by the MTS method after six days of observation.

7. MTS Detection

The number of cells was measured by the MTS method. 20 μL of MTS/PMS mixture (Promega) was added to individual wells of 96-well plates containing the cells to be tested, then mixed, and incubated at 37° C. for 1 to 4 hours. The plate was shaken for 10 seconds after color development to get uniform color. The absorbance (OD value) at a wavelength of 490 nm was measured by using a microplate reader. A standard curve was established. The number of cells of interest was detected by reference of the standard curve.

8. Cytokine Detection

The biological activity of cytokines can be detected via proliferation of cytokine-dependent cell line. The biological activities of IL-2, mGM-CSF and hGM-CSF were detected by using CTLL-2, FDC-P1 and TF-1 cell lines, respectively. After collected, 1×10⁴ cytokine-dependent cells to be tested were seeded in 96-well plates, infected with the sample containing the cytokine to be tested, and thermostatically cultured at 37° C. for 24 hours or 48 hours. The amplified cells were measured by the MTS method. A dose-proliferation standard curve was established. The bioactivity of cytokine in the measured sample was estimated by using the standard curve.

9. Co-Immunoprecipitation Assay

Co-immunoprecipitation was performed as follows. An appropriate amount of protein lysis buffer (Biyuntian Co., Ltd.) was added into the collected cells to be tested, and centrifuged to remove cell debris. A small amount of the protein lysis buffer was left for subsequent Western blot analysis. The remaining protein lysis buffer plus 10-30 μL of protein A/G-agarose beads conjugated with corresponding tag antibody (Thermo Fisher Scientific Co., Ltd.) was added into the obtained cell lysate, and incubated at 4° C. overnight with slightly shaking, then centrifuged to remove the supernatant. After the precipitated beads were washed three times, the antibody and captured protein were eluted from the agarose beads by using an eluent, followed by addition of SDS-PAGE loading buffer (Shanghai Shenggong Co., Ltd.) and boiled. The enrichment of related proteins was detected by the Western blot method.

10. Animal Studies

The experimental animals were purchased from HuafuKang Co., Ltd. or Weitonglihua Co., Ltd. The tumor model was constructed by using 6-week old male or female mice, kept in SPF environment. For the human tumor model, six-week old nude mice were subcutaneously injected with 1×10⁷ human hepatoma cells HepG2 in matrigel (BD Co., Ltd.) on the right flank. For the mouse tumor model, 1×10⁶ mouse hepatoma cells Hepa1-6 were subcutaneously injected into the right flank of six-week old wild-type C57BL/6J mice. Subcutaneous tumor size was measured by vernier calipers every 3 days, and calculated by using the formula: Volume=0.5×length×width. After tumor volume exceeded 100 mm³, oncolytic adenoviruses or control treatment was administrated. Tumor-bearing mice were euthanized after the tumor size reached 15 mm in diameter, according to the requirements of animal experiment ethics.

Experimental Results

1. Construction of Mutually Repressive Switch in Response to Various Biomarkers to Distinguish Cancer Cells from Non-Cancer Cells by Use of Synthetic Biology

Related studies in recent years have shown that oncolytic viruses are very effective for treating a cancer, thus many clinical studies on oncolytic adenovirus have been developed. According to the existing clinical research results, there is a relatively high proportion of leakage on oncolytic adenovirus, so how to improve the safety of oncolytic adenovirus in clinical stage is an important issue. Based on the analysis of existing oncolytic adenoviruses, most oncolytic adenoviruses are regulated depending on a single biomarker, such as a tumor-specific promoter that regulates the expression of gene E1 key to adenoviral replication. However, only one single biomarker may cause a relatively high probability of leakage. Therefore, the present inventors have designed a mutually repressive switch in response to a variety of biomarkers and to regulate the expression of adenovirus E1A gene at various levels in this example, thereby improving the specificity of the oncolytic adenovirus on recognition and enhancing safety.

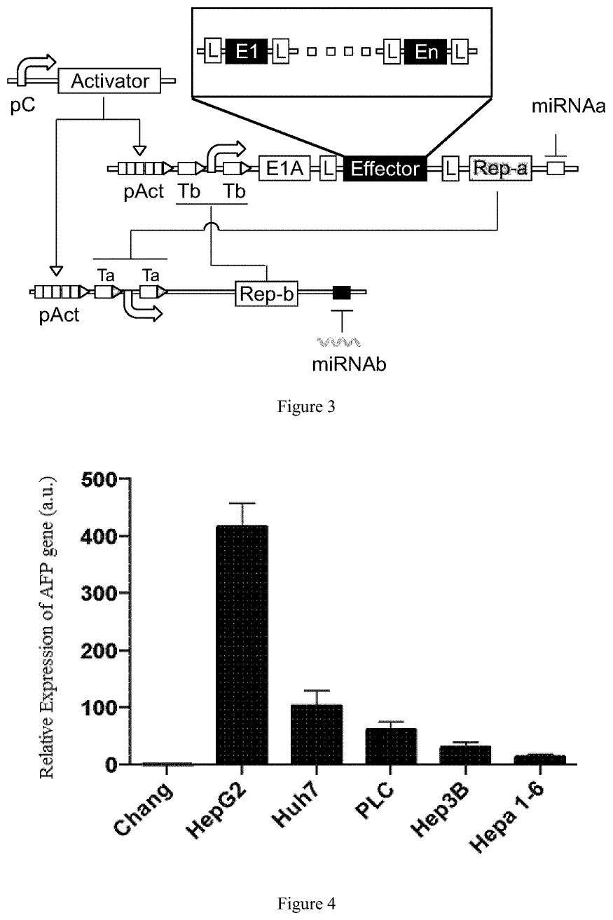

As shown in FIG. 3, the mutually repressive switch has the following features. The tumor specific promoter (pC) regulates the master activator of the mutually repressive switch. The master activator acts on the activator response promoter (pAct) to regulate the expression of downstream genes. The first side of the mutually repressive switch expresses the adenovirus packaging-related E1A gene, effectors and the repressor a (Rep-a), as well as a target sequence of microRNA a (miRNA a) highly expressed in non-tumor cells, which further comprises a recognition sequence of repressor b expressed on the second side and a target sequence of microRNA b (miRNA b) highly expressed in tumor cells, and where the recognition sequence of the repressor b is located at two sides of the first promoter in the first side respectively. The second side of the mutually repressive switch expresses the repressor b (Rep-b), which further comprises a recognition sequence of the repressor a expressed on the first side, and where the recognition sequence of the repressor a is located at two sides of the second promoter in the second side respectively. In tumor cells, the tumor specific promoter (pC) initiates the expression of the whole system. The repressor b is degraded via RNA interference due to the high expression of miRNA b and the low expression of miRNA a in tumor cells, thus the inhibition of the repressor b to the gene expression on the first side is eliminated. Besides, the high expression of repressor a can further inhibit the expression of repressor b, thus allowing the mutually repressive switch to switch into the state of E1A expression quickly and efficiently, thereby initiating the packaging of adenovirus and the expression of effectors. Conversely, when the mutually repressive switch enters non-tumor cells, E1A and effectors cannot be expressed due to the combination of the tumor-specific promoter, miRNA markers and the logic circuit, thereby effectively closing the packaging process of adenovirus, and ensuring the specificity and safety of this logic circuit. Therefore, such a mutually repressive switch can respond to a variety of input signals and distinguish target cells from non-target cells at transcriptional and post-transcriptional levels. In addition, the present expression system proposed by the inventors can express the E1A gene only, with the E1B gene removed, thus improving the recognition ability of oncolytic virus to target cells from the viewpoint of complementation of functional defects. Therefore, the expression system designed by the inventors is capable of distinguishing target cells at multiple levels, with effectively improved adenovirus safety.

2. Verification and Analysis of Biomarkers Distinguishing Hepatoma Cells from Normal Cells

2.1 Construction of Hepatoma-Specific Alpha-Fetoprotein (AFP) Promoter

Alpha-fetoprotein (αFP or AFP) is mainly synthesized in liver of a fetus, which is not expressed in a normal adult, however it will be expressed again when liver cells become cancerous, and the amount of AFP in serum will increase dramatically with the progress of liver cancer. At present, alpha-fetoprotein is a specific clinical indicator for the diagnosis of primary liver cancer, thus the inventors selected the alpha-fetoprotein promoter as a promoter marker for distinguishing hepatoma cells from non-hepatoma cells in the example.

The inventors further detected the specific expression of AFP by experiments. First, the expression of human AFP gene in a series of non-hepatoma cells and hepatoma cells was detected by the qPCR method. As shown in FIG. 4, RNAs of Chang, HepG2, Huh7, PLC, Hep3B and Hepa1-6 cell lines were respectively extracted, and the expression level of AFP gene in these cells was detected by the quantitative PCR method, where Chang cells are liver cells of a normal human, and HepG2, Huh7, PLC and Hep3B cells are human hepatoma cells, and Hepa1-6 cells are mouse hepatoma cells. The results show that the human AFP gene is highly expressed in the HepG2 and Huh7 cell lines, and low-expressed in the PLC and Hep3B cell lines, as well as slightly expressed in the mouse Hepa1-6 hepatoma cells, suggesting that the AFP promoter can be employed by the inventors as a promoter marker in the hepatoma cells due to its specific expression.

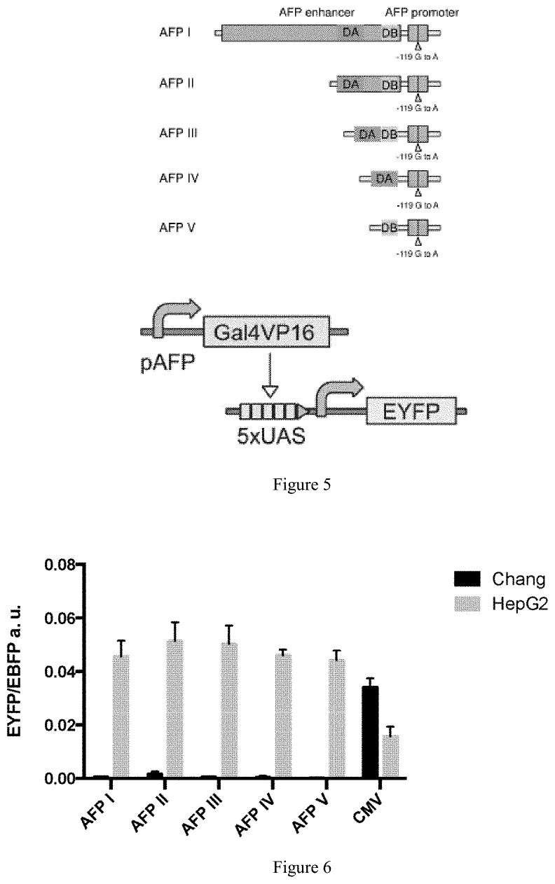

The currently widely used AFP promoter consists of an enhancer and a promoter. The enhancer contains two sequences which can activate the promoter, i.e. domain A (DA) and domain B (DB). In order to increase the efficiency of the promoter, the inventors mutated guanine (G) at position-119 of the promoter to adenine (A). In addition, the enhancer region was truncated to different extent by the inventors. As shown in FIG. 5, AFP I comprises an enhancer sequence of AFP in a length of 1.8 K (as shown in SEQ ID NO: 21); AFP II comprises an activation region A (DA), an activation region B (DB) and a sequence between the two activation regions (as shown in SEQ ID NO: 22); AFP III comprises an activation region A (DA) and an activation region B (DB) only (as shown in SEQ ID NO: 23); AFP IV comprises an activation region A (DA) only (as shown in SEQ ID NO: 24); and AFP V comprises an activation region B (DB) only (as shown in SEQ ID NO: 25). To detect the expression efficiency and specificity of the AFP promoter, these five AFP promoter reporter system plasmids were transiently transfected into normal hepatocytes Chang and hepatoma cells HepG2 respectively. The results in FIG. 6 show that the five AFP promoters are all highly expressed in hepatoma cell lines, even exceeding expression level of the commonly expressed CMV promoter. Based on the analysis of specificity, other four promoters except for AFP II promoter are not obviously leaked in Chang cells, with slight expression of the AFP II reporter gene in Chang cells. In consideration of the length of sequence to be inserted, and the efficiency and specificity of a promoter, the inventors selected the AFP III promoter to regulate the present mutually repressive switch in subsequent experiments.

(SEQ ID NO: 21)
TTTAGAAATATGGGGGTAGGGGTGGTGGTGGGGCCTGGATAAAGCTGAGTGGTAT
GAATGAGTTAGAAATATGGGGGTAGGGGTGGTGGTGGTAATTCTGTTTTCTCCCCATAG
GTGAGATAAGCATTGGGTTAAATGTGCTTTCTCTCTCTCCCTCTCCTTTCTTAAGAATTA
AGGGACAGACTATGGGCTGGAGGACTTTGAGGATGTCTGTCTCATAACACTTGGG
TTGTATCTGTTCTATGGGGCTTGTTTTAAGCTTGGCAACTTGCAACAGGGTTCACTGAC
TTTCTCCCCAGGCCCAAGGTACTGTCCTCTTTTCATATCTGTTTTGGGGCCTCTGGGGC
AATATCTGAGAAAATATAAACATTTCAATAATGTTCTGTGGTGAGATGAGTATGAGAGA
TGTGTCATTCATTTGTATCAATGAATGAATGAGGACAATTAGTGTATAAATCCTTAGTAC
AACAATCTGAGGGTAGGGGTGGTACTATTCAATTTCTATTTATAAAGATACTTATTTCTAT
TTATTTATGCTTGTGACAAATGTTTTGTTCGGGACCACAGGAATCACAAAGATGAGTCT
TTGAATTTAAGAAGTTAATGGTCCAGGAATAATTACATAGCTTACAAATGACTATGATAT
ACCATCAAACAAGAGGTTCCATGAGAAAATAATCTGAAAGGTTTAATAAGTTGTCAAA
GGTGAGAGGGCTCTTCTCTAGCTAGAGACTAATCAGAAATACATTCAGGGATAATTATT
TGAATAGACCTTAAGGGTTGGGTACATTTTGTTCAAGCATTGATGGAGAAGGAGAGTG
AATATTTGAAAACATTTCAACTAACCAACCACCCAATCCAACAAACAAAAAATGAAAA
GAATCTCAGAAACAGTGAGATAAGAGAAGGAATTTTCTCACAACCCACACGTATAGCT
CAACTGCTCTGAAGAAGTATATATCTAATATTTAACACTAACATCATGCTAATAATGATAA
TAATTACTGTCATTTTTTAATGTCTATAAGTACCAGGCATTTAGAAGATATTATTCCATTTA
TATATCAAAATAAACTTGAGGGGATAGATCATTTTCATGATATATGAGAAAAATTAAAAA
TCAGATTGAATTATTTGCCTGTCATACAGCTAATAATTGACCATAAGACAATTAGATTTA
AATTAGTTTTGAATCTTTCTAATACCAAAGTTCAGTTTACTGTTCCATGTTGCTTCTGAG
TGGCTTCACAGACTTATGAAAAAGTAAACGGAATCAGAATTACATCAATGCAAAAGCA
TTGCTGTGAACTCTGTACTTAGGACTAAACTTTGAGCAATAACACATATAGATTGAGGA
TTGTTTGCTGTTAGTATACAAACTCTGGTTCAAAGCTCCTCTTTATTGCTTGTCTTGGAA
AATTTGCTGTTCTTCATGGTTTCTCTTTTCACTGCTATCTATTTTTCTCAACCACTCACAT
GGCTACAATAACTGTCTGCAAGCTTATGATTCCCAAATATCTATCTCTAGCCTCAATCTT
GTTCCAGAAGATAAAAAGTAGTATTCAAATGCACATCAACGTCTCCACTTGGAGGGCT
TAAAGACGTTTCAACATACAAACCGGGGAGTTTTGCCTGGAATGTTTCCTAAAATGTG
TCCTGTAGCACATAGGGTCCTCTTGTTCCTTAAAATCTAATTACTTTTAGCCCAGTGCTC
ATCCCACCTATGGGGAGATGAGAGTGAAAAGGGAGCCTGATTAATAATTACACTAAGT
CAATAGGCATAGAGCCAGGACTGTTTGGGTAAACTGGTCACTTTATCTTAAACTAAATA
TATCCAAAACTGAACATGTACTTAGTTACTAAGTCTTTGACTTTATCTCATTCATACCAC
TCAGCTTTATCCAGGCCTCTAGAAGTTTGAGGAGAATATTTGTTATATTTGCAAAATAAA
ATAAGTTTGCAAGTTTTTTTTTTCTGCCCCAAAGAGCTCTGTGTCCTTGAACATAAAAT
ACAAATAACCGCTATGCTGTTAATTATTGACAAATGTCCCATTTTCAACCTAAGGAAATA
CCATAAAGTAACAGATATACCAACAAAAGGTTACTAGTTAACAGGCATTGCCTGAA
AAGAGTATAAAAGAATTTCAGCATGATTTTCCATATTGTGCTTCCACCACTGCCAATAA
CAAAATAACTAGCAA.
(SEQ ID NO: 22)
CAGATTGAATTATTTGCCTGTCATACAGCTAATAATTGACCATAAGACAATTAGATT
TAAATTAGTTTTGAATCTTTCTAATACCAAAGTTCAGTTTACTGTTCCATGTTGCTTCTG
AGTGGCTTCACAGACTTATGAAAAAGTAAACGGAATCAGAATTACATCAATGCAAAAG
CATTGCTGTGAACTCTGTACTTAGGACTAAACTTTGAGCAATAACACATATAGATTGAG
GATTGTTTGCTGTTAGTATACAAACTCTGGTTCAAAGCTCCTCTTTATTGCTTGTCTTGG
AAAATTTGCTGTTCTTCATGGTTTCTCTTTTCACTGCTATCTATTTTTCTCAACCACTCAC
ATGGCTACAATAACTGTCTGCAAGCTTATGATTCCCAAATATCTATCTCTAGCCTCAATC
TTGTTCCAGAAGATAAAAAGTAGTATTCAAATGCACATCAACGTCTCCACTTGGAGGG
CTTAAAGACGTTTCAACATACAAACCGGGGAGTTTTGCCTGGAATGTTTCCTAAAATGT
GTCCTGTAGCACATAGGGTCCTCTTGTTCCTTAAAATCTAATTACTTTTAGCCCAGTGCT
CATCCCACCTATGGGGAGATGAGAGTGAAAAGGGAGCCTGATTAATAATTACACTAAG
TCAATAGGCATAGAGCCAGGACTGTTTGGGTAAACTGGTCACTTTATCTTAAACTAAAT
ATATCCAAAACTGAACATGTACTTAGTTACTAAGTCTTTGACTTTATCTCATTCATACCA
CTCAGCTTTATCCAGGCCACTTATTTGACAGTATTATTGCGAAAACTTCCTATCTAGAAG
TTTGAGGAGAATATTTGTTATATTTGCAAAATAAAATAAGTTTGCAAGTTTTTTTTTTCT
GCCCCAAAGAGCTCTGTGTCCTTGAACATAAAATACAAATAACCGCTATGCTGTTAATT
ATTGACAAATGTCCCATTTTCAACCTAAGGAAATACCATAAAGTAACAGATATACCAAC
AAAAGGTTACTAGTTAACAGGCATTGCCTGAAAAGAGTATAAAAGAATTTCAGCATGA
TTTTCCATATTGTGCTTCCACCACTGCCAATAACAAAATAACTAGCAA.
(SEQ ID NO: 23)
CAGATTGAATTATTTGCCTGTCATACAGCTAATAATTGACCATAAGACAATTAGATT
TAAATTAGTTTTGAATCTTTCTAATACCAAAGTTCAGTTTACTGTTCCATGTTGCTTCTG
AGTGGCTTCACAGACTTATGAAAAAGTAAACGGAATCAGAATTACATCAATGCAAAAG
CATTGCTGTGAACTCTGTACTTAGGACTAAACTTTGAGCAATAACACATATAGATTGAG
GATTGTTTGCTGTTAGTATACAAACTCTGGTTCAAAGCTCCTCTTTATTGCTTGTCTTGG
AAAATTTGCTGTTCTTCATGGTTTCTCTTTTCACTGCTATCTATTTTTCTCAACCACTCAC
ATGGCTACAAAAGCTTCCTGATTAATAATTACACTAAGTCAATAGGCATAGAGCCAGGA
CTGTTTGGGTAAACTGGTCACTTTATCTTAAACTAAATATATCCAAAACTGAACATGTAC
TTAGTTACTAAGTCTTTGACTTTATCTCATTCATACCACTCAGCTTTATCCAGGCCACTTA
TTTGACAGTATTATTGCGAAAACTTCCTATCTAGAAGTTTGAGGAGAATATTTGTTATAT
TTGCAAAATAAAATAAGTTTGCAAGTTTTTTTTTTCTGCCCCAAAGAGCTCTGTGT
CCTTGAACATAAAATACAAATAACCGCTATGCTGTTAATTATTGACAAATGTCCCATTTT
CAACCTAAGGAAATACCATAAAGTAACAGATATACCAACAAAAGGTTACTAGTTAACA
GGCATTGCCTGAAAAGAGTATAAAAGAATTTCAGCATGATTTTCCATATTGTGCTTCCA
CCACTGCCAATAACAAAATAACTAGCAA.
(SEQ ID NO: 24)
CAGATTGAATTATTTGCCTGTCATACAGCTAATAATTGACCATAAGACAATTAGATT
TAAATTAGTTTTGAATCTTTCTAATACCAAAGTTCAGTTTACTGTTCCATGTTGCTTCTG
AGTGGCTTCACAGACTTATGAAAAAGTAAACGGAATCAGAATTACATCAATGCAAAAG
CATTGCTGTGAACTCTGTACTTAGGACTAAACTTTGAGCAATAACACATATAGATTGAG
GATTGTTTGCTGTTAGTATACAAACTCTGGTTCAAAGCTCCTCTTTATTGCTTGTCTTGG
AAAATTTGCTGTTCTTCATGGTTTCTCTTTTCACTGCTATCTATTTTTCTCAACCACTCAC
ATGGCTACAAAAGCTTTCTAGAAGTTTGAGGAGAATATTTGTTATATTTGCAAAATAAA
ATAAGTTTGCAAGTTTTTTTTTTCTGCCCCAAAGAGCTCTGTGTCCTTGAACATAAAAT
ACAAATAACCGCTATGCTGTTAATTATTGACAAATGTCCCATTTTCAACCTAAGGAAATA
CCATAAAGTAACAGATATACCAACAAAAGGTTACTAGTTAACAGGCATTGCCTGAAAA
GAGTATAAAAGAATTTCAGCATGATTTTCCATATTGTGCTTCCACCACTGCCAATAACA
AAATAACTAGCAA.
(SEQ ID NO: 25)
CCTGATTAATAATTACACTAAGTCAATAGGCATAGAGCCAGGACTGTTTGGGTAAA
CTGGTCACTTTATCTTAAACTAAATATATCCAAAACTGAACATGTACTTAGTTACTAAGT
CTTTGACTTTATCTCATTCATACCACTCAGCTTTATCCAGGCCACTTATTTGACAGTATTA
TTGCGAAAACTTCCTATCTAGAAGTTTGAGGAGAATATTTGTTATATTTGCAAAATAAAA
TAAGTTTGCAAGTTTTTTTTTTCTGCCCCAAAGAGCTCTGTGTCCTTGAACATAAAATA
CAAATAACCGCTATGCTGTTAATTATTGACAAATGTCCCATTTTCAACCTAAGGAAATAC
CATAAAGTAACAGATATACCAACAAAAGGTTACTAGTTAACAGGCATTGCCTGAAAAG
AGTATAAAAGAATTTCAGCATGATTTTCCATATTGTGCTTCCACCACTGCCAATAACAA
AATAACTAGCAA.

2.2 Construction of microRNA (miRNA) Fluorescent Plasmid Reporter System for Detection of Expression Levels of Different Cell Lines

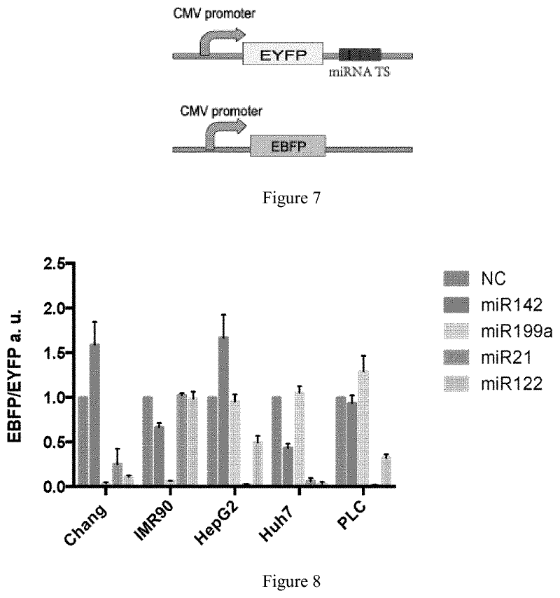

Recent research shows that the formation and growth of a tumor will greatly change the expression level of microRNA (miRNA). As shown in Table 1, various microRNAs are highly expressed in different cancer cells. The inventors selected miR199a as a microRNA marker to characterize the high expression of normal hepatocytes, and correspondingly selected miR21 and miR122 as microRNA markers to characterize the high expression of hepatoma cells based on the analysis of the known research results. First, the inventors have designed and constructed a microRNA fluorescent plasmid reporter system. As shown in FIG. 7, target sequences of microRNAs were inserted into the 3′ untranslated region of commonly-expressed EYFP by the inventors, with another commonly-expressed EBFP as a fluorescence control. Several different cell lines were transfected with the EYFP and EBFP fluorescent plasmids respectively to detect the expression of the microRNA target sequences. The experimental results are shown in FIG. 8, indicating that miR199a is highly expressed in normal hepatocytes compared to hepatoma cell lines, while miR21 exhibits significantly increased expression level in hepatoma cell lines, thus the miR199a and the miR21 can be used as effective markers of the mutually repressive switch designed by the inventors to distinguish hepatoma cells from normal cells.

2.3 Mutually Repressive Switch can Effectively Respond to microRNA Input Signals and Stably Function in Different Cell Lines

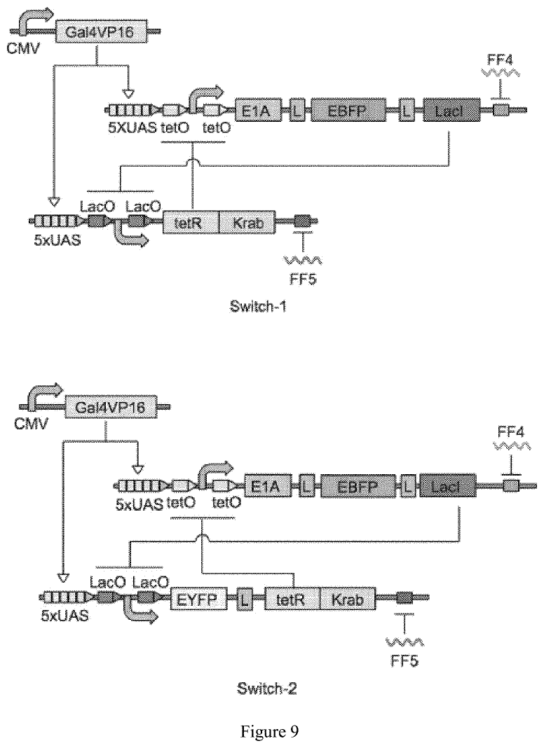

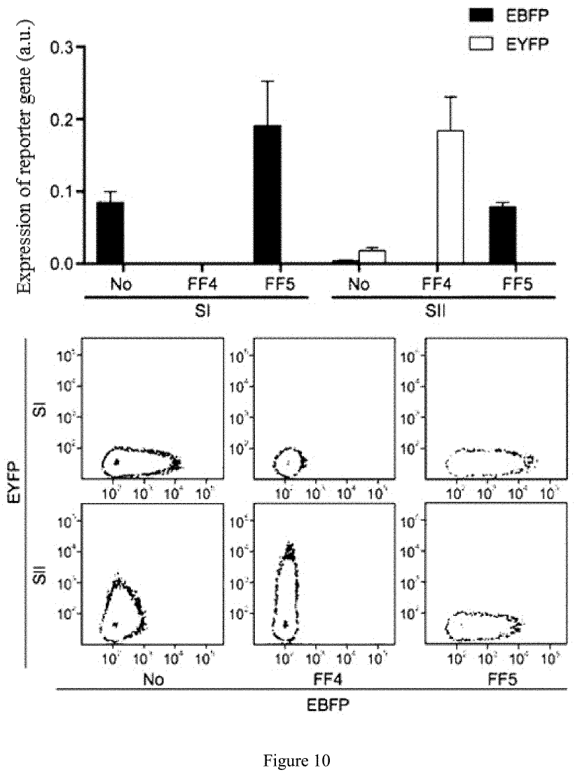

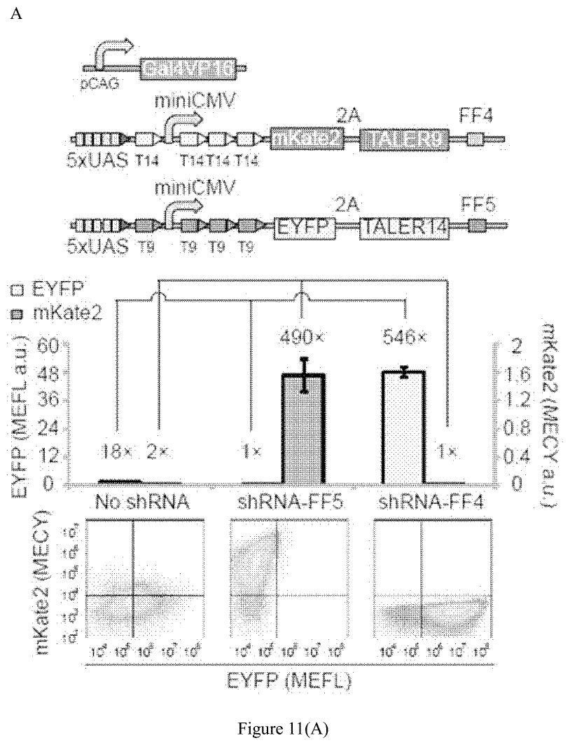

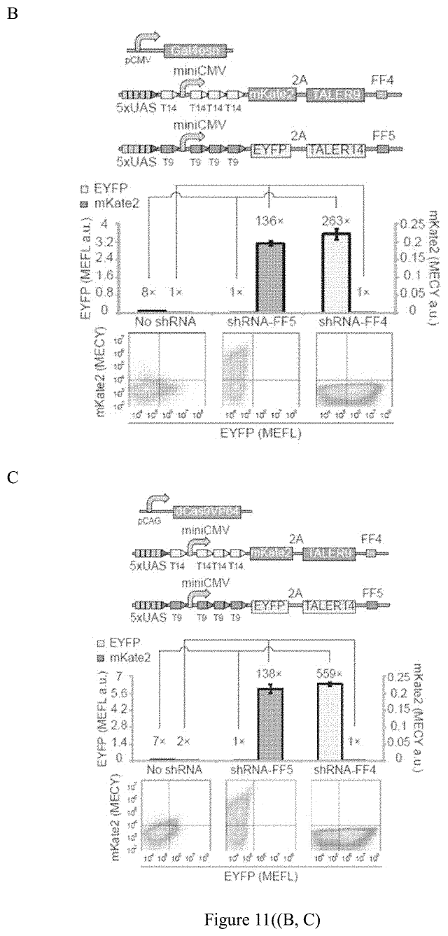

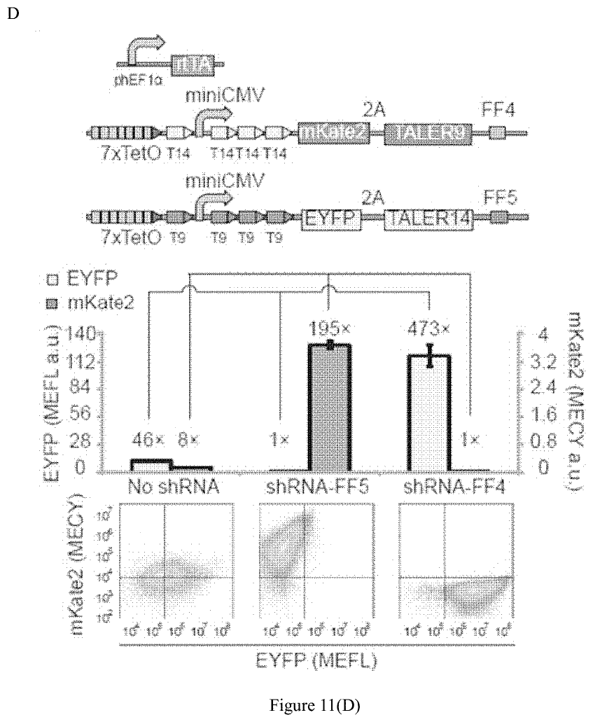

Because of limited packaging capacity of the oncolytic adenovirus (only 8 K foreign genes can be packaged at most), the inventors have selected short and effective inhibition elements as much as possible in this example. The inventors selected Lad and tetR-KRAB in this example. As shown in FIG. 9, the inventors constructed two logic circuits depending on whether the EYFP gene is co-expressed on the tetR-KRAB side, that is Switch I (SI) which does not express EYFP, and Switch II (SII) which expresses EYFP. The two switches were transiently transfected into HEK293 cells individually and each were cotransfected with shRNA-FF4 and shRNA-FF5 respectively. As shown in FIG. 10, the present two switches both can efficiently reverse under different input signals. The SI expresses at a higher level on the Lad side, but exhibits a higher leakage level on the tetR-KRAB side than SII. The inventors after comprehensive consideration have chosen the SI system as a backbone for constructing the present oncolytic adenovirus carrying different effectors in subsequent experiments, since the SI switch system is capable of responding to different input signals and efficiently expressing the E1 gene in cells of interest. Based on the previous results in laboratory, the inventors tested the regulation effects of different activators on the mutually repressive switch SI. As shown in FIG. 11, the inventors tested the regulation efficiency of activators Gal4VP16, Gal4esn, dCas9-VP64 and rtTA. The experimental results show that the gene circuit can effectively respond to those different activators, proving the stability of such a gene circuit.

2.4 Construction of Oncolytic Adenovirus by Using the CGGA Method

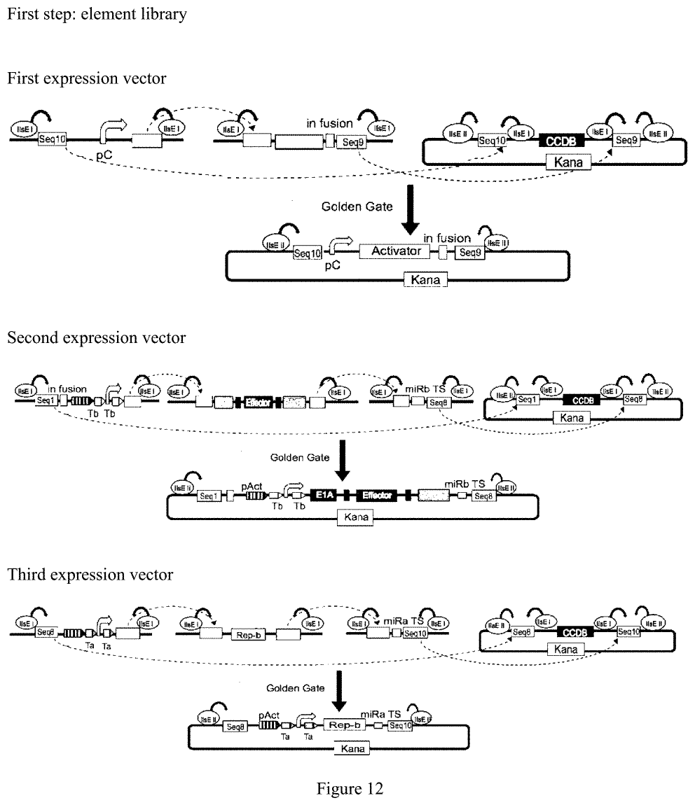

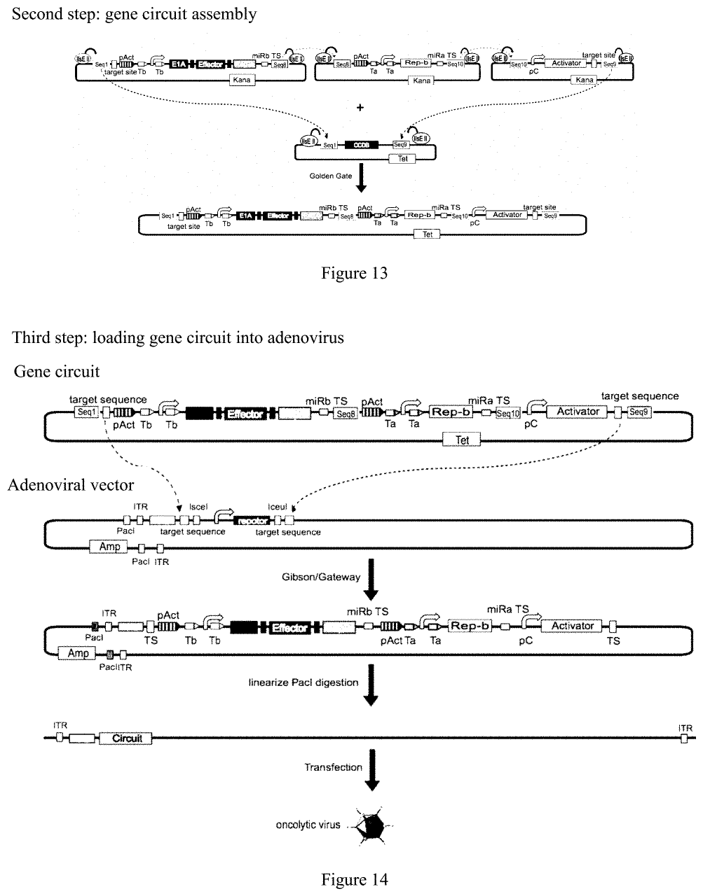

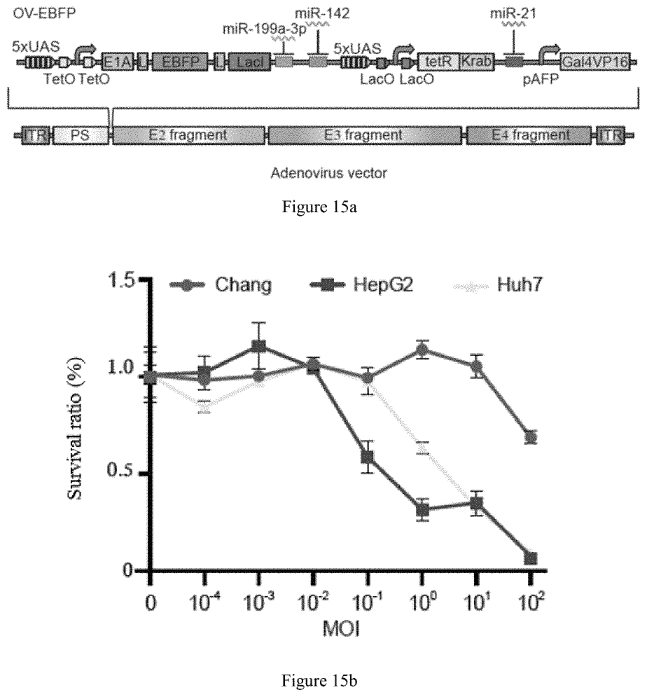

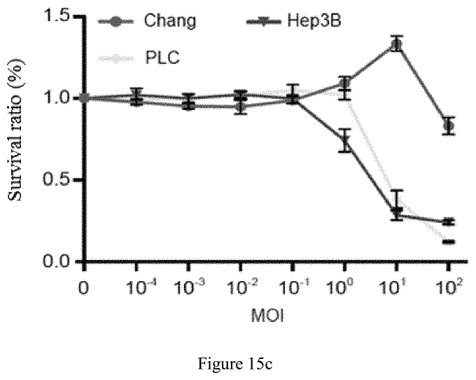

A large amount of clinical information indicates that the expression of tumor markers varies greatly among different individuals. Therefore, different markers may be chosen by the inventors for different individuals. In order to achieve the precise treatment for individual patients, the inventors need to prepare a variety of oncolytic adenoviruses carrying different biomarkers. However, it is very difficult to rapidly construct these oncolytic adenoviruses through traditional enzyme digestion and ligation method. In the present example, the inventors designed and constructed a three-stage library, and rapidly modified oncolytic adenovirus by the CGGA method (Cascade Golden-Gate Gateway/Gibson Assemble method). First, marker elements associated with distinguishing of cancer cells from normal cells (such as a tumor-specific promoter, a logic circuit-related repression element, and a cancer cell-distinguishing miRNA recognition sequence) are constructed on a primary plasmid, thereby forming a primary element library, as shown in FIG. 12. Two ends of the primary elements are each inserted with the recognition site of EI (a restriction enzyme of which recognition site and cleavage site are different, such as Esp3I and BsaI), so as to construct multiple primary elements into one secondary expression system by the Golden Gate method. An expression system is constructed as follow. Specifically, multiple primary elements are selected from the primary element library and are assembled into three expression systems by the Golden Gate method, such three expression systems including a tumor-specific promoter system, a first regulatory element-mediated repression system and a second regulatory element-mediated repression system. Then, a complete logic circuit is formed by randomly assembling the three expression systems by the Golden Gate method, as shown in FIG. 13. Two ends of the logic circuit are each inserted with Gibson homologous sequence or Gateway recognition site (target sequence) for further experiments. Finally, the logic circuit is constructed into an adenoviral vector (with E1 and part of E3 removed) by the Gibson or Gateway method, as shown in FIG. 14. The engineered adenoviral vector is linearized by PacI digestion, and then transfected into a specific cell line so as to package adenoviral particles. Thus, one or more tumor biomarkers can be replaced by the inventors.

2.5 Detection of Oncolytic Adenovirus on Effectively Killing of Hepatoma Cells at Cellular Level

In order to detect whether the mutually repressive switch in the oncolytic adenovirus constructed by the inventors can effectively distinguish and kill hepatoma cells, the inventors designed an in vitro cell killing assay for oncolytic adenovirus. Different cell lines were infected with the oncolytic adenovirus in different multiplicity of infection, where Chang cells are normal liver cells, and Huh7 and HepG2 cells are human hepatoma cell lines. The viability of cells was measured by the MTS method after 6 days of incubation. As shown in FIG. 15, it is found that the hepatoma cells show severe death due to significant cytotoxicity when the multiplicity of infection is 100, 10 and 1 respectively, and the hepatoma cells exhibit slight cell death at the multiplicity of infection of 0.1. Correspondingly, Chang cells are in normal growth state without cell death when the multiplicity of infection is 10 or less; while they exhibit a slight cytotoxic response at the multiplicity of infection of 100, with enlarged cell volume and slowed cell growth. This data indicates that the packaged adenovirus within a specific range of multiplicity of infection can effectively distinguish and kill hepatoma cells, without interference of growth and proliferation of normal liver cells.

2.6 In Vitro Detection of Expression Level of Effector Carried by Oncolytic Adenovirus

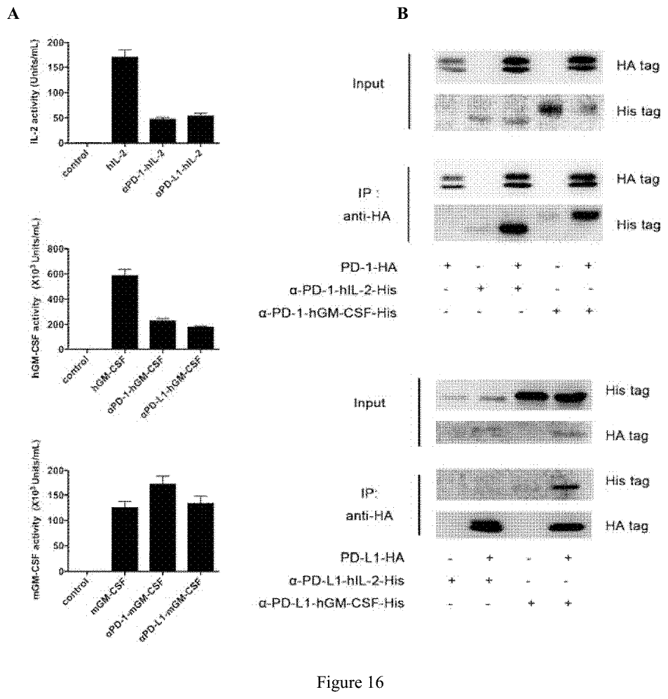

The adenoviral vectors currently used in clinical practice are human Ad2 or Ad5 type adenoviruses which are widely present in nature. Further, neutralizing antibodies that antagonize these adenoviruses have been known to exist in the human body. Thus, clinical data showed a poor treatment effect for the tumor treatment relying on the oncolytic effect of adenovirus alone. Therefore, the inventors in the example used the oncolytic adenovirus as a delivery system to carry effector genes to tumor sites, so as to cause a systemic immune response. As shown in FIG. 16, the inventors first constructed these effector genes into a frequently-expressed plasmid vector, and detected the activity of these effectors by transient transfection. The results demonstrate that the effector genes constructed by the inventors can efficiently express immune factors having biological function, and repressors of immunological checkpoint genes. The inventors further constructed these effector genes on an oncolytic adenoviral vector, thus obtaining the oncolytic adenovirus which can express these effectors after packaging and purification. After infection of the hepatoma cells HepG2, the inventors extracted the supernatant to detect the activity of immune factors in the supernatant.

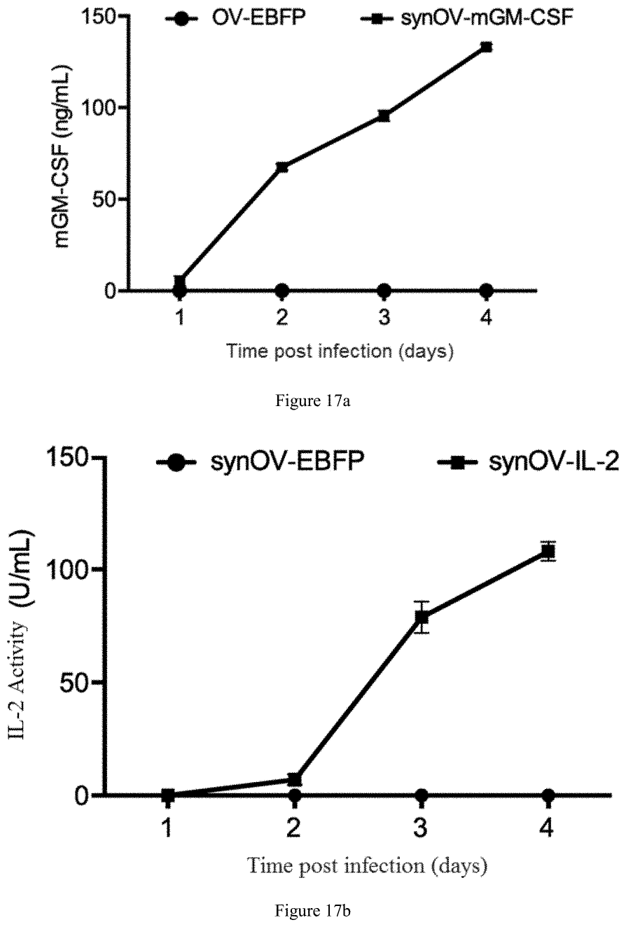

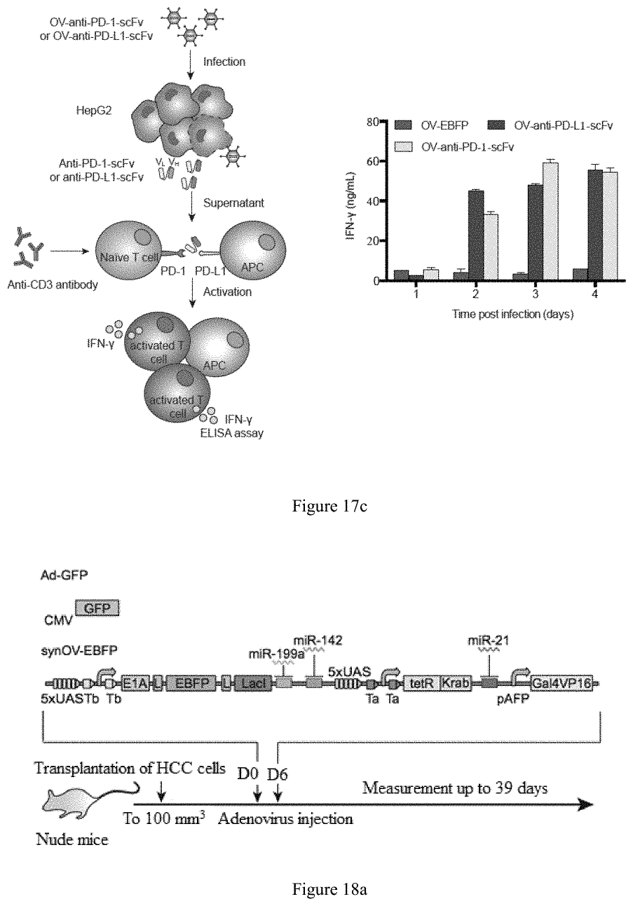

In the meantime, the activity of the effectors expressed by the effector genes in the adenoviral vector has been also detected by the inventors. As shown in FIG. 17, the effectors expressed by the adenovirus are effectively enhanced over time. The three cytokines are detected by the method as follows. In FIG. 17(a), 3×10⁶ HepG2 cells were seeded into 12-well plates, infected with synOV-EBFP and synOV-mGM-SCF at the multiplicity of infection of 10 respectively, and collected the supernatant at day 1, 2, 3 and 4 post-infection. The mouse GM-CSF content in the supernatant was detected by an ELISA kit. In FIG. 17(b), 3×10⁶ HepG2 cells were seeded into 12-well plates, infected with synOV-EBFP and synOV-hIL-2 at the multiplicity of infection of 10 respectively, and collected the supernatant at day 1, 2, 3 and 4 post-infection. The supernatant was added into a medium containing IL-2 dependent cells, and the IL-2 content in the supernatant was detected by the MTS method. In FIG. 17(c), 3×10⁶ HepG2 cells were seeded into 12-well plates, infected with synOV-EBFP, synOV-anti-PD-1 scFv and synOV-anti-PD-L1 scFv at the multiplicity of infection of 10 respectively, and collected the supernatant at day 1, 2, 3 and 4 post-infection. The supernatant obtained was added into a spleen cell system (formed by addition of 2 μg/mL anti-mouse CD3-antibody in isolated spleen cells for T cell activation) at a dilution ratio of 1:10, and the IFN-γ production in the spleen cell system was detected by the ELISA method after 48 hours of incubation.

2.7 Detection of Therapeutic Effect of Oncolytic Adenovirus by Animal Studies

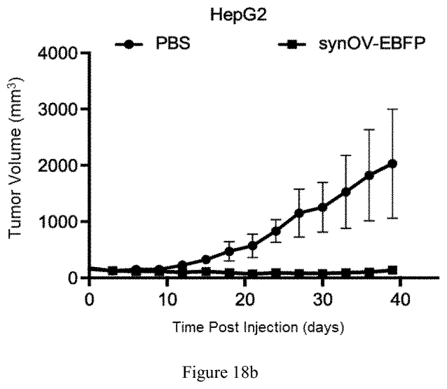

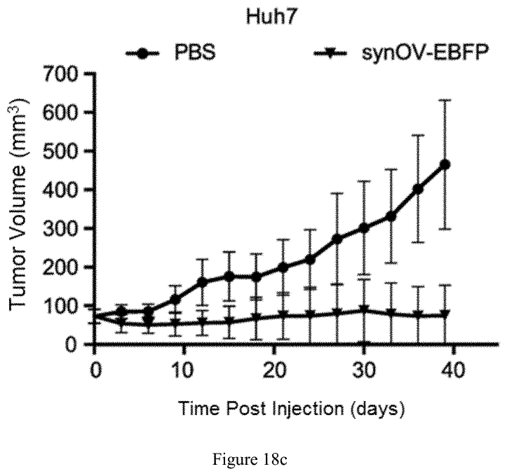

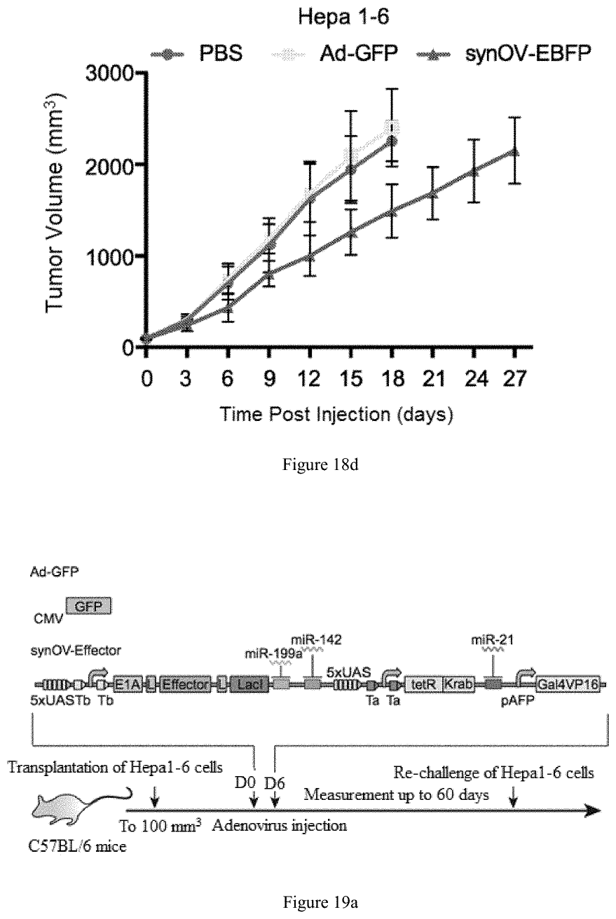

As shown in FIG. 18, 1×10⁷ HepG2, Huh7 and Hepa1-6 hepatoma cells were subcutaneously transplanted into the right flank of nude mice, with measurement of subcutaneous tumor volume every three days. HepG2 and Huh7 cells are human hepatoma cells, and Hepa1-6 cells are mouse hepatoma cells. After tumor volume exceeded 100 mm³, mice were divided randomly into two groups, where the treatment group was intratumorally injected with 1×10⁹ oncolytic adenovirus and the control group was injected with PBS. The change of tumor volume after treatment was continuously observed. It can be seen that the tumor in the control group injected with PBS continues to grow, with increasingly increased tumor volume, while the tumor in the treatment group is significantly inhibited after the injection of oncolytic adenovirus, indicating that the oncolytic adenovirus can inhibit the tumor growth in vivo through lysis of tumor cells, thereby exhibiting a significant therapeutic effect on the formed tumor. In the treatment of mouse liver cancer, the inventors used a non-oncolytic virus (the second generation adenoviral vector, GFP gene-bearing virus, Ad-GFP) as another virus control. It can be seen from the experimental results that the tumors in the PBS and Ad-GFP treatment groups continue growing, while the oncolytic virus treatment groups can inhibit the growth of tumors to some extent and slightly inhibit the growth of tumor. It is probably resulted from a faster growth rate of Hepa1-6 cells than HepG2 and Huh7 cells, as well as a relatively-low expression level of AFP in the Hepa1-6 cells. Therefore, the inventors employed the immune effector-carrying oncolytic adenovirus to treat the Hepa1-6 tumor model in subsequent experiments, which achieved greatly effective results.

After two weeks or one month of treatment of the tumor-bearing mice with oncolytic adenovirus, tissue samples of various organs of the mice were taken, and the virus DNA was extracted after homogenization. The viral titer was detected by the qPCR method. It is found that the viruses are only replicated in the tumor cells and do not spread into other tissues after virus infection. The results demonstrate that oncolytic adenovirus in vivo has a strong specificity and is safe to normal tissue cells.

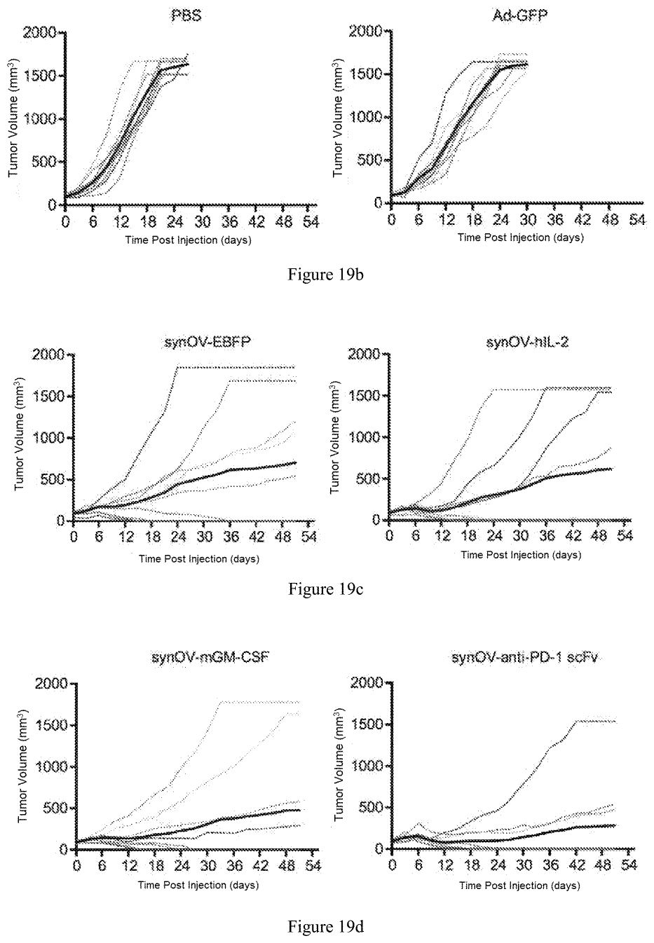

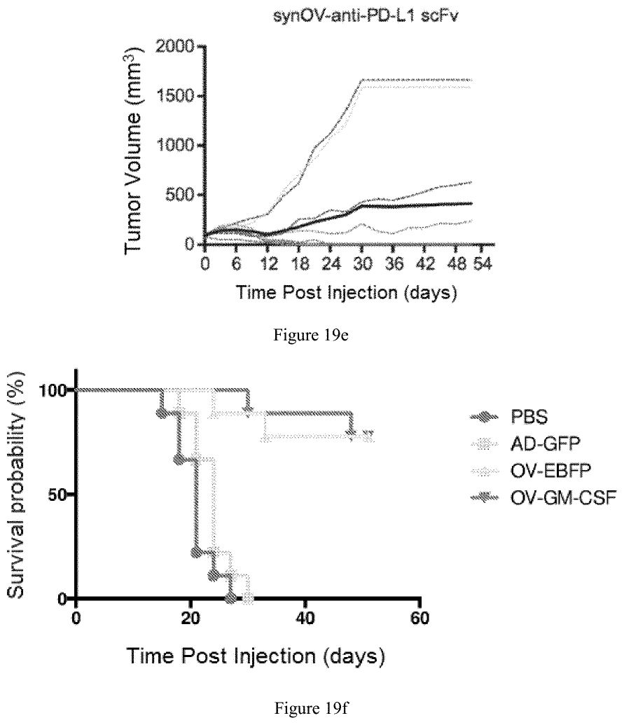

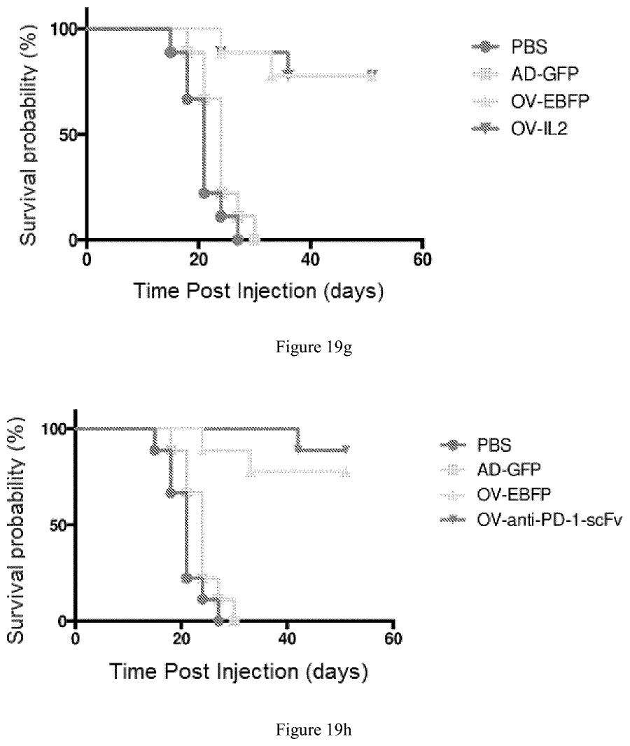

The therapeutic effect of the present oncolytic adenovirus on a C57 mouse model with an immune system is further demonstrated. As shown in FIG. 19, 1×10⁶ mouse hepatoma cells Hepa1-6 were subcutaneously injected into the right flank of wild-type C57BL/6J mice. After the tumor volume reached 100 mm³, 1×10⁹ oncolytic adenoviruses expressing different cytokines were intratumorally injected into each group, with PBS injection as a negative control. The tumor volume after treatment was observed, and the survival time of the tumor-bearing mice was analyzed by the Kaplan-Meier method. The experimental results demonstrate that oncolytic adenovirus can still inhibit the tumor growth and significantly prolong the survival time of tumor-bearing mice with an intact immune system. Some tumor-bearing mice were cured after treatment, with totally disappeared tumor. A month after the initial treatment with oncolytic adenovirus, the mice survived were re-challenged with 1×10⁶ mouse hepatoma cells Hepa1-6. It is found that a tumor mass cannot be formed when the one same mouse was transplanted with the tumor cells same as those first injected at a site away from the first transplantation site. These results demonstrate that the mice treated with oncolytic virus have generated specific immunity against tumor cells in body, which can resist the same tumor cells from growing again in body.

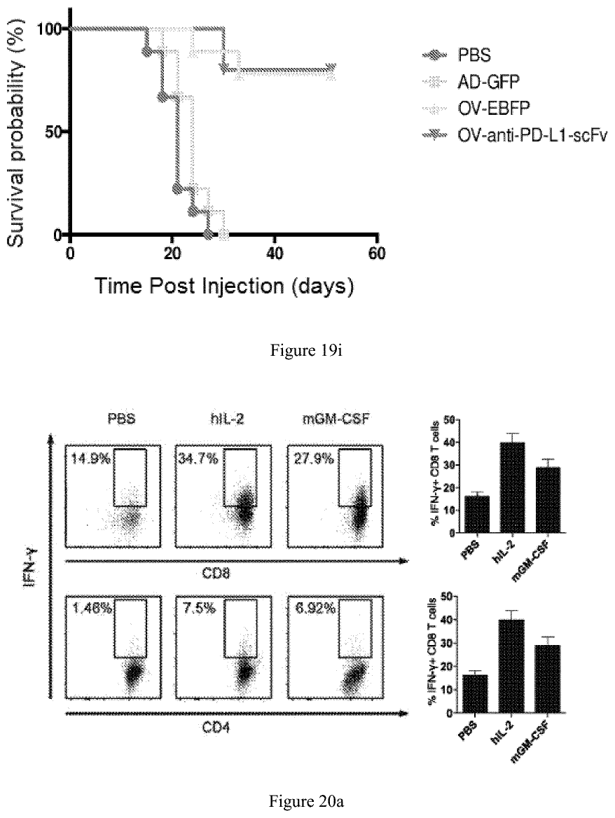

Tumors of tumor-bearing mice were taken, fixed, sectioned and HE stained for detection of lymphocyte infiltration in tumor tissues. It can be found that lymphocyte infiltration in tumor tissues of mice which is treated with oncolytic adenovirus is significantly increased compared with the PBS control group, demonstrating that the treatment of oncolytic adenovirus can recruit more lymphocytes in tumor tissues. These results indicated that expression of immune stimulators can promote the cytotoxic T cell response during oncolytic virus therapies. The infiltrating lymphocytes in tumors were isolated and purified, and the phenotype of the infiltrating T cells in mouse tumors was detected by the flow cytometry. It is found that a greater proportion of Ki-67⁺ cells among infiltrating T cells in tumors of mice treated with oncolytic virus, which indicates treatment with oncolytic adenovirus promotes the proliferation of T cells inside the tumor and triggers a stronger immune response. Tumor-infiltrating lymphocytes were added to RPMI 1640 medium containing PMA (20 ng/mL) and Ionomycin (1 μg/ml L), cultured at 37° C. for 4 hours in the presence of Brevedlin A, followed by fixation and staining of cells, and detecting the expression of γ-interferon in CD4⁺ and CD8⁺ T cells by flow cytometry. We found a higher expression of γ-interferon in T cells in tumors of the oncolytic adenovirus treatment group, which changed the immune microenvironment in tumor and promoted the killing of tumor cells, as shown in FIG. 20(a) and FIG. 20(b).

Systemic Anti-Tumor Immune Response by Treatment of Oncolytic Adenovirus

C57BL/6 mice were subcutaneously injected with mouse hepatoma cells Hepa-1-6 on both left and right flanks. After the formation of tumor mass, the oncolytic adenoviruses expressing cytokine hIL-2, mGM-CSF and anti-PD-1 scFv respectively were injected into the tumor on the right flank, with PBS and AdGFP injections as controls. The tumor growth was measured continuously. The infiltrating lymphocytes in tumors on the left and right flanks of the tumor-bearing mice after 14 days of treatment were isolated and purified, and the phenotype of infiltrating T cells in the tumors was detected by the flow cytometry method. We found a higher proportion of T cells expressing Ki-67⁺ T cells or interferon-y are present not only in the tumor injected with oncolytic adenovirus on the right flank but also in the tumor on the left flank without oncolytic adenovirus injection, compared with the control groups. It is demonstrated that the immune response caused by the treatment of oncolytic adenovirus can not only function on the tumor in the injection site, but also on the tumor in the distal side. This suggests that the oncolytic adenovirus not only has therapeutic effect on the tumor in the injection site, but also exhibits therapeutic effect on the tumor in distal metastasis.

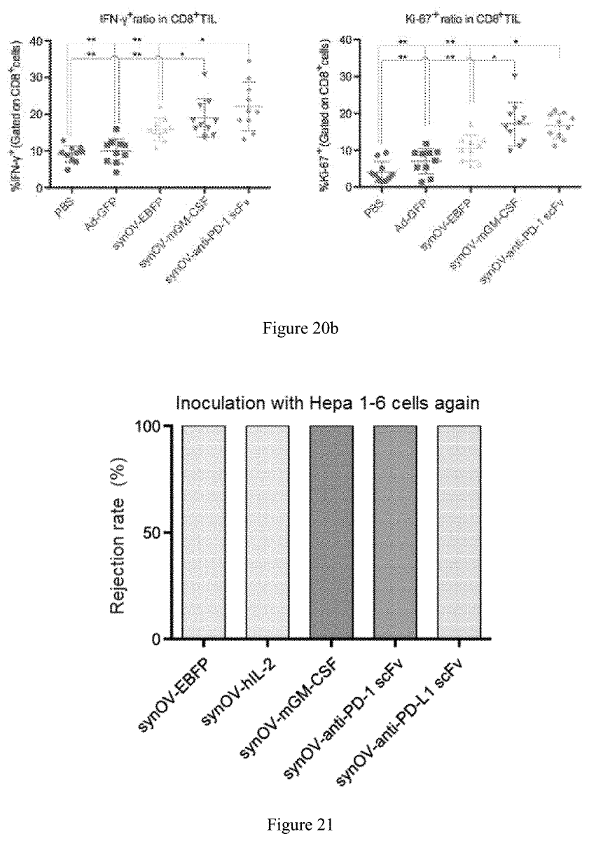

At the same time, in order to detect whether the treatment of oncolytic adenovirus has generated an effective systemic immune response in survived mice, Hepa1-6 was subcutaneously injected again into the mice after treatment by the inventors. It is found that the rejection rate of all the mice is 100%, as shown in FIG. 21.

2.8 Modeling of the Treatment Process of Oncolytic Adenovirus

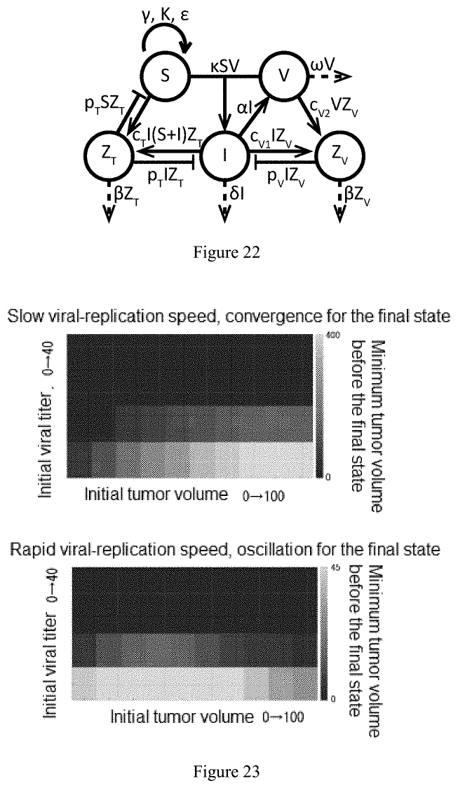

The treatment process of oncolytic adenovirus is abstracted into a tumor-virus-immune system, including uninfected tumor cells, infected tumor cells, free viruses, oncolytic adenovirus antigen activated-immune cells and tumor antigen activated-immune cells, such five components represented by S, I, V, Z_{V} and Z_{T} respectively, as shown in FIG. 22. The model assumes that the growth of uninfected tumor cells conforms to the generalized logistic growth model, where γ indicates the growth rate, K indicates the carrying capacity and c indicates the non-linearity. Uninfected tumor cells can be infected with free viruses and transformed into infected tumor cells at a rate of K. The infected tumor cells will lyse at a rate of 6, and release large amount of free viruses at a rate of α. The free virus naturally decays in the environment at a rate of ω. Z_{V} will be increased by the activation of oncolytic adenovirus or the surface antigen of tumor cells infected with viruses at a rate of c_{V1} and c_{V2} respectively, while it will inhibit the production of the infected tumor cells at a rate of p_{V}. Z_{T} will be increased by the activation of antigens released by lysed tumor cells at a rate of c_{T}, which will inhibit the production of the infected and uninfected tumor cells at a rate of P_{T}. Z_{V} and Z_{T} will naturally decay in the environment at a rate of β.

Without considering the immune system, the final state of the tumor-virus system will be converted from a state of convergence to oscillation with the increase of the virus replication speed. The simulations show the change of the minimal tumor volume in the tumor-virus system from an initial state to a final state over time under different initial virus titer, initial tumor size and viral replication rate. It can be seen from the simulation results that increase of initial viral titer contributes to the control of tumor size. In practical applications, the appropriate initial administration dose can be chose in combination of the initial tumor size and administration dose limit to clean tumors at predicted time points or provide other adjuvant therapies, as shown in FIG. 23.

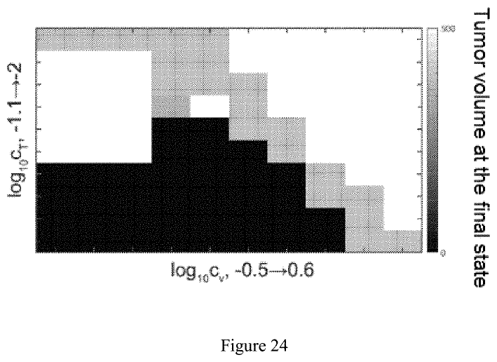

Tumor size in the tumor-virus-immune system at the final state during the duration of simulation under different inhibition effects of immune cells on tumors and viruses. It can be seen from the simulation results that the system exhibits several dynamic behaviors including tending to complete tumor elimination, tumor growth towards saturation, stable tumor volume, and unstable tumor volume. In general, stronger the inhibition effect of immune cells on tumors is, more tumors are cleaned by the system, while stronger the inhibition effect of immune cells on viruses is, more tumors in the system are out of control. However, the system exhibits non-monotonic changes on the tendencies of stability and non-stability of tumor, as shown in FIG. 24.

In the specification of the present disclosure, the terms “an embodiment”, “some embodiments”, “a specific embodiment”, “an example”, “a specific example”, “some examples” and the like are intended to refer to particular features, structures, materials or characteristics described by way of example or embodiment are contained in at least one embodiment or example of the disclosure. In this specification, the schematic representation of the above terms does not necessarily refer to the same embodiment or example. Moreover, the particular features, structures, materials or characteristics described may be combined in any suitable manner in one or more embodiments or examples. In addition, various embodiments or examples described in the specification and features of various embodiments or examples may be combined by skilled in the art without contradiction.

Although embodiments of the present disclosure have been described in detail, it will be understood the embodiments are illustrative and are not to be construed as limiting the scope of the disclosure. Changes, modifications, substitutions and variations can be made in these embodiments by those skilled in the art within the scope of the present disclosure.

What is claimed is:

1. An expression system comprising:
a first nucleic acid molecule, incorporating a cell-specific promoter;
a second nucleic acid molecule, operably linked to the first nucleic acid molecule and encoding a transcriptional activator;
a third nucleic acid molecule, incorporating a first recognition sequence of the transcriptional activator;
a fourth nucleic acid molecule, operably linked to the third nucleic acid molecule and incorporating a first promoter and a first regulatory element;
a fifth nucleic acid molecule, operably linked to the fourth nucleic acid molecule and encoding a first regulatory protein;
a sixth nucleic acid molecule, incorporating a second recognition sequence of the transcriptional activator;
a seventh nucleic acid molecule, operably linked to the sixth nucleic acid molecule and incorporating a second promoter and a second regulatory element;
an eighth nucleic acid molecule, operably linked to the seventh nucleic acid and encoding a second regulatory protein; and
at least one selected from the group consisting of:
a ninth nucleic acid molecule, operably linked to the fifth nucleic acid molecule and configured to conditionally inhibit expression of the first regulatory protein;
a tenth nucleic acid molecule, operably linked to the eighth nucleic acid molecule and configured to conditionally inhibit expression of the second regulatory protein,
wherein
the first regulatory element is adapted to inhibit the function of the first promoter by binding to the second regulatory protein, and
the second regulatory element is adapted to inhibit the function of the second promoter by binding to the first regulatory protein.

2. The expression system according to claim 1, wherein the cell-specific promoter is a tumor cell-specific promoter, and the tumor cell-specific promoter is at least one selected from the group consisting of an alpha-fetoprotein-specific promoter, a Survivin promoter, a human telomerase reverse transcriptase gene promoter, a cholecystokinin A receptor gene promoter, a carcinoembryonic antigen promoter, a proto-oncogene human epidermal growth factor receptor 2 promoter, a prostaglandin endoxygenase reductase 2 promoter, a chemokine receptor-4, an E2F-1 gene promoter, a mucin promoter, a prostate specific antigen, a human tyrosinase-related protein 1, and a tyrosinase promoter.

3. The expression system according to claim 1, wherein the transcriptional activator is at least one selected from the group consisting of Gal4VP16, Gal4vp64, dCas9-VPR, dCas9-VP64, dCas9-VP16, dCas9-VTR, and rtTA.

4. The expression system according to claim 1, wherein the first recognition sequence and the second recognition sequence are each independently selected from at least one of 5×UAS, 7×tetO and a target sequence of dCas9.

5. The expression system according to claim 1, wherein the first promoter and the second promoter are each independently selected from a miniCMV and a TATA box.

6. The expression system according to claim 1, wherein the first regulatory protein and the second regulatory protein are each independently selected from at least one of Lad, tetR, zinc finger, KRAB, tetR-KRAB and dCas9-KRAB.

7. The expression system according to claim 6, wherein the first regulatory element and the second regulatory element are each independently selected from at least one of a tetO, a LacO, a zinc finger target site, and a target sequence of dCas9.

8. The expression system according to claim 7, wherein the first regulatory protein is LacI, and the second regulatory element comprises a plurality of repeated LacO sequences, wherein at least one of the pluralities of repeated LacO sequences is set downstream of the second promoter.

9. The expression system according to claim 6, wherein the second regulatory protein is tetR-KRAB, and the first regulatory element comprises a plurality of repeated tetO sequences, wherein at least one of the pluralities of repeated tetO sequences is set downstream of the first promoter.

10. The expression system according to claim 1, wherein at least one of the fifth nucleic acid molecule and the ninth nucleic acid molecule further comprises a sequence encoding a protein of interest.

11. The expression system according to claim 10, wherein the fifth nucleic acid molecule comprises a sequence encoding the protein of interest, and the protein of interest comprises at least one selected from the group consisting of a viral replication and packaging protein and an immune effector.

12. The expression system according to claim 11, wherein the virus replication and packaging protein comprises at least one selected from the group consisting of an adenovirus E1 gene, an adenovirus E1A gene, an adenovirus E1B gene, an adenovirus E2 gene and an adenovirus E4 gene.

13. The expression system according to claim 11, wherein the immune effector comprises at least one sequence selected from the group consisting of an inhibitory sequence that antagonizes PD-1 gene, an inhibitory sequence that antagonizes PD-L1 gene, an inhibitory sequence that antagonizes CTLA4 gene, an inhibitory sequence that antagonizes Tim-3 gene, GM-CSF, IL-2, IL-12, and IL-15.

14. The expression system according to claim 10, wherein the protein of interest and the first regulatory protein are expressed in a form of a fusion protein, and the protein of interest and the first regulatory protein are linked by a cleavable linker peptide.

15. The expression system according to claim 1, wherein the ninth nucleic acid molecule and the tenth nucleic acid molecule independently inhibit expression of the first regulatory protein or the second regulatory protein, respectively, by RNA interference.

16. The expression system according to claim 15, wherein the ninth nucleic acid molecule comprises a nucleic acid sequence specifically recognized by a first microRNA, the tenth nucleic acid molecule comprises a nucleic acid sequence specifically recognized by a second microRNA, the first microRNA is a normal cell-specific microRNA, and the second microRNA is an abnormal cell-specific microRNA.

17. The expression system according to claim 16, wherein the first microRNA comprises at least one selected from the group consisting of miR199a, miR95, miR125, miR25b, Let-7, miR143, miR145, and miR200C.

18. The expression system according to claim 16, wherein the second microRNA comprises at least one selected from the group consisting of miR21, miR223, miR224, miR221, miR18, miR214, miR146a, and miR1792.

19. The expression system according to claim 1, wherein the first nucleic acid molecule and the second nucleic acid molecule are loaded in a first expression vector; the third nucleic acid molecule, the fourth nucleic acid molecule, the fifth nucleic acid molecule and optionally the ninth nucleic acid molecule are loaded in a second expression vector; and the sixth nucleic acid molecule, the seventh nucleic acid molecule, the eighth nucleic acid molecule and optionally the tenth nucleic acid molecules are loaded in a third expression vector.

20. The expression system according to claim 19, wherein the first expression vector, the second expression vector and the third expression vector are each independently selected from at least one of a plasmid, a virus, a nanomaterial, a liposome, a molecularly coupled vector, naked DNA, a chromosomal vector, and a polymer.

21-40. (canceled)


SEQUENCE LISTING (25 sequences)

SEQ ID NO: 1 - DNA, 2126 bp, Artificial Sequence
  Artificial Sequence created by rational design
ggtctctgctccagattgaattatttgcctgtcatacagctaataattgaccataagacaattagatttaaattagttttgaatctttctaataccaaagttcagtttactgttccatgttgcttctgagtggcttcacagacttatgaaaaagtaaacggaatcagaattacatcaatgcaaaagcattgctgtgaactctgtacttaggactaaactttgagcaataacacatatagattgaggattgtttgctgttagtatacaaactctggttcaaagctcctctttattgcttgtcttggaaaatttgctgttcttcatggtttctcttttcactgctatctatttttctcaaccactcacatggctacaaaagcttcctgattaataattacactaagtcaataggcatagagccaggactgtttgggtaaactggtcactttatcttaaactaaatatatccaaaactgaacatgtacttagttactaagtctttgactttatctcattcataccactcagctttatccaggccacttatttgacagtattattgcgaaaacttcctatctagaagtttgaggagaatatttgttatatttgcaaaataaaataagtttgcaagttttttttttctgccccaaagagctctgtgtccttgaacataaaatacaaataaccgctatgctgttaattattgacaaatgtcccattttcaacctaaggaaataccataaagtaacagatataccaacaaaaggttactagttaacaggcattgcctgaaaagagtataaaagaatttcagcatgattttccatattgtgcttccaccactgccaataacaaaataactagcaaggatccggccaccatggcccccccgaccgatgtcagcctgggggacgagctccacttagacggcgaggacgtggcgatggcgcatgccgacgcgctagacgatttcgatctggacatgttgggggacggggattccccgggtccgggatttaccccccacgactccgccccctacggcgctctggatatggccgacttcgagtttgagcagatgtttaccgatgcccttggaattgacgagtacggtgggacgcgtatgaagctactgtcttctatcgaacaagcatgcgatatttgccgacttaaaaagctcaagtgctccaaagaaaaaccgaagtgcgccaagtgtctgaagaacaactgggagtgtcgctactctcccaaaaccaaaaggtctccgctgactagggcacatctgacagaagtggaatcaaggctagaaagactggaacagctatttctactgatttttcctcgagaagaccttgacatgattttgaaaatggattctttacaggatataaaagcattgttaacaggattatttgtacaagataatgtgaataaagatgccgtcacagatagattggcttcagtggagactgatatgcctctaacattgagacagcatagaataagtgcgacatcatcatcggaagagagtagtaacaaaggtcaaagacagttgactgtataattcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatcggagaaagaggtaatttaattaagtcgatgagacc

SEQ ID NO: 2 - DNA, 3510 bp, Artificial Sequence
  Artificial Sequence created by rational design
ggtctctctatttaattaagtaactataacggtcgctccgaatttctcgagttaattaagattacgccaagctacgggcggagtactgtcctccgagcggagtactgtcctccgagcggagtactgtcctccgagcggagtactgtcctccgagcggagttctgtcctccgagcggagactctagactccctatcagtgatagagataggcgtgtacggtgggaggcctatataagcagagctcgtttagtgaaccgtcagatcgctccctatcagtgatagagagaattcgaccgccaccatgtcggaattcatgagacatattatctgccacggaggtgttattaccgaagaaatggccgccagtcttttggaccagctgatcgaagaggtactggctgataatcttccacctcctagccattttgaaccacctacccttcacgaactgtatgatttagacgtgacggcccccgaagatcccaacgaggaggcggtttcgcagatttttcccgactctgtaatgttggcggtgcaggaagggattgacttactcacttttccgccggcgcccggttctccggagccgcctcacctttcccggcagcccgagcagccggagcagagagccttgggtccggtttctatgccaaaccttgtaccggaggtgatcgatcttacctgccacgaggctggctttccacccagtgacgacgaggatgaagagggtgaggagtttgtgttagattatgtggagcaccccgggcacggttgcaggtcttgtcattatcaccggaggaatacgggggacccagatattatgtgttcgctttgctatatgaggacctgtggcatgtttgtctacagtaagtgaaaattatgggcagtgggtgatagagtggtgggtttggtgtggtaattttttttttaatttttacagttttgtggtttaaagaattttgtattgtgatttttttaaaaggtcctgtgtctgaacctgagcctgagcccgagccagaaccggagcctgcaagacctacccgccgtcctaaaatggcgcctgctatcctgagacgcccgacatcacctgtgtctagagaatgcaatagtagtacggatagctgtgactccggtccttctaacacacctcctgagatacacccggtggtcccgctgtgccccattaaaccagttgccgtgagagttggtgggcgtcgccaggctgtggaatgtatcgaggacttgcttaacgagcctgggcaacctttggacttgagctgtaaacgccccaggccactcgagggtaccgggtccggagctacaaatttttccctcctcaaacaggctggagatgtcgaagaaaatcctgggcctaccggtcccatggtgagcaagggcgaggagctgttcaccggggtggtgcccatcctggtcgagctggacggcgacgtaaacggccacaagttcagcgtgaggggcgagggcgagggcgatgccaccaacggcaagctgaccctgaagttcatctgcaccaccggcaagctgcccgtgccctggcccaccctcgtgaccaccctgagccacggcgtgcagtgcttcgcccgctaccccgaccacatgaagcagcacgacttcttcaagtccgccatgcccgaaggctacgtccaggagcgcaccatcttcttcaaggacgacggcacctacaagacccgcgccgaggtgaagttcgagggcgacaccctagtgaaccgcatcgagctgaagggcgtcgacttcaaggaggacggcaacatcctggggcacaagctggagtacaacttcaacagccacaacatctatatcatggccgtcaagcagaagaacggcatcaaggtgaacttcaagatccgccacaacgtggaggacggcagcgtgcagctcgccgaccactaccagcagaacacccccatcggcgacggccccgtgctgctgcccgacagccactacctgagcacccagtccgtgctgagcaaagaccccaacgagaagcgcgatcacatggtcctgctggagttccgcaccgccgccgggatcactctcggcatggacgagctgtacaagagatctgagggccgcggcagcctgctgacctgcggcgacgtggaggaaaaccccggccccggatccatgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagagcagcctgaggcctcctaagaagaagaggaaggttgcggccgctaaccaatgtgcagactactgttaaccaatgtgcagactactgtgaattctaaccaatgtgcagactactgttaaccaatgtgcagactactgtaagcttcgaattgagtagtgctttctactttatgagtagtgctttctactttatgagtagtgctttctactttatgagtagtgctttctactttatggtcgacaatactagttaagaaatgagacc

SEQ ID NO: 3 - DNA, 3242 bp, Artificial Sequence
  Artificial Sequence created by rational design
ggtctctctatttaattaagtaactataacggtcgctccgaatttctcgagttaattaagattacgccaagctacgggcggagtactgtcctccgagcggagtactgtcctccgagcggagtactgtcctccgagcggagtactgtcctccgagcggagttctgtcctccgagcggagactctagactccctatcagtgatagagataggcgtgtacggtgggaggcctatataagcagagctcgtttagtgaaccgtcagatcgctccctatcagtgatagagagaattcgaccgccaccatgtcggaattcatgagacatattatctgccacggaggtgttattaccgaagaaatggccgccagtcttttggaccagctgatcgaagaggtactggctgataatcttccacctcctagccattttgaaccacctacccttcacgaactgtatgatttagacgtgacggcccccgaagatcccaacgaggaggcggtttcgcagatttttcccgactctgtaatgttggcggtgcaggaagggattgacttactcacttttccgccggcgcccggttctccggagccgcctcacctttcccggcagcccgagcagccggagcagagagccttgggtccggtttctatgccaaaccttgtaccggaggtgatcgatcttacctgccacgaggctggctttccacccagtgacgacgaggatgaagagggtgaggagtttgtgttagattatgtggagcaccccgggcacggttgcaggtcttgtcattatcaccggaggaatacgggggacccagatattatgtgttcgctttgctatatgaggacctgtggcatgtttgtctacagtaagtgaaaattatgggcagtgggtgatagagtggtgggtttggtgtggtaattttttttttaatttttacagttttgtggtttaaagaattttgtattgtgatttttttaaaaggtcctgtgtctgaacctgagcctgagcccgagccagaaccggagcctgcaagacctacccgccgtcctaaaatggcgcctgctatcctgagacgcccgacatcacctgtgtctagagaatgcaatagtagtacggatagctgtgactccggtccttctaacacacctcctgagatacacccggtggtcccgctgtgccccattaaaccagttgccgtgagagttggtgggcgtcgccaggctgtggaatgtatcgaggacttgcttaacgagcctgggcaacctttggacttgagctgtaaacgccccaggccactcgaggagggccgcggcagcctgctgacctgcggcgacgtggaggaaaaccccggccccaccggtcaccatggatgtacaggatgcaactcctgtcttgcattgcactaagtcttgcacttgtcacaaacagtgcacctacttcaagttctacaaagaaaacacagctacaactggagcatttactgctggatttacagatgattttgaatggaattaataattacaagaatcccaaactcaccgcgatgctcacagctaagtttgccatgcccaagaaggccacagaactgaaacatcttcagtgtctagaagaagcactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactgacttgaagatctgagggccgcggcagcctgctgacctgcggcgacgtggaggaaaaccccggccccggatccatgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagagcagcctgaggcctcctaagaagaagaggaaggttgcggccgctaaccaatgtgcagactactgttaaccaatgtgcagactactgtgaattctaaccaatgtgcagactactgttaaccaatgtgcagactactgtaagcttcgaattgagtagtgctttctactttatgagtagtgctttctactttatgagtagtgctttctactttatgagtagtgctttctactttatggtcgacaatactagttaagaaatgagacc

SEQ ID NO: 4 - DNA, 3233 bp, Artificial Sequence
  Artificial Sequence created by rational design
ggtctctctatttaattaagtaactataacggtcgctccgaatttctcgagttaattaagattacgccaagctacgggcggagtactgtcctccgagcggagtactgtcctccgagcggagtactgtcctccgagcggagtactgtcctccgagcggagttctgtcctccgagcggagactctagactccctatcagtgatagagataggcgtgtacggtgggaggcctatataagcagagctcgtttagtgaaccgtcagatcgctccctatcagtgatagagagaattcgaccgccaccatgtcggaattcatgagacatattatctgccacggaggtgttattaccgaagaaatggccgccagtcttttggaccagctgatcgaagaggtactggctgataatcttccacctcctagccattttgaaccacctacccttcacgaactgtatgatttagacgtgacggcccccgaagatcccaacgaggaggcggtttcgcagatttttcccgactctgtaatgttggcggtgcaggaagggattgacttactcacttttccgccggcgcccggttctccggagccgcctcacctttcccggcagcccgagcagccggagcagagagccttgggtccggtttctatgccaaaccttgtaccggaggtgatcgatcttacctgccacgaggctggctttccacccagtgacgacgaggatgaagagggtgaggagtttgtgttagattatgtggagcaccccgggcacggttgcaggtcttgtcattatcaccggaggaatacgggggacccagatattatgtgttcgctttgctatatgaggacctgtggcatgtttgtctacagtaagtgaaaattatgggcagtgggtgatagagtggtgggtttggtgtggtaattttttttttaatttttacagttttgtggtttaaagaattttgtattgtgatttttttaaaaggtcctgtgtctgaacctgagcctgagcccgagccagaaccggagcctgcaagacctacccgccgtcctaaaatggcgcctgctatcctgagacgcccgacatcacctgtgtctagagaatgcaatagtagtacggatagctgtgactccggtccttctaacacacctcctgagatacacccggtggtcccgctgtgccccattaaaccagttgccgtgagagttggtgggcgtcgccaggctgtggaatgtatcgaggacttgcttaacgagcctgggcaacctttggacttgagctgtaaacgccccaggccactcgagggtaccgggtccggagctacaaatttttccctcctcaaacaggctggagatgtcgaagaaaatcctgggcctaccggtcaccatggatgtggctgcagagcctgctgctcttgggcactgtggcctgcagcatctctgcacccgcccgctcgcccagccccagcacgcagccctgggagcatgtgaatgccatccaggaggcccggcgtctcctgaacctgagtagagacactgctgctgagatgaatgaaacagtagaagtcatctcagaaatgtttgacctccaggagccgacctgcctacagacccgcctggagctgtacaagcagggcctgcggggcagcctcaccaagctcaagggccccttgaccatgatggccagccactacaagcagcactgccctccaaccccggaaacttcctgtgcaacccagattatcacctttgaaagtttcaaagagaacctgaaggactttctgcttgtcatcccctttgactgctgggagccagtccaggagtgaagatctgagggccgcggcagcctgctgacctgcggcgacgtggaggaaaaccccggccccggatccatgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagagcagcctgaggcctcctaagaagaagaggaaggttgcggccgctaaccaatgtgcagactactgttaaccaatgtgcagactactgtgaattctaaccaatgtgcagactactgttaaccaatgtgcagactactgtaagcttcgaattgagtagtgctttctactttatgagtagtgctttctactttatgagtagtgctttctactttatgagtagtgctttctactttatggtcgacaatactagttaagaaatgagacc

SEQ ID NO: 5 - DNA, 3224 bp, Artificial Sequence
  Artificial Sequence created by rational design
ggtctctctatttaattaagtaactataacggtcgctccgaatttctcgagttaattaagattacgccaagctacgggcggagtactgtcctccgagcggagtactgtcctccgagcggagtactgtcctccgagcggagtactgtcctccgagcggagttctgtcctccgagcggagactctagactccctatcagtgatagagataggcgtgtacggtgggaggcctatataagcagagctcgtttagtgaaccgtcagatcgctccctatcagtgatagagagaattcgaccgccaccatgtcggaattcatgagacatattatctgccacggaggtgttattaccgaagaaatggccgccagtcttttggaccagctgatcgaagaggtactggctgataatcttccacctcctagccattttgaaccacctacccttcacgaactgtatgatttagacgtgacggcccccgaagatcccaacgaggaggcggtttcgcagatttttcccgactctgtaatgttggcggtgcaggaagggattgacttactcacttttccgccggcgcccggttctccggagccgcctcacctttcccggcagcccgagcagccggagcagagagccttgggtccggtttctatgccaaaccttgtaccggaggtgatcgatcttacctgccacgaggctggctttccacccagtgacgacgaggatgaagagggtgaggagtttgtgttagattatgtggagcaccccgggcacggttgcaggtcttgtcattatcaccggaggaatacgggggacccagatattatgtgttcgctttgctatatgaggacctgtggcatgtttgtctacagtaagtgaaaattatgggcagtgggtgatagagtggtgggtttggtgtggtaattttttttttaatttttacagttttgtggtttaaagaattttgtattgtgatttttttaaaaggtcctgtgtctgaacctgagcctgagcccgagccagaaccggagcctgcaagacctacccgccgtcctaaaatggcgcctgctatcctgagacgcccgacatcacctgtgtctagagaatgcaatagtagtacggatagctgtgactccggtccttctaacacacctcctgagatacacccggtggtcccgctgtgccccattaaaccagttgccgtgagagttggtgggcgtcgccaggctgtggaatgtatcgaggacttgcttaacgagcctgggcaacctttggacttgagctgtaaacgccccaggccactcgagggtaccgggtccggagctacaaatttttccctcctcaaacaggctggagatgtcgaagaaaatcctgggcctaccggtcaccatggatgtggctgcagaatttacttttcctgggcattgtggtctacagcctctcagcacccacccgctcacccatcactgtcacccggccttggaagcatgtagaggccatcaaagaagccctgaacctcctggatgacatgcctgtcacgttgaatgaagaggtagaagtcgtctctaacgagttctccttcaagaagctaacatgtgtgcagacccgcctgaagatattcgagcagggtctacggggcaatttcaccaaactcaagggcgccttgaacatgacagccagctactaccagacatactgccccccaactccggaaacggactgtgaaacacaagttaccacctatgcggatttcatagacagccttaaaacctttctgactgatatcccctttgaatgcaaaaaaccaggccaaaaatgaagatctgagggccgcggcagcctgctgacctgcggcgacgtggaggaaaaccccggccccggatccatgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagagcagcctgaggcctcctaagaagaagaggaaggttgcggccgctaaccaatgtgcagactactgttaaccaatgtgcagactactgtgaattctaaccaatgtgcagactactgttaaccaatgtgcagactactgtaagcttcgaattgagtagtgctttctactttatgagtagtgctttctactttatgagtagtgctttctactttatgagtagtgctttctactttatggtcgacaatactagttaagaaatgagacc

SEQ ID NO: 6 - DNA, 3558 bp, Artificial Sequence
  Artificial Sequence created by rational design
ggtctctctatttaattaagtaactataacggtcgctccgaatttctcgagttaattaagattacgccaagctacgggcggagtactgtcctccgagcggagtactgtcctccgagcggagtactgtcctccgagcggagtactgtcctccgagcggagttctgtcctccgagcggagactctagactccctatcagtgatagagataggcgtgtacggtgggaggcctatataagcagagctcgtttagtgaaccgtcagatcgctccctatcagtgatagagagaattcgaccgccaccatgtcggaattcatgagacatattatctgccacggaggtgttattaccgaagaaatggccgccagtcttttggaccagctgatcgaagaggtactggctgataatcttccacctcctagccattttgaaccacctacccttcacgaactgtatgatttagacgtgacggcccccgaagatcccaacgaggaggcggtttcgcagatttttcccgactctgtaatgttggcggtgcaggaagggattgacttactcacttttccgccggcgcccggttctccggagccgcctcacctttcccggcagcccgagcagccggagcagagagccttgggtccggtttctatgccaaaccttgtaccggaggtgatcgatcttacctgccacgaggctggctttccacccagtgacgacgaggatgaagagggtgaggagtttgtgttagattatgtggagcaccccgggcacggttgcaggtcttgtcattatcaccggaggaatacgggggacccagatattatgtgttcgctttgctatatgaggacctgtggcatgtttgtctacagtaagtgaaaattatgggcagtgggtgatagagtggtgggtttggtgtggtaattttttttttaatttttacagttttgtggtttaaagaattttgtattgtgatttttttaaaaggtcctgtgtctgaacctgagcctgagcccgagccagaaccggagcctgcaagacctacccgccgtcctaaaatggcgcctgctatcctgagacgcccgacatcacctgtgtctagagaatgcaatagtagtacggatagctgtgactccggtccttctaacacacctcctgagatacacccggtggtcccgctgtgccccattaaaccagttgccgtgagagttggtgggcgtcgccaggctgtggaatgtatcgaggacttgcttaacgagcctgggcaacctttggacttgagctgtaaacgccccaggccactcgagggtaccgggtccggagctacaaatttttccctcctcaaacaggctggagatgtcgaagaaaatcctgggcctaccggtcccatgagtgtgcccactcaggtcctggggttgctgctgctgtggcttacagacgctcgctgccaggtgcagctggtggagagcggcggcggcgtggtgcagcccggcaggagcctgaggctggactgcaaggccagcggcatcaccttcagcaacagcggcatgcactgggtgaggcaggcccccggcaagggcctggagtgggtggccgtgatctggtacgacggcagcaagaggtactacgccgacagcgtgaagggcaggttcaccatcagcagggacaacagcaagaacaccctgttcctgcagatgaacagcctgagggccgaggacaccgccgtgtactactgcgccaccaacgacgactactggggccagggcaccctggtgaccgtgagcagcggtggaggcggttcaggcggaggtggctctggcggtggcggatcggagatcgtgctgacccagagccccgccaccctgagcctgagccccggcgagagggccaccctgagctgcagggccagccagagcgtgagcagctacctggcctggtaccagcagaagcccggccaggcccccaggctgctgatctacgacgccagcaacagggccaccggcatccccgccaggttcagcggcagcggcagcggcaccgacttcaccctgaccatcagcagcctggagcccgaggacttcgccgtgtactactgccagcagagcagcaactggcccaggaccttcggccagggcaccaaggtggagatcaagagatctgagggccgcggcagcctgctgacctgcggcgacgtggaggaaaaccccggccccggatccatgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagagcagcctgaggcctcctaagaagaagaggaaggttgcggccgctaaccaatgtgcagactactgttaaccaatgtgcagactactgtgaattctaaccaatgtgcagactactgttaaccaatgtgcagactactgtaagcttcgaattgagtagtgctttctactttatgagtagtgctttctactttatgagtagtgctttctactttatgagtagtgctttctactttatggtcgacaatactagttaagaaatgagacc

SEQ ID NO: 7 - DNA, 3573 bp, Artificial Sequence
  Artificial Sequence created by rational design
ggtctctctatttaattaagtaactataacggtcgctccgaatttctcgagttaattaagattacgccaagctacgggcggagtactgtcctccgagcggagtactgtcctccgagcggagtactgtcctccgagcggagtactgtcctccgagcggagttctgtcctccgagcggagactctagactccctatcagtgatagagataggcgtgtacggtgggaggcctatataagcagagctcgtttagtgaaccgtcagatcgctccctatcagtgatagagagaattcgaccgccaccatgtcggaattcatgagacatattatctgccacggaggtgttattaccgaagaaatggccgccagtcttttggaccagctgatcgaagaggtactggctgataatcttccacctcctagccattttgaaccacctacccttcacgaactgtatgatttagacgtgacggcccccgaagatcccaacgaggaggcggtttcgcagatttttcccgactctgtaatgttggcggtgcaggaagggattgacttactcacttttccgccggcgcccggttctccggagccgcctcacctttcccggcagcccgagcagccggagcagagagccttgggtccggtttctatgccaaaccttgtaccggaggtgatcgatcttacctgccacgaggctggctttccacccagtgacgacgaggatgaagagggtgaggagtttgtgttagattatgtggagcaccccgggcacggttgcaggtcttgtcattatcaccggaggaatacgggggacccagatattatgtgttcgctttgctatatgaggacctgtggcatgtttgtctacagtaagtgaaaattatgggcagtgggtgatagagtggtgggtttggtgtggtaattttttttttaatttttacagttttgtggtttaaagaattttgtattgtgatttttttaaaaggtcctgtgtctgaacctgagcctgagcccgagccagaaccggagcctgcaagacctacccgccgtcctaaaatggcgcctgctatcctgagacgcccgacatcacctgtgtctagagaatgcaatagtagtacggatagctgtgactccggtccttctaacacacctcctgagatacacccggtggtcccgctgtgccccattaaaccagttgccgtgagagttggtgggcgtcgccaggctgtggaatgtatcgaggacttgcttaacgagcctgggcaacctttggacttgagctgtaaacgccccaggccactcgagggtaccgggtccggagctacaaatttttccctcctcaaacaggctggagatgtcgaagaaaatcctgggcctaccggtcccatgagtgtgcccactcaggtcctggggttgctgctgctgtggcttacagacgctcgctgcgaggtgcagctggtggagagcggcggcggcctggtgcagcccggcggcagcctgaggctgagctgcgccgccagcggcttcaccttcagcgacagctggatccactgggtgaggcaggcccccggcaagggcctggagtgggtggcctggatcagcccctacggcggcagcacctactacgccgacagcgtgaagggcaggttcaccatcagcgccgacaccagcaagaacaccgcctacctgcagatgaacagcctgagggccgaggacaccgccgtgtactactgcgccaggaggcactggcccggcggcttcgactactggggccagggcaccctggtgaccgtgagcagcggtggaggcggttcaggcggaggtggctctggcggtggcggatcggacatccagatgacccagagccccagcagcctgagcgccagcgtgggcgacagggtgaccatcacctgcagggccagccaggacgtgagcaccgccgtggcctggtaccagcagaagcccggcaaggcccccaagctgctgatctacagcgccagcttcctgtacagcggcgtgcccagcaggttcagcggcagcggcagcggcaccgacttcaccctgaccatcagcagcctgcagcccgaggacttcgccacctactactgccagcagtacctgtaccaccccgccaccttcggccagggcaccaaggtggagatcaagagatctgagggccgcggcagcctgctgacctgcggcgacgtggaggaaaaccccggccccggatccatgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagagcagcctgaggcctcctaagaagaagaggaaggttgcggccgctaaccaatgtgcagactactgttaaccaatgtgcagactactgtgaattctaaccaatgtgcagactactgttaaccaatgtgcagactactgtaagcttcgaattgagtagtgctttctactttatgagtagtgctttctactttatgagtagtgctttctactttatgagtagtgctttctactttatggtcgacaatactagttaagaaatgagacc

SEQ ID NO: 8 - DNA, 1594 bp, Artificial Sequence
  Artificial Sequence created by rational design
ggtctccgaaatcgccaagctacgggcggagtactgtcctccgagcggagtactgtcctccgagcggagtactgtcctccgagcggagtactgtcctccgagcggagtactgtcctccgagcggagactctagaaattgtgagcggataacaatttaggcgtgtacggtgggaggcctatataagcagagctcgtttagtgaaccgtcagatcgccagctgacaaaattgtgagcgctcacaattactagaagatcttgaattcgccaccatggctagattagataaaagtaaagtgattaacagcgcattagagctgcttaatgaggtcggaatcgaaggtttaacaacccgtaaactcgcccagaagctaggtgtagagcagcctacattgtattggcatgtaaaaaataagcgggctttgctcgacgccttagccattgagatgttagataggcaccatactcacttttgccctttagaaggggaaagctggcaagattttttacgtaataacgctaaaagttttagatgtgctttactaagtcatcgcgatggagcaaaagtacatttaggtacacggcctacagaaaaacagtatgaaactctcgaaaatcaattagcctttttatgccaacaaggtttttcactagagaatgcattatatgcactcagcgctgtggggcattttactttaggttgcgtattggaagatcaagagcatcaagtcgctaaagaagaaagggaaacacctactactgatagtatgccgccattattacgacaagctatcgaattatttgatcaccaaggtgcagagccagccttcttattcggccttgaattgatcatatgcggattagaaaaacaacttaaatgtgaaagtgggtcgccaaaaaagaagagaaaggtcgacggcggtggtgctttgtctcctcagcactctgctgtcactcaaggaagtatcatcaagaacaaggagggcatggatgctaagtcactaactgcctggtcccggacactggtgaccttcaaggatgtatttgtggacttcaccagggaggagtggaagctgctggacactgctcagcagatcgtgtacagaaatgtgatgctggagaactataagaacctggtttccttgggttatcagcttactaagccagatgtgatcctccggttggagaagggagaagagccctggctggtggagagagaaattcaccaagagacccatcctgattcagagactgcatttgaaatcaaatcatcagtttaatacaaggcggccgcaaatcaacatcagtctgataagctatcaacatcagtctgataagctatcaacatcagtctgataagctatcaacatcagtctgataagctaaagcttcgaattctgataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcagctctgagacc

SEQ ID NO: 9 - DNA, 7155 bp, Artificial Sequence
  Artificial Sequence created by rational design
gtaactataacggtcgctccgaatttctcgagttaattaagattacgccaagctacgggcggagtactgtcctccgagcggagtactgtcctccgagcggagtactgtcctccgagcggagtactgtcctccgagcggagttctgtcctccgagcggagactctagactccctatcagtgatagagataggcgtgtacggtgggaggcctatataagcagagctcgtttagtgaaccgtcagatcgctccctatcagtgatagagagaattcgaccgccaccatgtcggaattcatgagacatattatctgccacggaggtgttattaccgaagaaatggccgccagtcttttggaccagctgatcgaagaggtactggctgataatcttccacctcctagccattttgaaccacctacccttcacgaactgtatgatttagacgtgacggcccccgaagatcccaacgaggaggcggtttcgcagatttttcccgactctgtaatgttggcggtgcaggaagggattgacttactcacttttccgccggcgcccggttctccggagccgcctcacctttcccggcagcccgagcagccggagcagagagccttgggtccggtttctatgccaaaccttgtaccggaggtgatcgatcttacctgccacgaggctggctttccacccagtgacgacgaggatgaagagggtgaggagtttgtgttagattatgtggagcaccccgggcacggttgcaggtcttgtcattatcaccggaggaatacgggggacccagatattatgtgttcgctttgctatatgaggacctgtggcatgtttgtctacagtaagtgaaaattatgggcagtgggtgatagagtggtgggtttggtgtggtaattttttttttaatttttacagttttgtggtttaaagaattttgtattgtgatttttttaaaaggtcctgtgtctgaacctgagcctgagcccgagccagaaccggagcctgcaagacctacccgccgtcctaaaatggcgcctgctatcctgagacgcccgacatcacctgtgtctagagaatgcaatagtagtacggatagctgtgactccggtccttctaacacacctcctgagatacacccggtggtcccgctgtgccccattaaaccagttgccgtgagagttggtgggcgtcgccaggctgtggaatgtatcgaggacttgcttaacgagcctgggcaacctttggacttgagctgtaaacgccccaggccactcgagggtaccgggtccggagctacaaatttttccctcctcaaacaggctggagatgtcgaagaaaatcctgggcctaccggtcccatggtgagcaagggcgaggagctgttcaccggggtggtgcccatcctggtcgagctggacggcgacgtaaacggccacaagttcagcgtgaggggcgagggcgagggcgatgccaccaacggcaagctgaccctgaagttcatctgcaccaccggcaagctgcccgtgccctggcccaccctcgtgaccaccctgagccacggcgtgcagtgcttcgcccgctaccccgaccacatgaagcagcacgacttcttcaagtccgccatgcccgaaggctacgtccaggagcgcaccatcttcttcaaggacgacggcacctacaagacccgcgccgaggtgaagttcgagggcgacaccctagtgaaccgcatcgagctgaagggcgtcgacttcaaggaggacggcaacatcctggggcacaagctggagtacaacttcaacagccacaacatctatatcatggccgtcaagcagaagaacggcatcaaggtgaacttcaagatccgccacaacgtggaggacggcagcgtgcagctcgccgaccactaccagcagaacacccccatcggcgacggccccgtgctgctgcccgacagccactacctgagcacccagtccgtgctgagcaaagaccccaacgagaagcgcgatcacatggtcctgctggagttccgcaccgccgccgggatcactctcggcatggacgagctgtacaagagatctgagggccgcggcagcctgctgacctgcggcgacgtggaggaaaaccccggccccggatccatgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagagcagcctgaggcctcctaagaagaagaggaaggttgcggccgctaaccaatgtgcagactactgttaaccaatgtgcagactactgtgaattctaaccaatgtgcagactactgttaaccaatgtgcagactactgtaagcttcgaattgagtagtgctttctactttatgagtagtgctttctactttatgagtagtgctttctactttatgagtagtgctttctactttatggtcgacaatactagttaagaaatcgccaagctacgggcggagtactgtcctccgagcggagtactgtcctccgagcggagtactgtcctccgagcggagtactgtcctccgagcggagtactgtcctccgagcggagactctagaaattgtgagcggataacaatttaggcgtgtacggtgggaggcctatataagcagagctcgtttagtgaaccgtcagatcgccagctgacaaaattgtgagcgctcacaattactagaagatcttgaattcgccaccatggctagattagataaaagtaaagtgattaacagcgcattagagctgcttaatgaggtcggaatcgaaggtttaacaacccgtaaactcgcccagaagctaggtgtagagcagcctacattgtattggcatgtaaaaaataagcgggctttgctcgacgccttagccattgagatgttagataggcaccatactcacttttgccctttagaaggggaaagctggcaagattttttacgtaataacgctaaaagttttagatgtgctttactaagtcatcgcgatggagcaaaagtacatttaggtacacggcctacagaaaaacagtatgaaactctcgaaaatcaattagcctttttatgccaacaaggtttttcactagagaatgcattatatgcactcagcgctgtggggcattttactttaggttgcgtattggaagatcaagagcatcaagtcgctaaagaagaaagggaaacacctactactgatagtatgccgccattattacgacaagctatcgaattatttgatcaccaaggtgcagagccagccttcttattcggccttgaattgatcatatgcggattagaaaaacaacttaaatgtgaaagtgggtcgccaaaaaagaagagaaaggtcgacggcggtggtgctttgtctcctcagcactctgctgtcactcaaggaagtatcatcaagaacaaggagggcatggatgctaagtcactaactgcctggtcccggacactggtgaccttcaaggatgtatttgtggacttcaccagggaggagtggaagctgctggacactgctcagcagatcgtgtacagaaatgtgatgctggagaactataagaacctggtttccttgggttatcagcttactaagccagatgtgatcctccggttggagaagggagaagagccctggctggtggagagagaaattcaccaagagacccatcctgattcagagactgcatttgaaatcaaatcatcagtttaatacaaggcggccgcaaatcaacatcagtctgataagctatcaacatcagtctgataagctatcaacatcagtctgataagctatcaacatcagtctgataagctaaagcttcgaattctgataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcagctccagattgaattatttgcctgtcatacagctaataattgaccataagacaattagatttaaattagttttgaatctttctaataccaaagttcagtttactgttccatgttgcttctgagtggcttcacagacttatgaaaaagtaaacggaatcagaattacatcaatgcaaaagcattgctgtgaactctgtacttaggactaaactttgagcaataacacatatagattgaggattgtttgctgttagtatacaaactctggttcaaagctcctctttattgcttgtcttggaaaatttgctgttcttcatggtttctcttttcactgctatctatttttctcaaccactcacatggctacaaaagcttcctgattaataattacactaagtcaataggcatagagccaggactgtttgggtaaactggtcactttatcttaaactaaatatatccaaaactgaacatgtacttagttactaagtctttgactttatctcattcataccactcagctttatccaggccacttatttgacagtattattgcgaaaacttcctatctagaagtttgaggagaatatttgttatatttgcaaaataaaataagtttgcaagttttttttttctgccccaaagagctctgtgtccttgaacataaaatacaaataaccgctatgctgttaattattgacaaatgtcccattttcaacctaaggaaataccataaagtaacagatataccaacaaaaggttactagttaacaggcattgcctgaaaagagtataaaagaatttcagcatgattttccatattgtgcttccaccactgccaataacaaaataactagcaaggatccggccaccatggcccccccgaccgatgtcagcctgggggacgagctccacttagacggcgaggacgtggcgatggcgcatgccgacgcgctagacgatttcgatctggacatgttgggggacggggattccccgggtccgggatttaccccccacgactccgccccctacggcgctctggatatggccgacttcgagtttgagcagatgtttaccgatgcccttggaattgacgagtacggtgggacgcgtatgaagctactgtcttctatcgaacaagcatgcgatatttgccgacttaaaaagctcaagtgctccaaagaaaaaccgaagtgcgccaagtgtctgaagaacaactgggagtgtcgctactctcccaaaaccaaaaggtctccgctgactagggcacatctgacagaagtggaatcaaggctagaaagactggaacagctatttctactgatttttcctcgagaagaccttgacatgattttgaaaatggattctttacaggatataaaagcattgttaacaggattatttgtacaagataatgtgaataaagatgccgtcacagatagattggcttcagtggagactgatatgcctctaacattgagacagcatagaataagtgcgacatcatcatcggaagagagtagtaacaaaggtcaaagacagttgactgtataattcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatcggagaaagaggtaat

SEQ ID NO: 10 - DNA, 6900 bp, Artificial Sequence
  Artificial Sequence created by rational design
gtaactataacggtcgctccgaatttctcgagttaattaagattacgccaagctacgggcggagtactgtcctccgagcggagtactgtcctccgagcggagtactgtcctccgagcggagtactgtcctccgagcggagttctgtcctccgagcggagactctagactccctatcagtgatagagataggcgtgtacggtgggaggcctatataagcagagctcgtttagtgaaccgtcagatcgctccctatcagtgatagagagaattcgaccgccaccatgtcggaattcatgagacatattatctgccacggaggtgttattaccgaagaaatggccgccagtcttttggaccagctgatcgaagaggtactggctgataatcttccacctcctagccattttgaaccacctacccttcacgaactgtatgatttagacgtgacggcccccgaagatcccaacgaggaggcggtttcgcagatttttcccgactctgtaatgttggcggtgcaggaagggattgacttactcacttttccgccggcgcccggttctccggagccgcctcacctttcccggcagcccgagcagccggagcagagagccttgggtccggtttctatgccaaaccttgtaccggaggtgatcgatcttacctgccacgaggctggctttccacccagtgacgacgaggatgaagagggtgaggagtttgtgttagattatgtggagcaccccgggcacggttgcaggtcttgtcattatcaccggaggaatacgggggacccagatattatgtgttcgctttgctatatgaggacctgtggcatgtttgtctacagtaagtgaaaattatgggcagtgggtgatagagtggtgggtttggtgtggtaattttttttttaatttttacagttttgtggtttaaagaattttgtattgtgatttttttaaaaggtcctgtgtctgaacctgagcctgagcccgagccagaaccggagcctgcaagacctacccgccgtcctaaaatggcgcctgctatcctgagacgcccgacatcacctgtgtctagagaatgcaatagtagtacggatagctgtgactccggtccttctaacacacctcctgagatacacccggtggtcccgctgtgccccattaaaccagttgccgtgagagttggtgggcgtcgccaggctgtggaatgtatcgaggacttgcttaacgagcctgggcaacctttggacttgagctgtaaacgccccaggccactcgagggtaccgggtccggagctacaaatttttccctcctcaaacaggctggagatgtcgaagaaaatcctgggcctaccggtcccatgtacaggatgcaactcctgtcttgcattgcactaagtcttgcacttgtcacaaacagtgcacctacttcaagttctacaaagaaaacacagctacaactggagcatttactgctggatttacagatgattttgaatggaattaataattacaagaatcccaaactcaccgcgatgctcacagctaagtttgccatgcccaagaaggccacagaactgaaacatcttcagtgtctagaagaagcactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactgacttgaagatctgagggccgcggcagcctgctgacctgcggcgacgtggaggaaaaccccggccccggatccatgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagagcagcctgaggcctcctaagaagaagaggaaggttgcggccgctaaccaatgtgcagactactgttaaccaatgtgcagactactgtgaattctaaccaatgtgcagactactgttaaccaatgtgcagactactgtaagcttcgaattgagtagtgctttctactttatgagtagtgctttctactttatgagtagtgctttctactttatgagtagtgctttctactttatggtcgacaatactagttaagaaatcgccaagctacgggcggagtactgtcctccgagcggagtactgtcctccgagcggagtactgtcctccgagcggagtactgtcctccgagcggagtactgtcctccgagcggagactctagaaattgtgagcggataacaatttaggcgtgtacggtgggaggcctatataagcagagctcgtttagtgaaccgtcagatcgccagctgacaaaattgtgagcgctcacaattactagaagatcttgaattcgccaccatggctagattagataaaagtaaagtgattaacagcgcattagagctgcttaatgaggtcggaatcgaaggtttaacaacccgtaaactcgcccagaagctaggtgtagagcagcctacattgtattggcatgtaaaaaataagcgggctttgctcgacgccttagccattgagatgttagataggcaccatactcacttttgccctttagaaggggaaagctggcaagattttttacgtaataacgctaaaagttttagatgtgctttactaagtcatcgcgatggagcaaaagtacatttaggtacacggcctacagaaaaacagtatgaaactctcgaaaatcaattagcctttttatgccaacaaggtttttcactagagaatgcattatatgcactcagcgctgtggggcattttactttaggttgcgtattggaagatcaagagcatcaagtcgctaaagaagaaagggaaacacctactactgatagtatgccgccattattacgacaagctatcgaattatttgatcaccaaggtgcagagccagccttcttattcggccttgaattgatcatatgcggattagaaaaacaacttaaatgtgaaagtgggtcgccaaaaaagaagagaaaggtcgacggcggtggtgctttgtctcctcagcactctgctgtcactcaaggaagtatcatcaagaacaaggagggcatggatgctaagtcactaactgcctggtcccggacactggtgaccttcaaggatgtatttgtggacttcaccagggaggagtggaagctgctggacactgctcagcagatcgtgtacagaaatgtgatgctggagaactataagaacctggtttccttgggttatcagcttactaagccagatgtgatcctccggttggagaagggagaagagccctggctggtggagagagaaattcaccaagagacccatcctgattcagagactgcatttgaaatcaaatcatcagtttaatacaaggcggccgcaaatcaacatcagtctgataagctatcaacatcagtctgataagctatcaacatcagtctgataagctatcaacatcagtctgataagctaaagcttcgaattctgataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcagctccagattgaattatttgcctgtcatacagctaataattgaccataagacaattagatttaaattagttttgaatctttctaataccaaagttcagtttactgttccatgttgcttctgagtggcttcacagacttatgaaaaagtaaacggaatcagaattacatcaatgcaaaagcattgctgtgaactctgtacttaggactaaactttgagcaataacacatatagattgaggattgtttgctgttagtatacaaactctggttcaaagctcctctttattgcttgtcttggaaaatttgctgttcttcatggtttctcttttcactgctatctatttttctcaaccactcacatggctacaaaagcttcctgattaataattacactaagtcaataggcatagagccaggactgtttgggtaaactggtcactttatcttaaactaaatatatccaaaactgaacatgtacttagttactaagtctttgactttatctcattcataccactcagctttatccaggccacttatttgacagtattattgcgaaaacttcctatctagaagtttgaggagaatatttgttatatttgcaaaataaaataagtttgcaagttttttttttctgccccaaagagctctgtgtccttgaacataaaatacaaataaccgctatgctgttaattattgacaaatgtcccattttcaacctaaggaaataccataaagtaacagatataccaacaaaaggttactagttaacaggcattgcctgaaaagagtataaaagaatttcagcatgattttccatattgtgcttccaccactgccaataacaaaataactagcaaggatccggccaccatggcccccccgaccgatgtcagcctgggggacgagctccacttagacggcgaggacgtggcgatggcgcatgccgacgcgctagacgatttcgatctggacatgttgggggacggggattccccgggtccgggatttaccccccacgactccgccccctacggcgctctggatatggccgacttcgagtttgagcagatgtttaccgatgcccttggaattgacgagtacggtgggacgcgtatgaagctactgtcttctatcgaacaagcatgcgatatttgccgacttaaaaagctcaagtgctccaaagaaaaaccgaagtgcgccaagtgtctgaagaacaactgggagtgtcgctactctcccaaaaccaaaaggtctccgctgactagggcacatctgacagaagtggaatcaaggctagaaagactggaacagctatttctactgatttttcctcgagaagaccttgacatgattttgaaaatggattctttacaggatataaaagcattgttaacaggattatttgtacaagataatgtgaataaagatgccgtcacagatagattggcttcagtggagactgatatgcctctaacattgagacagcatagaataagtgcgacatcatcatcggaagagagtagtaacaaaggtcaaagacagttgactgtataattcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatcggagaaagaggtaat

SEQ ID NO: 11 - DNA, 6873 bp, Artificial Sequence
  Artificial Sequence created by rational design
gtaactataacggtcgctccgaatttctcgagttaattaagattacgccaagctacgggcggagtactgtcctccgagcggagtactgtcctccgagcggagtactgtcctccgagcggagtactgtcctccgagcggagttctgtcctccgagcggagactctagactccctatcagtgatagagataggcgtgtacggtgggaggcctatataagcagagctcgtttagtgaaccgtcagatcgctccctatcagtgatagagagaattcgaccgccaccatgtcggaattcatgagacatattatctgccacggaggtgttattaccgaagaaatggccgccagtcttttggaccagctgatcgaagaggtactggctgataatcttccacctcctagccattttgaaccacctacccttcacgaactgtatgatttagacgtgacggcccccgaagatcccaacgaggaggcggtttcgcagatttttcccgactctgtaatgttggcggtgcaggaagggattgacttactcacttttccgccggcgcccggttctccggagccgcctcacctttcccggcagcccgagcagccggagcagagagccttgggtccggtttctatgccaaaccttgtaccggaggtgatcgatcttacctgccacgaggctggctttccacccagtgacgacgaggatgaagagggtgaggagtttgtgttagattatgtggagcaccccgggcacggttgcaggtcttgtcattatcaccggaggaatacgggggacccagatattatgtgttcgctttgctatatgaggacctgtggcatgtttgtctacagtaagtgaaaattatgggcagtgggtgatagagtggtgggtttggtgtggtaattttttttttaatttttacagttttgtggtttaaagaattttgtattgtgatttttttaaaaggtcctgtgtctgaacctgagcctgagcccgagccagaaccggagcctgcaagacctacccgccgtcctaaaatggcgcctgctatcctgagacgcccgacatcacctgtgtctagagaatgcaatagtagtacggatagctgtgactccggtccttctaacacacctcctgagatacacccggtggtcccgctgtgccccattaaaccagttgccgtgagagttggtgggcgtcgccaggctgtggaatgtatcgaggacttgcttaacgagcctgggcaacctttggacttgagctgtaaacgccccaggccactcgagggtaccgggtccggagctacaaatttttccctcctcaaacaggctggagatgtcgaagaaaatcctgggcctaccggtcccatgtggctgcagagcctgctgctcttgggcactgtggcctgcagcatctctgcacccgcccgctcgcccagccccagcacgcagccctgggagcatgtgaatgccatccaggaggcccggcgtctcctgaacctgagtagagacactgctgctgagatgaatgaaacagtagaagtcatctcagaaatgtttgacctccaggagccgacctgcctacagacccgcctggagctgtacaagcagggcctgcggggcagcctcaccaagctcaagggccccttgaccatgatggccagccactacaagcagcactgccctccaaccccggaaacttcctgtgcaacccagattatcacctttgaaagtttcaaagagaacctgaaggactttctgcttgtcatcccctttgactgctgggagccagtccaggagtgaagatctgagggccgcggcagcctgctgacctgcggcgacgtggaggaaaaccccggccccggatccatgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagagcagcctgaggcctcctaagaagaagaggaaggttgcggccgctaaccaatgtgcagactactgttaaccaatgtgcagactactgtgaattctaaccaatgtgcagactactgttaaccaatgtgcagactactgtaagcttcgaattgagtagtgctttctactttatgagtagtgctttctactttatgagtagtgctttctactttatgagtagtgctttctactttatggtcgacaatactagttaagaaatcgccaagctacgggcggagtactgtcctccgagcggagtactgtcctccgagcggagtactgtcctccgagcggagtactgtcctccgagcggagtactgtcctccgagcggagactctagaaattgtgagcggataacaatttaggcgtgtacggtgggaggcctatataagcagagctcgtttagtgaaccgtcagatcgccagctgacaaaattgtgagcgctcacaattactagaagatcttgaattcgccaccatggctagattagataaaagtaaagtgattaacagcgcattagagctgcttaatgaggtcggaatcgaaggtttaacaacccgtaaactcgcccagaagctaggtgtagagcagcctacattgtattggcatgtaaaaaataagcgggctttgctcgacgccttagccattgagatgttagataggcaccatactcacttttgccctttagaaggggaaagctggcaagattttttacgtaataacgctaaaagttttagatgtgctttactaagtcatcgcgatggagcaaaagtacatttaggtacacggcctacagaaaaacagtatgaaactctcgaaaatcaattagcctttttatgccaacaaggtttttcactagagaatgcattatatgcactcagcgctgtggggcattttactttaggttgcgtattggaagatcaagagcatcaagtcgctaaagaagaaagggaaacacctactactgatagtatgccgccattattacgacaagctatcgaattatttgatcaccaaggtgcagagccagccttcttattcggccttgaattgatcatatgcggattagaaaaacaacttaaatgtgaaagtgggtcgccaaaaaagaagagaaaggtcgacggcggtggtgctttgtctcctcagcactctgctgtcactcaaggaagtatcatcaagaacaaggagggcatggatgctaagtcactaactgcctggtcccggacactggtgaccttcaaggatgtatttgtggacttcaccagggaggagtggaagctgctggacactgctcagcagatcgtgtacagaaatgtgatgctggagaactataagaacctggtttccttgggttatcagcttactaagccagatgtgatcctccggttggagaagggagaagagccctggctggtggagagagaaattcaccaagagacccatcctgattcagagactgcatttgaaatcaaatcatcagtttaatacaaggcggccgcaaatcaacatcagtctgataagctatcaacatcagtctgataagctatcaacatcagtctgataagctatcaacatcagtctgataagctaaagcttcgaattctgataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcagctccagattgaattatttgcctgtcatacagctaataattgaccataagacaattagatttaaattagttttgaatctttctaataccaaagttcagtttactgttccatgttgcttctgagtggcttcacagacttatgaaaaagtaaacggaatcagaattacatcaatgcaaaagcattgctgtgaactctgtacttaggactaaactttgagcaataacacatatagattgaggattgtttgctgttagtatacaaactctggttcaaagctcctctttattgcttgtcttggaaaatttgctgttcttcatggtttctcttttcactgctatctatttttctcaaccactcacatggctacaaaagcttcctgattaataattacactaagtcaataggcatagagccaggactgtttgggtaaactggtcactttatcttaaactaaatatatccaaaactgaacatgtacttagttactaagtctttgactttatctcattcataccactcagctttatccaggccacttatttgacagtattattgcgaaaacttcctatctagaagtttgaggagaatatttgttatatttgcaaaataaaataagtttgcaagttttttttttctgccccaaagagctctgtgtccttgaacataaaatacaaataaccgctatgctgttaattattgacaaatgtcccattttcaacctaaggaaataccataaagtaacagatataccaacaaaaggttactagttaacaggcattgcctgaaaagagtataaaagaatttcagcatgattttccatattgtgcttccaccactgccaataacaaaataactagcaaggatccggccaccatggcccccccgaccgatgtcagcctgggggacgagctccacttagacggcgaggacgtggcgatggcgcatgccgacgcgctagacgatttcgatctggacatgttgggggacggggattccccgggtccgggatttaccccccacgactccgccccctacggcgctctggatatggccgacttcgagtttgagcagatgtttaccgatgcccttggaattgacgagtacggtgggacgcgtatgaagctactgtcttctatcgaacaagcatgcgatatttgccgacttaaaaagctcaagtgctccaaagaaaaaccgaagtgcgccaagtgtctgaagaacaactgggagtgtcgctactctcccaaaaccaaaaggtctccgctgactagggcacatctgacagaagtggaatcaaggctagaaagactggaacagctatttctactgatttttcctcgagaagaccttgacatgattttgaaaatggattctttacaggatataaaagcattgttaacaggattatttgtacaagataatgtgaataaagatgccgtcacagatagattggcttcagtggagactgatatgcctctaacattgagacagcatagaataagtgcgacatcatcatcggaagagagtagtaacaaaggtcaaagacagttgactgtataattcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatcggagaaagaggtaat

SEQ ID NO: 12 - DNA, 6864 bp, Artificial Sequence
  Artificial Sequence created by rational design
gtaactataacggtcgctccgaatttctcgagttaattaagattacgccaagctacgggcggagtactgtcctccgagcggagtactgtcctccgagcggagtactgtcctccgagcggagtactgtcctccgagcggagttctgtcctccgagcggagactctagactccctatcagtgatagagataggcgtgtacggtgggaggcctatataagcagagctcgtttagtgaaccgtcagatcgctccctatcagtgatagagagaattcgaccgccaccatgtcggaattcatgagacatattatctgccacggaggtgttattaccgaagaaatggccgccagtcttttggaccagctgatcgaagaggtactggctgataatcttccacctcctagccattttgaaccacctacccttcacgaactgtatgatttagacgtgacggcccccgaagatcccaacgaggaggcggtttcgcagatttttcccgactctgtaatgttggcggtgcaggaagggattgacttactcacttttccgccggcgcccggttctccggagccgcctcacctttcccggcagcccgagcagccggagcagagagccttgggtccggtttctatgccaaaccttgtaccggaggtgatcgatcttacctgccacgaggctggctttccacccagtgacgacgaggatgaagagggtgaggagtttgtgttagattatgtggagcaccccgggcacggttgcaggtcttgtcattatcaccggaggaatacgggggacccagatattatgtgttcgctttgctatatgaggacctgtggcatgtttgtctacagtaagtgaaaattatgggcagtgggtgatagagtggtgggtttggtgtggtaattttttttttaatttttacagttttgtggtttaaagaattttgtattgtgatttttttaaaaggtcctgtgtctgaacctgagcctgagcccgagccagaaccggagcctgcaagacctacccgccgtcctaaaatggcgcctgctatcctgagacgcccgacatcacctgtgtctagagaatgcaatagtagtacggatagctgtgactccggtccttctaacacacctcctgagatacacccggtggtcccgctgtgccccattaaaccagttgccgtgagagttggtgggcgtcgccaggctgtggaatgtatcgaggacttgcttaacgagcctgggcaacctttggacttgagctgtaaacgccccaggccactcgagggtaccgggtccggagctacaaatttttccctcctcaaacaggctggagatgtcgaagaaaatcctgggcctaccggtcccatgtggctgcagaatttacttttcctgggcattgtggtctacagcctctcagcacccacccgctcacccatcactgtcacccggccttggaagcatgtagaggccatcaaagaagccctgaacctcctggatgacatgcctgtcacgttgaatgaagaggtagaagtcgtctctaacgagttctccttcaagaagctaacatgtgtgcagacccgcctgaagatattcgagcagggtctacggggcaatttcaccaaactcaagggcgccttgaacatgacagccagctactaccagacatactgccccccaactccggaaacggactgtgaaacacaagttaccacctatgcggatttcatagacagccttaaaacctttctgactgatatcccctttgaatgcaaaaaaccaggccaaaaatgaagatctgagggccgcggcagcctgctgacctgcggcgacgtggaggaaaaccccggccccggatccatgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagagcagcctgaggcctcctaagaagaagaggaaggttgcggccgctaaccaatgtgcagactactgttaaccaatgtgcagactactgtgaattctaaccaatgtgcagactactgttaaccaatgtgcagactactgtaagcttcgaattgagtagtgctttctactttatgagtagtgctttctactttatgagtagtgctttctactttatgagtagtgctttctactttatggtcgacaatactagttaagaaatcgccaagctacgggcggagtactgtcctccgagcggagtactgtcctccgagcggagtactgtcctccgagcggagtactgtcctccgagcggagtactgtcctccgagcggagactctagaaattgtgagcggataacaatttaggcgtgtacggtgggaggcctatataagcagagctcgtttagtgaaccgtcagatcgccagctgacaaaattgtgagcgctcacaattactagaagatcttgaattcgccaccatggctagattagataaaagtaaagtgattaacagcgcattagagctgcttaatgaggtcggaatcgaaggtttaacaacccgtaaactcgcccagaagctaggtgtagagcagcctacattgtattggcatgtaaaaaataagcgggctttgctcgacgccttagccattgagatgttagataggcaccatactcacttttgccctttagaaggggaaagctggcaagattttttacgtaataacgctaaaagttttagatgtgctttactaagtcatcgcgatggagcaaaagtacatttaggtacacggcctacagaaaaacagtatgaaactctcgaaaatcaattagcctttttatgccaacaaggtttttcactagagaatgcattatatgcactcagcgctgtggggcattttactttaggttgcgtattggaagatcaagagcatcaagtcgctaaagaagaaagggaaacacctactactgatagtatgccgccattattacgacaagctatcgaattatttgatcaccaaggtgcagagccagccttcttattcggccttgaattgatcatatgcggattagaaaaacaacttaaatgtgaaagtgggtcgccaaaaaagaagagaaaggtcgacggcggtggtgctttgtctcctcagcactctgctgtcactcaaggaagtatcatcaagaacaaggagggcatggatgctaagtcactaactgcctggtcccggacactggtgaccttcaaggatgtatttgtggacttcaccagggaggagtggaagctgctggacactgctcagcagatcgtgtacagaaatgtgatgctggagaactataagaacctggtttccttgggttatcagcttactaagccagatgtgatcctccggttggagaagggagaagagccctggctggtggagagagaaattcaccaagagacccatcctgattcagagactgcatttgaaatcaaatcatcagtttaatacaaggcggccgcaaatcaacatcagtctgataagctatcaacatcagtctgataagctatcaacatcagtctgataagctatcaacatcagtctgataagctaaagcttcgaattctgataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcagctccagattgaattatttgcctgtcatacagctaataattgaccataagacaattagatttaaattagttttgaatctttctaataccaaagttcagtttactgttccatgttgcttctgagtggcttcacagacttatgaaaaagtaaacggaatcagaattacatcaatgcaaaagcattgctgtgaactctgtacttaggactaaactttgagcaataacacatatagattgaggattgtttgctgttagtatacaaactctggttcaaagctcctctttattgcttgtcttggaaaatttgctgttcttcatggtttctcttttcactgctatctatttttctcaaccactcacatggctacaaaagcttcctgattaataattacactaagtcaataggcatagagccaggactgtttgggtaaactggtcactttatcttaaactaaatatatccaaaactgaacatgtacttagttactaagtctttgactttatctcattcataccactcagctttatccaggccacttatttgacagtattattgcgaaaacttcctatctagaagtttgaggagaatatttgttatatttgcaaaataaaataagtttgcaagttttttttttctgccccaaagagctctgtgtccttgaacataaaatacaaataaccgctatgctgttaattattgacaaatgtcccattttcaacctaaggaaataccataaagtaacagatataccaacaaaaggttactagttaacaggcattgcctgaaaagagtataaaagaatttcagcatgattttccatattgtgcttccaccactgccaataacaaaataactagcaaggatccggccaccatggcccccccgaccgatgtcagcctgggggacgagctccacttagacggcgaggacgtggcgatggcgcatgccgacgcgctagacgatttcgatctggacatgttgggggacggggattccccgggtccgggatttaccccccacgactccgccccctacggcgctctggatatggccgacttcgagtttgagcagatgtttaccgatgcccttggaattgacgagtacggtgggacgcgtatgaagctactgtcttctatcgaacaagcatgcgatatttgccgacttaaaaagctcaagtgctccaaagaaaaaccgaagtgcgccaagtgtctgaagaacaactgggagtgtcgctactctcccaaaaccaaaaggtctccgctgactagggcacatctgacagaagtggaatcaaggctagaaagactggaacagctatttctactgatttttcctcgagaagaccttgacatgattttgaaaatggattctttacaggatataaaagcattgttaacaggattatttgtacaagataatgtgaataaagatgccgtcacagatagattggcttcagtggagactgatatgcctctaacattgagacagcatagaataagtgcgacatcatcatcggaagagagtagtaacaaaggtcaaagacagttgactgtataattcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatcggagaaagaggtaat

SEQ ID NO: 13 - DNA, 7203 bp, Artificial Sequence
  Artificial Sequence created by rational design
gtaactataacggtcgctccgaatttctcgagttaattaagattacgccaagctacgggcggagtactgtcctccgagcggagtactgtcctccgagcggagtactgtcctccgagcggagtactgtcctccgagcggagttctgtcctccgagcggagactctagactccctatcagtgatagagataggcgtgtacggtgggaggcctatataagcagagctcgtttagtgaaccgtcagatcgctccctatcagtgatagagagaattcgaccgccaccatgtcggaattcatgagacatattatctgccacggaggtgttattaccgaagaaatggccgccagtcttttggaccagctgatcgaagaggtactggctgataatcttccacctcctagccattttgaaccacctacccttcacgaactgtatgatttagacgtgacggcccccgaagatcccaacgaggaggcggtttcgcagatttttcccgactctgtaatgttggcggtgcaggaagggattgacttactcacttttccgccggcgcccggttctccggagccgcctcacctttcccggcagcccgagcagccggagcagagagccttgggtccggtttctatgccaaaccttgtaccggaggtgatcgatcttacctgccacgaggctggctttccacccagtgacgacgaggatgaagagggtgaggagtttgtgttagattatgtggagcaccccgggcacggttgcaggtcttgtcattatcaccggaggaatacgggggacccagatattatgtgttcgctttgctatatgaggacctgtggcatgtttgtctacagtaagtgaaaattatgggcagtgggtgatagagtggtgggtttggtgtggtaattttttttttaatttttacagttttgtggtttaaagaattttgtattgtgatttttttaaaaggtcctgtgtctgaacctgagcctgagcccgagccagaaccggagcctgcaagacctacccgccgtcctaaaatggcgcctgctatcctgagacgcccgacatcacctgtgtctagagaatgcaatagtagtacggatagctgtgactccggtccttctaacacacctcctgagatacacccggtggtcccgctgtgccccattaaaccagttgccgtgagagttggtgggcgtcgccaggctgtggaatgtatcgaggacttgcttaacgagcctgggcaacctttggacttgagctgtaaacgccccaggccactcgagggtaccgggtccggagctacaaatttttccctcctcaaacaggctggagatgtcgaagaaaatcctgggcctaccggtcccatgagtgtgcccactcaggtcctggggttgctgctgctgtggcttacagacgctcgctgccaggtgcagctggtggagagcggcggcggcgtggtgcagcccggcaggagcctgaggctggactgcaaggccagcggcatcaccttcagcaacagcggcatgcactgggtgaggcaggcccccggcaagggcctggagtgggtggccgtgatctggtacgacggcagcaagaggtactacgccgacagcgtgaagggcaggttcaccatcagcagggacaacagcaagaacaccctgttcctgcagatgaacagcctgagggccgaggacaccgccgtgtactactgcgccaccaacgacgactactggggccagggcaccctggtgaccgtgagcagcggtggaggcggttcaggcggaggtggctctggcggtggcggatcggagatcgtgctgacccagagccccgccaccctgagcctgagccccggcgagagggccaccctgagctgcagggccagccagagcgtgagcagctacctggcctggtaccagcagaagcccggccaggcccccaggctgctgatctacgacgccagcaacagggccaccggcatccccgccaggttcagcggcagcggcagcggcaccgacttcaccctgaccatcagcagcctggagcccgaggacttcgccgtgtactactgccagcagagcagcaactggcccaggaccttcggccagggcaccaaggtggagatcaagagatctgagggccgcggcagcctgctgacctgcggcgacgtggaggaaaaccccggccccggatccatgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagagcagcctgaggcctcctaagaagaagaggaaggttgcggccgctaaccaatgtgcagactactgttaaccaatgtgcagactactgtgaattctaaccaatgtgcagactactgttaaccaatgtgcagactactgtaagcttcgaattgagtagtgctttctactttatgagtagtgctttctactttatgagtagtgctttctactttatgagtagtgctttctactttatggtcgacaatactagttaagaaatcgccaagctacgggcggagtactgtcctccgagcggagtactgtcctccgagcggagtactgtcctccgagcggagtactgtcctccgagcggagtactgtcctccgagcggagactctagaaattgtgagcggataacaatttaggcgtgtacggtgggaggcctatataagcagagctcgtttagtgaaccgtcagatcgccagctgacaaaattgtgagcgctcacaattactagaagatcttgaattcgccaccatggctagattagataaaagtaaagtgattaacagcgcattagagctgcttaatgaggtcggaatcgaaggtttaacaacccgtaaactcgcccagaagctaggtgtagagcagcctacattgtattggcatgtaaaaaataagcgggctttgctcgacgccttagccattgagatgttagataggcaccatactcacttttgccctttagaaggggaaagctggcaagattttttacgtaataacgctaaaagttttagatgtgctttactaagtcatcgcgatggagcaaaagtacatttaggtacacggcctacagaaaaacagtatgaaactctcgaaaatcaattagcctttttatgccaacaaggtttttcactagagaatgcattatatgcactcagcgctgtggggcattttactttaggttgcgtattggaagatcaagagcatcaagtcgctaaagaagaaagggaaacacctactactgatagtatgccgccattattacgacaagctatcgaattatttgatcaccaaggtgcagagccagccttcttattcggccttgaattgatcatatgcggattagaaaaacaacttaaatgtgaaagtgggtcgccaaaaaagaagagaaaggtcgacggcggtggtgctttgtctcctcagcactctgctgtcactcaaggaagtatcatcaagaacaaggagggcatggatgctaagtcactaactgcctggtcccggacactggtgaccttcaaggatgtatttgtggacttcaccagggaggagtggaagctgctggacactgctcagcagatcgtgtacagaaatgtgatgctggagaactataagaacctggtttccttgggttatcagcttactaagccagatgtgatcctccggttggagaagggagaagagccctggctggtggagagagaaattcaccaagagacccatcctgattcagagactgcatttgaaatcaaatcatcagtttaatacaaggcggccgcaaatcaacatcagtctgataagctatcaacatcagtctgataagctatcaacatcagtctgataagctatcaacatcagtctgataagctaaagcttcgaattctgataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcagctccagattgaattatttgcctgtcatacagctaataattgaccataagacaattagatttaaattagttttgaatctttctaataccaaagttcagtttactgttccatgttgcttctgagtggcttcacagacttatgaaaaagtaaacggaatcagaattacatcaatgcaaaagcattgctgtgaactctgtacttaggactaaactttgagcaataacacatatagattgaggattgtttgctgttagtatacaaactctggttcaaagctcctctttattgcttgtcttggaaaatttgctgttcttcatggtttctcttttcactgctatctatttttctcaaccactcacatggctacaaaagcttcctgattaataattacactaagtcaataggcatagagccaggactgtttgggtaaactggtcactttatcttaaactaaatatatccaaaactgaacatgtacttagttactaagtctttgactttatctcattcataccactcagctttatccaggccacttatttgacagtattattgcgaaaacttcctatctagaagtttgaggagaatatttgttatatttgcaaaataaaataagtttgcaagttttttttttctgccccaaagagctctgtgtccttgaacataaaatacaaataaccgctatgctgttaattattgacaaatgtcccattttcaacctaaggaaataccataaagtaacagatataccaacaaaaggttactagttaacaggcattgcctgaaaagagtataaaagaatttcagcatgattttccatattgtgcttccaccactgccaataacaaaataactagcaaggatccggccaccatggcccccccgaccgatgtcagcctgggggacgagctccacttagacggcgaggacgtggcgatggcgcatgccgacgcgctagacgatttcgatctggacatgttgggggacggggattccccgggtccgggatttaccccccacgactccgccccctacggcgctctggatatggccgacttcgagtttgagcagatgtttaccgatgcccttggaattgacgagtacggtgggacgcgtatgaagctactgtcttctatcgaacaagcatgcgatatttgccgacttaaaaagctcaagtgctccaaagaaaaaccgaagtgcgccaagtgtctgaagaacaactgggagtgtcgctactctcccaaaaccaaaaggtctccgctgactagggcacatctgacagaagtggaatcaaggctagaaagactggaacagctatttctactgatttttcctcgagaagaccttgacatgattttgaaaatggattctttacaggatataaaagcattgttaacaggattatttgtacaagataatgtgaataaagatgccgtcacagatagattggcttcagtggagactgatatgcctctaacattgagacagcatagaataagtgcgacatcatcatcggaagagagtagtaacaaaggtcaaagacagttgactgtataattcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatcggagaaagaggtaat

SEQ ID NO: 14 - DNA, 7218 bp, Artificial Sequence
  Artificial Sequence created by rational design
gtaactataacggtcgctccgaatttctcgagttaattaagattacgccaagctacgggcggagtactgtcctccgagcggagtactgtcctccgagcggagtactgtcctccgagcggagtactgtcctccgagcggagttctgtcctccgagcggagactctagactccctatcagtgatagagataggcgtgtacggtgggaggcctatataagcagagctcgtttagtgaaccgtcagatcgctccctatcagtgatagagagaattcgaccgccaccatgtcggaattcatgagacatattatctgccacggaggtgttattaccgaagaaatggccgccagtcttttggaccagctgatcgaagaggtactggctgataatcttccacctcctagccattttgaaccacctacccttcacgaactgtatgatttagacgtgacggcccccgaagatcccaacgaggaggcggtttcgcagatttttcccgactctgtaatgttggcggtgcaggaagggattgacttactcacttttccgccggcgcccggttctccggagccgcctcacctttcccggcagcccgagcagccggagcagagagccttgggtccggtttctatgccaaaccttgtaccggaggtgatcgatcttacctgccacgaggctggctttccacccagtgacgacgaggatgaagagggtgaggagtttgtgttagattatgtggagcaccccgggcacggttgcaggtcttgtcattatcaccggaggaatacgggggacccagatattatgtgttcgctttgctatatgaggacctgtggcatgtttgtctacagtaagtgaaaattatgggcagtgggtgatagagtggtgggtttggtgtggtaattttttttttaatttttacagttttgtggtttaaagaattttgtattgtgatttttttaaaaggtcctgtgtctgaacctgagcctgagcccgagccagaaccggagcctgcaagacctacccgccgtcctaaaatggcgcctgctatcctgagacgcccgacatcacctgtgtctagagaatgcaatagtagtacggatagctgtgactccggtccttctaacacacctcctgagatacacccggtggtcccgctgtgccccattaaaccagttgccgtgagagttggtgggcgtcgccaggctgtggaatgtatcgaggacttgcttaacgagcctgggcaacctttggacttgagctgtaaacgccccaggccactcgagggtaccgggtccggagctacaaatttttccctcctcaaacaggctggagatgtcgaagaaaatcctgggcctaccggtcccatgagtgtgcccactcaggtcctggggttgctgctgctgtggcttacagacgctcgctgcgaggtgcagctggtggagagcggcggcggcctggtgcagcccggcggcagcctgaggctgagctgcgccgccagcggcttcaccttcagcgacagctggatccactgggtgaggcaggcccccggcaagggcctggagtgggtggcctggatcagcccctacggcggcagcacctactacgccgacagcgtgaagggcaggttcaccatcagcgccgacaccagcaagaacaccgcctacctgcagatgaacagcctgagggccgaggacaccgccgtgtactactgcgccaggaggcactggcccggcggcttcgactactggggccagggcaccctggtgaccgtgagcagcggtggaggcggttcaggcggaggtggctctggcggtggcggatcggacatccagatgacccagagccccagcagcctgagcgccagcgtgggcgacagggtgaccatcacctgcagggccagccaggacgtgagcaccgccgtggcctggtaccagcagaagcccggcaaggcccccaagctgctgatctacagcgccagcttcctgtacagcggcgtgcccagcaggttcagcggcagcggcagcggcaccgacttcaccctgaccatcagcagcctgcagcccgaggacttcgccacctactactgccagcagtacctgtaccaccccgccaccttcggccagggcaccaaggtggagatcaagagatctgagggccgcggcagcctgctgacctgcggcgacgtggaggaaaaccccggccccggatccatgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagagcagcctgaggcctcctaagaagaagaggaaggttgcggccgctaaccaatgtgcagactactgttaaccaatgtgcagactactgtgaattctaaccaatgtgcagactactgttaaccaatgtgcagactactgtaagcttcgaattgagtagtgctttctactttatgagtagtgctttctactttatgagtagtgctttctactttatgagtagtgctttctactttatggtcgacaatactagttaagaaatcgccaagctacgggcggagtactgtcctccgagcggagtactgtcctccgagcggagtactgtcctccgagcggagtactgtcctccgagcggagtactgtcctccgagcggagactctagaaattgtgagcggataacaatttaggcgtgtacggtgggaggcctatataagcagagctcgtttagtgaaccgtcagatcgccagctgacaaaattgtgagcgctcacaattactagaagatcttgaattcgccaccatggctagattagataaaagtaaagtgattaacagcgcattagagctgcttaatgaggtcggaatcgaaggtttaacaacccgtaaactcgcccagaagctaggtgtagagcagcctacattgtattggcatgtaaaaaataagcgggctttgctcgacgccttagccattgagatgttagataggcaccatactcacttttgccctttagaaggggaaagctggcaagattttttacgtaataacgctaaaagttttagatgtgctttactaagtcatcgcgatggagcaaaagtacatttaggtacacggcctacagaaaaacagtatgaaactctcgaaaatcaattagcctttttatgccaacaaggtttttcactagagaatgcattatatgcactcagcgctgtggggcattttactttaggttgcgtattggaagatcaagagcatcaagtcgctaaagaagaaagggaaacacctactactgatagtatgccgccattattacgacaagctatcgaattatttgatcaccaaggtgcagagccagccttcttattcggccttgaattgatcatatgcggattagaaaaacaacttaaatgtgaaagtgggtcgccaaaaaagaagagaaaggtcgacggcggtggtgctttgtctcctcagcactctgctgtcactcaaggaagtatcatcaagaacaaggagggcatggatgctaagtcactaactgcctggtcccggacactggtgaccttcaaggatgtatttgtggacttcaccagggaggagtggaagctgctggacactgctcagcagatcgtgtacagaaatgtgatgctggagaactataagaacctggtttccttgggttatcagcttactaagccagatgtgatcctccggttggagaagggagaagagccctggctggtggagagagaaattcaccaagagacccatcctgattcagagactgcatttgaaatcaaatcatcagtttaatacaaggcggccgcaaatcaacatcagtctgataagctatcaacatcagtctgataagctatcaacatcagtctgataagctatcaacatcagtctgataagctaaagcttcgaattctgataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcagctccagattgaattatttgcctgtcatacagctaataattgaccataagacaattagatttaaattagttttgaatctttctaataccaaagttcagtttactgttccatgttgcttctgagtggcttcacagacttatgaaaaagtaaacggaatcagaattacatcaatgcaaaagcattgctgtgaactctgtacttaggactaaactttgagcaataacacatatagattgaggattgtttgctgttagtatacaaactctggttcaaagctcctctttattgcttgtcttggaaaatttgctgttcttcatggtttctcttttcactgctatctatttttctcaaccactcacatggctacaaaagcttcctgattaataattacactaagtcaataggcatagagccaggactgtttgggtaaactggtcactttatcttaaactaaatatatccaaaactgaacatgtacttagttactaagtctttgactttatctcattcataccactcagctttatccaggccacttatttgacagtattattgcgaaaacttcctatctagaagtttgaggagaatatttgttatatttgcaaaataaaataagtttgcaagttttttttttctgccccaaagagctctgtgtccttgaacataaaatacaaataaccgctatgctgttaattattgacaaatgtcccattttcaacctaaggaaataccataaagtaacagatataccaacaaaaggttactagttaacaggcattgcctgaaaagagtataaaagaatttcagcatgattttccatattgtgcttccaccactgccaataacaaaataactagcaaggatccggccaccatggcccccccgaccgatgtcagcctgggggacgagctccacttagacggcgaggacgtggcgatggcgcatgccgacgcgctagacgatttcgatctggacatgttgggggacggggattccccgggtccgggatttaccccccacgactccgccccctacggcgctctggatatggccgacttcgagtttgagcagatgtttaccgatgcccttggaattgacgagtacggtgggacgcgtatgaagctactgtcttctatcgaacaagcatgcgatatttgccgacttaaaaagctcaagtgctccaaagaaaaaccgaagtgcgccaagtgtctgaagaacaactgggagtgtcgctactctcccaaaaccaaaaggtctccgctgactagggcacatctgacagaagtggaatcaaggctagaaagactggaacagctatttctactgatttttcctcgagaagaccttgacatgattttgaaaatggattctttacaggatataaaagcattgttaacaggattatttgtacaagataatgtgaataaagatgccgtcacagatagattggcttcagtggagactgatatgcctctaacattgagacagcatagaataagtgcgacatcatcatcggaagagagtagtaacaaaggtcaaagacagttgactgtataattcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatcggagaaagaggtaat

SEQ ID NO: 15 - DNA, 24 bp, Artificial Sequence
  Artificial Sequence created by rational design
caaagctgaaaatgcagttgaatg

SEQ ID NO: 16 - DNA, 22 bp, Artificial Sequence
  Artificial Sequence created by rational design
ttccccatcctgcagacaatcc

SEQ ID NO: 17 - DNA, 20 bp, Artificial Sequence
  Artificial Sequence created by rational design
agaaggctggggctcatttg

SEQ ID NO: 18 - DNA, 20 bp, Artificial Sequence
  Artificial Sequence created by rational design
aggggccatccacagtcttc

SEQ ID NO: 19 - DNA, 22 bp, Artificial Sequence
  Artificial Sequence created by rational design
ttgtggttcttgcagatatggc

SEQ ID NO: 20 - DNA, 16 bp, Artificial Sequence
  Artificial Sequence created by rational design
tcggaatcccggcacc

SEQ ID NO: 21 - DNA, 2203 bp, Artificial Sequence
  Artificial Sequence created by rational design
tttagaaatatgggggtaggggtggtggtggggcctggataaagctgagtggtatgaatgagttagaaatatgggggtaggggtggtggtggtaattctgttttctccccataggtgagataagcattgggttaaatgtgctttctctctctccctctcctttcttaagaattaagggacagactatgggctggaggactttgaggatgtctgtctcataacacttgggttgtatctgttctatggggcttgttttaagcttggcaacttgcaacagggttcactgactttctccccaggcccaaggtactgtcctcttttcatatctgttttggggcctctggggcttgaatatctgagaaaatataaacatttcaataatgttctgtggtgagatgagtatgagagatgtgtcattcatttgtatcaatgaatgaatgaggacaattagtgtataaatccttagtacaacaatctgagggtaggggtggtactattcaatttctatttataaagatacttatttctatttatttatgcttgtgacaaatgttttgttcgggaccacaggaatcacaaagatgagtctttgaatttaagaagttaatggtccaggaataattacatagcttacaaatgactatgatataccatcaaacaagaggttccatgagaaaataatctgaaaggtttaataagttgtcaaaggtgagagggctcttctctagctagagactaatcagaaatacattcagggataattatttgaatagaccttaagggttgggtacattttgttcaagcattgatggagaaggagagtgaatatttgaaaacatttcaactaaccaaccacccaatccaacaaacaaaaaatgaaaagaatctcagaaacagtgagataagagaaggaattttctcacaacccacacgtatagctcaactgctctgaagaagtatatatctaatatttaacactaacatcatgctaataatgataataattactgtcattttttaatgtctataagtaccaggcatttagaagatattattccatttatatatcaaaataaacttgaggggatagatcattttcatgatatatgagaaaaattaaaaatcagattgaattatttgcctgtcatacagctaataattgaccataagacaattagatttaaattagttttgaatctttctaataccaaagttcagtttactgttccatgttgcttctgagtggcttcacagacttatgaaaaagtaaacggaatcagaattacatcaatgcaaaagcattgctgtgaactctgtacttaggactaaactttgagcaataacacatatagattgaggattgtttgctgttagtatacaaactctggttcaaagctcctctttattgcttgtcttggaaaatttgctgttcttcatggtttctcttttcactgctatctatttttctcaaccactcacatggctacaataactgtctgcaagcttatgattcccaaatatctatctctagcctcaatcttgttccagaagataaaaagtagtattcaaatgcacatcaacgtctccacttggagggcttaaagacgtttcaacatacaaaccggggagttttgcctggaatgtttcctaaaatgtgtcctgtagcacatagggtcctcttgttccttaaaatctaattacttttagcccagtgctcatcccacctatggggagatgagagtgaaaagggagcctgattaataattacactaagtcaataggcatagagccaggactgtttgggtaaactggtcactttatcttaaactaaatatatccaaaactgaacatgtacttagttactaagtctttgactttatctcattcataccactcagctttatccaggcctctagaagtttgaggagaatatttgttatatttgcaaaataaaataagtttgcaagttttttttttctgccccaaagagctctgtgtccttgaacataaaatacaaataaccgctatgctgttaattattgacaaatgtcccattttcaacctaaggaaataccataaagtaacagatataccaacaaaaggttactagttaacaggcattgcctgaaaagagtataaaagaatttcagcatgattttccatattgtgcttccaccactgccaataacaaaataactagcaa

SEQ ID NO: 22 - DNA, 1113 bp, Artificial Sequence
  Artificial Sequence created by rational design
cagattgaattatttgcctgtcatacagctaataattgaccataagacaattagatttaaattagttttgaatctttctaataccaaagttcagtttactgttccatgttgcttctgagtggcttcacagacttatgaaaaagtaaacggaatcagaattacatcaatgcaaaagcattgctgtgaactctgtacttaggactaaactttgagcaataacacatatagattgaggattgtttgctgttagtatacaaactctggttcaaagctcctctttattgcttgtcttggaaaatttgctgttcttcatggtttctcttttcactgctatctatttttctcaaccactcacatggctacaataactgtctgcaagcttatgattcccaaatatctatctctagcctcaatcttgttccagaagataaaaagtagtattcaaatgcacatcaacgtctccacttggagggcttaaagacgtttcaacatacaaaccggggagttttgcctggaatgtttcctaaaatgtgtcctgtagcacatagggtcctcttgttccttaaaatctaattacttttagcccagtgctcatcccacctatggggagatgagagtgaaaagggagcctgattaataattacactaagtcaataggcatagagccaggactgtttgggtaaactggtcactttatcttaaactaaatatatccaaaactgaacatgtacttagttactaagtctttgactttatctcattcataccactcagctttatccaggccacttatttgacagtattattgcgaaaacttcctatctagaagtttgaggagaatatttgttatatttgcaaaataaaataagtttgcaagttttttttttctgccccaaagagctctgtgtccttgaacataaaatacaaataaccgctatgctgttaattattgacaaatgtcccattttcaacctaaggaaataccataaagtaacagatataccaacaaaaggttactagttaacaggcattgcctgaaaagagtataaaagaatttcagcatgattttccatattgtgcttccaccactgccaataacaaaataactagcaa

SEQ ID NO: 23 - DNA, 856 bp, Artificial Sequence
  Artificial Sequence created by rational design
cagattgaattatttgcctgtcatacagctaataattgaccataagacaattagatttaaattagttttgaatctttctaataccaaagttcagtttactgttccatgttgcttctgagtggcttcacagacttatgaaaaagtaaacggaatcagaattacatcaatgcaaaagcattgctgtgaactctgtacttaggactaaactttgagcaataacacatatagattgaggattgtttgctgttagtatacaaactctggttcaaagctcctctttattgcttgtcttggaaaatttgctgttcttcatggtttctcttttcactgctatctatttttctcaaccactcacatggctacaaaagcttcctgattaataattacactaagtcaataggcatagagccaggactgtttgggtaaactggtcactttatcttaaactaaatatatccaaaactgaacatgtacttagttactaagtctttgactttatctcattcataccactcagctttatccaggccacttatttgacagtattattgcgaaaacttcctatctagaagtttgaggagaatatttgttatatttgcaaaataaaataagtttgcaagttttttttttctgccccaaagagctctgtgtccttgaacataaaatacaaataaccgctatgctgttaattattgacaaatgtcccattttcaacctaaggaaataccataaagtaacagatataccaacaaaaggttactagttaacaggcattgcctgaaaagagtataaaagaatttcagcatgattttccatattgtgcttccaccactgccaataacaaaataactagcaa

SEQ ID NO: 24 - DNA, 663 bp, Artificial Sequence
  Artificial Sequence created by rational design
cagattgaattatttgcctgtcatacagctaataattgaccataagacaattagatttaaattagttttgaatctttctaataccaaagttcagtttactgttccatgttgcttctgagtggcttcacagacttatgaaaaagtaaacggaatcagaattacatcaatgcaaaagcattgctgtgaactctgtacttaggactaaactttgagcaataacacatatagattgaggattgtttgctgttagtatacaaactctggttcaaagctcctctttattgcttgtcttggaaaatttgctgttcttcatggtttctcttttcactgctatctatttttctcaaccactcacatggctacaaaagctttctagaagtttgaggagaatatttgttatatttgcaaaataaaataagtttgcaagttttttttttctgccccaaagagctctgtgtccttgaacataaaatacaaataaccgctatgctgttaattattgacaaatgtcccattttcaacctaaggaaataccataaagtaacagatataccaacaaaaggttactagttaacaggcattgcctgaaaagagtataaaagaatttcagcatgattttccatattgtgcttccaccactgccaataacaaaataactagcaa

SEQ ID NO: 25 - DNA, 485 bp, Artificial Sequence
  Artificial Sequence created by rational design
cctgattaataattacactaagtcaataggcatagagccaggactgtttgggtaaactggtcactttatcttaaactaaatatatccaaaactgaacatgtacttagttactaagtctttgactttatctcattcataccactcagctttatccaggccacttatttgacagtattattgcgaaaacttcctatctagaagtttgaggagaatatttgttatatttgcaaaataaaataagtttgcaagttttttttttctgccccaaagagctctgtgtccttgaacataaaatacaaataaccgctatgctgttaattattgacaaatgtcccattttcaacctaaggaaataccataaagtaacagatataccaacaaaaggttactagttaacaggcattgcctgaaaagagtataaaagaatttcagcatgattttccatattgtgcttccaccactgccaataacaaaataactagcaa